SEQUENCE LISTING (78 sequences)

SEQ ID NO: 1 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 2 - protein, 325 aa, Homo sapiens
GRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVV

SEQ ID NO: 3 - protein, 113 aa, Homo sapiens
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLR

SEQ ID NO: 4 - protein, 90 aa, Homo sapiens
PPLPSLNPGPALEEGQGLTLAASCTAEGSPAPSVTWDTEVKGTTSSRSFKHSRSAAVTSEFHLVPSRSMNGQPLTCVVSHPGLLQDQRIT

SEQ ID NO: 5 - protein, 103 aa, Homo sapiens
ASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASV

SEQ ID NO: 6 - protein, 116 aa, synthetic construct
  REGION 1..116: note = artificial
QVQLKQSGPGLVQPSQSLSITCTVSGFSLTNYGVHWVRQSPGKGLEWLGVIWSGGSTDYNAAFISRLSISKDTSKSQVFFKMNSLQADDTAIYYCARELIHAMDNWGQGTSVTVSS

SEQ ID NO: 7 - protein, 106 aa, synthetic construct
  REGION 1..106: note = Artificial
DIQMTQSPASLSVSVGETVTITCRASENIYSNLAWYQQKQGNSPQLLVFAATNLADGVPSRFSGSGSGTQYSLKINSLQSEDFGTYYCQHFWGTPTFGGGTKLEIK

SEQ ID NO: 8 - protein, 339 aa, synthetic construct
  REGION 1..339: note = Artificial
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASHHHHHHLHHILDAQKMVWNHR

SEQ ID NO: 9 - protein, 479 aa, synthetic construct
  REGION 1..479: note = Artificial
GELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 10 - protein, 363 aa, synthetic construct
  REGION 1..363: note = Artificial
PPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 11 - protein, 263 aa, synthetic construct
  REGION 1..263: note = Artifical
ASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 12 - protein, 477 aa, Mus musculus
ELETSDVVTVVLGQDAKLPCFYRGDPDEQVGQVAWARVDPNEGIRELALLHSKYGLHVNPAYEDRVEQPPPPRDPLDGSVLLRNAVQADEGEYECRVSTFPAGSFQARMRLRVLVPPLPSLNPGPPLEEGQGLTLAASCTAEGSPAPSVTWDTEVKGTQSSRSFTHPRSAAVTSEFHLVPSRSMNGQPLTCVVSHPGLLQDRRITHTLQVAFLAEASVRGLEDQNLWQVGREGATLKCLSEGQPPPKYNWTRLDGPLPSGVRVKGDTLGFPPLTTEHSGVYVCHVSNELSSRDSQVTVEVLDPEDPGKQVDLVSASVIIVGVIAALLFCLLVVVVVLMSRYHRRKAQQMTQKYEEELTLTRENSIRRLHSHHSDPRSQPEESVGLRAEGHPDSLKDNSSCSVMSEEPEGRSYSTLTTVREIETQTELLSPGSGRTEEDDDQDEGIKQAMNHFVQENGTLRAKPTGNGIYINGRGHLV

SEQ ID NO: 13 - protein, 508 aa, Rattus rattus
MPLSLGAEMWGPEAWLLLLFLASFTGRYSAGELETSDLVTVVLGQDAKLPCFYRGDPDEQVGQVAWARVDPNEGTRELALLHSKYGLHVSPAYEDRVEQPPPPRDPLDGSILLRNAVQADEGEYECRVSTFPAGSFQARMRLRVLVPPLPSLNPGPPLEEGQGLTLAASCTAEGSPAPSVTWDTEVKGTQSSRSFKHSRSAAVTSEFHLVPSRSMNGQPLTCVVSHPGLLQDQRITHTLQVAFLAEASVRGLEDQNLWHVGREGATLKCLSEGQPPPKYNWTRLDGPLPSGVRVKGDTLGFPPLTTEHSGVYVCHVSNELSSRASQVTVEVLDPEDPGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRTEEEDDQDEGIKQAMNHFVQENGTLRAKPTGNGIYINGRGHLV

SEQ ID NO: 14 - protein, 479 aa, Cynomolgus macaque
GELETSDVVTVVLGQDAKLPCFYRGDSGEQVGQVAWARADAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHVGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRTEEEEDQDEGIKQAMNHFVQENGTLRAKPTGNGIYINGRGHLV

SEQ ID NO: 15 - protein, 514 aa, Homo sapiens
MGLAGAAGRWWGLALGLTAFFLPGVHSQVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGTYICEATNPIGTRSGQVEVNITEFPYTPSPPEHGRRAGPVPTAIIGGVAGSILLVLIVVGGIVVALRRRRHTFKGDYSTKKHVYGNGYSKAGIPQHHPPMAQNLQYPDDSDDEKKAGPLGGSSYEEEEEEEEGGGGGERKVGGPHPKYDEDAKRPYFTVDEAEARQDGYGDRTLGYQYDPEQLDLAENMVSQNDGSFISKKEWYV

SEQ ID NO: 16 - protein, 538 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 17 - protein, 549 aa, Homo sapiens
MARTLRPSPLCPGGGKAQLSSASLLGAGLLLQPPTPPPLLLLLFPLLLFSRLCGALAGPIIVEPHVTAVWGKNVSLKCLIEVNETITQISWEKIHGKSSQTVAVHHPQYGFSVQGEYQGRVLFKNYSLNDATITLHNIGFSDSGKYICKAVTFPLGNAQSSTTVTVLVEPTVSLIKGPDSLIDGGNETVAAICIAATGKPVAHIDWEGDLGEMESTTTSFPNETATIISQYKLFPTRFARGRRITCVVKHPALEKDIRYSFILDIQYAPEVSVTGYDGNWFVGRKGVNLKCNADANPPPFKSVWSRLDGQWPDGLLASDNTLHFVHPLTFNYSGVYICKVTNSLGQRSDQKVIYISDPPTTTTLQPTIQWHPSTADIEDLATEPKKLPFPLSTLATIKDDTIATIIASVVGGALFIVLVSVLAGIFCYRRRRTFRGDYFAKNYIPPSDMQKESQIDVLQQDELDSYPDSVKKENKNPVNNLIRKDYLEEPEKTQWNNVENLNRFERPMDYYEDLKMGMKFVSDEHYDENEDDLVSHVDGSVISRREWYV

SEQ ID NO: 18 - protein, 390 aa, Homo sapiens
DVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 19 - protein, 123 aa, Mus musculus
QVQLQQPGAELVKPGASVKLSCKASGYIFTSYWMHWVKQRPGQGLEWIGEIDPSDSYTNYNQKFKGKATLTLDKSSSTTYMQLSSLTSEDSAVYYCVRGYGNYGDYWGQGTTLTVSSASTKGP

SEQ ID NO: 20 - protein, 118 aa, Mus musculus
DVVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIKRTVAAP

SEQ ID NO: 21 - protein, 5 aa, Mus musculus
SYWMH

SEQ ID NO: 22 - protein, 17 aa, Mus musculus
EIDPSDSYTNYNQKFKG

SEQ ID NO: 23 - protein, 8 aa, Mus musculus
GYGNYGDY

SEQ ID NO: 24 - protein, 16 aa, Mus musculus
RSSKSLLHSNGITYLY

SEQ ID NO: 25 - protein, 7 aa, Mus musculus
QMSNLAS

SEQ ID NO: 26 - protein, 9 aa, Mus musculus
AQNLELPWT

SEQ ID NO: 27 - protein, 8 aa, Mus musculus
GYIFTSYW

SEQ ID NO: 28 - protein, 8 aa, Mus musculus
IDPSDSYT

SEQ ID NO: 29 - protein, 10 aa, Mus musculus
VRGYGNYGDY

SEQ ID NO: 30 - protein, 11 aa, Mus musculus
KSLLHSNGITY

SEQ ID NO: 31 - skipped

SEQ ID NO: 32 - protein, 7 aa, Mus musculus
GYIFTSY

SEQ ID NO: 33 - protein, 4 aa, Mus musculus
PSDS

SEQ ID NO: 34 - protein, 6 aa, Mus musculus
YGNYGD

SEQ ID NO: 35 - protein, 12 aa, Mus musculus
SKSLLHSNGITY

SEQ ID NO: 36 - protein, 6 aa, Mus musculus
NLELPW

SEQ ID NO: 37 - protein, 117 aa, synthetic construct
  REGION 1..117: note = Artificial
QVQLVQSGAEVKKPGASVKVSCKASGYTFTSYWMHWVRQAPGQGLEWMGEIDPSDSYTNYNQKFKGRVTMTRDTSTSTVYMELSSLRSEDTAVYYCARGYGNYGDYWGQGTLVTVSS

SEQ ID NO: 38 - DNA, 351 bp, synthetic construct
  misc_feature 1..351: note = Artificial
  source 1..351: mol_type = other DNA
caggttcagctggttcagtcaggtgctgaggtgaagaagcctggagctagcgtgaaagtgtcctgcaaagcctctggctacacctttacctcctattggatgcactgggtacgacaggcaccaggacaagggctggaatggatgggcgaaatcgatccctctgacagctacacgaactacaaccagaagttcaagggtagggtcactatgactcgcgacacatccaccagtaccgtctacatggagctctccagtttgcggtctgaggatacagccgtgtactactgtgccagaggctatggcaattatggggactattggggacaagggacactggtcactgtgagctca

SEQ ID NO: 39 - protein, 117 aa, synthetic construct
  REGION 1..117: note = Artificial
QVQLVQSGAEVKKPGASVKVSCKASGYTFTSYWMHWVRQAPGQGLEWMGEIDPSDSYTNYNQKFKGRVTMTLDTSTSTTYMELSSLRSEDTAVYYCARGYGNYGDYWGQGTLVTVSS

SEQ ID NO: 40 - DNA, 351 bp, synthetic construct
  misc_feature 1..351: note = Artificial
  source 1..351: mol_type = other DNA
caggttcagctggtccagagtggtgcagaagtgaagaaacctggcgcttcagtgaaggtatcctgcaaagcctctgggtacacctttacctcctattggatgcactgggttcgccaagctccaggacagggcttggaatggatgggagagatcgatccctctgactcctacaccaactacaaccagaagttcaagggaagggtgacgatgactctcgacactagcaccagcacaacctacatggagctgagttcacttcggtctgaggatactgccgtctactactgtgccagaggctatgggaattatggggactattggggtcaaggcacactggtgacagtgtccagc

SEQ ID NO: 41 - protein, 117 aa, synthetic construct
  REGION 1..117: note = Artificial
QVQLVQSGAEVKKPGASVKVSCKASGYTFTSYWMHWVRQAPGQGLEWMGEIDPSDSYTNYNQKFKGRVTMTLDKSTSTTYMELSSLRSEDTAVYYCARGYGNYGDYWGQGTLVTVSS

SEQ ID NO: 42 - DNA, 351 bp, synthetic construct
  misc_feature 1..351: note = Artificial
  source 1..351: mol_type = other DNA
caggtgcagctggttcagtcaggagcagaggtcaagaaacctggtgcttctgtgaaggtgtcctgcaaagccagtggctatacctttaccagctactggatgcactgggttcggcaagctccaggtcagggcttggaatggatgggagagatcgatccctcagactcctacacgaactacaaccagaagttcaaagggagggtaaccatgacactggacaagtctacatccaccacttacatggagctgagttctcttcgctccgaagatactgccgtgtactactgtgccagaggctatgggaattatggcgactattggggacaagggactctcgtgacagtcagcagc

SEQ ID NO: 43 - protein, 117 aa, synthetic construct
  REGION 1..117: note = Artificial
QVQLVQSGAEVKKPGASVKVSCKASGYIFTSYWMHWVRQAPGQGLEWMGEIDPSDSYTNYNQKFKGRVTMTLDKSTSTTYMELSSLRSEDTAVYYCARGYGNYGDYWGQGTLVTVSS

SEQ ID NO: 44 - protein, 351 aa, synthetic construct
  REGION 1..351: note = Artificial
CAGGTTCAGCTGGTCCAGTCAGGTGCAGAAGTGAAGAAACCTGGAGCTAGCGTGAAGGTGTCCTGCAAAGCCTCTGGCTACATCTTTACCTCCTATTGGATGCACTGGGTACGCCAAGCTCCAGGTCAGGGGTTGGAATGGATGGGAGAGATTGATCCCTCTGACAGCTACACCAACTACAACCAGAAGTTCAAAGGACGGGTTACCATGACACTGGACAAGTCCACTTCAACGACCTACATGGAGCTTTCCTCTCTGAGGAGTGAGGATACTGCCGTGTACTACTGTGCCAGAGGGTATGGCAATTATGGGGACTATTGGGGCCAAGGCACACTCGTCACAGTGAGCAGT

SEQ ID NO: 45 - protein, 117 aa, synthetic construct
  REGION 1..117: note = Artificial
QVQLVQSGAEVKKPGASVKVSCKASGYIFTSYWMHWVKQAPGQGLEWMGEIDPSDSYTNYNQKFKGRVTMTLDKSTSTTYMELSSLRSEDTAVYYCARGYGNYGDYWGQGTLVTVSS

SEQ ID NO: 46 - DNA, 351 bp, synthetic construct
  misc_feature 1..351: note = Artificial
  source 1..351: mol_type = other DNA
caggttcagctcgttcagtctggagccgaagtgaagaaaccaggtgctagcgtgaaagtgagttgcaaggcttccggctacatctttaccagctattggatgcactgggtcaaacaggcacctggacaagggttggagtggatgggagagattgatccctcagactcctacaccaactacaaccagaagttcaagggcagggtaaccatgacactggacaagtccacgagtactacctacatggaactgtcctctcttcggtctgaggatacagccgtgtactactgtgccagagggtatggcaattatggggactattggggtcaaggcactctggtcacagtgagctca

SEQ ID NO: 47 - protein, 117 aa, synthetic construct
  REGION 1..117: note = Artificial
QVQLVQSGAEVKKPGASVKVSCKASGYIFTSYWMHWVKQRPGQGLEWMGEIDPSDSYTNYNQKFKGRVTMTLDKSTSTTYMELSSLRSEDTAVYYCARGYGNYGDYWGQGTLVTVSS

SEQ ID NO: 48 - DNA, 351 bp, synthetic construct
  misc_feature 1..351: note = Artificial
  source 1..351: mol_type = other DNA
caggttcagctcgttcagtcaggtgcagaagtcaagaaaccaggagctagcgtgaaggtttcctgcaaggcttctgggtacatctttaccagctactggatgcactgggtgaaacagcgccctggtcaaggactggaatggatgggcgagattgatcccagtgacagctacaccaactacaaccagaagttcaaaggcagggtgaccatgacactggacaagtccacttcaaccacgtacatggagctcagttcccttcggtctgaggatacagccgtctactattgtgccagagggtatggcaattatggggactattggggacaaggcacactggtcacagtgagctca

SEQ ID NO: 49 - protein, 117 aa, synthetic construct
  REGION 1..117: note = Artificial
QVQLVQSGAEVKKPGASVKVSCKASGYIFTSYWMHWVKQRPGQGLEWIGEIDPSDSYTNYNQKFKGRVTMTLDKSTSTTYMELSSLRSEDTAVYYCARGYGNYGDYWGQGTLVTVSS

SEQ ID NO: 50 - DNA, 351 bp, synthetic construct
  misc_feature 1..351: note = Artificial
  source 1..351: mol_type = other DNA
caggttcagctcgttcagagtggagcagaggtgaagaaaccaggagcttcagtcaaggtatcctgcaaagcctctggctacatctttaccagctactggatgcactgggtgaaacaacgccctggacaagggttggagtggattggggaaatcgatccctccgattcctacaccaactacaaccagaagttcaagggcagagtgacgatgacactggacaagtctacatccactacctacatggaactcagctcacttcggagtgaggacacagccgtctattactgtgctaggggctatgggaattatggcgactattggggtcagggtactctggtcacagtgagctca

SEQ ID NO: 51 - protein, 117 aa, synthetic construct
  REGION 1..117: note = Artificial
QVQLVQSGAEVKKPGASVKVSCKASGYIFTSYWMHWVKQRPGQGLEWIGEIDPSDSYTNYNQKFKGRVTLTLDKSTSTTYMELSSLRSEDTAVYYCARGYGNYGDYWGQGTLVTVSS

SEQ ID NO: 52 - DNA, 351 bp, synthetic construct
  misc_feature 1..351: note = Artificial
  source 1..351: mol_type = other DNA
caggttcagctcgttcagtcaggcgctgaagtcaagaaacccggagcatccgtgaaggtgtcctgtaaggcctctgggtacatctttacgagctactggatgcactgggtgaaacagaggcctggtcaagggcttgagtggattggagagatcgatccaagcgactcatacaccaactacaaccagaagttcaaaggtcgcgttacactgactctggacaagagtacctccaccacctacatggaactgagttctttgcggtctgaggatacagccgtgtattactgcgctagagggtatggcaattatggcgactattggggacaaggcacactggtcacagtgagctca

SEQ ID NO: 53 - protein, 117 aa, synthetic construct
  REGION 1..117: note = Artificial
QVQLVQSGAEVKKPGASVKVSCKASGYIFTSYWMHWVKQAPGQGLEWIGEIDPSDSYTNYNQKFKGRVTMTLDKSTSTTYMELSSLRSEDTAVYYCARGYGNYGDYWGQGTLVTVSS

SEQ ID NO: 54 - DNA, 381 bp, synthetic construct
  misc_feature 1..381: note = Artificial
  source 1..381: mol_type = other DNA
acaggcgtgcattcgcaggttcagctggtacagtcaggtgccgaagtgaagaaacctggtgcatccgtcaaagtgagctgcaaagccagtgggtacatctttacctcctattggatgcactgggttaagcaggctccaggacaagggttggagtggattggcgaaatcgatccctcagactcctacacgaactacaaccagaagttcaagggcagagtgacaatgactctggacaagtctacctctaccacctatatggagctgtctagccttcggagtgaggatacagccgtgtactactgtgctaggggatacgggaattatggcgactattggggacaaggcacactcgtcactgtgtccagcgcgagcaccaagggc

SEQ ID NO: 55 - protein, 117 aa, synthetic construct
  REGION 1..117: note = Artificial
QVQLVQSGAEVKKPGASVKVSCKASGYIFTSYWMHWVKQAPGQGLEWIGEIDPSDSYTNYNQKFKGRVTMTLDKSTSTVYMELSSLRSEDTAVYYCARGYGNYGDYWGQGTLVTVSS

SEQ ID NO: 56 - DNA, 381 bp, synthetic construct
  misc_feature 1..381: note = Artificial
  source 1..381: mol_type = other DNA
acaggcgtgcattcgcaggttcagctggttcagtctggcgctgaagtcaagaaacctggagccagtgtcaaggtgtcatgcaaagcttccgggtacatctttacgagctattggatgcactgggtgaaacaggcaccaggacaagggttggagtggattggtgagatcgatccctcagacagctacaccaactacaaccagaagttcaagggtagggtaaccatgacactggacaagtccacaagcaccgtgtacatggaactctcctctcttcggagtgaggatactgccgtgtactactgtgccagaggctatggcaattatggggactattggggacaaggcactctggtcacagtgtcctctgcgagcaccaagggc

SEQ ID NO: 57 - protein, 117 aa, synthetic construct
  REGION 1..117: note = Artificial
QVQLVQSGAEVKKPGASVKVSCKASGYIFTSYWMHWVKQAPGQGLEWVGEIDPSDSYTNYNQKFKGRVTMTLDKSTSTTYMELSSLRSEDTAVYYCARGYGNYGDYWGQGTLVTVSS

SEQ ID NO: 58 - DNA, 381 bp, synthetic construct
  misc_feature 1..381: note = Artificial
  source 1..381: mol_type = other DNA
acaggcgtgcattcgcaggtacagctggttcagtcaggtgctgaggtgaagaagccaggtgcatccgtgaaggtcagctgcaaagcctctgggtacatctttaccagctattggatgcactgggtgaaacaggctcctggacaaggactggaatgggttggggagattgaccccagtgactcctataccaactacaaccagaagttcaaagggagggtcacgatgactctcgacaagtcaacctccacaacctacatggaactgtcctctttgcggagtgaggatacagccgtgtactactgtgccagaggctatggcaattatggcgattactggggacaaggcactcttgtcacagtgagctctgcgagcaccaagggc

SEQ ID NO: 59 - protein, 112 aa, synthetic construct
  REGION 1..112: note = artificial
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIK

SEQ ID NO: 60 - DNA, 336 bp, synthetic construct
  misc_feature 1..336: note = Artificial
  source 1..336: mol_type = other DNA
gacatcgtgatgacacagagtcccttgtccttgcctgtgactcctggagaacccgctagcattagctgcagaagcagcaagtcccttctccactctaacggcataacctatctgtactggtatctgcagaaaccagggcagagtccccaactcctgatctaccagatgtccaacctggcatctggcgttcctgatcggttctcagggtcaggttctggcactgacttcacactgaagatctccagggtagaggccgaagatgtcggagtgtactattgtgcccagaatctggagcttccatggacctttggaggtggcaccaaagtcgagattaag

SEQ ID NO: 61 - protein, 112 aa, synthetic construct
  REGION 1..112: note = Artificial
DVVMTQSPLSLPVTPGEPASISCRSSKSLLHSNGITYLYWYLQKPGQSPQLLIYQMSNLASGVPDRFSSSGSGTDFTLKISRVEAEDVGVYYCAQNLELPWTFGGGTKVEIK

SEQ ID NO: 62 - DNA, 336 bp, synthetic construct
  misc_feature 1..336: note = Artificial
  source 1..336: mol_type = other DNA
gatgtcgtgatgacccagagtccactgtcccttcctgtcactcctggtgaaccagccagtatttcctgtcggtcaagcaaatccctccttcactccaacgggattacctatctgtactggtatctgcagaaacccggccaatctccccagttgctcatctaccagatgagcaatctggcctctggagtacccgacaggttcagctctagtggatcagggacagacttcacactgaagatcagcagagtggaagctgaggatgttggcgtgtactattgcgcacagaacctggagttgccttggacctttggaggtggcactaaggtggagataaag

SEQ ID NO: 63 - protein, 112 aa, synthetic construct
  REGION 1..112: note = Artificial
DVVMTQSPLSNPVTPGEPASISCRSSKSLLHSNGITYLYWYLQKPGQSPQLLIYQMSNLASGVPDRFSSSGSGTDFTLKISRVEAEDVGVYYCAQNLELPWTFGGGTKVEIK

SEQ ID NO: 64 - DNA, 336 bp, synthetic construct
  misc_feature 1..336: note = Artificial
  source 1..336: mol_type = other DNA
gacgtcgtgatgactcagtcccctctctctaatcccgttactcctggagaaccagccagcatttcctgcagaagcagcaaatccctgctgcacagtaacggcataacctatctgtactggtatctgcagaaaccagggcaaagtccccagttgctgatctaccagatgtccaacttggcttcaggtgtgcctgatcggttcagtagctctgggtctggcacagacttcaccctgaagatctcaagggtcgaagcagaggatgtaggcgtgtattactgtgcccagaatctcgagcttccctggacctttggaggtggaactaaggtggagataaag

SEQ ID NO: 65 - protein, 112 aa, synthetic construct
  REGION 1..112: note = Artificial
DVVMTQSALSNPVTPGEPASISCRSSKSLLHSNGITYLYWYLQKPGQSPQLLIYQMSNLASGVPDRFSSSGSGTDFTLKISRVEAEDVGVYYCAQNLELPWTFGGGTKVEIK

SEQ ID NO: 66 - DNA, 336 bp, synthetic construct
  misc_feature 1..336: note = Artificial
  source 1..336: mol_type = other DNA
gatgtggtcatgactcagtctgccttgagcaatcccgtaactccaggtgaaccagccagcataagttgtcggtcaagcaagagtctccttcactccaatggcattacctatctctactggtatctgcagaaacctggtcagtctccccaactgctgatctaccagatgagcaacctggcaagtggagtccctgacaggttctcctcctcaggatctgggacagacttcaccctgaagatctccagagtggaagctgaggatgttggcgtgtactattgcgctcagaacctggagttgccctggacctttggcggagggactaaggtggagataaag

SEQ ID NO: 67 - protein, 222 aa, Homo sapiens
QVQLQQPGAELVKPGASVKLSCKASGYIFTSYWMHWVKQRPGQGLEWIGEIDPSDSYTNYNQKFKGKATLTLDKSSSTTYMQLSSLTSEDSAVYYCVRGYGNYGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 68 - protein, 219 aa, Homo sapiens
DVVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 222 aa, synthetic construct
  REGION 1..222: note = Artificial sequence
QVQLVQSGAEVKKPGASVKVSCKASGYTFTSYWMHWVRQAPGQGLEWMGEIDPSDSYTNYNQKFKGRVTMTRDTSTSTVYMELSSLRSEDTAVYYCARGYGNYGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 70 - protein, 219 aa, synthetic construct
  REGION 1..219: note = Artificial sequence
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 5 aa, synthetic construct
  REGION 1..5: note = Artificial sequence
DGGFG

SEQ ID NO: 72 - protein, 6 aa, synthetic construct
  REGION 1..6: note = Artificial sequence
DDGGFG

SEQ ID NO: 73 - protein, 6 aa, synthetic construct
  REGION 1..6: note = Artificial sequence
KDGGFG

SEQ ID NO: 74 - protein, 7 aa, synthetic construct
  REGION 1..7: note = Artificial sequence
GGFGGGF

SEQ ID NO: 75 - protein, 5 aa, synthetic construct
  REGION 1..5: note = Artificial sequence
GGFGG

SEQ ID NO: 76 - protein, 6 aa, synthetic construct
  REGION 1..6: note = Articial sequence
GGFGGG

SEQ ID NO: 77 - protein, 447 aa, synthetic construct
  REGION 1..447: note = Artificial sequence
QVQLVQSGAEVKKPGASVKVSCKASGYIFTSYWMHWVKQRPGQGLEWMGEIDPSDSYTNYNQKFKGRVTMTLDKSTSTTYMELSSLRSEDTAVYYCARGYGNYGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 219 aa, synthetic construct
  REGION 1..219: note = Artificial Sequence
DVVMTQSALSNPVTPGEPASISCRSSKSLLHSNGITYLYWYLQKPGQSPQLLIYQMSNLASGVPDRFSSSGSGTDFTLKISRVEAEDVGVYYCAQNLELPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0297004 A1
(43) Pub. Date: Sep. 25, 2025

(54) NECTIN-4 BINDING AGENTS

(71) Applicant: INNATE PHARMA, MARSEILLE (FR)

(72) Inventors: MANEL KRAIEM, MARSEILLE (FR); STÉPHANIE CHANTEUX, MARSEILLE (FR); BENJAMIN ROSSI, MARSEILLE (FR); LAURENT GAUTHIER, MARSEILLE (FR)

(21) Appl. No.: 18/867,898

(22) PCT Filed: May 24, 2023
(86) PCT No.: PCT/EP2023/063912

§ 371 Date: Nov. 21, 2024

Related U.S. Application Data
(60) Provisional application No. 63/345,453, filed on May 25, 2022.

(51) Int. Cl. C07K 16/28; A61K 47/68; A61P 35/00

(52) U.S. Cl. CPC C07K 16/2803; A61K 47/68037; A61K 47/6851; A61P 35/00; C07K 2317/24; C07K 2317/33; C07K 2317/34; C07K 2317/77; C07K 2317/92

(57) ABSTRACT

The invention relates to antibodies and antibody fragment that bind to Nectin-4 polypeptides. The invention also relates to antibody-drug conjugates that comprise the antibodies or antibody fragments, and to methods of making the conjugates, pharmaceutical compositions, and method of using them to diagnose, treat or prevent diseases, e.g. cancer characterized by Nectin-4 expressing tumor cells.

### CROSS-REFERENCE TO RELATED APPLICATIONS

This application is the U.S. national stage application of International Patent Application No. PCT/EP2023/063912, filed May 24, 2023, which claims the benefit of U.S. Provisional Application No. 63/345,453 filed 25 May 2022, which is incorporated herein by reference in its entirety; including any drawings.

### REFERENCE TO SEQUENCE LISTING

The present application is being filed along with a Sequence Listing in electronic format. The Sequence Listing is provided as a file entitled “Nectin-4-3 PCT”, created 22 May 2023, which is 99,303 bytes in size. The information in the electronic format of the Sequence Listing is incorporated herein by reference in its entirety.

### FIELD OF THE INVENTION

The present invention relates to antibodies, and fragment thereof, that binds to Nectin-4 polypeptides. The invention also relates to cells producing such compounds, method of making such compounds, conjugates and method of making conjugates, pharmaceutical compositions comprising the same, method of using such compounds to diagnose, treat or prevent diseases, e.g. cancer characterized by Nectin-4 expressing tumor cells.

### BACKGROUND

Nectin-4 is a surface molecule of the nectin proteins family that play a key role in various biological processes such as polarity, proliferation, differentiation and migration for epithelial, endothelial, immune and neuronal cells, during development and adult life. Nectin-4 was initially cloned from human trachea by the Lopez group in 2001 (see Reymond et al. (2001) J. Biol. Chem. 276 (46): 43205-15). Nectins are the main receptors for polio, herpes simplex and measles viruses and are further involved in several pathological processes in humans. Nectin-4 is expressed at significantly higher levels in several tumors, and it is particularly over-expressed in breast cancer, including triple negative breast cancer (TNBC) (See M-Rabet et al. Ann Oncol. 2017 Apr. 1; 28 (4): 769-776), pancreatic cancer and in Urothelial cancer. In addition, nectin-4 is also expressed in non-small cell lung cancer, ovarian cancer, head and neck squamous cell carcinoma and esophageal cancer tumor specimens. Challita-Eid et al. (2016) Cancer Res. 76 (10): 3003-3013 reported that moderate to strong staining by immunohistochemistry (H-score≥100) in bladder (60%) and breast (53%) tumor tissues. Zeindler et al. 2019 Front. Med. 6:200 reported that a high expression of Nectin-4 was present in 86 (58%) of the 148 TNBC cases. In the serum of patients with these cancers, the detection of soluble forms of Nectin-4 is associated with a poor prognosis. Levels of serum Nectin-4 increase during metastatic progression and decrease after treatment. These results suggest that Nectin-4 could be a reliable target for the treatment of cancer.

Accordingly, several anti-Nectin-4 antibodies have been described in the prior art. In particular, Enfortumab Vedotin (ASG-22ME) is an antibody-drug conjugate (ADC) targeting Nectin-4 and is currently in clinical investigation for the treatment of patients suffering from solid tumors. Challita-Eid et al. (2016), supra, developed an anti-Nectin-4 antibody conjugated to the highly potent microtubule-disrupting agent MMAE based on antibody AGS-22. The work gave rise to the ADC drug candidate enfortumab vedotin (see U.S. Pat. No. 8,637,642 and PCT publication No. WO2012/047724, Agensys Inc.) which has yielded promising results in human clinical trials in treatment of patients with locally advanced or metastatic urothelial cancer who have previously received a platinum-containing chemotherapy in the neoadjuvant/adjuvant, locally advanced, or metastatic setting and a PD-1/PD-L1 checkpoint inhibitor. Several other groups have also proposed anti-Nectin-4 agents bound to a variety of toxic agents. PCT patent application WO2018/158398 (INSERM) reports several anti-Nectin-4 antibodies and proposes potential coupling to a range of cytotoxic agents. Similarly, U.S. Pat. No. 8,637,642 (Agensys Inc.) also provides anti-Nectin-4 antibodies and proposes potential coupling to a range of cytotoxic agents. Yet further, Bicycle Therapeutics has reported development of an anti-Nectin-4 targeting agent comprised of a Nectin-4 binding protein conjugated to a cytotoxic auristatin (MMAE) payload via a valine-citrulline (val-cit), cleavable linker. To date, all the anti-Nectin-4 antibodies that have been reported as active when conjugated to chemotherapeutic agents have targeted the Ig-like V type domain of Nectin-4, consequently the Ig-like V type domain is believed to provides the strongest internalization of anti-Nectin-4 ADCs.

Enfortumab vedotin (anti-Nectin-4 ADC) binds to the Ig-like V type domain of Nectin-4 which is associated with highly-internalizing anti-Nectin-4 antibodies. Enfortumab vedotin has shown impressive therapeutic responses with an ORR (objective response rate) of 44% and CR (complete response rate) of 12% in UC in the EV-201 Phase 2 study (2019), about half of the patients discontinued treatment. Most of the discontinuation was due to progressive disease as assessed by RECIST (48%) or clinical symptoms (5%). Also, 18% patients that discontinued experienced adverse events, notably neuropathy. The Nectin-4-targeted ADCs therefore have limitations, and there is a need in the art for improved benefit to patients afflicted with UC and other cancers.

### SUMMARY OF THE INVENTION

Provided herein are variable heavy (VH) and variable light (VL) domains for use in Nectin-4 binding proteins. One object of the present disclosure is to provide an anti-Nectin-4 antibody, or antibody fragment comprising a heavy chain variable region having at least about 80% sequence identity (e.g., at least about 85%, 90%, 95%, 97%, 98% or 99% identity, or 100% identity) to a heavy chain comprising the amino acid sequences of SEQ ID NO: 37, 39, 41, 43, 45, 47, 49, 51, 53, 55 or 57 and a light chain variable region having at least about 80% sequence identity (e.g., at least about 85%, 90%, 95%, 97%, 98% or 99% identity, or 100% identity) to a light chain comprising the amino acid sequence of SEQ ID NO: 59, 61, 63 or 65.

In one embodiment, provided is an anti-Nectin-4 antibody or antibody fragment, comprising a heavy chain variable region having at least about 80% sequence identity (e.g., at least about 85%, 90%, 95%, 97%, 98% or 99% identity, or 100% identity) to the heavy chain comprising the amino acid sequence of SEQ ID NO: 69 and a light chain variable region having at least about 80% sequence identity (e.g., at least about 85%, 90%, 95%, 97%, 98% or 99% identity, or 100% identity) to the amino acid sequence of SEQ ID NO: 70.

In one embodiment, provided is an anti-Nectin-4 antibody or antibody fragment, comprising a heavy chain variable region (VH) comprising a CDR1, CDR2 and CDR3 having the amino acid sequences of SEQ ID NOS: 21, 22 and 23 respectively and framework FR1, FR2 and FR3 amino acid sequences from the human IGHV1-46*01 gene (and optionally further framework FR4 amino acid sequences from the human IGHJ4*01 gene); and a light chain variable region (VL) CDR1, CDR2 and CDR3 having the amino acid sequences of SEQ ID NOS: 24, 25 and 26 respectively, and framework FR1, FR2 and FR3 amino acid sequences from the human IGKV2-28*01 gene (and optionally further framework FR4 amino acid sequences from the human IGKJ4*01 gene). The framework sequences from the particular human IGHV, IGHJ, IGKV and IGKJ genes may comprise one or more amino acid substitutions as further described herein.

In one embodiment, provided is an anti-Nectin-4 antibody or antibody fragment, comprising a heavy chain variable region that comprises one, two, three, four, five, six, seven or eight amino acid substitutions at a Kabat position selected from 28, 38, 40, 48, 69, 71, 73, 78. Optionally, the threonine residue at position 28 is substituted by an isoleucine residue. Optionally, the arginine residue at position 38 is substituted by a lysine residue. Optionally, the alanine residue at position 40 is substituted by an arginine residue. Optionally, the methionine residue at position 48 is substituted by an isoleucine residue. Optionally, the methionine residue at position 69 is substituted by a leucine residue. Optionally, the arginine residue at position 71 is substituted by a leucine residue. Optionally, the threonine residue at position 73 is substituted by a lysine residue. Optionally, the valine residue at position 78 is substituted by a threonine residue.

In one embodiment, provided is an anti-Nectin-4 antibody or antibody fragment, comprising a light chain variable region that comprises one, two, three or four amino acid substitutions at a kabat position selected from 2, 8, 11, 64. Optionally, the isoleucine residue at position 2 is substituted by a valine residue. Optionally, the proline residue at position 8 is substituted by an alanine residue. Optionally, the leucine residue at position 11 is substituted by an asparagine. Optionally, the glycine residue at position 64 is substituted by a serine residue.

In one embodiment, provided is an anti-Nectin-4 antibody, comprising a heavy chain having at least about 80% sequence identity (e.g., at least about 70%, 85%, 90%, 95%, 97%, 98% or 99% identity, or 100% identity) to a heavy chain comprising the amino acid sequence of SEQ ID NO: 77; and a light chain having at least about 80% sequence identity (e.g., at least about 70%, 85%, 90%, 95%, 97%, 98% or 99% identity, or 100% identity) to a light chain comprising the amino acid sequence of SEQ ID NO: 78.

In one embodiment, provided is an anti-Nectin-4 antibody or antibody fragment, comprising a heavy chain variable region having at least about 80% sequence identity (e.g., at least about 85%, 90%, 95%, 97%, 98% or 99% identity, or 100% identity) to the heavy chain comprising the amino acid sequence of SEQ ID NO: 47 and a light chain variable region having at least about 80% sequence identity (e.g., at least about 85%, 90%, 95%, 97%, 98% or 99% identity, or 100% identity) to the amino acid sequence of SEQ ID NO: 65.

In one embodiment, provided is a Nectin-4 binding protein that comprises an antibody or antibody fragment of the disclosure, and optionally further an additional antigen-binding domain.

In any embodiment, the antibody or antibody fragment can optionally be characterized as being monoclonal, humanized and/or in isolated form.

In one embodiment, an anti-Nectin-4 antibody or antibody fragment binds the VC1 bridging domain of a Nectin-4 polypeptide.

In any aspect, the antibody or antibody fragment of the disclosure is for use in preparation of an antibody drug conjugate (ADC). In any aspect, the antibody of the disclosure is for use in (e.g., for use in a method of) reducing cell-cell adhesion between Nectin-4 expressing tumor cells, inhibiting a Nectin-4:Nectin-1 interaction, inhibiting a Nectin-4:Nectin-4 interaction, reducing growth of Nectin-4 expressing tumor cells and/or reducing cluster formation of Nectin-4 expressing tumor cells.

According to one of the present invention, provided is an antibody drug conjugate comprising an anti-Nectin-4 antibody or antibody fragment, conjugated to a cytotoxic agent, optionally wherein the cytotoxic agent is conjugated to the antibody or antibody fragment via a linker, and optionally further wherein the linker or linker-toxin comprises any one of Formulae III to XIV.

In one embodiment, the antibody drug conjugate (ADC) comprises an anti-Nectin-4 antibody or antibody fragment connected to at least one cytotoxic drug moiety, wherein the antibody or antibody fragment comprises a heavy chain variable region having at least about 80% sequence identity (e.g., at least about 85%, 90%, 95%, 97%, 98% or 99% identity, or 100% identity) to the amino acid sequence of SEQ ID NO: 69 and a light chain variable region having at least about 80% sequence identity (e.g., at least about 85%, 90%, 95%, 97%, 98% or 99% identity, or 100% identity) to the amino acid sequence of SEQ ID NO: 70. In one embodiment, the antibody drug conjugate (ADC) comprises (a) at least one antigen binding domain that binds to Nectin-4 polypeptide, said antigen binding domain comprising (i) a heavy chain variable region that has at least 80% sequence identity (e.g., at least about 85%, 90%, 95%, 97%, 98% or 99% identity, or 100% identity) to the amino acid sequences of SEQ ID NO: 37, 39, 41, 43, 45, 47, 49, 51, 53, 55 or 57 and (ii) a light chain variable region having at least about 80% sequence identity (e.g., at least about 85%, 90%, 95%, 97%, 98% or 99% identity, or 100% identity) to the amino acid sequence of SEQ ID NO: 59, 61, 63 or 65; and (b) at least one cytotoxic drug moiety.

In a preferred embodiment, the antibody drug conjugate (ADC) comprises (a) an anti-Nectin-4 humanized antibody comprising (i) a heavy chain variable region that has at least 80% sequence identity (e.g., at least about 85%, 90%, 95%, 97%, 98% or 99% identity, or 100% identity) to the amino acid sequences of SEQ ID NO: 47, and (ii) a light chain variable region having at least about 80% sequence identity (e.g., at least about 85%, 90%, 95%, 97%, 98% or 99% identity, or 100% identity) to the amino acid sequences of SEQ ID NO: 65.

In some aspects, the antibody (e.g. a full-length antibody, an antibody fragment) that binds a human Nectin-4 polypeptide is capable of reducing cell-cell adhesion between Nectin-4 expressing tumor cells, and/or reducing growth and/or cluster formation of Nectin-4 expressing tumor cells (as assessed, for example, using 3-dimensional or non-adherent tumor cell culture; tumor spheroid assays), for use in preparation of an antibody drug conjugate.

In some aspects, the antibody (e.g. a full-length antibody, an antibody fragment) is capable of inhibiting a Nectin-4:Nectin-1 interaction and/or a Nectin-4:Nectin-4 interaction (e.g., the antibody is capable of reducing the interaction of Nectin-4 on a first cell with Nectin-1 and/or Nectin-4 on a second cell, optionally wherein the cell(s) is a tumor cell).

In any aspect herein, the anti-Nectin-4 antibody or antibody fragment, or the antibody-drug conjugate comprising such antibody or fragment, is capable, upon binding to Nectin-4 on the surface of a tumor cell, of undergoing intracellular internalization.

In some aspects, provided are methods of preparing an antibody drug conjugate comprising conjugating an anti-Nectin-4 antibody or antibody fragment (e.g. an antibody or antibody fragment according to the disclosure) to a cytotoxic agent (e.g. a linker or linker-toxin according to the disclosure).

In some aspects, preparation of an antibody drug conjugate comprises a step of conjugating the antibody to a cytotoxic agent (e.g. via a linker moiety, a linker moiety further comprising an intracellularly cleavable moiety).

In one aspect, the anti-Nectin-4 antibody or antibody fragment is conjugated to a cytotoxic agent via an intracellularly-cleavable (e.g., protease cleavable) oligo-peptide (e.g. di-, tri, tetra- or penta-peptide). In one aspect, the anti-Nectin-4 antibody or antibody fragment is conjugated to a cytotoxic agent (e.g. a camptothecin derivative) via an intracellularly-cleavable (e.g. protease-cleavable) di-, tri-, tetra- or penta-peptide and a self-eliminating spacer. In one aspect, the anti-Nectin-4 antibody or antibody fragment is conjugated to a cytotoxic agent (e.g. a camptothecin derivative) via an intracellularly-cleavable (e.g., protease cleavable) tetra- or penta-peptide and a self- or non-self-eliminating spacer.

In one aspect, the cytotoxic agent is a highly potent chemotherapeutic agent, optionally a cytotoxic agent selected from the group consisting of taxanes, anthracyclines, camptothecins, epothilones, mytomycins, combretastatins, vinca alkaloids, nitrogen mustards, maytansinoids, duocarmycins, tubulysins, dolastatins and auristatins, enediynes (e.g., calicheamicins, esperamicins, shishijimicins and namenamicins), pyrrolobenzodiazepines (e.g., pyrrolobenzodiazepine dimers and indolino-pyrrolobenzodiazepine dimers), amatoxins and ethylenimines. In one embodiment, the cytotoxic agent is a DNA damaging agent, include for example a DNA intercalating agent, e.g. an agent that inserts itself into the DNA structure of a cell and binds to the DNA, in turn causing DNA damage (e.g. daunorubicin). Compounds include topoisomerase inhibitors, chemical compounds that block the action of topoisomerase (topoisomerase I and II). Such compounds are used for a wide range of solid tumor and hematological malignancies, notably lymphomas. Topoisomerase I inhibitors include camptothecins, for example irinotecan (approved for treatment of colon cancer), topotecan (approved for treatment of ovarian and lung cancer), camptothecin, lamellarin D, indenoisoquinoline, indimitecan. Further camptothecins include silatecan, cositecan, exatecan, lurtotecan, gimatecan, belotecan, and rubitecan. Topoisomerase II inhibitors include for example etoposide (VP-16), teniposide, doxorubicin, daunorubicin, mitoxantrone, amsacrine, ellipticines, aurintricarboxylic acid, and HU-331, a quinolone synthesized from cannabidiol.

Optionally the antibody or antibody fragment is functionalized with (e.g. conjugated to, covalently bound to) a linker-toxin of any one of Formulae III to XIV.

In one aspect, the cytotoxic agent is camptothecin analogue, e.g., an exatecan, Dxd or SN-38 molecule.

In one aspect, the present invention provides a Nectin-4 binding antibody or antibody fragment of the disclosure conjugated (e.g. covalently bound to) to a camptothecin, e.g. a camptothecin analogue, an exatecan or exatecan derivative, a Dxd molecule or a SN-38 molecule.

In any embodiment herein, a Nectin-4 binding antibody or antibody fragment of the disclosure conjugated (e.g. covalently bound to) to a camptothecin analogue, e.g. an exatecan or SN-38 molecule, can be characterized as comprising an antibody that specifically binds to a human Nectin-4 polypeptide having one or more amino acid residues (e.g. cysteine, lysine, glutamine residues, a non-natural amino acid residue) functionalized, via a linker, with a molecule comprising the structure of Compounds 1 or 2. In any embodiment herein, a Nectin-4 antibody or antibody fragment can be characterized as being functionalized with a linker-camptothecin molecule having a structure of Formulas III, IV, V, VI, VII, VII, IX, X, XI, XII, XIII, or XIV, or with any of Compounds 3 to 16.

In one embodiment, the Nectin-4 antibody or antibody fragment conjugated to a camptothecin derivative is a Nectin-4 antibody or antibody fragment conjugated to an exatecan molecule, e.g. a molecule having the structure of Compound 1 (1a or 1b). In any embodiment, a cleavable linker or an immunoconjugate or ADC comprising a linker can be characterized as releasing an exatecan molecule, e.g. a molecule having the structure of Compound 1 (1a or 1b), e.g. upon enzymatic cleavage of the linker. In one embodiment, the humanized Nectin-4 antibody or antibody fragment conjugated to a camptothecin derivative is a Nectin-4 antibody or antibody fragment conjugated to a SN-38 molecule, e.g., a molecule having the structure of Compound 2. In one embodiment, the Nectin-4 antibody or antibody fragment can be characterized as comprising an antibody that specifically binds to a human Nectin-4 polypeptide having one or more amino acid residues (e.g. cysteine, lysine, glutamine or non-natural amino acid residues) functionalized, via a linker (e.g. a cleavable linker molecule with or without an additional spacer, for example spacer (Y′) described herein), with a molecule having the structure:

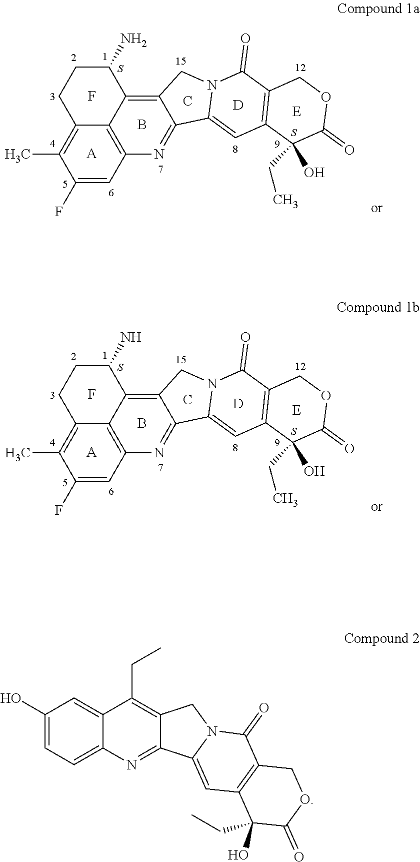

In one embodiment, the Nectin-4 antibody or antibody fragment conjugated to a cytotoxic agent can be specified as being an immunoconjugate represented by Formula (I):

Ab-X-Z Formula (I)
wherein,
Ab is an antibody or antibody fragment that specifically binds to a human Nectin-4 polypeptide (e.g. any antibody or antibody fragment of the disclosure, optionally the antibody binds the VC1 bridging domain of human Nectin-4 and/or displays decreased binding to a mutant human Nectin-4 polypeptide comprising an amino acid substitution at residues K197 and/or S199 (with reference to SEQ ID NO: 1), compared to binding to a wild-type human Nectin-4 polypeptide);
X is a linker molecule which connects Ab and Z (e.g., is covalently bound to each of Ab and Z), wherein X comprises a moiety that is cleavable, e.g., under physiological conditions, optionally under intracellular conditions, optionally a protease-cleavable di-, tri-, tetra- or penta-peptide, optionally wherein X further comprises a self-eliminating or non-self-eliminating spacer system (Y′) positioned between the cleavable moiety and Z, optionally wherein X further comprises a spacer (Y) positioned between the Ab and the cleavable moiety; and
Z is a cytotoxic agent, optionally wherein Z is a camptothecin analogue, optionally wherein Z is exatecan, optionally wherein Z is exatecan and cleavage of the linker results in the release of a compound having the structure of Compound 1 (exatecan).

In one embodiment, the Nectin-4 antibody or antibody fragment conjugated to a cytotoxic agent can be specified as being an immunoconjugate represented by Formula (I):

Ab-X-Z Formula (I)
wherein,
Ab is an antibody or antibody fragment according to the disclosure that specifically binds to a human Nectin-4 polypeptide;
X is a linker molecule which connects Ab and Z (e.g., is covalently bound to each of Ab and Z), wherein X comprises a valine-citrulline, valine-alanine or phenylalanine-lysine dipeptide, wherein X further comprises a self-eliminating or non-self-eliminating spacer system (Y′) positioned between the cleavable moiety and Z, and wherein X further comprises a spacer (Y) positioned between the Ab and the cleavable moiety; and
Z is a cytotoxic agent, optionally a camptothecin analogue, optionally an exatecan molecule or a SN-38 molecule.

In one embodiment, provided is a method of delivering or targeting a cytotoxic agent (optionally a camptothecin analogue) to a tumor, a method of releasing a cytotoxic agent (optionally a camptothecin analogue, a Dxd, an exatecan) in a tumor (e.g. in a subject having cancer), or a method of sensitizing a tumor or cancer to a cytotoxic agent (optionally a camptothecin analogue), the method comprising administering to a subject having a cancer an immunoconjugate represented by Formula (I):

Ab-X-Z Formula (I)
wherein,
Ab is an antibody or antibody fragment that specifically binds to a human Nectin-4 polypeptide;
X is a linker molecule which connects Ab and Z (e.g., is covalently bound to each of Ab and Z), wherein X comprises a moiety that is cleavable, e.g., under physiological conditions, optionally under intracellular conditions, optionally a protease-cleavable di-, tri-, tetra- or penta-peptide, optionally wherein X further comprises a self-eliminating or non-self-eliminating spacer system (Y′) positioned between the cleavable moiety and Z, optionally wherein X further comprises a spacer (Y) positioned between the Ab and the cleavable moiety; and
Z is a cytotoxic agent, optionally a camptothecin analogue, optionally an exatecan molecule or a SN-38 molecule, optionally wherein Z is exatecan and wherein cleavage of the linker results in the release of a compound having the structure of Compound 1 (exatecan).

In one embodiment, the antibody or antibody fragment conjugated to a cytotoxic agent (optionally a camptothecin analogue) can be specified as being an immunoconjugate represented by Formula (II):

Ab-(X-(Z)ₙ)ₘ Formula (II)
wherein,
Ab is an antibody or antibody fragment according to the disclosure that specifically binds to a human Nectin-4 polypeptide;
X is a linker molecule which connects Ab and Z, wherein X comprises a moiety that is cleavable, e.g., under physiological conditions, optionally under intracellular conditions, optionally a protease-cleavable di-, tri-, tetra- or penta-peptide, optionally wherein X further comprises a self-eliminating or non-self-eliminating spacer system (Y′) positioned between the cleavable moiety and Z, optionally wherein X further comprises a spacer (Y) positioned between the Ab and the cleavable moiety;
Z is a cytotoxic agent, optionally a camptothecin analogue, optionally Z is a molecule comprising an exatecan molecule or a SN-38 molecule, e.g., a molecule having the structure of Compounds 1 or 2;
n is 1; and
m is from 4 to 8, or optionally m is an integer selected from among 4, 5, 6, 7 or 8.

In one embodiment, a Nectin-4 antibody or antibody fragment conjugated to a cytotoxic agent, (optionally a camptothecin analogue) can be characterized as a composition of immunoconjugates represented by Formula (II):

Ab-(X-(Z)ₙ)ₘ Formula (II)
wherein,
Ab is an antibody or antibody fragment according to the disclosure that specifically binds to a human Nectin-4 polypeptide;
X is a molecule which connects Ab and Z, wherein X comprises a moiety that is cleavable, e.g., under physiological conditions, optionally under intracellular conditions, optionally a protease-cleavable di-, tri-, tetra- or penta-peptide, optionally wherein X further comprises a self-eliminating or non-self-eliminating spacer system (Y′) positioned between the cleavable moiety and Z, optionally wherein X further comprises a spacer (Y) positioned between the Ab and the cleavable moiety;
Z is a cytotoxic agent, optionally a camptothecin analogue, optionally Z is a molecule comprising an exatecan molecule or a SN-38 molecule;
wherein n is 1, and at least 50%, 60%, 70%, 80%, 90%, 95%, 98% or 99% of immunoconjugates in the composition have an m (the number of X-Z moieties) that is between 2 and 4, between 4 and 8, optionally between 6 and 8. Optionally at least 50%, 60%, 70%, 80%, 90%, 95%, 98% or 99% of immunoconjugates in the composition have an m that is, or that is at least, 4, 6, 7 or 8.

In Formulae I or II, the (X-Z) moiety can optionally be characterized having a structure of any of Formulae III to XI or of any of Compounds 3-12.

In Formulae I or II, molecule X or spacer Y can optionally be specified as comprising a reactive group (R) or a residue of the reaction of a reactive group (R) with an amino acid of the antigen binding protein (e.g., antibody) or with a complementary reactive group (R′) that is attached to an amino acid of the antibody or antibody fragment.

In any embodiment herein, the exatecan molecule can be specified as being bound to linker (X) via the amine at position 1 of the exatecan (NH replaces NH2 at position 1 when the exatecan molecule is part of a linker). In one embodiment, the exatecan is bound to the linker (X), e.g. to the carbonyl of a p-aminobenzyloxycarbonyl (PAB) self-eliminating spacer moiety of X. In any embodiment herein, the SN-38 molecule can be specified as being bound to linker (X) via the OH at position 9 (O replaces OH at position 9 when the SN-38 molecule shown in Compound 2 is part of a linker).

In one embodiment, the Nectin-4 binding agent conjugated to an exatecan can be characterized as comprising an antibody that specifically binds to a human Nectin-4 polypeptide having one or more amino acid residues (e.g. cysteine residues, glutamine residues) functionalized, via a spacer (Y), with a linker-exatecan molecule comprising the following structure:

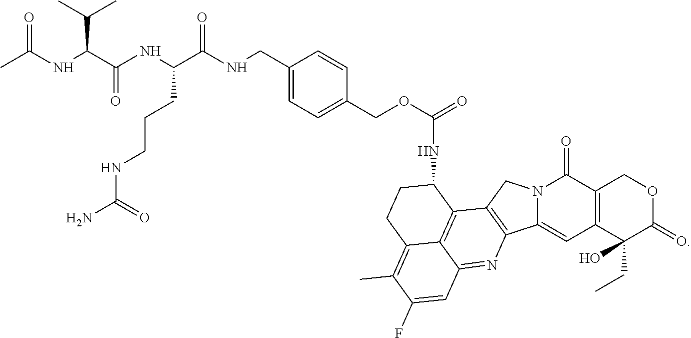 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](CCCNC(N)=O)NC(=O)[C@@H](NC(C)=O)C(C)C)cc1)CC3]

In one embodiment, the humanized Nectin-4 antibody or antibody fragment conjugated to an exatecan can be characterized as comprising an antibody that specifically binds to a human Nectin-4 polypeptide having one or more amino acid residues (e.g. cysteine residues, glutamine residues) functionalized, via a spacer (Y), with a linker-exatecan comprising the following structure:

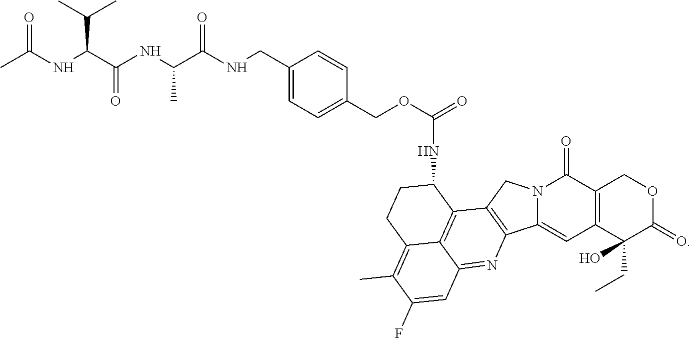 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](C)NC(=O)[C@@H](NC(C)=O)C(C)C)cc1)CC3]

In one embodiment, the humanized Nectin-4 antibody or antibody fragment conjugated to an exatecan can be characterized as comprising an antibody that specifically binds to a human Nectin-4 polypeptide having one or more amino acid residues (e.g. cysteine residues, glutamine residues) functionalized, via a spacer (Y), with a linker-exatecan molecule comprising the following structure:

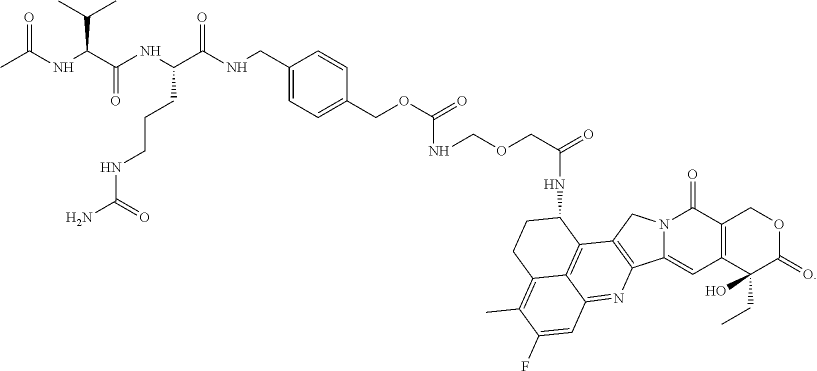 [SMILES: CC[C@@]1(O)C(=O)OCc2c1cc1n(c2=O)Cc2c-1nc1cc(F)c(C)c3c1c2[C@@H](NC(=O)COCNC(=O)OCc1ccc(CNC(=O)[C@H](CCCNC(N)=O)NC(=O)[C@@H](NC(C)=O)C(C)C)cc1)CC3]

In one embodiment, the humanized Nectin-4 antibody or antibody fragment conjugated to an exatecan can be characterized as comprising an antibody that specifically binds to a human Nectin-4 polypeptide having one or more amino acid residues (e.g. cysteine residues, glutamine residues) functionalized, via a spacer (Y), with a linker-exatecan molecule comprising the following structure:

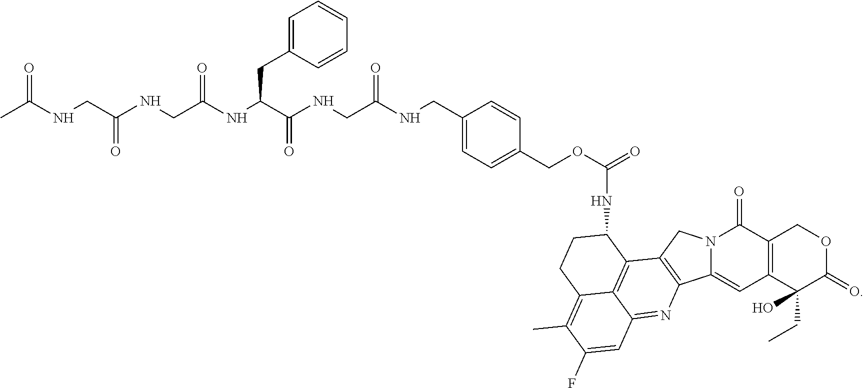 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)CNC(=O)[C@H](Cc2ccccc2)NC(=O)CNC(=O)CNC(C)=O)cc1)CC3]

Spacer (Y) can be specified as being or comprising a substituted or unsubstituted alkyl or heteroalkyl chain, optionally wherein Y has a chain length of 2-100 atoms, 2-40 atoms, optionally 2-30, 2-20, 4-40, 4-30 or 4-20 atoms, optionally where one or more atoms can be other than carbon, for example oxygen, sulfur, nitrogen, or other atoms, optionally wherein any carbon of the chain is substituted with an alkoxy, hydroxyl, alkylcarbonyloxy, alkyl-S—, thiol, alkyl-C(O)S—, amine, alkylamine, amide, or alkylamide. For example Y may comprise one or more ethylene oxide monomers, optionally Y comprises a polyethylene oxide moiety, optionally Y comprises a structure —(CH₂CH₂O)ₓ— where x is 1 to 24, optionally 1 to 12, optionally 1 to 8, optionally 1 to 6.

In some aspects, provided is a linker-exatecan molecule having the structure:

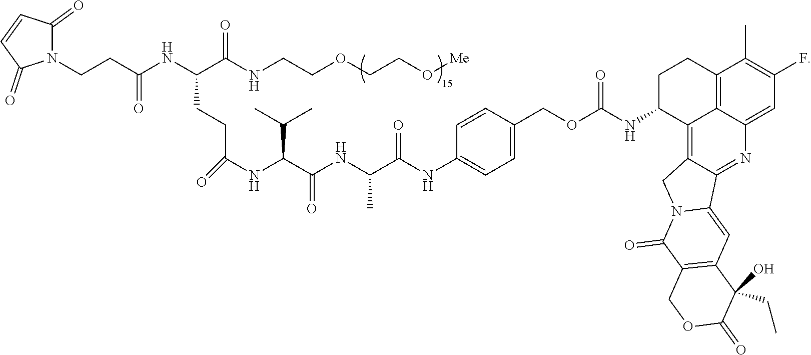

In certain embodiments, provided is an immunoconjugate that binds a human Nectin-4 polypeptide, wherein the immunoconjugate is represented by any one of the Formulae:

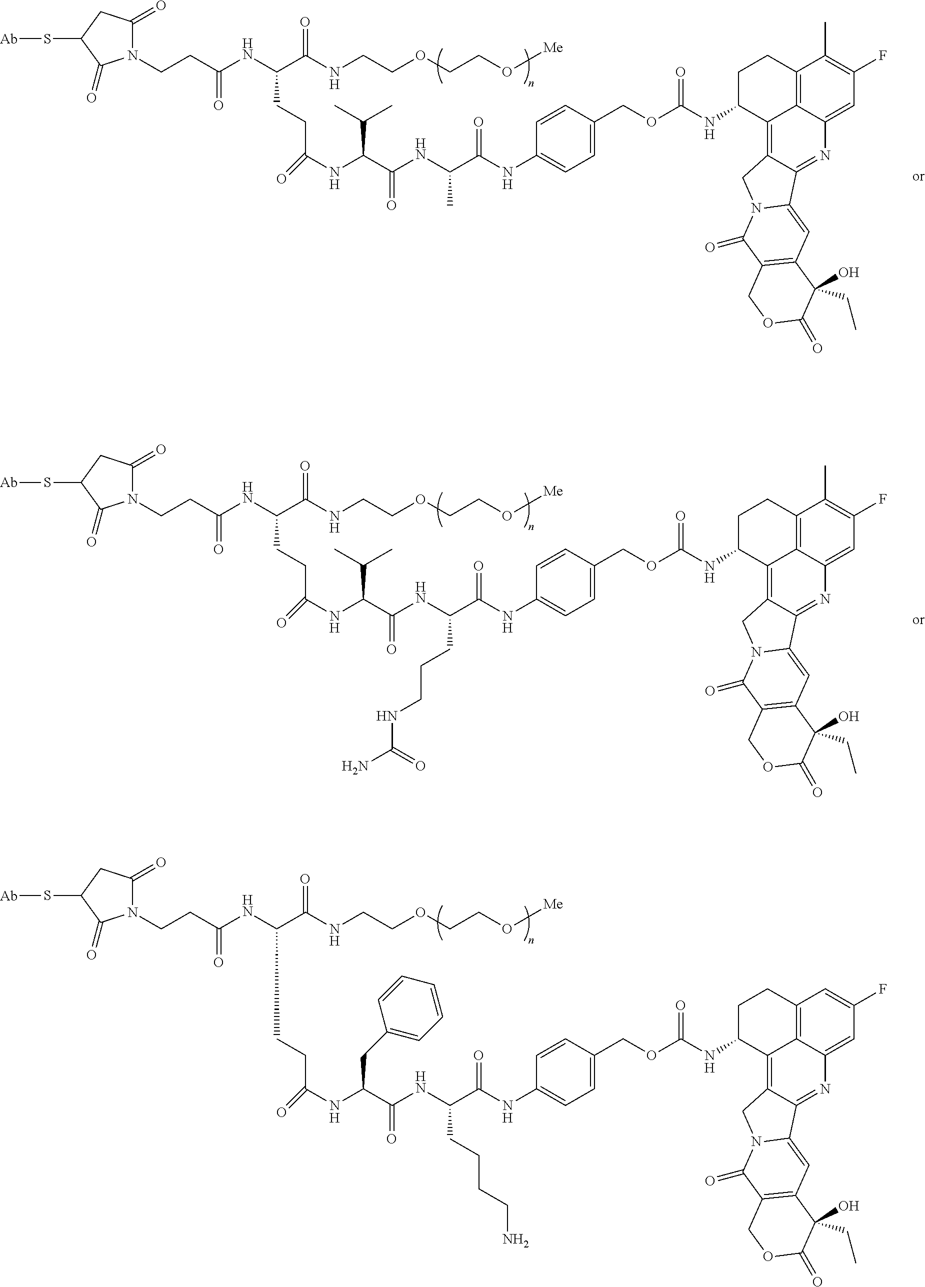

wherein n is 1-15, 5-15, 5-23 (e.g. 1, 2, 3, 4, 5, 6, 7, 8, 9, 10, 11, 12, 13, 14, 15, 16, 17, 18, 19, 20, 21, 22 or 23); and
wherein Ab is an antibody or antibody fragment thereof that specifically binds a human Nectin-4 polypeptide, optionally wherein the antibody or antibody fragment comprising an amino acid sequence at least 60%, optionally at least 70%, optionally 80% or optionally 90% identical to an amino acid sequence selected from the group consisting of SEQ ID NOS: 19, 47, 69 or 77; and an amino acid sequence at least 60%, optionally at least 70%, optionally 80% or optionally 90% identical to an amino acid sequence selected from the group consisting of SEQ ID NOS: 20, 65, 70 or 78. S can be specified as being an atom of a cysteine residue of the antibody (Ab).

In one embodiment, provided is an immunoconjugate that binds a human Nectin-4 polypeptide, wherein the immunoconjugate is represented by the Formula:

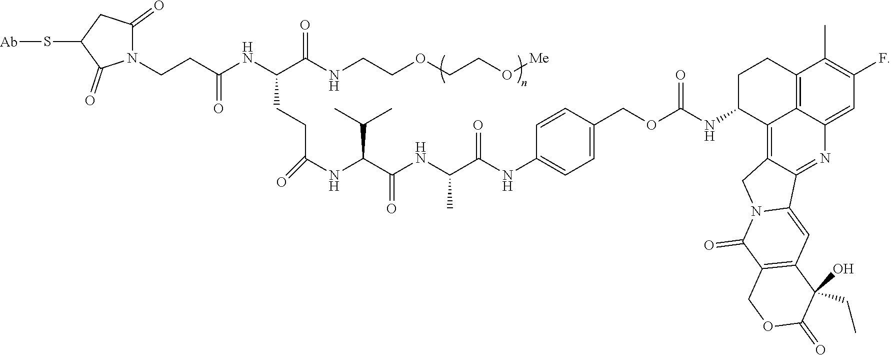

wherein Ab is an antibody or antibody fragment thereof that specifically binds a human Nectin-4 polypeptide. In one embodiment, n is 15 and the immunoconjugate is characterized by a DAR of between 6 and 8, optionally wherein the DAR is 6, optionally wherein the DAR is 8; and wherein S is an atom of a cysteine residue of the antibody (Ab). In one embodiment, the Ab comprises a heavy chain variable region (VH) comprising the amino acid sequence of SEQ ID NOS: 47; and a light chain variable region (VL) comprising the amino acid sequence of SEQ ID NOS: 65, optionally wherein the antibody or antibody fragment comprises a heavy chain comprising the amino acid sequence of SEQ ID NOS: 77; and a light chain comprising the amino acid sequence of SEQ ID NOS: 78.

In one aspect, the treatment shows improved efficacy and/or improved (lower) drug resistance compared to existing anti-Nectin-4 ADC therapies (e.g. an anti-Nectin-4 antibody or antibody fragment conjugated to an auristatin; enfortumab vedotin). In one aspect, provided is a method of treating and/or preventing a cancer and/or killing tumor cells in an individual in need thereof, wherein the treatment comprises 2, 3, 4, 5, 6, 7, 8, 9 or 10 or more administrations of a Nectin-4 binding agent conjugated to a camptothecin derivative (e.g. an exatecan or SN-38 molecule) at a frequency of 1 to 2 times per month (e.g. once every two weeks, once every three weeks or once every four weeks).

Also provided is a method of using the antibody, the fragment thereof, optionally conjugated with a cytotoxic drug moiety, in the treatment or prevention of disease, e.g., cancer, in an individual in need thereof. In one embodiment, the antibody or fragment, optionally conjugated with a cytotoxic drug moiety is administered to an individual having a cancer in an amount and frequency sufficient to kill Nectin-4 expressing tumor cells. In one embodiment, said individual has a Nectin-4 expressing tumor, optionally wherein the tumor is an HER-2 expressing tumor or a HER2-negative tumor, e.g. an urothelial cancer, head and neck squamous cell carcinoma or an esophageal cancer. In one embodiment, the cancer or tumor is advanced recurrent or metastatic cancer, optionally an advanced recurrent or metastatic urothelial cancer. In one embodiment, the cancer or tumor is a triple-negative breast cancer (TNBC).

In one aspect, the methods of treatment herein can be used in individuals having a Nectin-4-expressing cancer irrespective of the level of Nectin-4 expression on tumor cells.

In one aspect, the methods of treatment herein can be used advantageously in individuals whose tumor cells express P-glycoprotein (Pgp).

In one aspect, the methods of treatment of the disclosure can be used advantageously in individuals having received prior treatment with a chemotherapeutic agent (e.g. a chemotherapeutic agent transported by P-glycoprotein (Pgp), a platinum agent (e.g., oxaliplatin, cisplatin, carboplatin, nedaplatin, Phenanthriplatin, picoplatin, satraplatin), a taxane (e.g., Paclitaxel (Taxol™) and docetaxel (Taxotere™).

The increased anti-tumor potency and greater therapeutic window offered by an anti-Nectin-4 antibody of the disclosure (e.g., particularly when conjugated to a camptothecin derivative) provides the possibility for improved treatment outcomes in individuals having tumors having resistance to, that are not responsive to or that have progressed following treatment with a composition comprising an anti-HER2 agent (e.g. trastuzumab; an ADC comprising trastuzumab), or with a composition comprising another anti-Nectin-4 agent (e.g. an ADC comprising enfortumab; enfortumab vedotin). The improved therapeutic window provides the possibility for combination treatment with other agents, particularly chemotherapeutic agents and/or anti-HER2 agents.

In one aspect, the methods of treatment herein can be used advantageously in an individual whose tumor or cancer has resistance, that is not responsive to or that has progressed following treatment with a composition comprising an anti-HER2 antibody (e.g. trastuzumab; an ADC comprising traztuzumab) or a composition comprising another anti-Nectin-4 agent (e.g. an ADC comprising enfortumab; enfortumab vedotin).

In one aspect of the embodiment herein, the individual has received prior treatment with radiotherapy, surgery, chemotherapy, and/or therapy with a biological agent.

In one aspect of the embodiment herein, the individual has received prior treatment with an anti-Nectin-4 agent, optionally an anti-Nectin-4 agent comprising a cytotoxic moiety, other than a topoisomerase inhibitor, e.g. enfortumab vedotin.

In one aspect of the embodiment herein, provided is method of treating a cancer, killing tumor cells and/or delivering a cytotoxic agent to a tumor, in an individual in need thereof, comprising administering to an individual who has received prior treatment with a Nectin-4 binding agent conjugated to an auristatin, optionally enfortumab vedotin, a therapeutically effective amount of an immunoconjugate that binds a human Nectin-4 polypeptide, wherein the immunoconjugate is represented by any one of the Formulae:

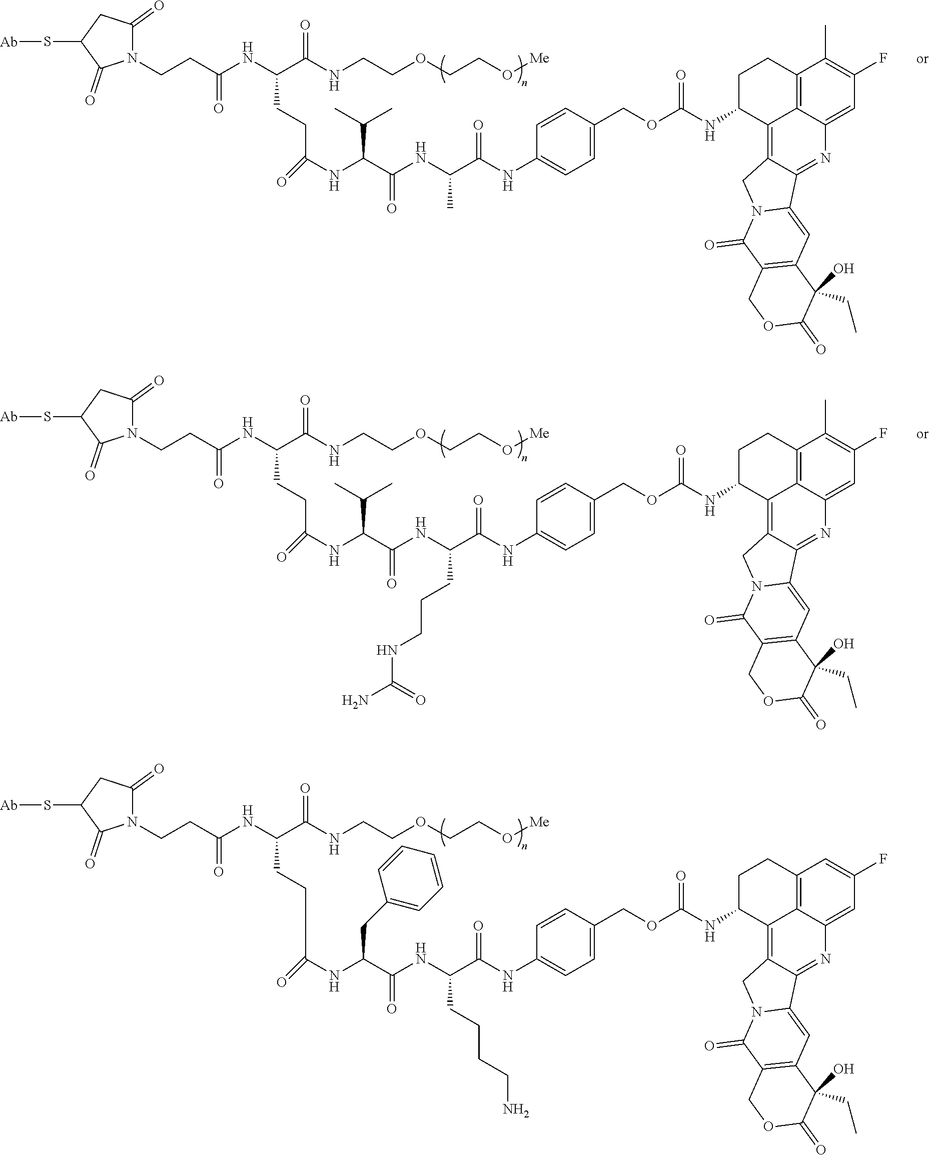

wherein n is 5-23, optionally n is 7-15, optionally n is 15, and wherein Ab is an antibody or antibody fragment thereof that specifically binds a human Nectin-4 polypeptide. Optionally, the immunoconjugate is characterized by a DAR of between 6 and 8, optionally wherein the DAR is 6, optionally wherein the DAR is 8.

In one aspect, the present invention provides methods of treatment that can be used to mediate an anti-tumor effect in an individual at doses that are low or lower than those employed for conventional anti-Nectin-4 ADCs, e.g. less than 3 mg/kg body weight, less than 1.25 mg/kg body weight, less than 1 mg/kg body weight, less than 125 mg flat dose.

In one aspect, the methods of treatment of the disclosure can be used in an individual who has existing neuropathy, diabetes or hyperglycemia, cardiac insufficiency, an ocular pathology.

In one aspect, the methods of treatment of the disclosure can be used in an individual having a Nectin-4-expressing cancer characterized by low or moderate levels of tumor cell expression of Nectin-4 polypeptides (e.g. expression of Nectin-4 polypeptides at the tumor cell membrane).

In any embodiment herein, the anti-Nectin-4 antibody conjugated to a cytotoxic agent is used in combination with a further cytotoxic agent (e.g. chemotherapeutic agent, a chemotherapeutic agent transported by P-glycoprotein (Pgp, the product of the human MDR1 gene), a platinum agent, a taxane), wherein the further cytotoxic agent is administered separately from the anti-Nectin-4 antibody conjugated to a cytotoxic agent.

In another aspect of the invention, provided is pharmaceutical compositions and kits comprising the anti-Nectin-4 antibody or, antibody fragment, optionally conjugated with a cytotoxic drug moiety, and typically one or more additional ingredients that can be active ingredients or inactive ingredients that promote formulation, delivery, stability, or other characteristics of the composition (e.g. various carriers).

These aspects are more fully described in, and additional aspects, features, and advantages will be apparent from, the description of the invention provided herein.

### DESCRIPTION OF THE DRAWINGS

FIG. 1 shows expression levels of HER2 and Nectin-4 polypeptides at the surface of SUM190 human breast cancer tumor cells, as determined by FACS (MFI: Mean of fluorescence intensity). The SUM190 tumor cells expressed HER2 at low to moderate levels (median fluorescence units 1777) as well as Nectin-4 at lower levels (median 991 fluorescence units).

FIG. 2 shows expression levels of HER2 and Nectin-4 polypeptides at the surface of SUM185 human breast cancer tumor cells as determined by FACS (MFI: Mean of fluorescence intensity). The SUM185 cells expressed HER2 at moderate to high levels (median fluorescence units 2880) as well as Nectin-4 at higher levels (median 4326 fluorescence units).

FIG. 3A shows killing of human breast cancer cells by the 5E7 antibody conjugated with the camptothecin analogues Dxd (via a GGFG-Dxd linker shown in Example 9) or exatecan (via the (PEG (8U)-Val-Ala-PAB-Exatecan) linker shown in Example 9), along with isotype control antibodies (IC), all at equivalent drug to antibody ratios (DAR=8), and compared to compared to V-domain binding Enhertu™ (trastuzumab deruxtecan (anti-HER2)). FIG. 3B shows efficacy of “5E7-exatecan” (5E7 conjugated to the exatecan linker (PEG (8U)-Val-Ala-PAB-Exatecan) shown in Example 9) as in causing the death of HER-2 and Nectin-4 expressing SUM185, SUM190, MDA-MB-468 (TNBC) human tumor cells and MC38 (colon cancer) and B16F10 (melanoma) murine tumor cells as well as EC50 values for ability of 5E7-exatecan to cause cell death.

FIG. 4 shows the efficacy at a single dose of 3 mg/kg of 5E7 antibody as a camptothecin ADC, in comparison to the same dose of enfortumab, N41 and an isotype control antibody (IC), in a mouse model of human breast cancer. Each antibody was conjugated with the same camptothecin analogue (Dxd) and tetrapeptide-containing linker (GGFG) at equivalent drug to antibody ratios (DAR=8). Only 5E7 was able to effectively control tumor growth.

FIG. 5 shows killing of SUM185 human breast cancer cells that express Nectin-4 at relatively high levels by the 5E7 antibody, compared to Padcev™ (enfortumab vedotin) (enfortumab conjugated to the auristatin compound MMAE at DAR=4) and Enhertu™. This setting is used as a model of anti-HER2 resistance.

FIGS. 6A and 6B show binding of anti-Nectin-4 antibody 5E7 on rat and cynomolgus Nectin-4 expressing CHO cell lines respectively, as determined by flow cytometry.

FIGS. 7A and 7B show the structure of the human Nectin-4 protein, with shading of substitutions; the white areas corresponding to the C1 domain residues substituted in mutants 7 (7A) and 7bis (7B) are indicated.

FIGS. 8A and 8B show several views of the structure of the human Nectin-4 protein, with shading of substitutions; the white areas corresponding to residues substituted in mutants 1, 2, 3, 4, 5, 6, 7, 8 and 9 are indicated.

FIGS. 9A, 9B and 9C show the evolution of tumor growth over time (days post tumor engraftment) in mice treated with ADCs. FIGS. 9A and 9B show treatment with a dose of 3 mg/kg body weight by i.v. of different anti-nectin-4 antibodies (5E7 or 6A7) conjugated to the same linker-payload at the same DAR. FIG. 9C shows treatment at a dose of 10 mg/kg body weight by i.v. with antibody 5E7 conjugated to linkers designed release either Dxd or exatecan upon cleavage of the linker.

FIG. 10A shows luminescence (indicating cell viability) of cells treated with Padcev™ (enfortumab vedotin), antibody 5E7 conjugated to Dxd or 5E7 conjugated to exatecan. The cells are MC-38 cells that endogenously express MDR1 p-glycoprotein and that are engineered to express Nectin-4. The ADC with exatecan as payload was highly potent to decrease cell viability in this setting of drug resistance. FIG. 10B shows tumor growth (area under the curve) of the MC38 cells treated, in the presence or absence of the Pgp inhibitor cyclosporine, with Padcev™ (enfortumab vedotin), antibody 5E7 conjugated to Dxd or 5E7 conjugated to exatecan, at 150 nM ADC and normalized to control antibody, suggesting that the anti-tumor activity of Padcev™ and antibody 5E7 conjugated to Dxd are negatively affected by Pgp.

FIGS. 11 and 12 show results of in vivo evaluation in mice of free toxin and ADCs at 3 mg/kg dose. Results for the IC (free toxins) are shown in FIG. 11. Results for the ADCs are shown in FIG. 12.

FIG. 13 shows results in vivo anti-tumor efficacy in mice of anti-Nectin-4 ADCs and plasma concentration of ADCs over time. The top left panel shows that PBS did not prevent an increase in tumor volume. Top right panel shows that the 1 mg/kg dose of ADC showed strong anti-tumor efficacy. The bottom panel shows the concentration of ADC in plasma over time.

FIG. 14 shows the in vivo plasma concentration of ADCs in rats. FIG. 14A shows 3 mg/kg dose (top panel) and 10 mg/kg dose (bottom panel) and FIG. 14B shows the results for the 30 mg/kg dose.

FIG. 15 shows the in vivo plasma concentration of ADCs in non-human primates.

FIG. 15A shows 3 mg/kg dose (top panel) and 10 mg/kg dose (bottom panel) and FIG. 15B shows the results for the 30 mg/kg dose.

Table 15 shows the nucleic acid and amino acid sequences disclosed in the application.

### DETAILED DESCRIPTION

### Definitions

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of” or by “consisting of”.

“Nectin-4” and “Nectin-4 polypeptide” refer to a protein or polypeptide encoded by the NECTIN4 gene (see Uniprot accession number Q96NY8) or by a cDNA prepared from such a gene. Any naturally occurring isoform, allele or variant is encompassed by the term Nectin-4 polypeptide (e.g., an Nectin-4 polypeptide 95%, 98% or 99% identical to SEQ ID NO: 1, or to a contiguous sequence of at least 100, 200, 300, 400 or 500 amino acid residues thereof). The 510 amino acid residue sequence of canonical human Nectin-4 (isoform 1), including the 31 amino acid signal peptide, is shown as follows:

(SEQ ID NO: 1)
MPLSLGAEMW GPEAWLLLLL LLASFTGRCP AGELETSDVV
TVVLGQDAKL PCFYRGDSGE QVGQVAWARV DAGEGAQELA
LLHSKYGLHV SPAYEGRVEQ PPPPRNPLDG SVLLRNAVQA
DEGEYECRVS TFPAGSFQAR LRLRVLVPPL PSLNPGPALE
EGQGLTLAAS CTAEGSPAPS VTWDTEVKGT TSSRSFKHSR
SAAVTSEFHL VPSRSMNGQP LTCVVSHPGL LQDQRITHIL
HVSFLAEASV RGLEDQNLWH IGREGAMLKC LSEGQPPPSY
NWTRLDGPLP SGVRVDGDTL GFPPLTTEHS GIYVCHVSNE
FSSRDSQVTV DVLDPQEDSG KQVDLVSASV VVVGVIAALL
FCLLVVVVVL MSRYHRRKAQ QMTQKYEEEL TLTRENSIRR
LHSHHTDPRS QPEESVGLRA EGHPDSLKDN SSCSVMSEEP
EGRSYSTLTT VREIETQTEL LSPGSGRAEE EEDQDEGIKQ
AMNHFVQENG TLRAKPTGNG IYINGRGHLV.

SEQ ID NO: 1 corresponds to UniProt KB identifier Q96NY8-1, the disclosure of which is incorporated herein by reference.

Certain aspects of the present disclosure provide anti-Nectin-4 antibodies that bind to a human Nectin-4, or a homolog thereof, including without limitation a mammalian Nectin-4 protein and Nectin-4 orthologs from other species, e.g. non-human primates, Macaca fascicularis.

The term “HER2” (also known as HER2/neu and ErbB-2) stands for “Human Epidermal growth factor Receptor 2”. It includes variants and isoforms of HER2.

The terms “immunoconjugate” and “antibody conjugate” are used interchangeably and refer to an antigen binding agent, e.g., an antibody binding polypeptide or an antibody that is conjugated to another molecule (e.g., a camptothecin derivative, an exatecan molecule, a SN-38 molecule). When an immunoconjugate comprises an antigen binding agent conjugated to a therapeutic agent, e.g., a cytotoxic agent or anti-cancer agent, the immunoconjugate can also be referred to as a “antibody drug conjugate” or an “ADC”. Examples of cytotoxic agents include camptothecins, taxanes, anthracyclines, camptothecins, epothilones, mytomycins, combretastatins, vinca alkaloids, nitrogen mustards, maytansinoids, calicheamycins, duocarmycins, tubulysins, dolastatins and auristatins, enediynes, pyrrolobenzodiazepines, and ethylenimines.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it such as a preventive early asymptomatic intervention; (b) inhibiting the disease, e.g., arresting its development; or relieving the disease, e.g., causing regression of the disease and/or its symptoms or conditions such as improvement or remediation of damage, for example in a subject who has been diagnosed as having the disease. Optionally, treatment may cause (e.g. may be characterized as a method of causing) a decrease in tumor burden, a decrease in the size and/or number of lesions, a decrease or delay in the progression of cancer (e.g., an increase in progression-free survival), a delay or prevention of cancer metastasis and/or an increase in survival. Optionally, treatment may cause or provide (e.g. may be characterized as a method of causing or providing) stable disease, a partial response or a complete response in a subject, e.g. according to standard criteria, optionally RECIST criteria.

Whenever “treatment of cancer” or the like is mentioned with reference to a Nectin-4 binding agent (e.g. an antibody or antibody fragment, an immunoconjugate), this can include:
(a) a method of treatment of cancer, said method comprising the step of administering (for at least one treatment) a Nectin-4 binding agent to an individual, a mammal, especially a human, in need of such treatment, in a dose that allows for the treatment of cancer, (a therapeutically effective amount), optionally in a dose (amount) as specified herein;
(b) the use of a Nectin-4 binding agent for the treatment of cancer;
(c) the Nectin-4 binding agent, for use in the treatment of cancer (especially in a human);
(d) the use of a Nectin-4 binding agent for the manufacture of a pharmaceutical preparation for the treatment of cancer;
(e) a method of using a Nectin-4 binding agent for the manufacture of a pharmaceutical preparation for the treatment of cancer, comprising admixing a Nectin-4 binding agent with a pharmaceutically acceptable carrier;
(f) a pharmaceutical preparation comprising an effective dose of a Nectin-4 binding agent that is appropriate for the treatment of cancer;
(g) any combination of (a), (b), (c), (d), (e) and (f), in accordance with the subject matter allowable for patenting in a country where this application is filed.

The term “biopsy” as used herein is defined as removal of a tissue for the purpose of examination, such as to establish diagnosis. Examples of types of biopsies include by application of suction, such as through a needle attached to a syringe; by instrumental removal of a fragment of tissue; by removal with appropriate instruments through an endoscope; by surgical excision, such as of the whole lesion; and the like.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The amino acid residues of an antibody that are responsible for antigen binding can also be referred to hypervariable region. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “specifically binds to” means that an antibody can bind preferably in a competitive binding assay to the binding partner, e.g. Nectin-4, as assessed using either recombinant forms of the proteins, epitopes therein, or native proteins present on the surface of isolated target cells. Competitive binding assays and other methods for determining specific binding are well-known in the art. For example, binding can be detected via radiolabels, physical methods such as mass spectrometry, or direct or indirect fluorescent labels detected using, e.g., cytofluorometric analysis (e.g. FACScan). Binding above the amount seen with a control, non-specific agent indicates that the agent binds to the target.

When an antibody is said to “compete with” a particular monoclonal antibody, it means that the antibody competes with the monoclonal antibody in a binding assay using either recombinant molecules (e.g., Nectin-4) or surface expressed molecules (e.g., Nectin-4). For example, if a test antibody reduces the binding of an antibody having a heavy chain variable region of any of SEQ ID NO: 19 and a respective light chain variable region of SEQ ID NO: 20 to a Nectin-4 polypeptide or Nectin-4-expressing cell in a binding assay, the antibody is said to “compete” respectively with such antibody.

The term “internalization”, used interchangeably with “intracellular internalization”, refers to the molecular, biochemical and cellular events associated with the process of translocating a molecule from the extracellular surface of a cell to the intracellular surface of a cell. The processes responsible for intracellular internalization of molecules are well-known and can involve, inter alia, the internalization of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule). Thus, “inducing and/or increasing internalization” comprises events wherein intracellular internalization is initiated and/or the rate and/or extent of intracellular internalization is increased.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of monoclonal antibodies can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of monoclonal antibodies is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “agent” is used herein to denote a chemical compound, a mixture of chemical compounds, a biological macromolecule, or an extract made from biological materials. The term “therapeutic agent” refers to an agent that has biological activity.

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD).

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

As used herein, “alkyl” refers to a straight or branched hydrocarbon chain that comprises a fully saturated (no double or triple bonds) hydrocarbon group. The alkyl group may have, for example, 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, the term “heteroalkyl” refers to a straight or branched alkyl group that contains one or more heteroatoms, that is, an element other than carbon (including but not limited to oxygen, sulfur, nitrogen, phosphorus) in place of one or more carbon atoms.

Whenever a group is described as being “substituted” that group substituted with one or more of the indicated substituents. If no substituents are indicated, it is meant that the indicated “substituted” group may be substituted with one or more group(s) individually and independently selected from alkyl, alkenyl, alkynyl, cycloalkyl, cycloalkenyl, cycloalkynyl, heteroalkyl, aryl, heteroaryl, heteroalicyclyl, aralkyl, heteroaralkyl, (heteroalicyclyl)alkyl, hydroxy, alkoxy, aryloxy, acyl, mercapto, alkylthio, arylthio, cyano, halogen, thiocarbonyl, carbamyl, thiocarbamyl, amido, sulfonamido, sulfonamido, carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group, and protected derivatives thereof.

Where the number of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

Reference to a “Compound” or “Formula” having a particular number (e.g. “Compound 1”, “Compound 2”, “Formula I” or “Formula II”), unless the context clearly indicates otherwise, designates all compounds derived from the Compound or Formula having the particular number. Compound 1, for example includes reference to Compound 1a and 1b.

### Producing Antibodies and Antibody Fragments

The hypervariable regions, heavy and light chain CDRs, heavy and light chain variable regions, and proteins e.g., full-length antibodies or antibody fragments, multispecific antigen-binding proteins, chimeric antigen receptors, that comprise them, will bind human Nectin-4 expressed on the surface of a cell, e.g., a tumor cell. In one embodiment, Nectin-4-binding by the antibody is mediated by a single antigen binding domain or by two identical antigen binding domains, optionally wherein each antigen binding domain comprises a VH and VL domain pair. In some embodiments, provided are a single VH or VL variable domains; such domains can for example be incorporated into polypeptides that are expressed and produced separately, and then can be combined to form an Nectin-4 binding domain.

When used in therapy for the elimination of Nectin-4-expressing tumor cells, either as an immunoconjugate or as non-conjugated (“naked”) antibody (e.g. optionally in combination with a cytotoxic agent administered separately), the Nectin-4 binding antibody (e.g. full-length antibody, antibody fragment) or a protein, conjugate or complex comprising such antibody may advantageously inhibit cell-cell interactions mediated by Nectin-4, e.g., as determined by assessing cell cluster formation (e.g., in a 3-dimensional cell culture system, by assessing tumor spheroid formation or growth) and/or anchorage-independent growth of Nectin-4 expressing cells. When used in therapy for the elimination of Nectin-4-expressing tumor cells, either as an immunoconjugate with a cytotoxic agent or as non-conjugated (“naked”) antibody in combination with a cytotoxic agent administered separately, the Nectin-4 binding antibody may advantageously be capable of sensitizing tumors to the cytotoxic agent, for example the antibody may enhance the ability of the cytotoxic agent to inhibit the proliferation or cause the death of tumor cells, for example to inhibit the proliferation or cause the death of tumor cells that are in clusters (e.g. in spheroids), or to inhibit the formation or growth of tumor cell clusters (e.g. spheroids). When used in therapy for the elimination of Nectin-4-expressing tumor cells as an immunoconjugate, the anti-Nectin-4 immunoconjugate may advantageously be capable of causing the death of Nectin-4-expressing tumor cells when conjugated to a cytotoxic molecule as disclosed herein.

Tumor cells that express Pgp can have decreased sensitivity to certain cytotoxic agents. Antibodies and cytotoxic agents (whether or not the cytotoxic agent is conjugated to the antibody) can optionally be tested using cells (e.g. tumor cells) that express Pgp.

Tumor spheroids are generally less sensitive to chemotherapy, in part due to the protection afforded by their structure, but also due to their slower proliferation rate, and consequently can be useful to assess the anti-tumor effect of the antibodies or immunoconjugates. Antibodies or immunoconjugates can be tested for the ability to inhibit spheroid formation in a concentration-dependent manner, or to disrupt cancer cells spheroid formation. Antibodies or immunoconjugates can for example be tested for their ability to alter spheroid formation in different cancer cell lines using real-time digital photography.

The antibodies or immunoconjugates can for example be characterized as being able to maintain (or increase maintenance of) the cancer cells as single cells, which cells may thereby be made more sensitive to a cytotoxic agent (e.g. a chemotherapy). Promoting maintenance of cancer cells as single cells to increase sensitivity to chemotherapy can be tested in vitro by adding different chemotherapeutic agents (e.g., doxorubicine, cisplatin, paclitaxel, camptothecin or a camptothecin analog) separately or in combination at different concentrations in the presence of antibodies or immunoconjugates, optionally further with comparison to antibodies or immunoconjugates that bind solely to the Ig-like V domain or the Ig-like C2-type 1 or 2 domains. Maintenance of cancer cells as single cells to increase sensitivity to chemotherapy can for example also be tested in vitro by adding different immunoconjugates targeting different domains of Nectin-4 with different toxins, where IC50 of chemotherapies or ADCs will be lower where the antibody binds to the VC1 domain of Nectin-4, or the dose used to control tumor growth will be lower for the ADCs wherein the antibody bind the VC1 domain of Nectin-4. Sensitivity to chemotherapy can be assessed as cell viability, cell proliferation or cytotoxicity, for example monitored using different read-outs such as CTG by luminescence, confluence using Incucyte or caspase 3/7 or Annexin V. In one embodiment, an anti-Nectin-4 antibody or antibody fragment binds to a wild-type human Nectin-4 polypeptide, e.g. a polypeptide having the amino acid sequence of SEQ ID NO: 1, as well to a modified human Nectin-4 polypeptide having the amino acid sequence of SEQ ID NO: 10 (Nectin-4 protein containing the Ig-like C2 type 1 and 2 domains but lacking the Ig-like V type domain. Optionally, the antibody retains partial binding to the polypeptide lacking the Ig-like V type domain, optionally wherein the antibody retains binding to the Nectin-4 protein lacking the Ig-like V type domain at a reduced level compared to binding to a wild-type Nectin-4 protein, for example wherein the reduced level is between 5-50%, optionally 5-30%, optionally 5-25%, optionally 5-15% of binding to wild-type Nectin-4. Optionally further the anti-Nectin-4 antibody does not bind a Nectin-4 polypeptide lacking both the Ig-like V type and the Ig-like C2 type 1 domains, e.g., a polypeptide having the amino acid sequence of SEQ ID NO: 11. Optionally, binding is assessed by flow cytometry using cells made to express the Nectin-4 polypeptide and fluorescence intensity level (e.g. MFI) is determined.

In one embodiment, the anti-Nectin-4 antibody or antibody fragment does not bind to any of a human Nectin 1 protein, a human Nectin 2 protein, a human Nectin 3 protein and a human PVR protein. The respective Nectin or PVR protein can be specified as being expressed at the surface of a cell.

In one embodiment, an anti-Nectin-4 antibody or antibody fragment binds:
a Nectin-4 polypeptide having the amino acid sequence of SEQ ID NO: 1 and
a Nectin-4 polypeptide having the amino acid sequence of SEQ ID NO: 10 (optionally at a reduced level, optionally a level between 5-50% compared to binding to SEQ ID NO: 1), wherein the anti-Nectin-4 antibody or antibody fragment does not bind any of:
a polypeptide having the amino acid sequence of SEQ ID NO: 13,
a polypeptide having the amino acid sequence of SEQ ID NO: 16,
a polypeptide having the amino acid sequence of SEQ ID NO: 17, and
a polypeptide having the amino acid sequence of SEQ ID NO: 18.

Optionally further, the anti-Nectin-4 antibody or antibody fragment does not bind a polypeptide having the amino acid sequence of SEQ ID NO: 11.

Optionally further, the anti-Nectin-4 antibody or antibody fragment binds a rat Nectin-4 polypeptide (e.g. the amino acid sequence of SEQ ID NO: 13).

In one embodiment, an anti-Nectin-4 antibody or antibody fragment binds to the VC1 bridging domain, epitope or determinant of Nectin-4.

In one embodiment, an anti-Nectin-4 antibody or antibody fragment binds to human Nectin-4 at 1, 2, 3, 4 or 5 or more residues selected from the group consisting of A72, G73, S195, K197, S199, L150, S152, Q234 and 1236 (with reference to SEQ ID NO: 1). In one embodiment, an anti-Nectin-4 antigen binding protein or antibody binds to human Nectin-4 at 1, 2 or 3 residues selected from the group consisting of K197, S199 and Q234, optionally residues K197 and/or S199. In one embodiment, an anti-Nectin-4 antibody or antibody fragment binds to residue K197 of human Nectin-4. In one embodiment, an anti-Nectin-4 antibody or antibody fragment binds to residue S199 of human Nectin-4. In one embodiment, an anti-Nectin-4 antibody or antibody fragment binds to residue Q234 of human Nectin-4. Binding can be determined, for example, by assessing whether the antibody or antibody fragment has decreased or loss of binding to a mutant Nectin-4 polypeptide (e.g. as expressed at the surface of a cell) in which said residue is substituted by a different residue.

In one embodiment, an anti-Nectin-4 antibody or antibody fragment is capable of binding to both the Ig-like V type domain and the Ig-like C2 type 1 domain, for example to a determinant or epitope on the Ig-like V type domain and to a determinant or epitope on the Ig-like C2 type 1 domain.

In one embodiment, an anti-Nectin-4 antibody or antibody fragment retains at least partial binding to a modified Nectin-4 polypeptide comprising the Ig-like C2 type 1 domain but lacking the Ig-like V type domain, e.g. a Nectin-4 polypeptide lacking all or part of the amino acid sequence of SEQ ID NO: 3. Partial binding can be characterized as retaining binding, but at a reduced level (e.g. as assessed by flow cytometry) compared to binding to a wild-type human Nectin-4 polypeptide having the amino acid sequence of residue of SEQ ID NO: 1. The Nectin-4 polypeptides can be specified as being expressed at the surface of a cell (e.g., a host cell made to express the polypeptide(s).

Antibodies may be produced by a variety of techniques known in the art. Typically, they are produced by immunization of a non-human animal, preferably a mouse, with an immunogen comprising a Nectin-4 polypeptide, preferably a human Nectin-4 polypeptide. The Nectin-4 polypeptide may comprise the full length sequence of a human Nectin-4 polypeptide, or a fragment or derivative thereof, typically an immunogenic fragment, i.e., a portion of the polypeptide comprising an epitope exposed on the surface of cells expressing a Nectin-4 polypeptide, for example the epitope recognized by the 5E7 antibody. The Nectin-4 polypeptide can be specified as comprising or consisting of VC1 bridging domain or a fragment or subsequence thereof. Such fragments typically contain at least about 7 consecutive amino acids of the mature polypeptide sequence, even more preferably at least about 10 consecutive amino acids thereof. Fragments typically are essentially derived from the extra-cellular domain of the receptor. In one embodiment, the immunogen comprises a wild-type human Nectin-4 polypeptide in a lipid membrane, typically at the surface of a cell. In a specific embodiment, the immunogen comprises intact cells, particularly intact human cells, optionally treated or lysed. In another preferred embodiment, the polypeptide is a recombinant Nectin-4 polypeptide. In a specific embodiment, the immunogen comprises intact Nectin-4-expressing cells.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, NY (1988), the entire disclosure of which is herein incorporated by reference).

Antibodies may also be produced by selection of combinatorial libraries of immunoglobulins, as disclosed for instance in (Ward et al. Nature, 341 (1989) p. 544, the entire disclosure of which is herein incorporated by reference).

Provided herein are modified human acceptor framework sequences into which antibody CDRs can be incorporated such that the resulting anti-Nectin-4 variable region has high potency in binding and internalizing in Nectin-4 expressing cells. The antibodies have the advantage of having low or decreased immunogenicity in humans, e.g. having lower ability or likelihood to cause an unwanted anti-Nectin-4 antibody directed immune response upon administration to a human. Examples of such antibodies of the disclosure include antibodies comprising the H0, H1, H2, H3, H4, H5, H6, H7, H8, H9 or H10 VH domain combined with a L0, L1, L2 or L3 VL domain. Examples of antibodies of the disclosure include antibodies comprising the VH and VL domain pairs of any one of the antibodies H0+L0, H1+L0, H2+L0, H3+L0, H3+L1, H3+L2, H3+L3, H4+L1, H4+L2, H4+L3, H5+L1, H5+L2, H5+L3, H6+L1, H6+L2, H6+L3, H7+L1, H7+L2, H7+L3, H8+L1, H9+L1 and H10+L1. In one aspect, an antibody or antibody fragment that binds a human Nectin-4 polypeptide comprises VH and VL frameworks (e.g., FR1, FR2, FR3 and FR4) of human origin (e.g., derived from human amino acid sequences). In one aspect, the antibody or antibody fragment comprises: a HCDR1 (heavy chain CDR1) comprising an amino acid sequence SYWMH as set forth in SEQ ID NO: 21; a HCDR2 (heavy chain CDR2) comprising an amino acid sequence EIDPSDSYTNYNQKFKG as set forth in SEQ ID NO: 22; a HCDR3 (heavy chain CDR3) comprising an amino acid sequence GYGNYGDY as set forth in SEQ ID NO: 23; a LCDR1 comprising an amino acid sequence RSSKSLLHSNGITYLY as set forth in SEQ ID NO: 24; a LCDR2 comprising an amino acid sequence QMSNLAS as set forth in SEQ ID NO: 25; a LCDR3 comprising an amino acid sequence AQNLELPWT as set forth in SEQ ID NO: 26.

In one embodiment, the antibody comprises a heavy chain framework derived from the human subgroup IGHV1-46 (optionally together with IGHJ4, preferably IGHJ4*01), optionally the IGHV1-46 is IGHV1-46*01. In one embodiment, the antibody comprises a light chain framework derived from the human subgroup IGKV2-28 (optionally together with IGKJ, preferably IGKJ4*01), optionally the IGKV2-28 is IGKV2-28*01.

The antibody may further comprise one, two, three, four, five or more amino acid substitutions across the human heavy and/or light chain frameworks, to, e.g., enhance affinity, stability, or other properties of the antibody.

Optionally, in any embodiment, the antibody can be specified as being an antibody other than a murine parental antibody, e.g. a murine parental antibody having the respective VH and VL of SEQ ID NO: 19 and 20.

Optionally, a human framework comprises one or more mutations, e.g., back mutations to introduce a residue present at the particular position in a non-human mammal (e.g., a mouse). Optionally, the human framework of the heavy chain variable region of the antibody comprises one, two, three, four, five, six, seven or eight mutations at a kabat position selected from 28, 38, 40, 48, 69, 71, 73 or 78.

In one aspect of any embodiment herein, the threonine residue at Kabat heavy chain position 28 is substituted by a threonine residue.

In one aspect of any embodiment herein, the arginine residue at Kabat heavy chain position 38 is substituted by a lysine residue.

In one aspect of any embodiment herein, the alanine residue at Kabat heavy chain position 40 is substituted by an arginine residue.

In one aspect of any embodiment herein, the methionine residue at Kabat heavy chain position 48 is substituted by an isoleucine residue.

In one aspect of any embodiment herein, the methionine residue at Kabat heavy chain position 69 is substituted by a leucine residue.

In one aspect of any embodiment herein, the arginine residue at Kabat heavy chain position 71 is substituted by a leucine residue.

In one aspect of any embodiment herein, the threonine residue at Kabat heavy chain position 73 is substituted by a lysine residue.

In one aspect of any embodiment herein, the valine residue at Kabat heavy chain position 78 is substituted by a threonine residue.

Optionally, the human framework of the light chain variable region of the antibody comprises one, two, three or four mutations at a kabat position selected from 2, 8, 11 or 64. In one aspect of any embodiment herein, the isoleucine residue at kabat light chain position 2 is substituted by a valine residue.

In one aspect of any embodiment herein, the proline residue at Kabat light chain position 8 is substituted by an alanine residue.

In one aspect of any embodiment herein, the leucine residue at Kabat light chain position 11 is substituted by an asparagine.

In one aspect of any embodiment herein, the glycine residue at Kabat light chain position 64 is substituted by a serine residue.

Positions in the VH and VL domains herein are described using the Kabat numbering system (Kabat et al. (1991) Sequences of Protein of Immunological Interest, 5th ed., United States Public Health Service, National Institute of Health, Bethesda, MD).

In one aspect, the isolated anti-Nectin-4 humanized monoclonal antibody, or fragment thereof, comprises a heavy chain variable region having at least about 80% sequence identity (e.g., at least about 85%, 90%, 95%, 97%, 98% or 99% identity, or 100% identity) to the amino acid sequences of any of SEQ ID NO: 37, 39, 41, 43, 45, 47, 49, 51, 53, 55 or 57 and a light chain variable region having at least about 80% sequence identity (e.g., at least about 85%, 90%, 95%, 97%, 98% or 99% identity, or 100% identity) to the amino acid sequence of any of SEQ ID NO: 59, 61, 63 or 65.

In one aspect, the isolated anti-Nectin-4 humanized monoclonal antibody, or fragment thereof, comprises a heavy chain variable region having at least about 80% sequence identity (e.g., at least about 85%, 90%, 95%, 97%, 98% or 99% identity, or 100% identity) to the amino acid sequence of SEQ ID NO: 47 (H5) and a light chain variable region comprising the amino acid sequence of SEQ ID NO: 65 (L3).

In one embodiment, provided is an anti-Nectin-4 antibody, comprising a heavy chain having at least about 80% sequence identity (e.g., at least about 85%, 90%, 95%, 97%, 98% or 99% identity, or 100% identity) to a heavy chain comprising the amino acid sequence of SEQ ID NO: 77; and a light chain having at least about 80% sequence identity (e.g., at least about 85%, 90%, 95%, 97%, 98% or 99% identity, or 100% identity) to a light chain comprising the amino acid sequence of SEQ ID NO: 78.

DNA encoding an antibody can be prepared and placed in an appropriate expression vector for transfection into an appropriate host. The host is then used for the recombinant production of the antibody, or variants thereof, such as a humanized version of that monoclonal antibody, active fragments of the antibody, chimeric antibodies comprising the antigen recognition portion of the antibody, or versions comprising a detectable moiety.

DNA encoding the monoclonal antibodies of the disclosure can be readily isolated and sequenced using conventional procedures (e.g., by using oligonucleotide probes that are capable of binding specifically to genes encoding the heavy and light chains of murine antibodies). In one aspect, provided is a nucleic acid encoding a heavy chain or a light chain of an anti-Nectin-4 antibody or antibody fragment of any embodiment herein. Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody. In one embodiment, provided is an isolated nucleic acid sequence encoding a light chain and/or a heavy chain of an antibody, as well as a recombinant host cell comprising (e.g., in its genome) such nucleic acid. Recombinant expression in bacteria of DNA encoding the antibody is well known in the art (see, for example, Skerra et al., Curr. Opinion in Immunol., 5, pp. 256 (1993); and Pluckthun, Immunol. 130, p. 151 (1992).

Fragments and derivatives of antibodies (which are encompassed by the term “antibody” or “antibodies” as used in this application, unless otherwise stated or clearly contradicted by context) can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”); and multispecific (e.g., bispecific) antibodies formed from antibody fragments. For example, a multispecific protein can comprise a hypervariable region (e.g., a VH and a VL) of an antibody of any of the embodiment herein and a hypervariable region (e.g., a VH and a VL) of a different antibody, for example an antibody that binds to an antigen of interest other than Nectin-4.

Typically, an anti-Nectin-4 antibody provided herein has an affinity for a Nectin-4 polypeptide (e.g., a Nectin-4 polypeptide as produced in the Examples herein) in the range of about 10⁴ to about 10¹¹ M⁻¹ (e.g., about 10⁷ to about 10¹⁰ M⁻¹). For example, in a particular aspect the disclosure provides anti-Nectin-4 antibody that have an average disassociation constant (K_{D}) of less than 1×10⁻⁷ M with respect to Nectin-4, as determined by, e.g., surface plasmon resonance (SPR) screening (such as by analysis with a BIAcore™ SPR analytical device). In a more particular exemplary aspect, the disclosure provides anti-Nectin-4 antibodies that have a K_{D} of about 1×10⁻⁷ M to about 1×10⁻¹⁰ M for Nectin-4.

Antibodies can be characterized for example by a mean KD of no more than about (i.e. better affinity than) 200, 150, 100, 80, 70, 60, 40, 30, or 25 nanomolar. KD can be determined for example for example by immobilizing recombinantly produced human Nectin-4 proteins on a chip surface, followed by application of the antibody to be tested in solution. In one embodiment, the method further comprises a step (d), selecting antibodies from (b) that are capable of competing for binding to Nectin-4 with control antibody.

The anti-Nectin-4 antibodies may or may not have substantial binding to human Fcγ receptors. In one embodiment, an anti-Nectin-4 antibody comprises an Fc domain of human IgG1 isotype that retains binding to human Fcγ receptor. In one embodiment, an anti-Nectin-4 antibody comprises an Fc domain of human IgG1 isotype that is modified (e.g. by introduction of substitutions) to increase binding to human Fcγ receptor, e.g. CD16A. In another embodiment, the anti-Nectin-4 antibodies can be prepared such that they do not have substantial binding to human Fcγ receptors, e.g., any one or more of CD16A, CD16B, CD32A, CD32B and/or CD64). Such antibodies may comprise constant regions of various heavy chains that are known to lack or have low binding to Fcγ receptors. Alternatively, antibody fragments that do not comprise (or comprise portions of) constant regions, such as F(ab′)2 fragments, can be used to avoid Fc receptor binding. Fc receptor binding can be assessed according to methods known in the art, including for example testing binding of an antibody to Fc receptor protein in a BIACORE assay. Also, generally any antibody IgG isotype can be used in which the Fc portion is modified (e.g., by introducing 1, 2, 3, 4, 5 or more amino acid substitutions) to minimize or eliminate binding to Fc receptors (see, e.g., WO 03/101485, the disclosure of which is herein incorporated by reference). Assays such to assess Fc receptor binding are well known in the art, and are described in, e.g., WO 03/101485. Examples of silent Fc IgG1 antibodies are the LALA mutant comprising L234A and L235A mutation in the IgG1 Fc amino acid sequence. Another example of an Fc silent mutation is a mutation at residue D265, or at D265 and P329 for example as used in an IgG1 antibody as the DAPA (D265A, P329A) mutation (U.S. Pat. No. 6,737,056). Another silent IgG1 antibody comprises a mutation at residue N297 (e.g., N297A, N297S mutation), which results in aglycosylated/non-glycosylated antibodies. Other silent mutations include: substitutions at residues L234 and G237 (L234A/G237A); substitutions at residues S228, L235 and R409 (S228P/L235E/R409K, T,M,L); substitutions at residues H268, V309, A330 and A331 (H268Q/V309L/A330S/A331S); substitutions at residues C220, C226, C229 and P238 (C220S/C226S/C229S/P238S); substitutions at residues C226, C229, E233, L234 and L235 (C226S/C229S/E233P/L234V/L235A; substitutions at residues K322, L235 and L235 (K322A/L234A/L235A); substitutions at residues L234, L235 and P331 (L234F/L235E/P331S); substitutions at residues 234, 235 and 297; substitutions at residues E318, K320 and K322 (L235E/E318A/K320A/K322A); substitutions at residues (V234A, G237A, P238S); substitutions at residues 243 and 264; substitutions at residues 297 and 299; substitutions such that residues 233, 234, 235, 237, and 238 defined by the EU numbering system, comprise a sequence selected from PAAAP, PAAAS and SAAAS (see WO2011/066501). In one embodiment, the antibody has a substitution in a heavy chain constant region at any one, two, three, four, five or more of residues selected from the group consisting of: 220, 226, 229, 233, 234, 235, 236, 237, 238, 243, 264, 268, 297, 298, 299, 309, 310, 318, 320, 322, 327, 330, 331 and 409 (numbering of residues in the heavy chain constant region is according to EU numbering according to Kabat).

In the shorthand notation used here, the format is: Wild type residue: Position in polypeptide: Mutant residues, wherein residue positions are indicated according to EU numbering according to Kabat.

In a specific embodiment, provided is an antibody that binds essentially the same epitope or determinant as monoclonal antibody 5E7; optionally the antibody comprises the hypervariable region of antibody 5E7. In any of the embodiments. herein, antibody 5E7 can be characterized by the amino acid sequences and/or nucleic acid sequences encoding it. In one embodiment, the monoclonal antibody comprises the Fab or F(ab′)₂ portion of 5E7. Also provided is an antibody or antibody fragment that comprises the heavy chain variable region of 5E7. According to one embodiment, the antibody or antibody fragment comprises the three CDRs of the heavy chain variable region of 5E7. Also provided is an antibody or antibody fragment that further comprises the variable light chain variable region of 5E7 or one, two or three of the CDRs of the light chain variable region of 5E7. The HCDR1, 2, 3 and LCDR1, 2, 3 sequences can optionally be specified as all (or each, independently) being those of the Kabat numbering system, those of the Chotia numbering system, those of the IMGT numbering, or any other suitable numbering system. Optionally any one or more of said light or heavy chain CDRs may contain one, two, three, four or five or more amino acid modifications (e.g. substitutions, insertions or deletions).

In one aspect, an anti-Nectin-4 antibody or antibody fragment comprises a V_{H} domain having at least about 60%, 70% or 80% sequence identity, optionally at least about 85%, 90%, 95%, 97%, 98% or 99% identity, to the V_{H} domain of SEQ ID NO: 19. In another aspect, the anti-Nectin-4 antibody comprises a V_{L} domain having at least about 60%, 70% or 80% sequence identity, optionally at least about 85%, 90%, 95%, 97%, 98% or 99% identity, to the VL domain of SEQ ID NO: 20.

The VH and VL comprise (e.g., are modified to incorporate) human acceptor frameworks. In one embodiment, an anti-Nectin-4 antibody comprises the VH CDR1, CDR2 and/or CDR3 (e.g., according to Kabat numbering) of the heavy chain variable region having the amino acid sequence of SEQ ID NO: 19. In one embodiment, an anti-Nectin-4 antibody comprise the VL CDR1, CDR2 and/or CDR3 (e.g., according to Kabat numbering) of the light chain variable region having the amino acid sequence of SEQ ID NO: 20. In one embodiment, an anti-Nectin-4 antibody comprises a VH comprising the Kabat CDR1, CDR2 and/or CDR3 of the heavy chain variable region having the amino acid sequence of SEQ ID NO: 19 and a VL comprising a Kabat CDR1, CDR2 and/or CDR3 of the light chain variable region having the amino acid sequence of SEQ ID NO: 20.

5E7 VH:
(SEQ ID NO: 19)
QVQLQQPGAELVKPGASVKLSCKASGYIFTSYWMHWVKQRPGQGLEWI
GEIDPSDSYTNYNQKFKGKATLTLDKSSSTTYMQLSSLTSEDSAVYYC
VRGYGNYGDYWGQGTTLTVSS
5E7 VL:
(SEQ ID NO: 20)
DVVMTQAAFSNPVTLGTSASISCRSSKSLLHSNGITYLYWYLQKPGQS
PQLLIYQMSNLASGVPDRFSSSGSGTDFTLRISRVEAEDVGVYYCAQN
LELPWTFGGGTKLEIK
5E7 CDRs:
Kabat:
CDR-H1
(SEQ ID NO: 21)
SYWMH
CDR-H2
(SEQ ID NO: 22)
EIDPSDSYTNYNQKFKG
CDR-H3
(SEQ ID NO: 23)
GYGNYGDY
CDR-L1
(SEQ ID NO: 24)
RSSKSLLHSNGITYLY
CDR-L2
(SEQ ID NO: 25)
QMSNLAS
CDR-L3
(SEQ ID NO: 26)
AQNLELPWT
IMGT:
CDR-H1
(SEQ ID NO: 27)
GYIFTSYW
CDR-H2
(SEQ ID NO: 28)
IDPSDSYT
CDR-H3
(SEQ ID NO: 29)
VRGYGNYGDY
CDR-L1
(SEQ ID NO: 30)
KSLLHSNGITY
CDR-L2
(SEQ ID NO: 31)
QMS
CDR-L3
(SEQ ID NO: 26)
AQNLELPWT
Chothia:
CDR-H1
(SEQ ID NO: 32)
GYIFTSY
CDR-H2
(SEQ ID NO: 33)
PSDS
CDR-H3
(SEQ ID NO: 34)
YGNYGD
CDR-L1
(SEQ ID NO: 35)
SKSLLHSNGITY
CDR-L2
(SEQ ID NO: 31)
QMS
CDR-L3
(SEQ ID NO: 36)
NLELPW

In one embodiment, an anti-Nectin-4 antibody may for example comprise a heavy chain variable region comprising the amino acid sequences of SEQ ID NOS: 37, 39, 41, 43, 45, 47, 49, 51, 53, 55 or 57 and a light chain variable region comprising the amino acid sequences of SEQ ID NOS: 59, 61, 63 or 65. An anti-Nectin-4 antibody may for example comprise: a HCDR1 comprising an amino acid sequence: SYWMH (SEQ ID NO: 21), or a sequence of at least 4 contiguous amino acids thereof, optionally wherein one or more of these amino acids may be substituted by a different amino acid; a HCDR2 comprising an amino acid sequence: EIDPSDSYTNYNQKFKG (SEQ ID NO: 22), or a sequence of at least 4, 5, 6, 7, 8, 9 or 10 contiguous amino acids thereof, optionally wherein one or more of these amino acids may be substituted by a different amino acid; a HCDR3 comprising an amino acid sequence: GYGNYGDY (SEQ ID NO: 23), or a sequence of at least 4, 5 or 6 contiguous amino acids thereof, optionally wherein one or more of these amino acids may be substituted by a different amino acid; a LCDR1 comprising an amino acid sequence: RSSKSLLHSNGITYLY (SEQ ID NO: 24), or a sequence of at least 4, 5, 6, 7, 8, 9 or 10 contiguous amino acids thereof, optionally wherein one or more of these amino acids may be substituted by a different amino acid; a LCDR2 region comprising an amino acid sequence: QMSNLAS (SEQ ID NO: 25) or a sequence of at least 4, 5 or 6 contiguous amino acids thereof, optionally wherein one or more of these amino acids may be substituted by a different amino acid; and/or a LCDR3 region comprising an amino acid sequence: AQNLELPWT (SEQ ID NO: 26), or a sequence of at least 4, 5, 6, 7 or 8 contiguous amino acids thereof, optionally wherein one or more of these amino acids may be deleted or substituted by a different amino acid. CDR positions may be according to Kabat numbering.

Advantageously, antibodies of the disclosure can be used in processes for preparing an antibody-conjugate. In one embodiment, a process for preparing an antibody-conjugate comprises conjugating a cytotoxic agent (Z) to an anti-Nectin-4 antibody or antibody fragment of the disclosure. In one embodiment, cytotoxic agent (Z) can be specified as being conjugated to the antibody or fragment via a linker (X). X is a linker which connects the antibody or fragment (Ab) and cytotoxic agent (Z), e.g., upon conjugation X is the residue of a linker following covalent linkage to one or both of Ab and Z.

In embodiments herein, a process for preparing the antibody-drug conjugates comprises a step of contacting and/or reacting an anti-Nectin-4 antibody or antibody fragment of the disclosure (Ab) with a cytotoxic agent (Z). The contacting can be carried out under conditions suitable such that an antibody drug conjugate of an aspect of the disclosure is formed or obtained. Z may for example be comprised in a compound comprising a cytotoxic agent (Z) and a linker (X) or portion of linker (X), such that the step comprises contacting an anti-Nectin-4 antibody or antibody fragment of the disclosure with a compound comprising a cytotoxic agent (Z) and a linker (X) or portion of linker (X). A process can optionally specify a step of isolating or recovering the antibody drug conjugate that is formed, and, optionally, further processing the composition for use as a medicament, optionally formulating said antibody (e.g., with a pharmaceutical excipient) for administration to a human subject.

Optionally, a method of making an ADC comprises conjugating the antibody or antibody fragment to 2, 3, 4, 5, 6, 7 or 8 molecules of cytotoxic agent. Optionally, the composition obtained is characterized by a DAR of between 2 and 4, between 4 and 6, between 6 and 8. In one embodiment, the method comprises conjugating the antibody to 4 molecules of cytotoxic agent. In one embodiment, the method comprises conjugating the antibody to 8 molecules of cytotoxic agent. Optionally, the method further comprises assessing the DAR, and if the DAR corresponds to a pre-determined specification (e.g. a DAR or DAR range as disclosed herein, a DAR of about 2, 4, 6, or 8, etc.), further processing the composition for use as a medicament, optionally formulating said antibody (e.g., with a pharmaceutical excipient) for administration to a human subject.

In some embodiments, the linker (X)-(Z) elements are prepared and isolated prior to contacting (and reacting) the compound comprising (X) and (Z) with the (Ab), thereby forming the antibody drug conjugate.

In some embodiments, the method comprises:
(a) contacting and/or reacting linker (X) or a portion of linker (X) with the (Ab) to form an Ab-X conjugate, and
(b) contacting and/or reacting Ab-X of step (a) with a cytotoxic agent (Z) or a compound comprising a second portion of linker (X) and (Z), thereby forming the antibody drug conjugate.

X can for example represent a molecule comprising a moiety that is cleavable, e.g., under physiological conditions, optionally under intracellular conditions. In one embodiment, X represents a molecule comprising (i) a spacer (Y), (ii) a cleavable moiety and (iii) an optional self-eliminating or non-self-eliminating spacer system (Y′). The cleavable moiety can for example be an oligopeptide (e.g. a di-, tri-, tetra- or penta-peptide). The spacer Y can be positioned between the Ab and the cleavable moiety, and the spacer system (Y′) can be positioned between the cleavable moiety and Z.

In some embodiments, linker X or spacer Y can optionally be specified as comprising a reactive group (R) capable of reacting (e.g. under suitable conditions, optionally after deprotection) with an amino acid of the antibody or with a complementary reactive group (R′) that is attached to an amino acid of the antibody. Optionally, R is a group reactive with a free amino, hydroxyl, sulfhydryl or carboxyl group on the antibody. For example R can be a maleimid-N-yl group having the structure below, which is reactive with a thiol group (also referred to a sulfhydryl group) on the antibody:

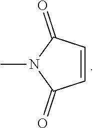

In some embodiments, linker X or spacer Y can optionally be specified as comprising the residue of the reaction of reactive group R with an amino acid of the antibody or with a complementary reactive group (R′) that is attached to an amino acid of the antibody. Optionally, R is the residue of the reaction of a group reactive with a free amino, hydroxyl, sulfhydryl or carboxyl group on the antibody and said free amino, hydroxyl, sulfhydryl or carboxyl group.

In any embodiment, prior to the step of contacting and/or reacting an anti-Nectin-4 antibody or antibody fragment with a compound (e.g. linker and/or cytotoxic agent), the method comprises a step of preparing, selecting or providing an anti-Nectin-4 antibody or antibody fragment. In one embodiment, the step comprises preparing, selecting or providing an anti-Nectin-4 antibody or antibody fragment and determining or testing whether the antibody or antibody fragment has feature(s) of an anti-Nectin-4 antibody or antibody fragment of the disclosure.

For example, an anti-Nectin-4 antibody or antibody fragment can be tested for the ability to bind to the VC1 bridging domain of Nectin-4. An antibody or antibody fragment that is determined to bind to the VC1 bridging domain of Nectin-4 is then contacted and/or reacted with the compound (e.g. linker (X) and/or cytotoxic agent (Z)). For example, an anti-Nectin-4 antibody or antibody fragment can be tested for the ability to bind to a mutant Nectin-4 polypeptide (e.g. a mutant Nectin-4 polypeptide comprising a substitution at residues K197 and/or S199). An antibody or antibody fragment that is determined to have decreased or loss of binding to the mutant Nectin-4 polypeptide (e.g. compared to binding a wild-type Nectin-4 polypeptide) is then contacted and/or reacted with the compound (e.g. linker (X) and/or cytotoxic agent (Z)).

In another example, an anti-Nectin-4 antibody or antibody fragment can be tested for the ability to reduce cell-cell adhesion between Nectin-4 expressing tumor cells (e.g. adherent tumor cells) and/or for the ability to reduce growth of Nectin-4 expressing tumor cells (e.g. in 3-dimensional cell culture, in a tumor spheroid formation assay). An antibody or antibody fragment that is determined to have the ability to reduce cell-cell adhesion between Nectin-4 expressing tumor cells and/or for the ability to reduce growth of Nectin-4 expressing tumor cells (e.g. adherent tumor cells) is then contacted and/or reacted with the compound (e.g. linker (X) and/or cytotoxic agent (Z)).

In another example, an anti-Nectin-4 antibody or antibody fragment can be tested for the ability to sensitize a tumor to a cytotoxic agent (e.g. a cytotoxic agent Z or of the same class of drugs as Z, for example a camptothecin agent). An antibody or antibody fragment that is determined to have the ability to sensitize a tumor to the cytotoxic agent is then contacted and/or reacted with the compound (e.g. linker (X) and/or cytotoxic agent (Z)).

Optionally, a method can comprise any two or more of the antibody testing steps prior to contacting the antibody with the compound (e.g. linker (X) and/or cytotoxic agent (Z)).

As further described herein, some well-known methods for conjugating cytotoxic agents to antibodies involve multiple reactions steps in which an antibody is first modified with a linker or part of a linker, followed by a reaction in which the cytotoxic agent is conjugated to the antibody-linker composition.

In one embodiment, provided is a process for preparing the antibody-conjugate comprising:
(i) obtaining anti-Nectin-4 antibodies by culturing a host cell transformed with an expression vector containing a polynucleotide encoding the antibody; collecting and purifying the antibody of interest from the cultures obtained in the preceding step;
(ii) contacting an anti-Nectin-4 antibody or antibody fragment with a compound (L) comprising (a) a first reactive group capable of reacting with an amino acid (e.g., a side chain or glycan of the amino acid, or a group attached to an amino acid or glycan of the amino acid) of the antibody and (b) a second reactive group (R′), to obtain a modified antibody comprising one or more amino acids functionalized with the compound (L); and
(iii) reacting the modified antibody of step (i) with a compound comprising (a) a reactive group (R) that is complementary to reactive group (R′), (b) an amino acid unit (e.g. a di-, tri-, tetra- or penta-peptide) that is cleaved by an intracellular peptidase or protease enzyme, (c) optionally a non-self-eliminating or a self-eliminating spacer (Y′), and (d) a cytotoxic agent (Z). Optionally, the compound of step (ii) further comprises a spacer (Y) placed between R and the amino acid unit.

Optionally, the step obtaining anti-Nectin-4 antibodies can further comprises steps of preparing antibody-producing cells by immunizing an animal by injection of the antigen, collecting the blood, assaying its antibody titer to determine when the spleen is excised; preparing myeloma cells; fusing the antibody-producing cells with the myeloma; screening a group of hybridomas producing a desired antibody; dividing the hybridomas into single cell clones (cloning); culturing the hybridoma or rearing an animal implanted with the hybridoma for producing a large amount of monoclonal antibody; and/or examining the thus produced monoclonal antibody for biological activity and binding specificity, or assaying the same for properties as a labeled reagent; and the like.

Optionally, the step obtaining anti-Nectin-4 antibodies can further comprise steps of preparing a cell library.

In one embodiment, R and R′ are capable of undergoing a click reaction or a cycloaddition, optionally wherein R comprises or is an alkyne moiety and R′ comprises or is an azide moiety, or wherein R′ comprises or is an alkyne moiety and R comprises or is an azide moiety, and wherein the reaction of step (ii) is a 1,3-dipolar cycloaddition.

In one embodiment, the reaction of step (i) is carried out in presence of a catalyst, optionally the catalyst is an enzyme (e.g., a transglutaminase).

In one embodiment, prior to contacting an anti-Nectin-4 antibody or antibody fragment with a compound (L), step (i) comprises a step of modifying the anti-Nectin-4 antibody or antibody fragment. For example, the antibody or antibody fragment can be modified by reacting or contacting it with an enzyme capable of modifying antibody glycosylation (e.g., at Kabat residue N297). In one example, the modification comprises the deglycosylation of an antibody glycan having a core N-acetylglucosamine, in the presence of an endoglycosidase, in order to obtain an antibody comprising a core N-acetylglucosamine substituent, wherein said core N-acetylglucosamine and said core N-acetylglucosamine substituent are optionally fucosylated. Examples of endoglycosidases include EndoS, EndoA, EndoE, Endo18A, EndoF, EndoM, EndoD, EndoH, EndoT and EndoSH and/or a combination thereof.

The antigen binding protein (e.g. antibody) molecule and cytotoxic agent (e.g. camptothecin derivative molecule) are connected by means of a linker. In such embodiments, the immunoconjugate can for example be represented by Formula (II):

Ab-(X-(Z)ₙ)ₘ Formula (II)
wherein,
Ab is an anti-Nectin4 antibody or antibody fragment);
X is a linker which connects Ab and Z, e.g., the residue of a linker following covalent linkage to one or both of Ab and Z;
Z a cytotoxic agent, e.g. camptothecin analogue, optionally Z comprises a structure of Compounds 1 or 2 (exatecan or a SN-38 molecule);
n is 1 or 2; and
when n is 1, m is from among 1 to 8, or optionally m is an integer selected from among 1 to 8 or 1 to 6, optionally m is an integer selected from among 1 to 4, optionally m is 2 or 4; optionally, m is 2, 3, 4, 5, 6, 7 or 8; and when n is 2, m is from among 1 to 4, or optionally m is an integer selected from among 1 to 4 or 1 to 3, optionally m is an integer selected from among 1 to 4, optionally m is 2 or 4; optionally, m is 1, 2, 4 or 4. Optionally, “n” can be specified to represent the degree of branching or polymerization. “n” and “m” can be specified to represent the average in a composition comprising a plurality of antibodies.

In one embodiment, X represents a molecule comprising a moiety that is cleavable, e.g., under physiological conditions, optionally under intracellular conditions. In one embodiment, X represents a molecule comprising (i) a spacer (Y), (ii) a cleavable moiety and (iii) an optional self-eliminating or non-self-eliminating spacer system (Y′). The spacer Y can be positioned between the Ab and the cleavable moiety, and the spacer system (Y′) can be positioned between the cleavable moiety and Z. Molecule X or spacer Y can optionally be specified as comprising a reactive group (R) or the residue of the reaction of reactive group R with an amino acid of the antibody or with a complementary reactive group (R′) that is attached to an amino acid of the antibody.

The variable m represents the number of -X-(Z)ₙ moieties per antibody molecule in an immunoconjugate. In a composition comprising a plurality of anti-Nectin-4 ADCs, the number “m” of number of -X-Z moieties per antibody molecule may vary. Thus, in exemplary compositions comprising a plurality of immunoconjugates of the formulae herein, m is the average number of -X-(Z)ₙ moieties per Ab, in which case m can also be referred to as the average drug loading or drug: antibody ratio (DAR). Average drug loading or DAR may advantageously range from 1 to about 8 (-X-(Z)ₙ) moieties per Ab. The number of Z moieties attached to a moiety X, “n”, can for example be 1 or 2. Typically, n is 1. In some embodiments, n is 1, and m represents the average drug loading, m is between 2 and 8. In some embodiments, n is 1, and m represents the average drug loading, m is between 2 and 6. In some embodiments, n is 1, and m represents the average drug loading, m is between 4 and 8. In some embodiments, n is 1, and m represents the average drug loading, m is between 6 and 8, optionally about 6, 7 or 8. In some embodiments, n is 1, and m represents the average drug loading, m is between 4 and 6, optionally about 4, 5 or 6.

The number of (-X-Z) moieties per Ab may be characterized by conventional means such as mass spectroscopy, ELISA assay, and HPLC. The quantitative distribution of immunoconjugates in terms of m may also be determined. In some instances, separation, purification, and characterization of homogeneous immunoconjugates where m is a certain value, as distinguished from immunoconjugates with other drug loadings, may be achieved by means such as reverse phase HPLC or electrophoresis.

In one embodiment, an anti-Nectin-4 composition used in the treatment methods of the disclosure is characterized as comprising a plurality immunoconjugates represented by Formula (I):

Ab-(X-(Z)ₙ)ₘ Formula (II)
wherein,
Ab is an anti-Nectin-4 antigen binding protein (e.g. an antibody or antibody fragment);
X is a molecule which connects Ab and Z, e.g., the residue of a linker following covalent linkage to one or both of Ab and Z;
Z is a cytotoxic agent, optionally a topoisomerase inhibitor, optionally a camptothecin analogue, optionally a camptothecin analogue comprising an exatecan or a SN-38 molecule, e.g., a molecule having the structure of Compounds 1 or 2;
n is 1 or 2; and
wherein at least 50%, 60%, 70%, 80%, 90%, 95%, 98% or 99% of immunoconjugates in an antibody sample have an m (the number of X-Z moieties) that is 2 or 4, at least 2, between 2 and 4, at least 4, between 4 and 6 or between 4 and 8, optionally wherein n is 1 and at least 50%, 60%, 70%, 80%, 90%, 95%, 98% or 99% of immunoconjugates in an antibody sample have an m (the number of X-Z moieties) that is 2 or 4, at least 2, between 2 and 4, at least 4, between 4 and 6 or between 4 and 8.

In one embodiment, an anti-Nectin-4 composition used in the treatment methods of the disclosure is characterized as comprising a plurality immunoconjugates represented by Formula (I):

Ab-(X-(Z)ₙ)ₘ Formula (II)
wherein,
Ab is an anti-Nectin-4 antigen binding protein (e.g. an antibody or antibody fragment);
X is a molecule which connects Ab and Z, e.g., the residue of a linker following covalent linkage to one or both of Ab and Z;
Z a camptothecin analogue comprising an exatecan or a SN-38 molecule, e.g., a molecule comprising the structure of Compounds 1 or 2;
n is 1; and
wherein at least 50%, 60%, 70%, 80%, 90%, 95%, 98% or 99% of immunoconjugates in an antibody sample have an m (the number of X-Z moieties) that is 6, at least 6, between 6 and 8, or 8.

It will be appreciated that a variety of methods can be used to covalently link the linker comprising the cytotoxic agent to the antibody or antigen binding protein, either non-specifically or specifically to a particular amino acid residue. The linker (X) can comprise a moiety that is cleavable, e.g., under physiological conditions, optionally as shown in the Examples under intracellular conditions, such that cleavage of the linker releases the cytotoxic agent (e.g. Compound 1, Compound 2, Compound 13, etc.) in the intracellular environment. The linker can be bonded to a chemically reactive group on the antibody molecule, e.g., to a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cysteinyl residues), to a carbohydrate, or generally to any reactive group introduced or engineered into an antibody. The site to which the linker is bound can be a natural residue in the amino acid sequence of the antibody molecule or it can be introduced into the antibody molecule, e.g., by DNA recombinant technology (e.g., by introducing a cysteine or protease cleavage site in the amino acid sequence, by introducing a non-natural amino acid residue) or by protein biochemistry (e.g., reduction, pH adjustment or proteolysis, by glycoengineering, enzymatic modification of an amino acid-bound glycan).

In some embodiments, the linker (X) comprises peptide residues comprising amino acid selected from phenylalanine, glycine, valine, alanine, lysine, citrulline, serine, glutamic acid, and aspartic acid, optionally the linker (X) comprises a dipeptide, a tripeptide, or a tetrapeptide residue.

In certain embodiments, an intermediate, which is the precursor of the linker (X), is reacted with the cytotoxic agent (Z) under appropriate conditions. In certain embodiments, reactive groups are used on the cytotoxic agent and/or the intermediate. In some embodiments, the product of the reaction between the cytotoxic agent and the intermediate, or the derivatized cytotoxic agent, is subsequently reacted with the antibody molecule under appropriate conditions. In other embodiments, a precursor of the linker (X) is first reacted with the antibody molecule under appropriate conditions so at to yield an antibody bound to the precursor of the linker (X), and the antibody is subsequently reacted with a molecule comprising the cytotoxic agent (Z).

In some embodiments, the linker (X) is cleavable by a cleaving agent that is present in the intracellular environment (e.g., within a lysosome or endosome or caveolea). The linker can comprise for example a peptidyl linker or amino acid unit that is cleaved by an intracellular peptidase or protease enzyme, including, but not limited to, a lysosomal or endosomal protease. In some embodiments, a peptidyl linker moiety is at least two amino acids long or at least three amino acids long. Cleaving agents can include cathepsins B and D and plasmin, all of which are known to hydrolyze dipeptide drug derivatives resulting in the release of active drug inside target cells. Most typical are peptidyl linkers that are cleavable by enzymes that are present in cells. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the valine-citrulline linker). A valine-citrulline (Val-Cit) element can have the structure shown below:

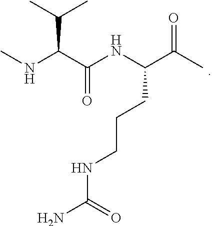 [SMILES: CN[C@H](C(=O)N[C@@H](CCCNC(N)=O)C(C)=O)C(C)C]

In another specific embodiment, the peptidyl linker cleavable by an intracellular protease is a valine-alanine (Val-Ala) linker. A val-ala element can have the structure is shown below:

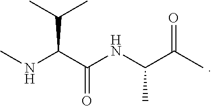

In another specific embodiment, the peptidyl linker cleavable by an intracellular protease is a glycine-containing oligopeptide linker, for example glycine- and phenylalanine-containing oligopeptide linker, optionally a GGF, DGGF, (D−)D-GGF, EGGF, SGGF, KGGF, DGGFG (SEQ ID NO: 71), DDGGFG (SEQ ID NO: 72), KDGGFG (SEQ ID NO: 73), GGFGGGF (SEQ ID NO: 74), GGFG, GGFGG (SEQ ID NO: 75) or GGFGGG (SEQ ID NO: 76) linker (see, e.g., U.S. Pat. No. 6,835,807, the disclosure of which is incorporated herein by reference), wherein “(D−)D represents D-aspartic acid.

In some embodiments, a linker can function to act as a spacer or stretcher to distance an antibody from Z in order to avoid interference with the ability of the antibody to bind Nectin-4 and/or inhibit cell-cell interactions mediated by Nectin-4. A linker may comprise a spacer unit (Y) and/or a spacer or spacer system (Y′). The spacer Y can thus be positioned between the Ab and the cleavable moiety. The spacer system (Y′) can be positioned between the cleavable moiety and Z. Molecule X or spacer Y can optionally be specified as comprising a reactive group (R) or the residue of the reaction of reactive group R with an amino acid of the antibody or with a complementary reactive group (R′) that is attached to an amino acid of the antibody. The spacer Y can for example be a molecule that forms a bond (e.g. via its reactive group R) with an amino acid of the antibody, e.g., a sulfur atom, a primary or secondary amino group or a carbohydrate group of the antibody, and which spacer or stretcher (Y) links the antibody to the cytotoxic agent (Z) or to a cleavable amino acid unit (e.g. the peptidyl linker, a cleavable di-, tri-, tetra- or penta-peptide, optionally further with a self-eliminating and/or non-self-eliminating spacer (Y′) which is in turn linked to Z. Thus, when the spacer (Y) is linked at one end to an amino acid unit (e.g. a cleavable di-, tri-, tetra- or penta-peptide), the cleavable amino acid unit can in turn be directly linked to Z or can comprise a further spacer (Y′) such as a non-self-eliminating or a self-eliminating spacer which links the amino acid unit and Z.

Spacer (Y) can optionally be specified as being or comprising a substituted or unsubstituted alkyl or heteroalkyl chain, optionally wherein Y has a chain length of 2-100 atoms, optionally 2-40, 2-30, 2-20, 4-40, 4-30 or 4-20 atoms, optionally where one or more atoms can be other than carbon, for example oxygen, sulfur, nitrogen, or other atoms, optionally wherein any carbon of the chain is substituted with an alkoxy, hydroxyl, alkylcarbonyloxy, alkyl-S—, thiol, alkyl-C(O)S—, amine, alkylamine, amide, or alkylamide.

Spacer (Y) can optionally be specified as comprising a stability-enhancing moiety. For example, spacer Y can orthogonal poly-ethyleneglycol (PEG) moieties or polysarcosine (poly-N-methylglycine or PSAR) moieties in the linker design (see, e.g., WO2019/081455, WO2015/057699 and WO2016/059377, the disclosure of which are incorporated herein by reference).

In some specific embodiments, spacer (Y) may comprise one or more ethylene oxide monomers, optionally Y comprises a polyethylene oxide moiety, optionally Y comprises between 1 and 24, optionally 1 and 12, optionally 1 and 8, optionally 6 and 24 polyethylene oxide moieties, optionally Y comprises a structure-(CH₂CH₂O)ₓ— where x is 1 to 24, optionally 1 to 12, optionally 1 to 6, optionally 6 to 24.

An example of a suitable stability-enhancing moiety, spacer chain Y can comprise a stability-enhancing moiety disclosed in PCT publication nos. WO2015/057699 or WO2019/081455. For example, spacer chain Y can comprise an orthogonal connector moiety and stability-enhancing moiety. The stability-enhancing moiety can be a PEG homopolymer, or generally any single molecular weight homopolymer (e.g. a PEG or polysarcosine homopolymer) bound to the orthogonal connector moiety. The homopolymer can have for example 1-4, 1-6, 1-7, 1-8, 1-10, 1-12, at least 6, 8 or 10, or 6-12, 6-24, 6-72, or no more than 6, 7, or 8 units of the PEG or other monomer. The term orthogonal connector refers to a branched linker unit component that connects a linker moiety (e.g. the chain of spacer Y) to a homopolymer unit and via a linker (e.g. a cleavable oligopeptide (Pep) and spacer Y′) to a cytotoxic agent (Z) so that the homopolymer unit is in a parallel configuration (as opposed to a series configuration) in relation to the cytotoxic agent (the homopolymer is in parallel to the Pep-Y′-Z moiety). The orthogonal connector moiety can for example be one or more natural or non-natural amino acids optionally selected from glutamic acid, lysine and glycine. Optionally, the amino acid orthogonal connector moiety is placed at the end of spacer chain Y such that the orthogonal connector moiety amino acid residue is connected, via a peptide bond between the α-carboxyl group of one amino acid to the α-amino group of the other amino acid, to an amino acid residue of the peptidyl linker (e.g. (Pep) in Formula V or VI). Y can for example comprise the result of the reaction of the orthogonal connector moiety with a moiety of Formula D:

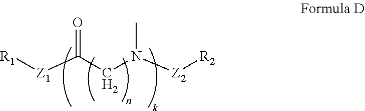

wherein R¹ and R² are different, and
one of R¹ and R² is H or an inert group, the other one of R¹ and R² being functionalized reactive group, said group being reactive for covalently binding to a bindable group of the orthogonal connector moiety, in such reaction conditions that the inert group is non-reactive, Z₁ and Z₂, identical or different, are optional spacers, and n is 1 or more and k is 2 or more.

Optionally, the orthogonal connector derives from a glutamic acid.

In another example, spacer Y comprises a group disclosed in US patent publication no. US2017/0072068A1, the disclosures of which are incorporated herein by reference, for example a group according to formula (E) or a salt thereof:

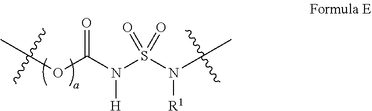

wherein
a is 0 or 1; and
R¹ is selected from the group consisting of hydrogen, C₁-C₂₄ alkyl groups, C₃-C₂₄ cycloalkyl groups, C₂-C₂₄ (hetero) aryl groups, C₃-C₂₄ alkyl(hetero) aryl groups and C₃-C₂₄ (hetero) arylalkyl groups, the C₁-C₂₄ alkyl groups, C₃-C₂₄ cycloalkyl groups, C₂-C₂₄ (hetero) aryl groups, C₃-C₂₄ alkyl(hetero) aryl groups and C₃-C₂₄ (hetero) arylalkyl groups optionally substituted and optionally interrupted by one or more heteroatoms selected from O, S and NR³ wherein R³ is independently selected from the group consisting of hydrogen and C₁-C₄ alkyl groups; and wherein the group according to formula E, or the salt thereof, is situated in between said the first end and the second end of the spacer chain Y. According to this aspect, spacer Y can comprise-(Succinimid-3-yl-N)—CH2CH2-C(═O)—, (Succinimid-3-yl-N)—CH2CH2CH2-C(═O)—, -(Succinimid-3-yl-N)—CH2CH2CH2CH2-C(═O)—, -(Succinimid-3-yl-N)—CH2CH2CH2CH2CH2-C(═O)—.

Optionally, such spacer Y can further comprise the following structure: —NH—(CH2CH2-O)n-CH 2CH 2-C(—O)—, wherein n is an integer of 1 to 6, preferably 2 to 4. Such structure includes for example —NH—CH₂CH₂—O—CH₂CH₂—C(═O)—, —NH—CH₂CH₂—O—CH₂CH₂—O—CH₂CH₂—C(═O)—, —NH—CH₂CH₂—O—CH₂CH₂—O—CH₂CH₂—O—CH₂CH₂—C(═O)—, —NH—CH₂CH₂—O—CH₂CH₂—O—CH₂CH₂—O—CH₂CH₂—O—CH₂CH₂—C(═O)—, —NH—CH₂CH₂—O—CH₂CH₂—O—CH₂CH₂—O—CH₂CH₂—O—CH₂CH₂—O—CH₂CH₂—C(═O)—, —NH—CH₂CH₂—O—CH₂CH₂—O—CH₂CH₂—O—CH₂CH₂—O—CH₂CH₂—O—CH₂CH₂—O—CH₂CH₂—O—CH₂CH₂—C(═O)—.

The spacer or spacer system (Y′) placed between the amino acid unit (e.g. a cleavable di-, tri-, tetra- or penta-peptide) and Z may be self-eliminating or non-self-eliminating. A spacer Y′ may for example comprise a substituted or unsubstituted alkyl or heteroalkyl chain, optionally wherein Y has a chain length of 2-30 atoms, optionally 2-20, 4-20, 2-10 or 4-20 atoms, optionally where one or more atoms can be other than carbon, for example oxygen, sulfur, nitrogen, or other atoms, optionally wherein any carbon of the chain is substituted with an alkoxy, hydroxyl, alkylcarbonyloxy, alkyl-S—, thiol, alkyl-C(O)S—, amine, alkylamine, amide, or alkylamide. In one embodiment, Y′ comprises a p-aminobenzyloxycarbonyl group. In one embodiment, Y′ is a non-self-eliminating spacer and comprises a structure of —(CH2)n-C(═O)—, wherein n is an integer of 0 to 5, said structure being connected to the cleavable moiety by a —O— or a single bound. For example, Y′ can be or can comprise a —C(═O)—, —O—C(═O)—, —O—CH2-C(═O)—, —O—CH2CH2-C(═O)—, —O—CH2CH2CH2-C(═O)—, —O—CH2CH2CH2CH2-C(═O)—, —O—CH2CH2CH2CH2CH2-C(═O)—, HO—O—CH2-C(═O)—, —CH2-C(═O)—, —CH2CH2-C(═O)—, —CH2CH2CH2-C(═O)—, —CH2CH2CH2CH2-C(═O)—, —CH2CH2CH2CH2CH2-C(═O)—, —CH2-O—CH2-C(═O)— or a —CH2CH2-O—CH2-C(═O)— group.

A “self-eliminating” spacer unit allows for release of the drug moiety without a separate hydrolysis step. When a self-eliminating spacer is used, after cleavage or transformation of the amino acid unit, the side of the spacer linked to the amino acid unit becomes unblocked, which results in eventual release of one or more moieties Z. The self-elimination spacer systems may for example be those described in WO02/083180 and WO2004/043493, the disclosures of which are incorporated herein by reference in their entirety, as well as other self-elimination spacers known to a person skilled in the art. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent. In one embodiment, the spacer unit is a p-aminobenzyloxycarbonyl (PAB) for example having the structure:

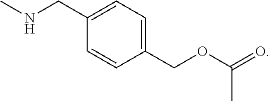

Examples of self-eliminating spacer units further include, but are not limited to, aromatic compounds that are electronically similar to p-aminobenzyl alcohol (see, e.g., US 2005/0256030 AI), such as 2-aminoimidazol-5-methanoi derivatives (Hay et al. (1999) Bioorg. Med. Chem. Lett. 9:2237) and ortho- or para-aminobenzylacetals. Spacers can be used mat undergo cyclization upon amide bond hydrolysis, such as substituted and unsubstituted 4-aminobutyric acid amides (Rodrigues et al., Chemistry Biology, 1995, 2, 223) and 2-aminophenylpropionic acid amides (Amsberry, et al., J. Org. Chem., 1990, 55. 5867). Elimination of amine-containing drugs that are substituted at the α-position of glycine (Kingsbury, et al., J. Med. Chem., 1984, 27, 1447) are also examples of self-eliminating spacers. A p-aminobenzyl self-eliminating spacer (e.g. PAB) is particularly suited for use together with a Phe-Lys, Val-Ala or Val-Cit cleavable dipeptide unit (the PAB is placed between the dipeptide and the camptothecin derivative (Z).

A “non-self-eliminating” spacer unit is one in which part or all of the spacer unit remains bound to the moiety Z upon enzymatic (e.g., proteolytic) cleavage of the antibody-moiety-of-interest conjugate. Examples of non-self-eliminating spacer units adapted for use as a spacer between a Gly-Gly-Phe-Gly amino acid unit and an exatecan molecule include, but are not limited to, include-O—CH₂—C(═O)—, HO—O—CH₂—C(═O)—, —CH₂CH₂—C(═O)—, —CH₂CH₂CH₂—C(═O)—, —CH₂—O—CH₂—C(═O)— and —CH₂CH₂—O—CH₂—C(═O)— (e.g., to form a GGFG-CH₂CH₂—O—CH₂—C(═O)-exatecan unit). Use of such a spacer between the GGFG amino acid unit and exatecan results in the release of a molecule having the structure of Compound 13. Other examples of non-self-eliminating spacer units include, but are not limited to, a glycine spacer unit and a glycine-glycine spacer unit. Other known combinations of peptidic spacers susceptible to sequence-specific enzymatic cleavage can be used in a similar manner. For example, enzymatic cleavage of an antibody-moiety-of-interest conjugate containing a glycine-glycine spacer unit by a tumor cell associated protease would result in release of a glycine-glycine-drug moiety from the remainder of the antibody-moiety-of-interest conjugate. In one such embodiment, the glycine-glycine-drug moiety is then subjected to a separate hydrolysis step in the tumor cell, thus cleaving the glycine-glycine spacer unit from the drug moiety.

An exemplary linker-cytotoxic drug moiety (X-Z) can comprise any of the structures shown below in Formulae Ill and IV.

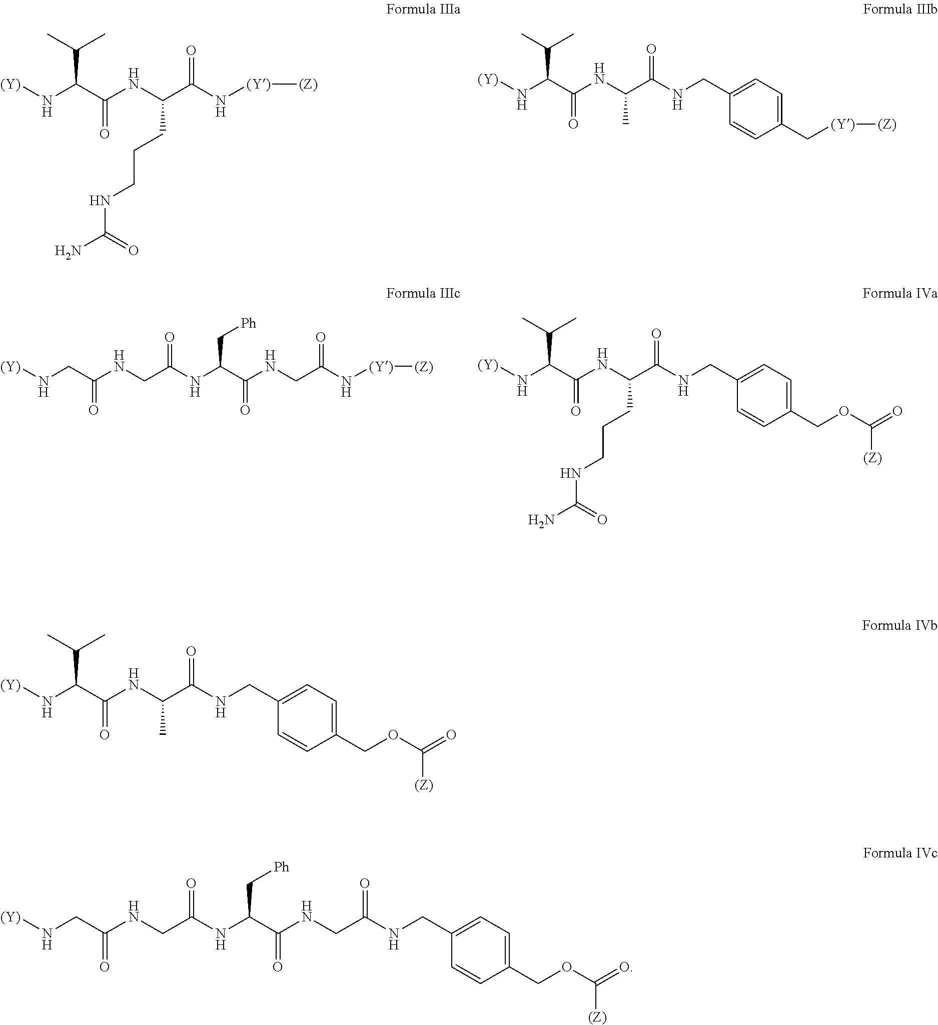

Spacers (Y) and (Y′) can optionally be specified as being are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR¹, wherein R¹ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Spacer (Y) and (Y′) can optionally be specified as being or comprising a C₁-C₁₀ alkylene-, —C₁-C₁₀ heteroalkylene-, —C₃-C₈ carbocyclo-, —O—(C₁-C₈ alkyl)-, -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈heterocyclo)-C₁-C₁₀ alkylene-, —C₁-C₁₀ alkylene-C(═O)—, —C₁-C₁₀ heteroalkylene-C(═O)—, —C₃-C₈carbocyclo-C(═O)—, —O—(C₁-C₈alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈carbocyclo)-C(═O)—, —(C₃-C₈carbocyclo)-C₁-C₁₀ alkylene-C(═O)—, —C₃-C₈heterocyclo-C(═O)—, —C₁-C₁₀ alkylene-(C₃-C₈heterocyclo)-C(═O)—, —(C₃-C₈heterocyclo)-C₁-C₁₀ alkylene-C(═O)—, —C₁-C₁₀ alkylene-NH—, —C₁-C₁₀ heteroalkylene-NH—, —C₃-C₈carbocyclo-NH—, —O—(C₁-C₈alkyl)-NH—, -arylene-NH—, -C₁—C₁₀ alkylene-arylene-NH—, -arylene-C₁-C₁₀ alkylene-NH—, —C₁-C₁₀ alkylene-(C₃-C₈ carbocyclo)-NH—, —(C₃-C₈carbocyclo)-C₁-C₁₀ alkylene-NH—, —C₃-C₈heterocyclo-NH—, —C₁-C₁₀ alkylene-(C₃-C₈heterocyclo)-NH—, —(C₃-C₈ heterocyclo)-C₁-C₁₀ alkylene-NH—, —C₁-C₁₀ alkylene-S—, —C₁-C₁₀ heteroalkylene-S—, —C₃-C₈carbocyclo-S—, —O—(C₁-C₈ alkyl)-)—S—, -arylene-S—, —C₁-C₁₀ alkylene-arylene-S—, -arylene-C₁-C₁₀ alkylene-S—, —C₁-C₁₀ alkylene-(C₃-C₈carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, —(C₃-C₈ heterocyclo)-C₁-C₁₀ alkylene-S—, —C₁-C₁₀ alkylene-O—C(═O)—, —C₃-C₈carbocyclo-O—C(═O)—, —O—(C₁-C₈alkyl)-O—C(═O)—, -arylene-O—C(═O)—, —C₁-C₁₀ alkylene-arylene-O—C(═O)—, -arylene-C₁-C₁₀ alkylene-O—C(═O)—, —C₁-C₁₀ alkylene-(C₃-C₈carbocyclo)-O—C(═O)—, —(C₃-C₈carbocyclo)-C₁-C₁₀ alkylene-O—C(═O)—, —C₃-C₈heterocyclo-O—C(═O)—, —C₁-C₁₀ alkylene-(C₃-C₈heterocyclo)-O—C(═O)—, —(C₃-C₈heterocyclo)-C₁-C₁₀ alkylene-O—C(═O)—, in each case optionally substituted with one or more of the substituents selected from: —X, —R′, —O, —OR′, ═O, —SR′, —S—, —NR′2, —NR′₃+, ═NR′, —CX₃, —CN, —OCN, —SCN, —N═C═O, —NCS, —NO, —NO₂, ═N₂, —N₃, —NR′C(═O)R′, —C(═O)R′, —C(═O)NR′₂, —SO₃, —SO₃H, —S(═O)₂R′, —OS(═O)₂OR′, —S(═O)₂NR′, —S(═O)R′, —OP(═O)(OR′)₂, —P(═O)(OR′)₂, —PO₃, —PO₃H₂, —C(═O)X, —C(═S)R′, —CO₂R′, —CO₂, —C(═S)OR′, C(═O)SR′, C(═S)SR′, C(═O)NR′₂, C(═S)NR′₂, and C(═NR′)NR′2, where each X is independently a halogen: —F, —Cl, —Br, or —I; and each R′ is independently-H, —C₁-C₂₀ alkyl, —C₆-C₂₀ aryl, or —C₃-C₁₄heterocycle.

In one embodiment, spacer (Y) can optionally be specified as comprising, e.g., at one end of the chain, a reactive group (R) that is reactive with a free amino, hydroxyl, sulfhydryl or carboxyl group, or carbohydrate, on the antibody. In some aspects, spacer Y comprises the R group -(Succinimid-3-yl-N)—(CH₂)ₙ³—C(═O), wherein n³ is an integer of 2 to 8, and -(Succinimid-3-yl-N) has a structure represented by the formula F hereinafter:

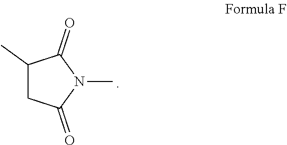

Position 3 of the above structure can be the connecting position to the antibody. The bond to the antibody at position 3 is characterized by bonding with thioether formation.

In one embodiment, spacer (Y) can optionally be specified as comprising, e.g., at one end of the chain, a reactive group (R) that is reactive with a complementary reactive group (R′) that is attached to an amino acid (e.g., via a free amino, hydroxyl, sulfhydryl or carboxyl group, or carbohydrate) of the antibody, or, upon conjugation to an anti-Nectin-4 antibody, the residue of the reaction of a reactive group (R) with a free amino, hydroxyl, sulfhydryl or carboxyl group on the antibody or with a complementary reactive group (R′) that is attached to an amino acid of the antibody. Examples of reactive group pairs R and R′ include a range of groups capable of biorthogonal reaction, preferably a cycloaddition, for example a Diels-Alder reaction or a 1,3-dipolar cycloaddition, for example between azides and cyclooctynes (copper-free click chemistry), between nitrones and cyclooctynes, oxime/hydrazone formation from aldehydes and ketones and the tetrazine ligation (see also WO2013/092983 or US2017/0072068A1, the disclosures of which are incorporated herein by reference). For example, R can be an alkyne and R′ can be an azide, or R can be an azide and R′ an alkyne. The resulting linker and functionalized antibody, or the Y element thereof, can thus in any embodiment comprise a group (RR′) resulting from the reaction of R and R′, for example RR′ can be or comprise a triazole resulting from the reaction of an alkyne and an azide.

In one embodiment, the reactive groups R and R′ are complementary reagents together capable of undergoing a “click” reaction (i.e., a Click Chemistry reagent or reactive group). For example, a 1,3-dipole-functional compound can react with an alkyne in a cyclization reaction to form a heterocyclic compound, preferably in the substantial absence of added catalyst (e.g., Cu(I)). A variety compounds having at least one 1,3-dipole group attached thereto (having a three-atom pi-electron system containing 4 electrons delocalized over the three atoms) can be used to react with the alkynes disclosed herein. Exemplary 1,3-dipole groups include, but are not limited to, azides, nitrile oxides, nitrones, azoxy groups, and acyl diazo groups.

Examples include o-phosphenearomatic ester, an azide, a fulminate, an alkyne (including any strained cycloalkyne), a cyanide, an anthracene, a 1,2,4,5-tetrazine, or a norbornene (or other strained cycloalkene).

In one embodiment, R is a moiety having a terminal alkyne or azide; such moieties are described for example in U.S. Pat. No. 7,763,736, the disclosure of which is incorporated herein by reference. Suitable reaction conditions for use of copper (and other metal salt) as catalysts of click-reactions between terminal alkynes and azides are provided in U.S. Pat. No. 7,763,736.

In one embodiment, R is a substituted or unsubstituted cycloalkyne. Cycloalkynes, including specific compounds, are described for example in U.S. Pat. No. 7,807,619, the disclosure of which is incorporated herein by reference.

In some embodiments, a cycloalkyne may be a compound of Formula A:

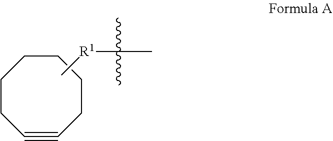

where:
R¹ is selected from a carbonyl, an alkyl ester, an aryl ester, a substituted aryl ester, an aldehyde, an amide, an aryl amide, an alkyl halide, a thioester, a sulfonyl ester, an alkyl ketone, an aryl ketone, a substituted aryl ketone, and a halosulfonyl;
R¹ can be at any position on the cyclooctyne group other than at the two carbons joined by the triple bond.

In some embodiments, the modified cycloalkyne is of Formula A, wherein one or more of the carbon atoms in the cyclooctyne ring, other than the two carbon atoms joined by a triple bond, is substituted with one or more electron-withdrawing groups, e.g., a halo (bromo, chloro, fluoro, iodo), a nitro group, a cyano group, a sulfone group, or a sulfonic acid group. Thus, e.g., in some embodiments, a subject modified cycloalkyne is of Formula B:

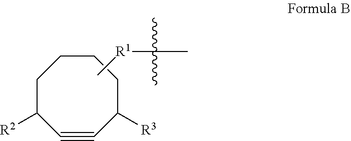

where:
each of R² and R³ is independently: (a) H; (b) a halogen atom (e.g., bromo, chloro, fluoro, iodo); (c) —W—(CH₂)ₙ—Z (where: n is an integer from 1-4 (e.g., n=1, 2, 3, or 4); W, if present, is O, N, or S; and Z is nitro, cyano, sulfonic acid, or a halogen); (d) —(CH₂)ₙ—W—(CH₂)ₘ—R⁴ (where: n and m are each independently 1 or 2; W is O, N, S, or sulfonyl; if W is O, N, or S, then R⁴ is nitro, cyano, or halogen; and if W is sulfonyl, then R⁴ is H); or (e) —CH₂)ₙ—R⁴ (where: n is an integer from 1-4 (e.g., n=1, 2, 3, or 4); and R⁴ is nitro, cyano, sulfonic acid, or a halogen); and
R¹ is selected from a carbonyl, an alkyl ester, an aryl ester, a substituted aryl ester, an aldehyde, an amide, an aryl amide, an alkyl halide, a thioester, a sulfonyl ester, an alkyl ketone, an aryl ketone, a substituted aryl ketone and a halosulfonyl. R¹ can be at any position on the cyclooctyne group other than at the two carbons linked by the triple bond.

In one embodiment, R is a substituted or unsubstituted heterocyclic strained alkyne. Cycloalkynes, including specific compounds, are described for example in U.S. Pat. No. 8,133,515, the disclosure of which is incorporated herein by reference. In one embodiment, the alkyne is of the Formula C:

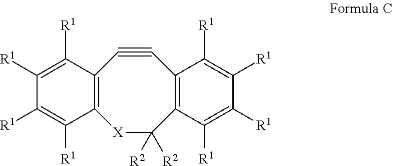

wherein:
each R¹ is independently selected from the group consisting of hydrogen, halogen, hydroxy, alkoxy, nitrate, nitrite, sulfate, and a C₁-C₁₀ alkyl or heteroalkyl;
each R² is independently selected from the group consisting of hydrogen, halogen, hydroxy, alkoxy, nitrate, nitrite, sulfate, and a C₁-C₁₀ organic group; X represents N—R³R⁴, NH—R⁴, CH—N—OR⁴, C—N—NR³R⁴, CHOR⁴, or CHNHR⁴; and each R³ represents hydrogen or an organic group and R⁴ represents linking moiety C of a linker. In one embodiment, R or R′ is a DBCO (dibenzycyclooctyl) group below:

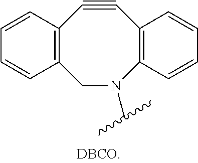 [SMILES: *N1Cc2ccccc2C#Cc2ccccc21]

Alkynes such as those described herein above can be reacted with at least one 1,3-dipole-functional compound in a cyclization reaction to form a heterocyclic compound, preferably in the substantial absence of added catalyst (e.g., Cu(I)). A wide variety compounds having at least one 1,3-dipole group attached thereto (having a three-atom pi-electron system containing 4 electrons delocalized over the three atoms) can be used to react with the alkynes disclosed herein. Exemplary 1,3-dipole groups include, but are not limited to, azides, nitrile oxides, nitrones, azoxy groups, and acyl diazo groups.

In the Formulae herein, Y′ can be optionally absent or can be a spacer, optionally a self-eliminating spacer, for example comprising p-aminobenzyl unit, or a non-self-eliminating spacer. Optionally, Y′ is or comprises a substituted or unsubstituted alkyl or heteroalkyl chain, optionally wherein Y′ has a chain length of 2-40 atoms, optionally 2-30, 2-20, 4-40, 4-30 or 4-20 atoms, optionally where one or more atoms can be other than carbon, for example oxygen, sulfur, nitrogen, or other atoms, optionally wherein any carbon of the chain is substituted with an alkoxy, hydroxyl, alkylcarbonyloxy, alkyl-S—, thiol, alkyl-C(O)S—, amine, alkylamine, amide, or alkylamide.

An exemplary linker-cytotoxic agent molecule (e.g. an X-Z moiety of Formulae I to XI) that can be conjugated to an anti-Nectin-4 antibody can optionally be represented by Formula V:

(R)-(Y)-(Pep)-(Y′)-(Z) Formula (V)
wherein,
R is a group reactive with a free amino, hydroxyl, sulfhydryl or carboxyl group on the antibody, or reactive with a complementary reactive group (R′) that is attached to an amino acid of the antibody, or, upon conjugation to the anti-Nectin-4 binding protein R is the residue of the reaction of a reactive group (R) with a free amino, hydroxyl, sulfhydryl or carboxyl group on the antibody or with a complementary reactive group (R′) that is attached to an amino acid of the antibody;
Y is optionally absent or is a spacer;
Pep is or comprises a peptidyl linker that is cleaved by an intracellular peptidase or protease enzyme, for example a valine-citrulline, valine-alanine or phenylalanine-lysine dipeptide;
Y′ is optionally absent or is a spacer, optionally a self-eliminating spacer or a non-self-eliminating spacer; and
Z is a cytotoxic agent, optionally a camptothecin analogue, optionally an exatecan,
Dxd or SN-38 molecule.

A resulting Nectin-4 binding immunoconjugate according to the invention can for example be represented by Formula (VI):

Ab-(Y)-(Pep)-(Y′)-(Z) Formula (VI)
wherein,
Ab is an anti-Nectin-4 an antibody;
Y is optionally absent or is a spacer. Optionally Formula VI comprises, between (Ab) and (Y) the residue of the reaction of a reactive group (e.g. a maleimide, a primary amine) with the side chain or carbohydrate of an amino acid of anti-Nectin-4 antigen binding protein (Ab). Alternatively, the residue of the reaction of a reactive group (e.g. a maleimide, a primary amine) with the side chain of an amino acid of anti-Nectin-4 antigen binding protein (Ab) can be specified as being comprised in Y;
Pep is or comprises an amino acid unit (e.g. peptidyl linker) that is cleaved by an intracellular peptidase or protease enzyme, (e.g., (Pep) is a protease-cleavable di-, tri-, tetra- or penta-peptide, for example a valine-citrulline, valine-alanine or phenylalanine-lysine unit);
Y′ is optionally absent or is a spacer, optionally a self-eliminating spacer or a non-self-eliminating spacer; and
Z is a cytotoxic agent, optionally a camptothecin derivative, optionally an exatecan, Dxd or SN-38 molecule.

Optionally, the formula comprises can be specified as comprising (e.g. between (Ab) and the end of Y (or (Pep or X) if Y is absent)) the residue (RR′) of the reaction of a reactive group (R) with a free amino, hydroxyl, sulfhydryl or carboxyl group on the antibody or with a complementary reactive group (R′) that is attached to an amino acid of the antibody.

In one example, where (RR′) is the residue of the reaction of a reactive group (R) with a complementary reactive group (R′) that is attached to the antibody (e.g. R′ is attached to a side chain or glycan of an amino acid of the antibody), the Nectin-4 binding immunoconjugate according to the invention can for example be represented by Formula (VI_{bis}):

Ab-(RR′)—(Y)-(Pep)-(Y′)-(Z) Formula (VI_{bis})
wherein, Ab, Y, Pep, Y′ and Z are as defined in Formula VI, and RR′ is the result of biorthogonal reaction, preferably a cycloaddition, for example a Diels-Alder reaction or a 1,3-dipolar cycloaddition. In one embodiment, RR′ has a structure selected from the group consisting of:

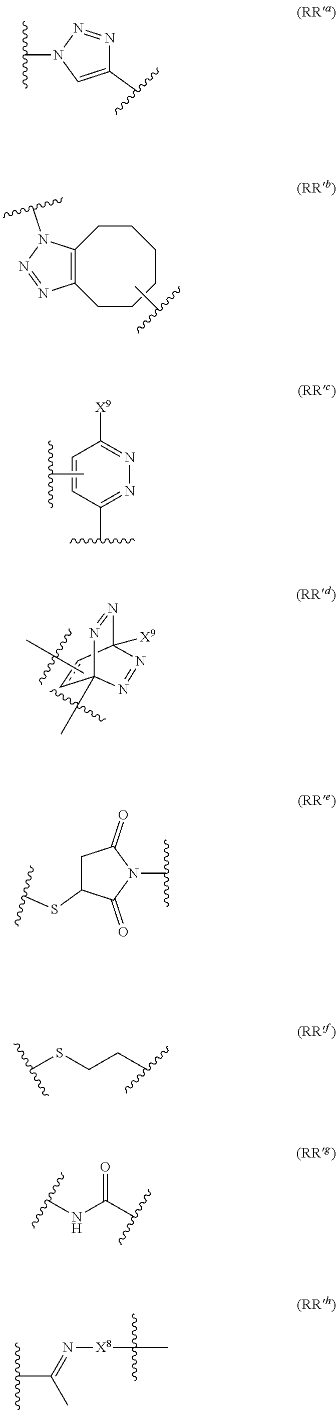

wherein X⁸ is O or NH, X⁹ is selected from H, methyl and pyridyl, and in structure (RR′^{c}) and (RR′^{d}), and the [symbol] bond represents either a single or a double bond.

In any embodiment, an exatecan molecule (or other 6-ring camptothecin) can be specified as being bound to Y′ (or (Pep) if Y′ is absent) via the amine at position 1 of exatecan.

In any embodiment, a SN-38 molecule (or other 5-ring camptothecin) can be specified as being bound to Y′ (or (Pep) if Y′ is absent) via the amine at position 9 of SN-38.

The cytotoxic agent, also referred to as the (Z) moiety, includes, for example, cytotoxic agents such as antineoplastic agents. Examples of cytotoxic agents are known in the art. For example, Z can be an alkylating agent, preferably a DNA alkylating agent. An alkylation agent is a compound that can replace a hydrogen atom with an alkyl group under physiological conditions (e.g. pH 7.4, 37 C, aqueous solution). Alkylation reactions are typically described in terms of substitution reactions by N, O and S heteroatomic nucleophiles with the electrophilic alkylating agent, although Michael addition reactions are also important. Examples of alkylating agents include nitrogen and sulfur mustards, ethylenimines, methanosulfonates, CC-1065 and duocarmycins, nitrosoureas, platinum-containing agents, agents that effectuate Topoisomerase II-mediated site dependent alkylation of DNA (e.g. psorospermin and related bisfuranoxanthones), ecteinascidin and other or related DNA minor groove alkylation agents.

In one embodiment, Z is a chelated metal, such as chelates of di- or tripositive metals having a coordination number from 2 to 8 inclusive. Particular examples of such metals include technetium (Tc), rhenium (Re), cobalt (Co), copper (Cu), gold (Au), silver (Ag), lead (Pb), bismuth (Bi), indium (In), gallium (Ga), yttrium (Y), terbium (Tb), gadolinium (Gd), and scandium (Sc). In general, the metal is preferably a radionuclide. Particular radionuclides include 99mTc, 186Re, 188Re, 58Co, 60Co, 67Cu, 195Au, 199Au, 110Ag, 203Pb, 206Bi, 207Bi, 111 In, 67Ga, 68Ga, 88Y, 90Y, 160Tb, 153Gd and 47Sc. The chelated metal may be for example one of the above types of metal chelated with any suitable polydentate chelating agent, for example acyclic or cyclic polyamines, polyethers, (e.g. crown ethers and derivatives thereof); polyamides; porphyrins; and carbocyclic derivatives.

An anti-Nectin-4 antibody or antibody fragment can also be used in diagnostics, e.g., to detect Nectin-4 tumor cells. In such embodiments, an antibody or antibody fragment can be conjugated to an effector molecule such as detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerytbrin; suitable luminescent materials include luminol; suitable bioluminescent materials include luciferase, luciferin, and aequorin; and suitable radioactive nuclides include 1251, 1311, 111 In and 99Tc.

In one embodiment, cytotoxic agent (Z) is a DNA minor groove binding and/or alkylating agent, e.g., a pyrrolobenzodiazepine, a duocarmycin, or derivatives thereof.

In a further embodiment, the cytotoxic agent is selected from the group consisting of taxanes, anthracyclines, camptothecins, epothilones, mytomycins, combretastatins, vinca alkaloids, nitrogen mustards, maytansinoids, calicheamycins, duocarmycins, tubulysins, dolastatins and auristatins, enediynes, amatoxins, pyrrolobenzodiazepines, ethylenimines, radioisotopes, therapeutic proteins and peptides, and toxins or fragments thereof.

In a further embodiment, the cytotoxic agent is selected from cyclophosphamide, ifosfamide, chlorambucil, 4-(bis(2-chloroethyl)amino)phenol, 4-(bis(2-fluoroethyl) ammo) phenol, N,N-bis(2-chloroethyl)-p-phenylenediamine, N,N-bis(2-fluoro-ethyl)-p-phenylenediamine, carmustine, lomustine, treosulfan, dacarbazine, cisplatin, carboplatin, vincristine, vinblastine, vindesine, vinorelbine, paclitaxel, docetaxel, etoposide, teniposide, topotecan, inirotecan, 9-aminocamptothecin, 9-nitrocamptothecin, 10-hydroxycamptothecin, lurtotecan, camptothecin, crisnatol, mitomycin C, mitomycin A, methotrexate, trimetrexate, mycophenolic acid, tiazofurin, ribavirin, hydroxyurea, deferoxamine, 5-fluorouracil, floxuridine, doxifluridine, raltitrexed, cytarabine, cytosine arabinoside, fludarabine, 6-mercaptopurine, thioguanine, raloxifen, megestrol, goserelin, leuprolide acetate, flutamide, bicalutamide, vertoporfin, phthalocyanine, photosensitizer Pc4, demethoxy-hypocrellin A, interferon-alpha, interferon-gamma, tumor necrosis factor, lovastatin, staurosporine, actinomycin D, bleomycin A2, bleomycin B2, peplomycin, daunorubicin, doxorubicin, N-(5,5-diacetoxypentyl) doxorubicin, morpholino doxorubicin, idarubicin, epirubicin, pirarubicin, zorubicin, mitoxantrone, thapsigargin, N⁸-acetylspermidine, tallysomycin, esperamycin, butyric acid, retinoic acid, I,8-dihydroxybicyclo[7.3.1]trideca-4-ene-2,6-diyne-13-one, anguidine, podophyllotoxin, combretastatin A-4, pancratistatin, tubulysin A, tubulysin D, carminomycin, streptonigrin, elliptmium acetate, maytansine, maytansinol, calicheamycin, mertansine (DM1), N-acetyl-γ₁^{I}-calicheamycin, calicheamycin-γ₁^{I}, calicheamycin-α₂^{I}, calicheamycin-α₃^{I}, duocarmycin SA, duocarmycin A, CC-1065, CBI-TMI, duocarmycin C2, duocarmycin B2, centanamycin, dolastatin, auristatin E, monomethylauristatin E (MMAE), monomethylauristatin F (MMAF), α-amanitin, β-amanitin, γ-amanitin, ε-amanitin, amanin, amaninamide, amanullin, and amanullinic acid and derivatives thereof.

Exemplary auristatin embodiments include the N-terminus linked monomethylauristatin drug moieties comprising a structure of any of Formulas (a1) and (a2) below:

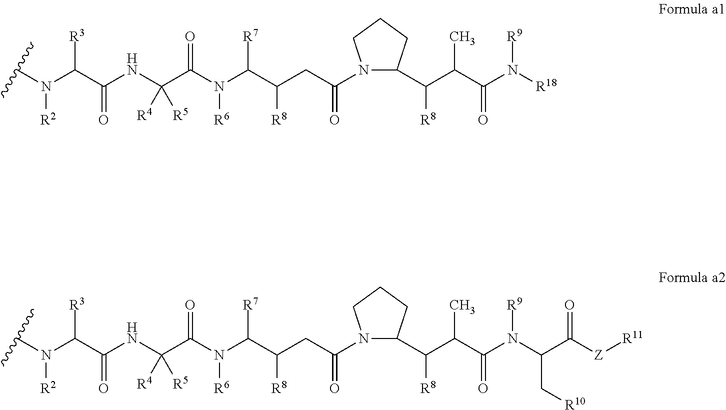

wherein the wavy line of (a1) and (a2) indicates the covalent attachment site to linker (e.g. linker X, or moiety Y, Pep or Y′), and independently at each location:
R² is selected from H and C₁-C₈alkyl;
R³ is selected from H, C₁-C₈ alkyl, C₁-C₈ carbocycle, aryl, C₁-C₈alkyl-aryl, C₁-C₈ alkyl-(C₃-C₈ carbocycle), Ca—C₈ heterocycle and C₁-C₈alkyl-(Ca—C₈ heterocycle);
R⁴ is selected from H, C₁-C₈alkyl, Ca—C₈ carbocycle, aryl, C₁-C₈alkyl-aryl, C₁-C₈ alkyl-(C₃-C₈ carbocycle), C₃-C₈ heterocycle and C₁-C₈alkyl-(Ca—C₈ heterocycle);
R⁵ is selected from H and methyl;
or R⁴ and R⁵ jointly form a carbocyclic ring and have the formula —(CR^{a}R^{b})ₙ wherein R^{a} and R^{b} are independently selected from H, C₁-C₈alkyl and C₃-C₈ carbocycle and n is selected from 2, 3, 4, 5 and 6;
R⁶ is selected from H and C₁-C₈alkyl;
R⁷ is selected from H, C₁-C₈alkyl, Ca—C₈ carbocycle, aryl, C₁-C₈ alkyl-aryl, C₁-C₈ alkyl-(C₃-C₈ carbocycle), Ca—C₈ heterocycle and C₁-C₈alkyl-(C₃-C₈ heterocycle);
each R⁸ is independently selected from H, OH, C₁-C₈alkyl, C₃-C₈ carbocycle and O—(C₁-C₈alkyl);
R⁹ is selected from H and C₁-C₈alkyl;
R¹⁰ is selected from aryl or C₃-C₈ heterocycle;
Z is O, S, NH, or NR¹² wherein R¹² is C₁-C₈ alkyl;
R¹¹ is selected from H, C₁-C₂₀ alkyl, aryl, C₃-C₈ heterocycle, —(R¹³⁰)ₘ—R¹⁴, or —(R¹³O)ₘ—CH(R¹⁵)₂; m is an integer ranging from 1-1000;
R¹³ is C₂-C₈ alkyl;
R¹⁴ is H or C₁-C₈ alkyl;
each occurrence of R¹⁵ is independently H, COOH, —(CH₂)ₙ—N(R¹⁶)₂, —(CH₂)ₙ—SO₃—C₁-C₈ alkyl;
each occurrence of R¹⁶ is independently H, C₁-C₈ alkyl, or —(CH₂)ₙ—COOH;
R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and
n is an integer ranging from 0 to 6.

In one embodiment, R³, R⁴ and R⁷ are independently isopropyl or sec-butyl and R⁵ is —H or methyl. In an exemplary embodiment. R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ—N(R¹⁶)₂, and R¹⁶ is —C₁-C₈ alkyl or —(CH₂)ₙ—COOH.

In another embodiment, when Z is —NH, R¹¹ is —CH(R¹⁵)₂, wherein R¹⁵ is —(CH₂)ₙ—SO₃H.

One exemplary auristatin embodiment of formula (a1) is MMAE, wherein the wavy line indicates the covalent attachment to a linker:

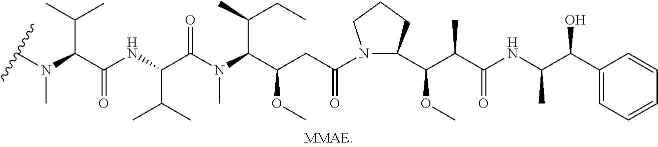 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula (a2) is MMAF, wherein the wavy line indicates the covalent attachment to a linker (L) of an antibody-drug conjugate (see US 2005/0238649 and Doronina et al. (2006) Bioconjugate Cfiem. 17:1 14-124):

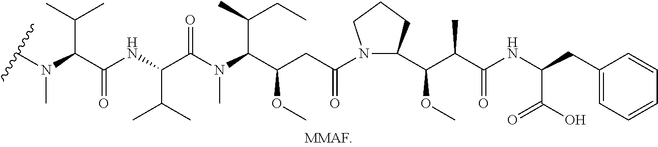 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

Other exemplary Z embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Other drug moieties include the following MMAF derivatives, wherein the wavy line indicates the covalent attachment to a linker:

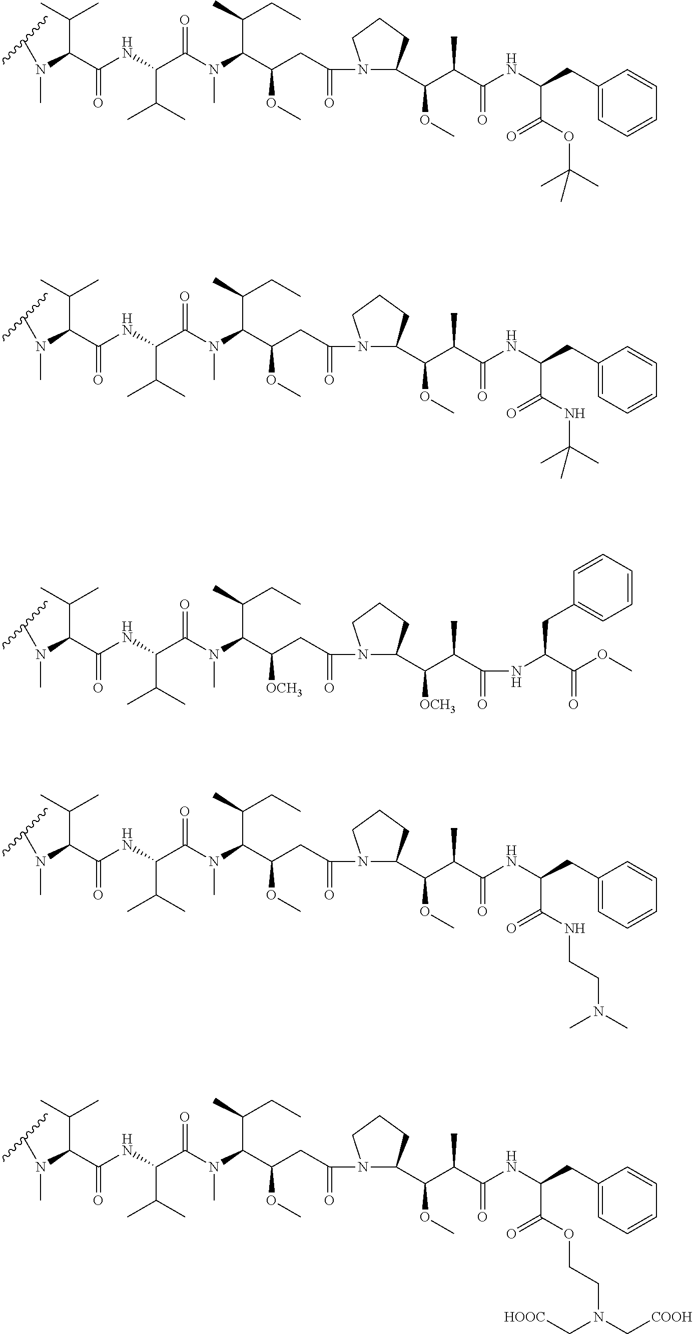

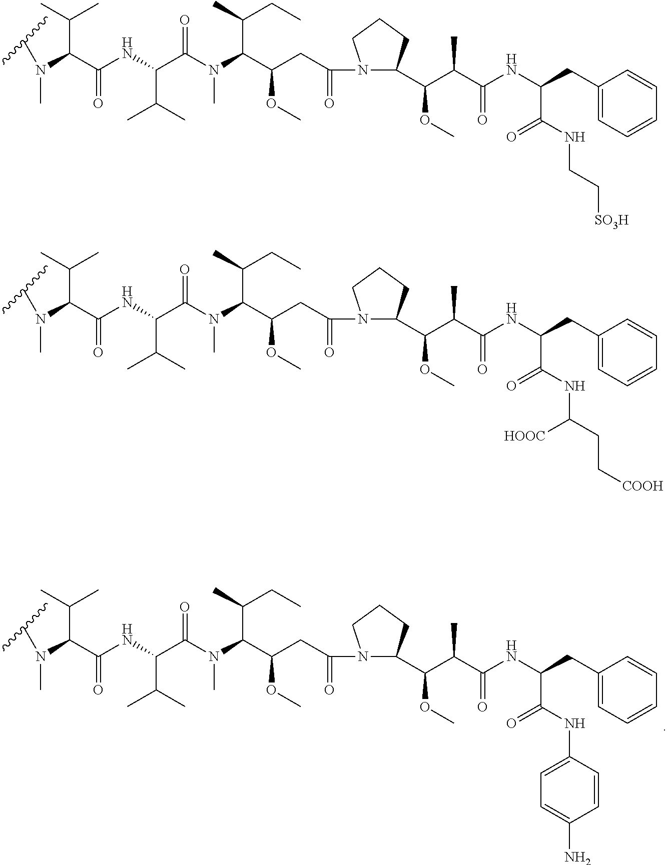 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)NCCS(=O)(=O)O)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)NC(CCC(=O)O)C(=O)O)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)Nc1ccc(N)cc1)OC)C(C)C)C(C)C]

An example of a linker comprising a spacer (Y) comprising a primary amine, a valine-citrulline as the (Pep) moiety, a PAB as the (Y′) moiety together with a MMAF as the (Z) moiety is shown below:

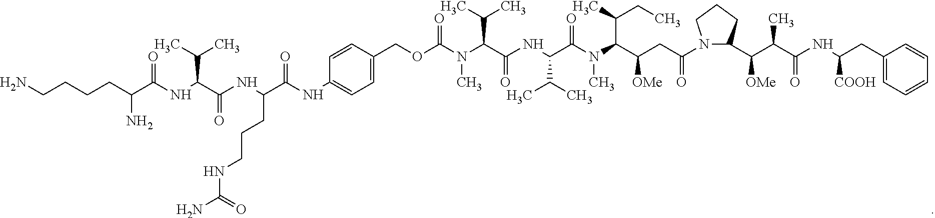

In one embodiment, the Z moiety is a DNA minor groove binding agent, optionally Z comprises a pyrrolobenzodiazepine (PBD). In one embodiment, Z is a pyrrolobenzodiazepine monomer. In one embodiment, Z is a pyrrolobenzodiazepine dimer comprising two pyrrolobenzodiazepine units. In one embodiment, Z is a pyrrolobenzodiazepine trimer comprising three pyrrolobenzodiazepine units. In one embodiment, Z is a pyrrolobenzodiazepine multimer comprising more than three pyrrolobenzodiazepine units. Structures of PBDs, as well as formulas and methods of producing them are described for example in PCT publications Nos: WO 2013/177481, WO 2011/130616, WO 2004/043880, WO 2005/085251, WO2012/112687 and WO 2011/023883, the disclosures of each of which are incorporated herein by reference.

The pyrrolo[2,1-c][1,4]benzodiazepines are a family of sequence-selective, minor-groove binding DNA-interactive agents that covalently attach to guanine residues. It has been reported that the(S)-chirality at the C11a-position of PBDs provides them with the appropriate 3-dimensional shape to fit perfectly into the DNA minor groove. PBDs can have different effects and modes of action. PBDs can be DNA-binders or DNA-alkylators that do not cause crosslinking of DNA, or PBDs can be DNA cross-linkers.

The pyrrolobenzodiazepine unit or monomer can have a general structure as follows:

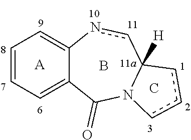

wherein the PBD can have different number, type and position of substituents, in both the aromatic A rings and pyrrolo C rings, and can vary in the degree of saturation of the C ring. In the B-ring there is either an imine (N═C), a carbinolamine (NH—CH(OH)), or a carbinolamine methyl ether (NH—CH(OMe)) at the N¹⁰—C¹¹ position which is the electrophilic centre responsible for alkylating DNA.

The biological activity of PBDs can be potentiated by joining two PBD monomers or units together, typically through their C8/C8′-hydroxyl functionalities via a flexible alkylene linker.

In one aspect of the any of the embodiments herein, a pyrrolobenzodiazepine monomer or unit is a pyrrolo[2,1-c][1,4]benzodiazepine. In one aspect of the any of the embodiments herein, a pyrrolobenzodiazepine dimer is a C8/C8′-linked pyrrolo[2,1-c][1,4]benzodiazepine dimer.

A PBD can be attached to a linker through any suitable position. For example, the PBD can be connected to a linker, via any of the positions in a PBD unit indicated below.

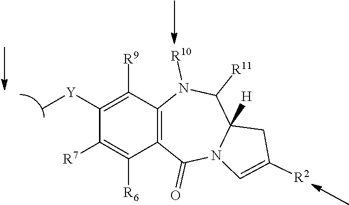

In one embodiment, a PBD dimer comprises the structure of the general formula below, with exemplary attachments points to other substituents or functionalities within a compound indicated by arrows:

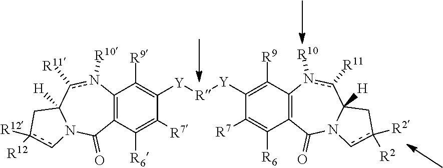

wherein:
   R¹² and R¹²′, and/or R² and R²′ together respectively form a double bond ═CH₂ or ═CH—CH₃; or
   R²′ and R¹²′ are absent and R² and R¹² are independently selected from:
   (iia) C₁₋₅ saturated aliphatic alkyl;
   (iib) C₃₋₆ saturated cycloalkyl;
   (iic)

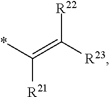

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

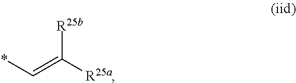

wherein one of R^{25a} and R^{25b} is H and the other is selected from:
phenyl, which phenyl is optionally substituted by a group selected from halo, methyl, methoxy; pyridyl; and thiophenyl; and

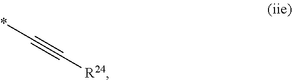

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
R⁶ and R⁹ are independently selected from H, R, OH, OR, SH, SR, NH₂, NHR, NRR′, nitro, Me₃Sn and halo; where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
either:
(a)R¹⁰ is H, and R¹¹ is OH, OR^{A}, where R^{A} is alkyl;
(b)R¹⁰ and R¹¹ form a nitrogen-carbon double bond between the nitrogen and carbon atoms to which they are bound; or
(c)R¹⁰ is H and R¹¹ is SO₂M, where z is 2 or 3 and M is a monovalent pharmaceutically acceptable cation;
R″ is a C₃₋₁₂ alkylene group, which chain may be interrupted by one or more heteroatoms, e.g., O, S, NR^{N2} (where R^{N2} is H or C₁₋₄ alkyl), and/or aromatic rings, e.g., benzene or pyridine;
Y and Y′ are selected from O, S, or NH; and
R⁶′, R⁷′, R⁹′ are selected from the same groups as R⁶, R⁷ and R⁹ respectively and R¹⁰′ and R¹¹′ are the same as R¹⁰ and R¹¹, wherein if R¹¹ and R¹¹ are SO₂M, M may represent a divalent pharmaceutically acceptable cation.

In another example, a PBD dimer comprises the structure of the general formula below:

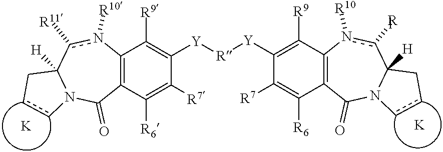

wherein R⁶, R⁷, R⁹, R⁶′, RT, R⁹′, R¹⁰, R¹¹, R¹⁰′ and R¹¹′ are as defined above, and wherein the “K” ring is a substituted or unsubstituted aromatic or non-aromatic ring, optionally a 6-member ring, optionally a phenyl.

In one embodiment, the cytotoxic agent (Z) is a camptothecin, e.g. it has or comprises the structure of camptothecin or of a camptothecin analog. Camptothecin is well known, as are a wide range of camptothecin analogues that share the core ring system with various substitutions, but preferably have modifications or substitutions in rings A and/or B of the basic camptothecin structure below:

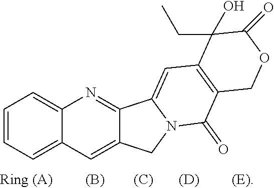 [SMILES: CCC1(O)C(=O)OCc2c1cc1n(c2=O)Cc2cc3ccccc3nc2-1]

Many camptothecin analogues have been reported including, topotecan, inirotecan, exatecan, DXd, 9-aminocamptothecin, 9-nitrocamptothecin, 10-hydroxycamptothecin, lurtotecan, camptothecin, gimatecan, belotecan, and rubitecan. Further camptothecin analogues are disclosed in Li et al., ACS Med. Chem. Lett. 2019, 10, 10, 1386-1392, Jpn. J. Cancer Res. 86:776-782 and in Takiguchi et al. 1997 Jpn. J. Cancer Res. 88:760-769, the disclosures of which are incorporated herein by reference. The four analogues topotecan, irinotecan, belotecan, and DXd (as part of trastuzumab deruxtecan) have been approved by the FDA. In one embodiment, the camptothecin analogue is a five-ring compound (e.g., the camptothecin lacks an F ring). In one embodiment, the camptothecin analogue is a six-ring compound, e.g., comprising the F ring.

Some examples such as the basic camptothecin structure, the SN-38 molecule (7-Ethyl-10-hydroxycamptothecin; active metabolite of irinotecan) and the camptothecin analogues disclosed in Li et al., ACS Med. Chem. Lett. 2019, 10, 10, 1386-139 have five rings (A, B, C, D and E rings) and can for example be attached to the linker (e.g. spacer Y or Y′, or linker X) via a substituent on the B ring.

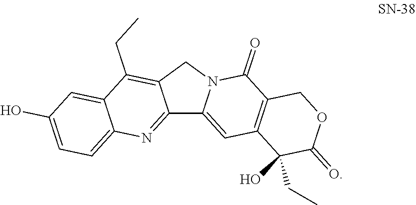 [SMILES: CCc1c2c(nc3ccc(O)cc13)-c1cc3c(c(=O)n1C2)COC(=O)[C@]3(O)CC]

Compounds of Li et al., ACS Med. Chem. Lett. 2019, 10, 10, 1386-139:

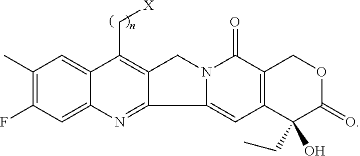

Optionally a camptothecin analogue is a six-ring compound (additionally an F ring), where the compound is attached to the linker through a substituent on such F ring. Examples of such six rings compounds include but are not limited to DXd (CAS No.: 1599440-33-1) and exatecan.

Camptothecin analogues thus include exatecan, SN-38, and any of a range of molecules that comprise such a moiety, for example an exatecan can unsubstituted or can be substituted at the position 1 amine, for example wherein the substituent is or comprises a —C(═O)—, —O—C(═O)—, —O—CH₂—C(═O)—, HO—O—CH₂—C(═O)—, —CH₂CH₂—C(═O)—, —CH₂CH₂CH₂—C(═O)—, —CH₂—O—CH₂—C(═O)—, —CH₂CH₂—O—CH₂—C(═O)— group or other group shown in U.S. Pat. No. 6,835,807, the disclosure of which is incorporated herein by reference.

In one embodiment, the antibody of the disclosure releases, in vivo or in vitro in presence of Nectin-4 expressing tumor cells (e.g. upon enzymatic cleavage of the cleavable moiety followed by self-elimination of the spacer Y′) an exatecan molecule having the structure of Compound 1.

The camptothecin derivative or analogue exatecan is described in Mitsui et al. 1995 Jpn. J. Cancer Res. 86:776-782 and in Takiguchi et al. 1997 Jpn. J. Cancer Res. 88:760-769, the disclosures of which are incorporated herein by reference. The structure of exatecan is shown below in Compound 1a:

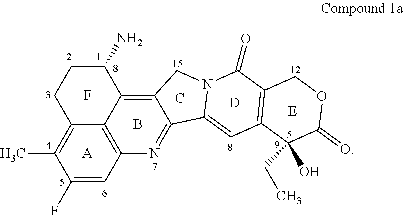

Exatecan can be coupled to a linker via the nitrogen atom of the amino group at position 1, such that the exatecan moiety, when bound to a linker or present within a linker-exatecan molecule (an (X-Z) molecule), for example as conjugated to an antibody, exatecan will have the structure of Compound 1b:

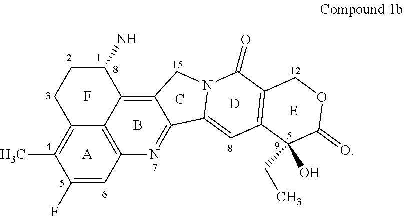

Thus, it will be understood that when exatecan of Compound 1a is attached to a linker (and, for example, when the linker is in turn attached to the antibody) via the amine at position 1, exatecan will be understood to be modified group at position 1 (i.e. the NH2 group at position 1 is replaced by an NH, or alternatively an OH or O group). For example, exatecan can be coupled to the antibody via a linker comprising a cleavable oligopeptide. Examples include di-, tri-, tetra- and penta-peptides such as the glycine and phenylalanine-containing peptides shown in U.S. Pat. No. 6,835,807, or the dipeptides valine-citrulline or valine-alanine attached to a PAB molecule, or the disclosures of which are incorporated herein by reference. Various suitable linker-Z structures are known that can liberate active exatecan or exatecan derivatives at the amino at position 1. Exatecan can be linked to a cleavable oligopeptide via a p-aminobenzyloxycarbonyl group (PAB) group attached to the position 1 amine, as shown in Formulae VII, VIII and IX, or in the (PEG (8U)-Val-Ala-PAB-Exatecan) linker of Example 7, which upon cleavage results in the liberation of the exatecan having the structure of Compound 1a. In one example, exatecan can be linked to a cleavable oligopeptide via a (CH₂—C(═O)) group attached to the position 1 amine, as shown in Formula III and Compound 13 (Dxd) as well as in the ggfg-Dxd linker of Example 7, which upon cleavage results in the liberation of the exatecan-containing Compound 13 (Dxd). Examples of substituents at the NH or NH2 of position 1 of exatecan of Compound 1 include —C(═O)—, —O—C(═O)—, or a (CH₂—C(═O)) comprising group such as —O—CH₂—C(═O)—, HO—O—CH₂—C(═O)—, —CH₂CH₂—C(═O)—, —CH₂CH₂CH₂—C(═O)—, —CH₂—O—CH₂—C(═O)— and —CH₂CH₂—O—CH₂—C(═O)—.

In one embodiment, a substituted exatecan derivative (e.g. derived at position 1) has the structure of compound 13.

The structure of Dxd is shown below:

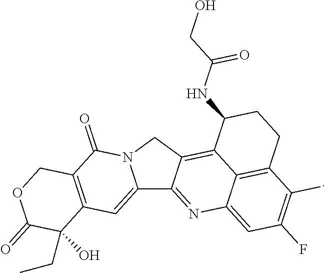

Dxd can be coupled to a linker via the oxygen atom at the terminus/of the carbon chain, such that the Dxd moiety, when bound to a linker or present within a linker-Dxd molecule (an (X-Z) molecule), for example as conjugated to an antibody, the carbon-chain-terminal OH will be replaced by an O.

In one embodiment, the linker moiety (X-Z) is or comprises the structure shown in Formula VII, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues). An anti-Nectin-4 binding protein functionalized with a linker comprising the structure of Formula VII or Compounds 3 or 4 will release or yield (e.g. intracellularly, in the presence of Nectin-4 expressing tumor cells) a compound having the structure of Compound 1a.

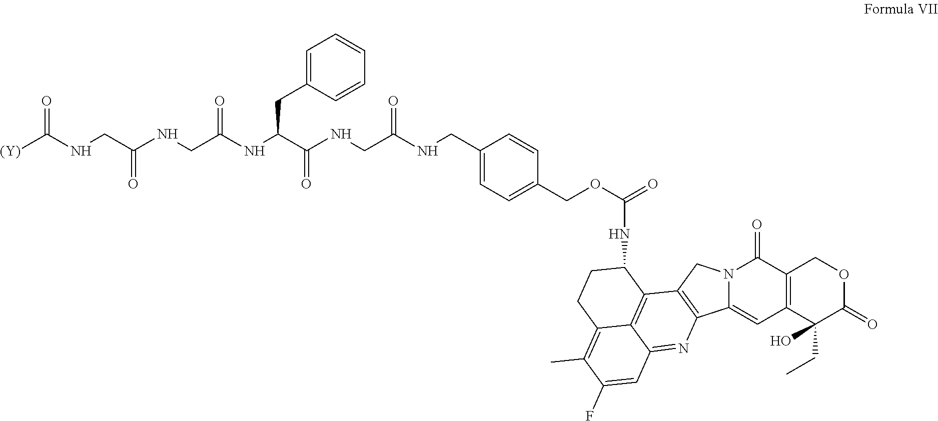

An exemplary linker having a maleimide as R group can have the structure of Compound 3, below. Such linker can be conjugated to an antibody via cysteine residues in the antibody after the interchain disulfide bounds are reduced with a reducing agent, e.g. tris(2-carboxyethyl) phosphine hydrochloride.

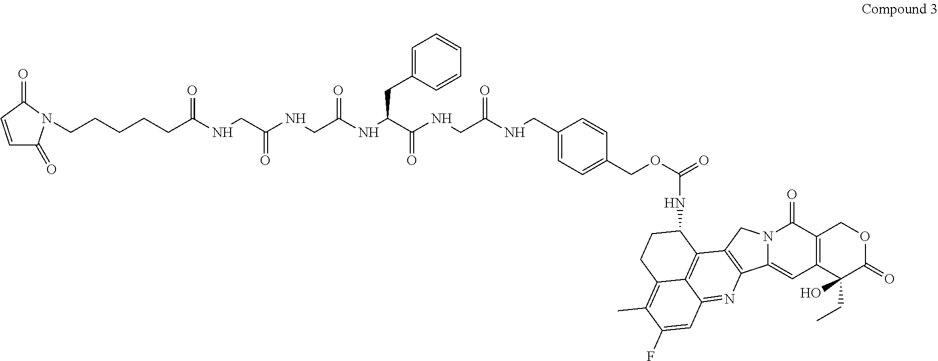 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)CNC(=O)[C@H](Cc2ccccc2)NC(=O)CNC(=O)CNC(=O)CCCCCN2C(=O)C=CC2=O)cc1)CC3]

The resulting antibody-drug conjugate will comprise an antibody comprising one or a plurality of cysteine residues functionalized with a compound having the structure of Compound 3 (wherein Compound 3 is bound via the S atom of the cysteine residue).

In another embodiment, a linker can have a primary amine as R group, and when reacted with an antibody in the presence of a transglutaminase enzyme, can yield an antibody comprising one or a plurality of acceptor glutamine residues functionalized with the linker. For example, a linker (X-Z), or an antibody functionalized therewith, has or comprises the structure shown as Compound 4, below:

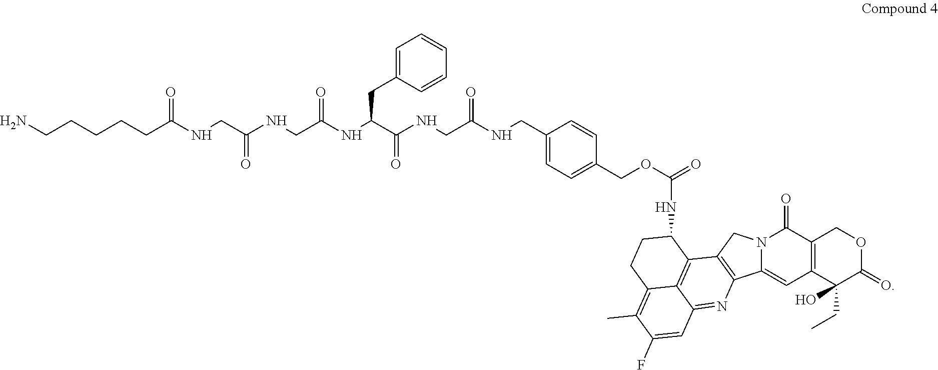 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)CNC(=O)[C@H](Cc2ccccc2)NC(=O)CNC(=O)CNC(=O)CCCCCN)cc1)CC3]

In one embodiment, the linker moiety (X-Z) is or comprises the structure shown in Formula VIII, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native, truncated or otherwise modified N-glycan bound to Kabat residue N297 of an antibody). An anti-Nectin-4 binding protein functionalized with a linker comprising the structure of Formula VIII or Compounds 5, 6 or 7 will release or yield (e.g. intracellularly, in the presence of Nectin-4 expressing tumor cells) a compound having the structure of Compound 1a.

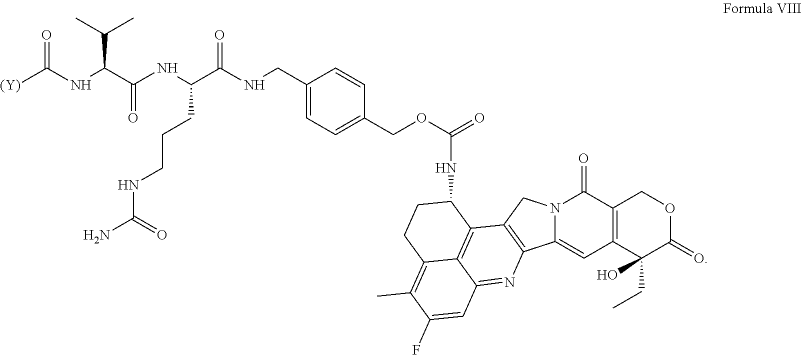

An exemplary linker having a maleimide as R group can have the structure of Compounds 5 or 6, below. Such linker can be conjugated to an antibody via cysteine residues in the antibody after the interchain disulfide bounds are reduced with a reducing agent.

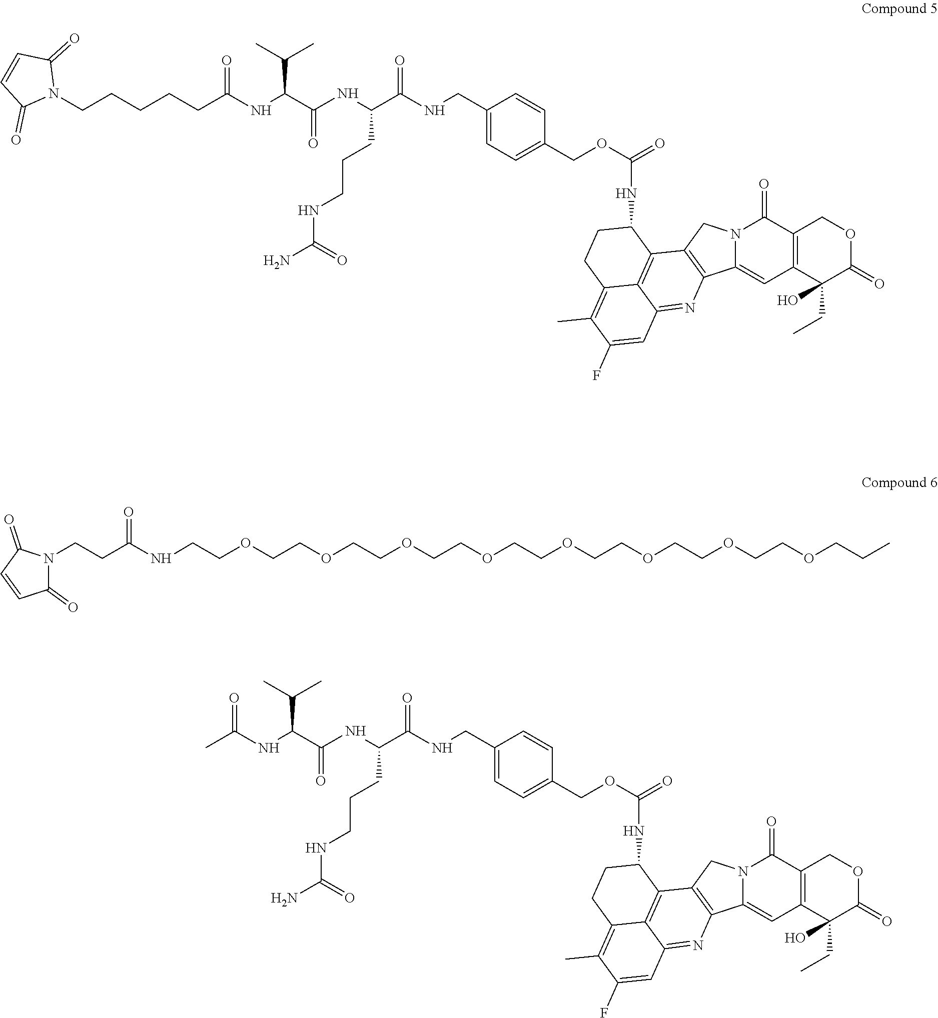

In another embodiment, a linker can have a primary amine as R group, and when reacted with an antibody in the presence of a transglutaminase enzyme, can yield an antibody comprising one or a plurality of acceptor glutamine residues functionalized with the linker. For example, a linker (X-Z), or an antibody functionalized therewith, has or comprises the structure shown as Compound 7, below:

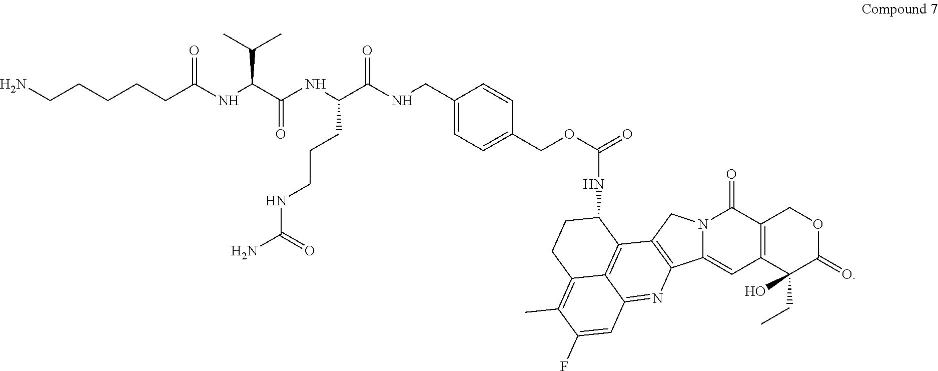 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](CCCNC(N)=O)NC(=O)[C@@H](NC(=O)CCCCCN)C(C)C)cc1)CC3]

In one embodiment, the linker moiety (X-Z) is or comprises the structure shown in Formula IX, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native, truncated or otherwise modified N-glycan bound to Kabat residue N297 of an antibody). An anti-Nectin-4 binding protein functionalized with a linker comprising the structure of Formula IX will release (e.g. intracellularly, in the presence of Nectin-4 expressing tumor cells) a compound having the structure of Compound 1a.

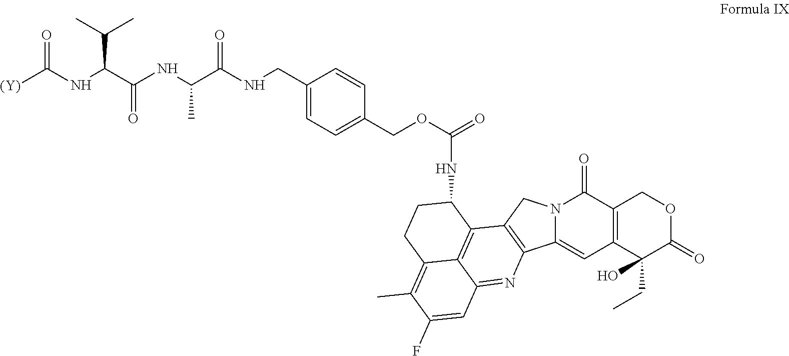

An exemplary linker having a maleimide as R group can have the structure of Compound 8, below. Such linker can be conjugated to an antibody via cysteine residues in the antibody after the interchain disulfide bounds are reduced with a reducing agent.

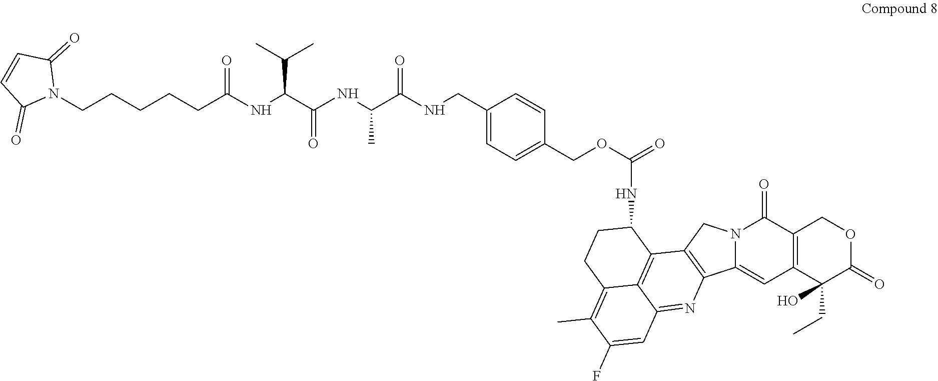 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](C)NC(=O)[C@@H](NC(=O)CCCCCN2C(=O)C=CC2=O)C(C)C)cc1)CC3]

In another embodiment, a linker can have a primary amine as R group, and when reacted with an antibody in the presence of a transglutaminase enzyme, can yield an antibody comprising one or a plurality of acceptor glutamine residues functionalized with the linker. For example, a linker (X-Z), or an antibody functionalized therewith, has or comprises the structure shown as Compound 9, below:

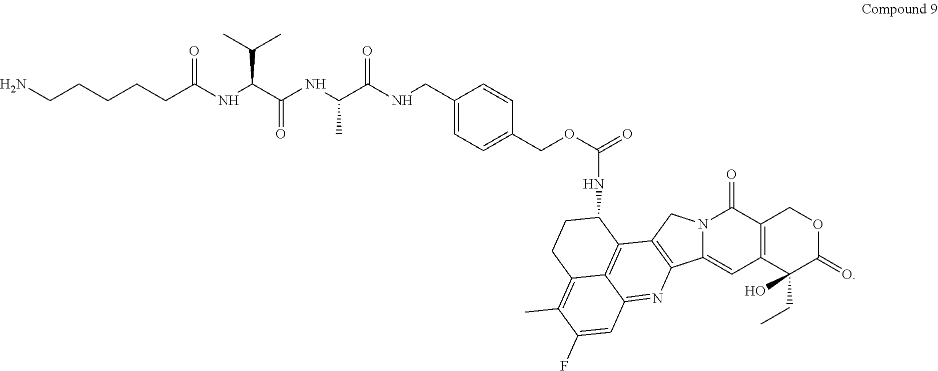 [SMILES: CC[C@@]1(O)C(=O)OCc2c1cc1n(c2=O)Cc2c-1nc1cc(F)c(C)c3c1c2[C@@H](NC(=O)OCc1ccc(CNC(=O)[C@H](C)NC(=O)[C@@H](NC(=O)CCCCCN)C(C)C)cc1)CC3]

In one embodiment, the linker moiety (X-Z) is or comprises the structure shown in Formula X, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native, truncated or otherwise modified N-glycan bound to Kabat residue N297 of an antibody).

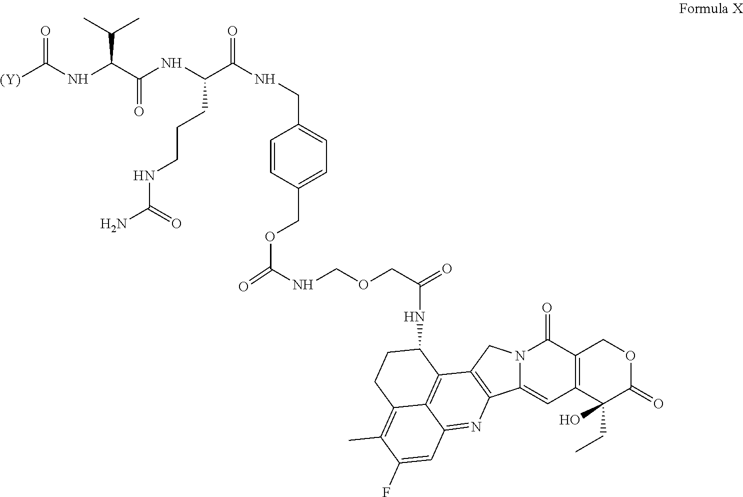

An exemplary linker having a maleimide as R group can have the structure of Compound 10, below. Such linker can be conjugated to an antibody via cysteine residues in the antibody after the interchain disulfide bounds are reduced with a reducing agent, e.g. tris(2-carboxyethyl) phosphine hydrochloride.

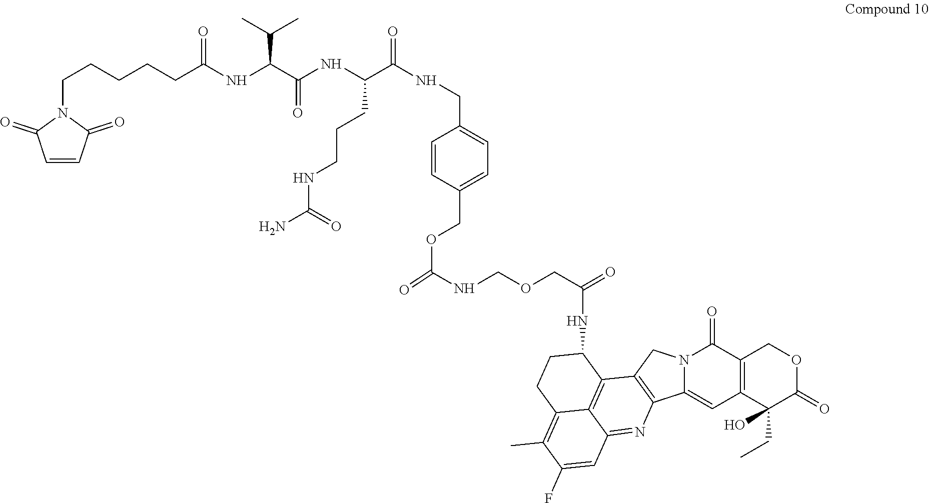 [SMILES: CC[C@@]1(O)C(=O)OCc2c1cc1n(c2=O)Cc2c-1nc1cc(F)c(C)c3c1c2[C@@H](NC(=O)COCNC(=O)OCc1ccc(CNC(=O)[C@H](CCCNC(N)=O)NC(=O)[C@@H](NC(=O)CCCCCN2C(=O)C=CC2=O)C(C)C)cc1)CC3]

In one embodiment, the linker moiety (X-Z) is or comprises the structure shown in Formula XI, below, wherein (Y) is a spacer comprising (e.g., at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native, truncated or otherwise modified N-glycan bound to Kabat residue N297 of an antibody).

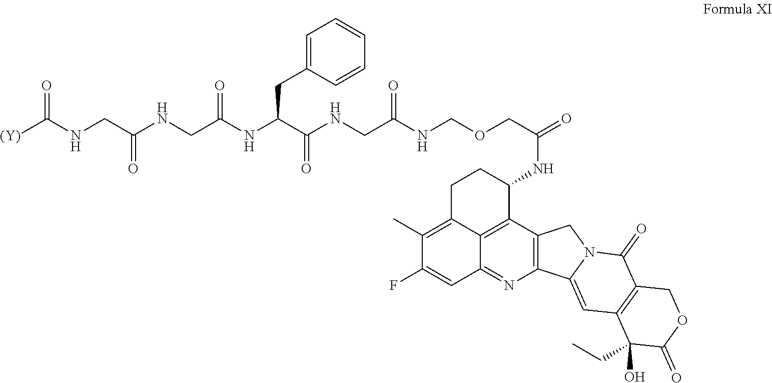

An exemplary linker having a maleimide as R group can have the structure of Compound 11, below. Such linker can be conjugated to an antibody via cysteine residues in the antibody after the interchain disulfide bounds are reduced with a reducing agent.

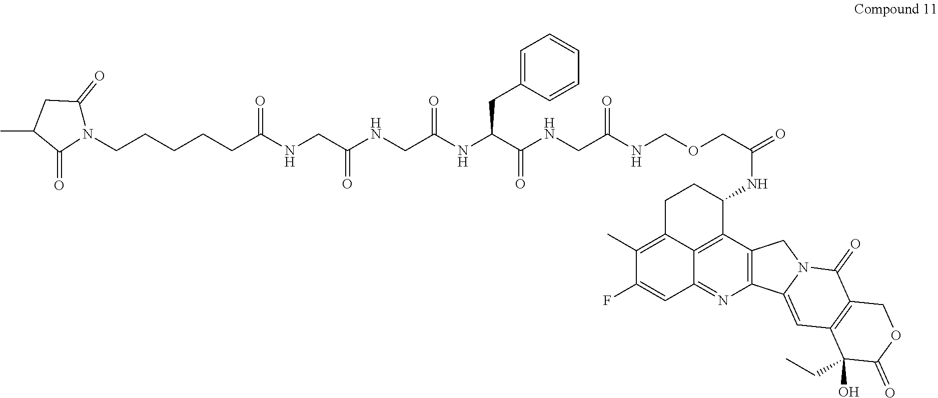 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

In one embodiment, the linker moiety (X-Z) is or comprises the structure according to Formula XII below, wherein (Y) is a spacer comprising (e.g. at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native truncated or otherwise modified N-glycan bound to Kabat residue N297 of an antibody). The linker may comprise for example 1-72, 1-16, 1-12, 1-8, 1-7, 1-6, 6-24, 6, 8, 16, 18 or 24 PEG units. In the formula below, the number n of PEG units can be for example 1-72, 5-23, 1-15, 1-16, 1-11, 1-12, 1-8, 1-7, 1-6, 7, 15, 17 or 23.

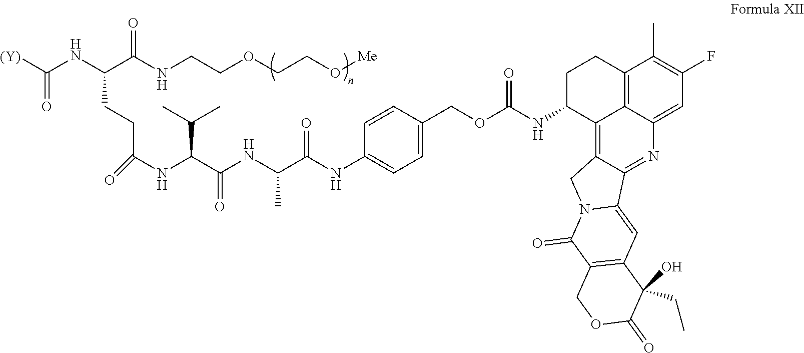

In such an embodiment, an immunoconjugate or an antibody-drug conjugate comprising a linker moiety having a maleimide as R group can have the structure of formula XII bis, wherein the maleimide moiety is bound to the sulfur group(S) of an amino acid (i.e. a cysteine) of the antibody (Ab) after the interchain disulfide bounds are reduced with a reducing agent.

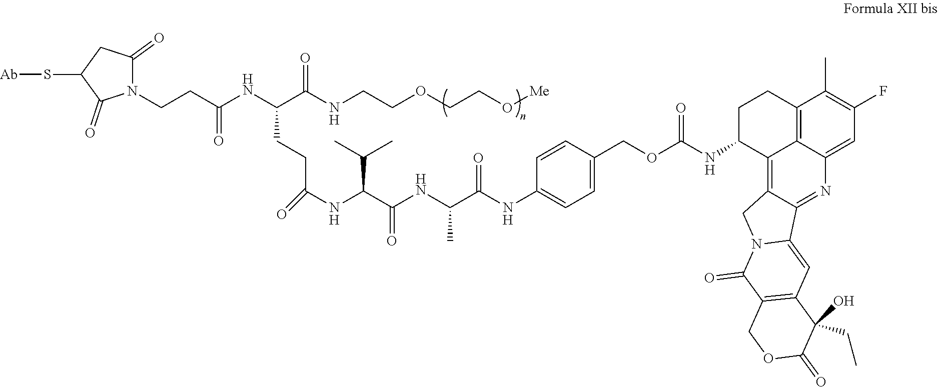

In a variant of Formula XII where the intracellularly cleavable peptide is valine-citrulline, an immunoconjugate or an antibody-drug conjugate comprising a linker moiety having a maleimide as R group can have the structure of formula XIIter.

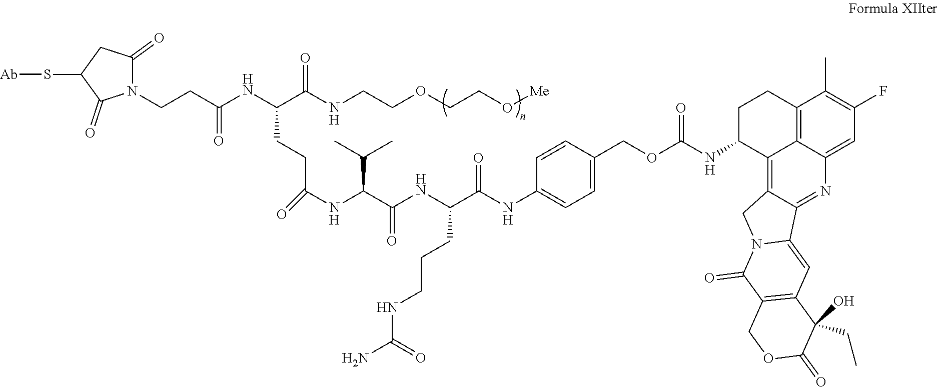

An exemplary linker having a maleimide as R group can have the structure of Compound 14a, below. Such linker can be conjugated to an antibody via cysteine residues in the antibody after the interchain disulfide bounds are reduced with a reducing agent. In this example, the linker moiety comprises 8 PEG units.

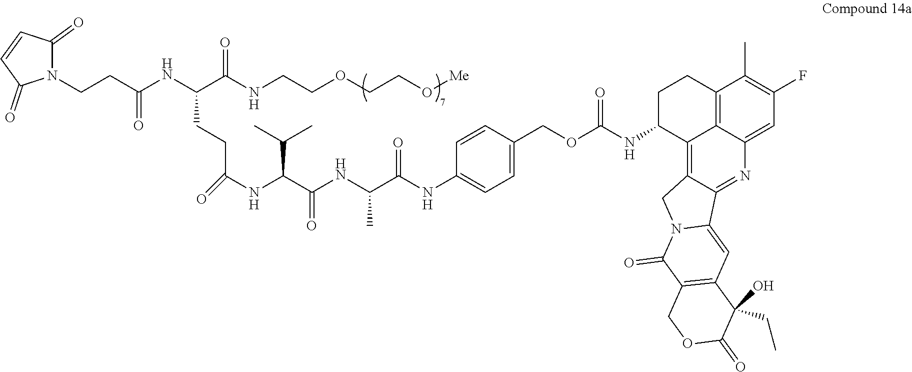

According to this example, one immunoconjugate or one antibody-drug conjugate comprising such a linker moiety can have the structure of Compound 14a bis, below.

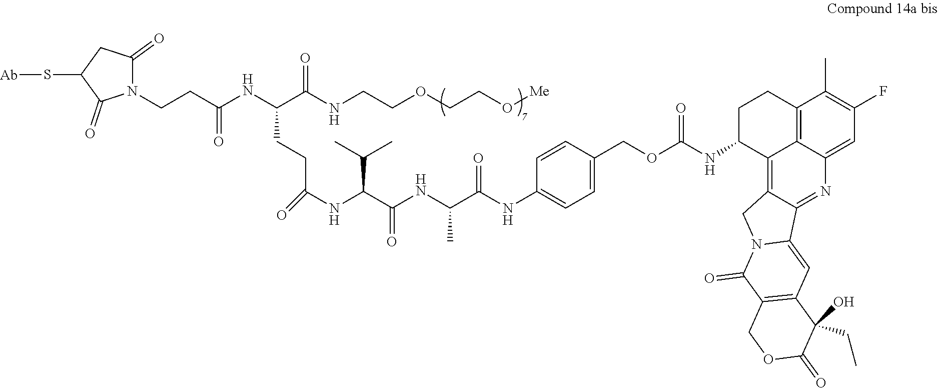

Another exemplary linker having a maleimide as R group can have the structure of Compound 14b, below. Such linker can be conjugated to an antibody via cysteine residues in the antibody after the interchain disulfide bounds are reduced with a reducing agent. In this example, the linker moiety comprises 7 PEG units.

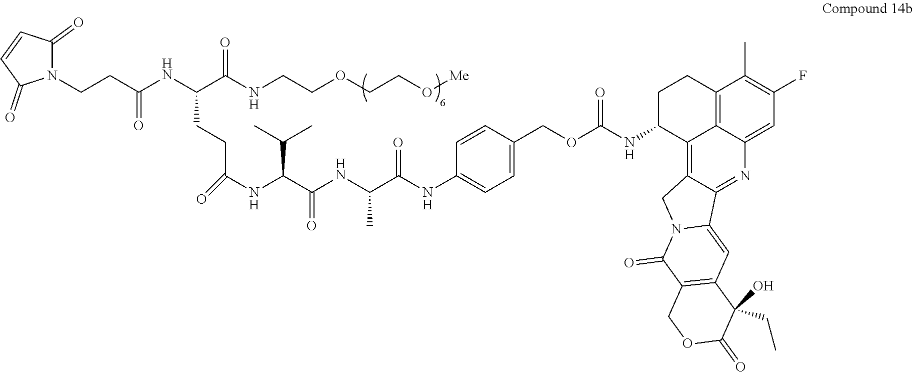

According to this example, one immunoconjugate or one antibody-drug conjugate comprising such a linker moiety can have the structure of Compound 14b bis, below.

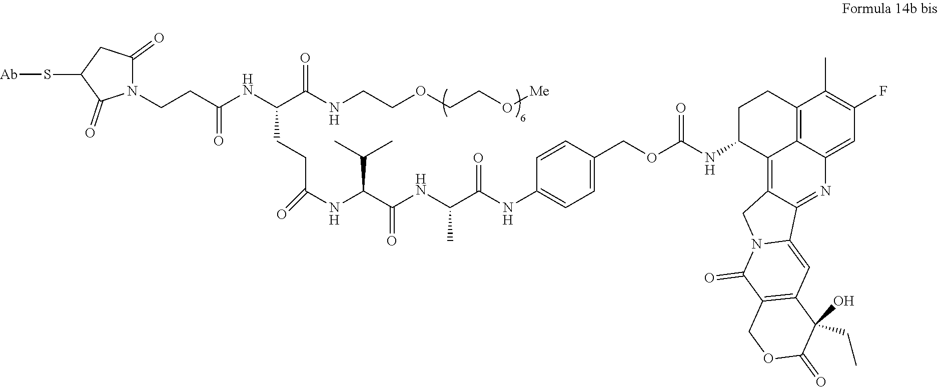

Another exemplary linker having a maleimide as R group can have the structure of Compound 14c, below. Such linker can be conjugated to an antibody via cysteine residues in the antibody after the interchain disulfide bounds are reduced with a reducing agent. In this example, the linker moiety comprises 16 PEG units.

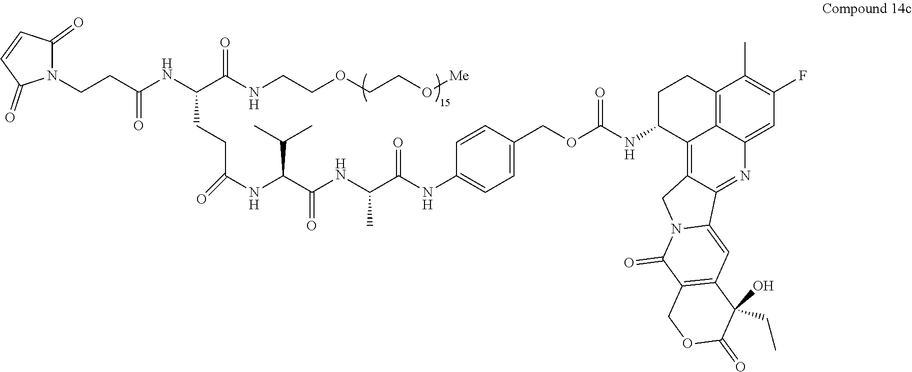

According to this example, an immunoconjugate or antibody-drug conjugate comprising such a linker moiety can have the structure of Compound 14c bis, below.

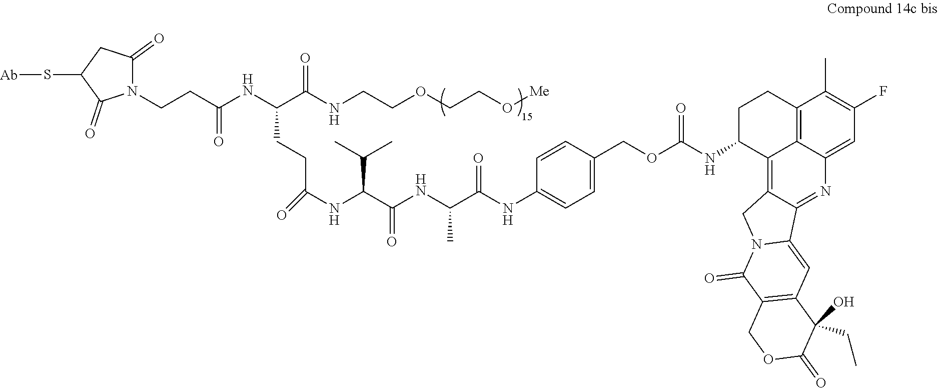

In one embodiment, the linker moiety (X-Z) is or comprises the structure according to Formula XIII below, wherein (Y) is a spacer comprising (e.g. at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native truncated or otherwise modified N-glycan bound to Kabat residue N297 of an antibody). The linker may comprise for example 1-72, 1-16, 1-12, 1-8, 1-7, 1-6, 6-24, 8, 16, 18 or 24 PEG units. In the formula below, the number n of PEG units can be for example 1-72, 5-23, 1-15, 1-16, 1-11, 1-12, 1-8, 1-7, 1-6, 7, 15, 17 or 23.

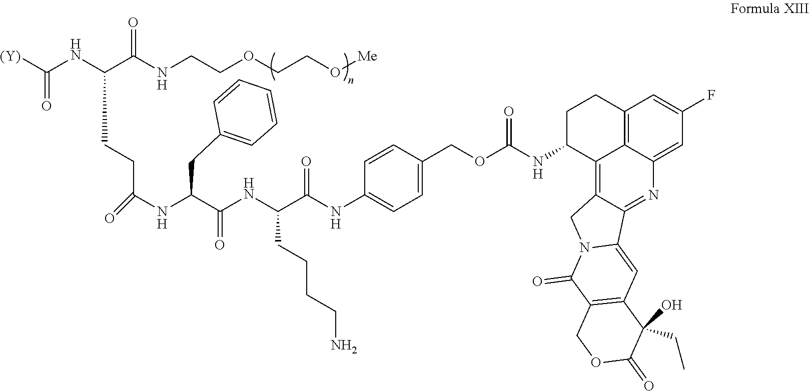

In such an embodiment, an immunoconjugate or antibody-drug conjugate comprising a linker moiety having a maleimide as R group can have the following formula XIII bis, wherein the maleimide moiety is bound to the sulfur group(S) of an amino acid (i.e. a cysteine) of the antibody (Ab) after the interchain disulfide bounds are reduced with a reducing agent.

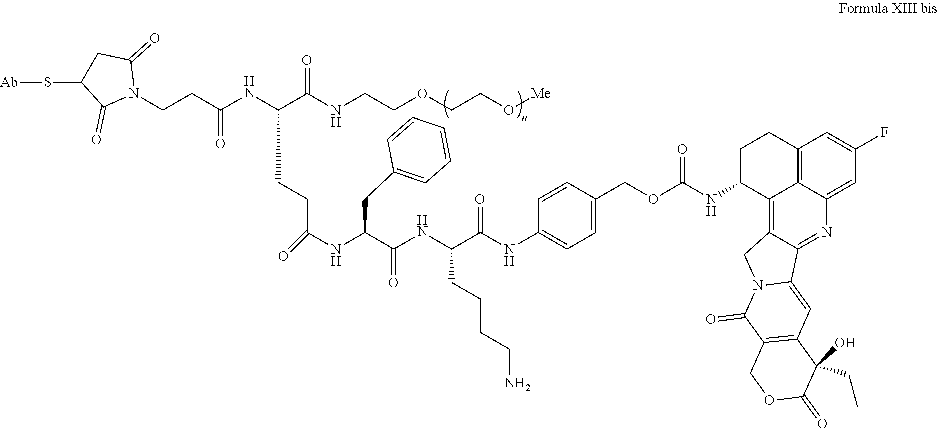

An exemplary linker having a maleimide as R group can have the structure of Compound 15a, below. Such linker can be conjugated to an antibody via cysteine residues in the antibody after the interchain disulfide bounds are reduced with a reducing agent. In this example, the linker moiety comprises 8 PEG units.

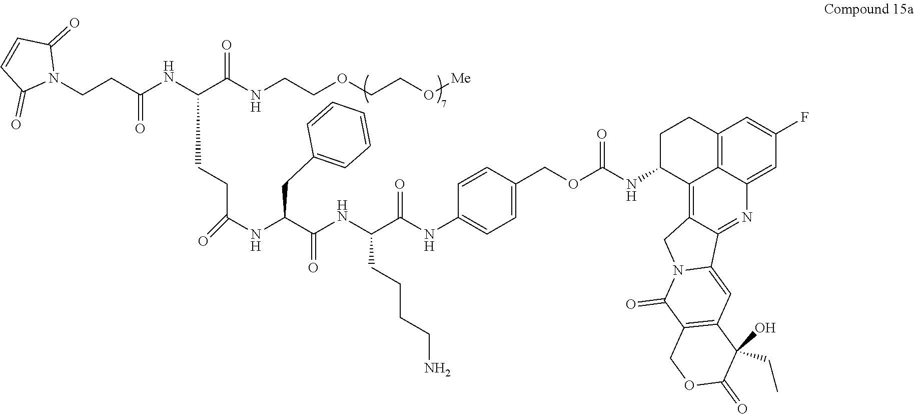

According to this example, an immunoconjugate or antibody-drug conjugate comprising such a linker moiety can have the structure of Compound 15a bis, below.

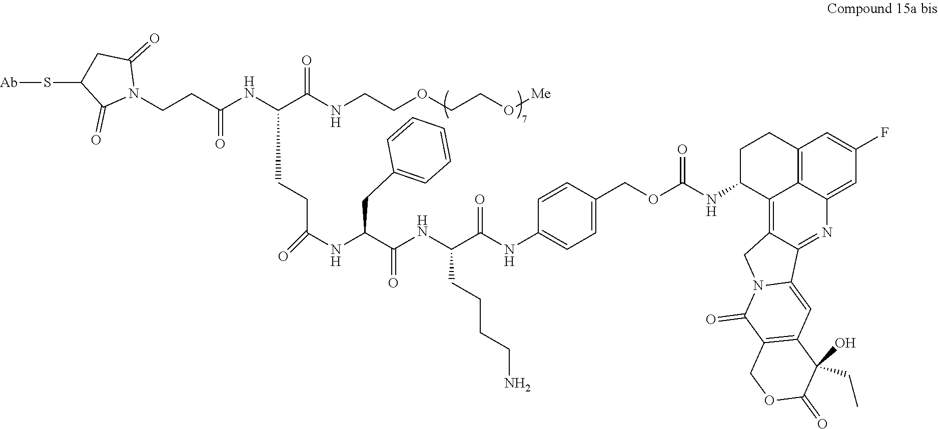

Another exemplary linker having a maleimide as R group can have the structure of Compound 15b, below. Such linker can be conjugated to an antibody via cysteine residues in the antibody after the interchain disulfide bounds are reduced with a reducing agent. In this example, the linker moiety comprises 7 PEG units.

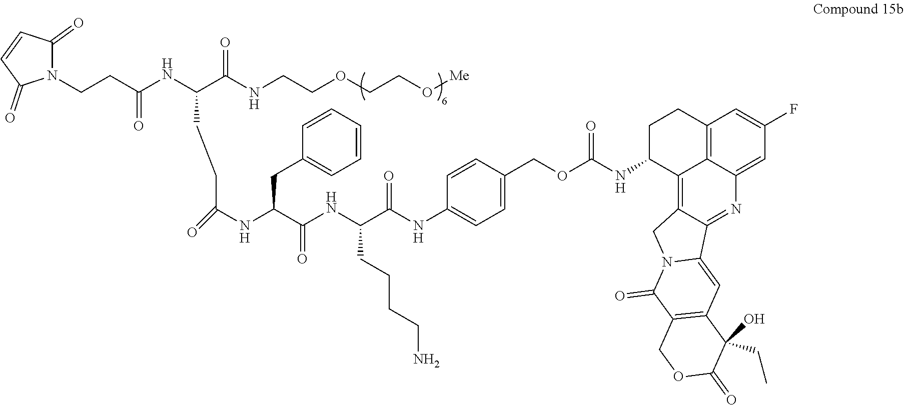

According to this example, animmunoconjugate or antibody-drug conjugate comprising such a linker moiety can have the structure of Compound 15b bis, below.

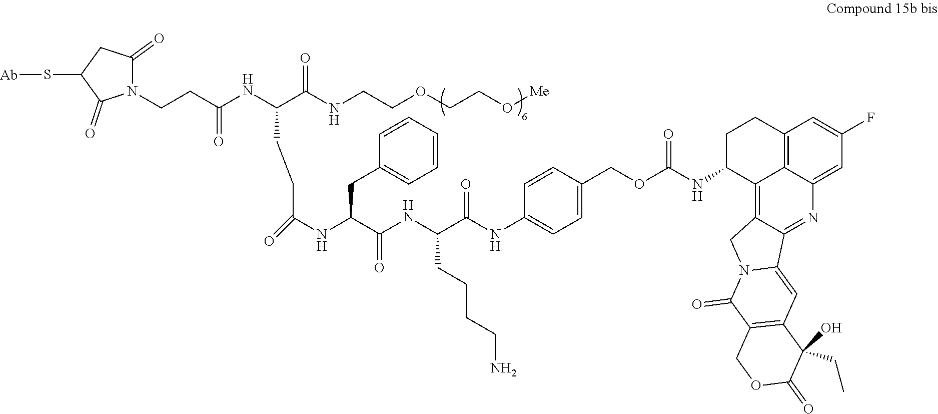

In one embodiment, the linker moiety (X-Z) is or comprises the structure according to Formula XIV below, wherein (Y) is a spacer comprising (e.g. at its terminus) the residue of the reaction of a reactive group (R) with an amino acid residue, for example a free amino, hydroxyl, sulfhydryl or carboxyl group on the antibody (e.g., on the epsilon amino group of one or more lysine residues, the free carboxylic acid group of one or more glutamic acid or aspartic acid residues, or to the S atom of one or more cysteinyl residues), or for example a glycan structure of a glycosylated amino acid residue (e.g. a native truncated or otherwise modified N-glycan bound to Kabat residue N297 of an antibody). In the formula below, the number n of PSAR units can be for example 1-72, 1-24, 1-16, 1-10, 1-12, 1-8, 1-6, 8, 16, 18 or 24.

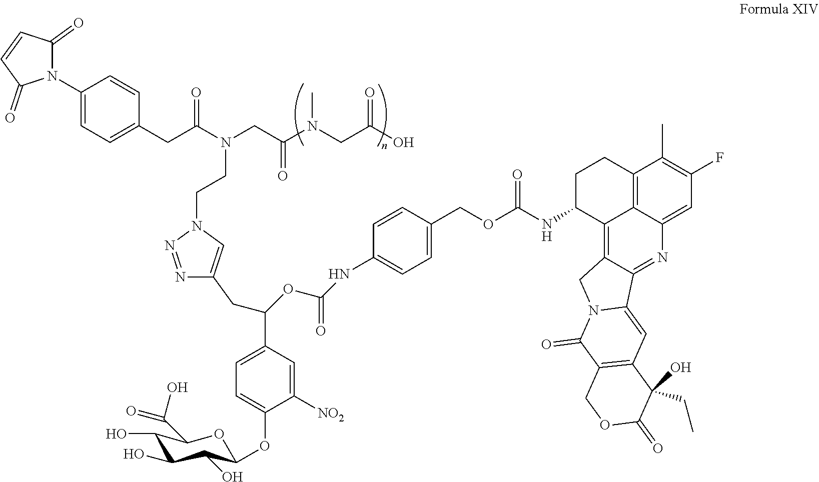

An exemplary linker having a maleimide as R group can have the structure of Compound 16, below. Such linker can be conjugated to an antibody via cysteine residues in the antibody after the interchain disulfide bounds are reduced with a reducing agent.

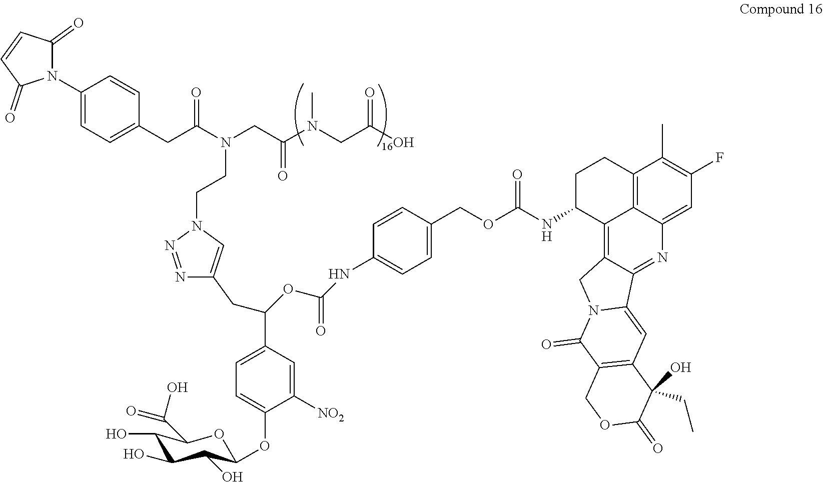

According to this example, an immunoconjugate or antibody-drug conjugate comprising such a linker moiety can have the structure of Compound 16 bis, below.

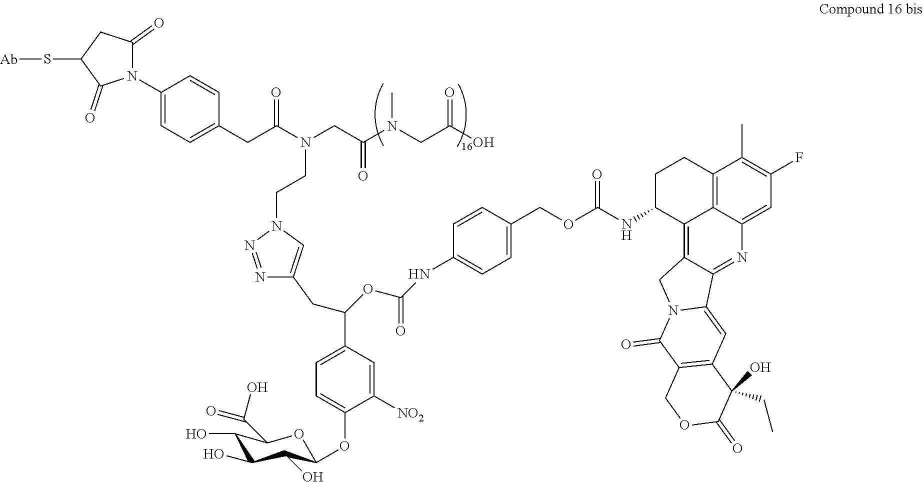

In another embodiment, a linker can have a primary amine as R group, and when reacted with an antibody in the presence of a transglutaminase enzyme, can yield an antibody comprising one or a plurality of acceptor glutamine residues functionalized with the linker. For example, a linker (X-Z), or an antibody functionalized therewith, has or comprises the structure shown as Compound 12, below:

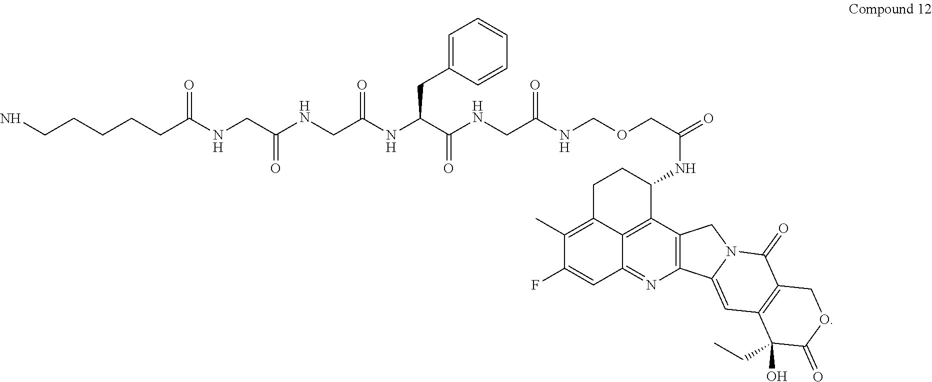 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN)CC3]

An anti-Nectin-4 binding protein functionalized with an oligopeptide-containing linker of Formulae XI or Compounds 11 and 12 result in the release (e.g. intracellularly, in the presence of Nectin-4 expressing tumor cells) of a substituted exatecan having a OH—CH2-C(═O) substituent present at position 1 amine, as shown in the structure below:

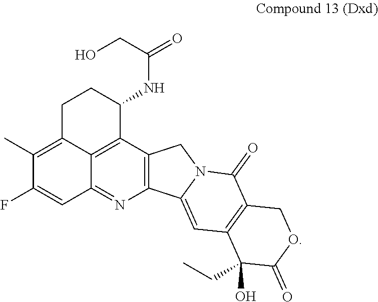 [SMILES: CC[C@@]1(O)C(=O)OCc2c1cc1n(c2=O)Cc2c-1nc1cc(F)c(C)c3c1c2[C@@H](NC(=O)CO)CC3]

The exemplary linkers of Formulae III, IV, V, VI, VII, VIII, IX, X, XI, XII, XIII, or XIV when prepared as a structure having a primary amine can reacted with antibody in the presence of a transglutamine enzyme (e.g. Bacterial Transglutaminase, BTG) such that the transglutaminase enzyme catalyzes the conjugation of the linker to an acceptor glutamine residue within the primary structure of the antibody, for example within an immunoglobulin constant domain or within a TGase recognition tag inserted or appended to (e.g., fused to) a constant region. Methods and linkers for use in BTG-mediated conjugation to antibodies is described in PCT publication no. WO2014/202773, the disclosure of which is incorporated by reference. Conjugation catalyzed by BTG permits precise control of the average drug: antibody ratio in a composition. The term “transglutaminase”, used interchangeably with “TGase” or “TG”, refers to an enzyme capable of cross-linking proteins through an acyl-transfer reaction between the γ-carboxamide group of peptide-bound glutamine and the ε-amino group of a lysine or a structurally related primary amine such as amino pentyl group, e.g. a peptide-bound lysine, resulting in a ε-(γ-glutamyl) lysine isopeptide bond. TGases include, inter alia, bacterial transglutaminase (BTG) such as the enzyme having EC reference EC 2.3.2.13 (protein-glutamine-γ-glutamyltransferase). The term “acceptor glutamine” residue, when referring to a glutamine residue of an antibody, means a glutamine residue that is recognized by a TGase and can be cross-linked by a TGase through a reaction between the glutamine and a lysine or a structurally related primary amine such as amino pentyl group. Preferably the acceptor glutamine residue is a surface-exposed glutamine residue. The term “TGase recognition tag” refers to a sequence of amino acids comprising an acceptor glutamine residue and that when incorporated into (e.g. appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the enzyme recognition tag. Examples of TGase recognition tags include the amino acid sequences disclosed in WO2012/059882 and WO2014/072482.

As exemplified in WO2013/092983 and WO2020/188061, the disclosures of which are incorporated herein by reference, the linker-drug moiety (X-Z) can be conjugated to glutamine residues in an antibody (acceptor glutamines) in two-step process comprising a first step in which a moiety comprising a primary amine and a first reactive group (R) is conjugated to the antibody in the presence of BTG, followed by a step of reacting the antibody-linker conjugate with a molecule comprising (i) a second reactive group (R′) that is reactive with the first reactive group and (ii) the cytotoxic agent (Z). Examples of reactive group pairs R and R′ include a range of groups capable of biorthogonal reaction, for example 1,3-dipolar cycloaddition between azides and cyclooctynes (copper-free click chemistry), between nitrones and cyclooctynes, oxime/hydrazone formation from aldehydes and ketones and the tetrazine ligation (see also WO2013/092983). The resulting linker and functionalized antibody, or the Y element thereof, can thus comprise a RR′ group resulting from the reaction of R and R′, for example a triazole.

An anti-Nectin-4 immunoconjugate can be incorporated in a pharmaceutical formulation in a concentration from 1 mg/ml to 500 mg/ml, wherein said formulation has a pH from 2.0 to 10.0. The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment, the pharmaceutical formulation is a freeze-dried formulation, whereto the physician or the patient adds solvents and/or diluents prior to use.

In another embodiment, the pharmaceutical formulation is a dried formulation (e.g. freeze-dried or spray-dried) ready for use without any prior dissolution.

In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0.

In another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5.

In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment, the formulation further comprises a pharmaceutically acceptable preservative. In a further embodiment, the formulation further comprises an isotonic agent. In a further embodiment, the formulation also comprises a chelating agent. In a further embodiment of the invention the formulation further comprises a stabilizer. In a further embodiment, the formulation further comprises a surfactant. For convenience reference is made to Remington: The Science and Practice of Pharmacy, 19^{th} edition, 1995. It is possible that other ingredients may be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Administration of pharmaceutical compositions according to the invention may be through several routes of administration, for example, intravenous. Suitable antibody formulations can also be determined by examining experiences with other already developed therapeutic ADCs.

In any embodiment, a composition can be characterized as comprising a plurality of Nectin-4 binding immunoconjugates of the disclosure, wherein at least 70%, 80%, 90%, 95%, 98% or 99% of the immunoconjugates in a sample have at least 4, 6 or 8 amino acid residues per antibody that are functionalized with a linker disclosed herein. In any embodiment, a composition can be characterized as comprising a plurality of Nectin-4 binding immunoconjugates of the disclosure, wherein at least 70%, 80%, 90%, 95%, 98% or 99% of the immunoconjugates in a sample have at least 2, 4, 6 or 8 amino acid residues per antibody that are functionalized with the linker-camptothecin moiety, e.g., the (X-Z) unit or the (—(Y)-(Pep)-(Y′)-(Z)) unit of the formulae herein. In any embodiment, a composition can be characterized as comprising a plurality of Nectin-4 binding immunoconjugates of the disclosure, wherein at least 70%, 80%, 90%, 95%, 98% or 99% of the immunoconjugates in a sample have the same number of functionalized amino acid per antibody, optionally wherein the number is 4, 6 or 8.

### Diagnostics, Prognostics, and Treatment of Malignancies

In some aspects, described are methods as well as antibodies, antibody fragments and immunoconjugates useful in the diagnosis, prognosis, monitoring and treatment of a cancer characterized by tumor cells that express at their surface Nectin-4. In therapeutic methods, the treatment comprises administering to a human subject or individual an antibody of the disclosure. In some embodiments, provided are improved methods for improved delivery of cytotoxic agents to Nectin-4-positive tumors, particular camptothecin analogues. The improved delivery of the cytotoxic agent may be the result of antibody-mediated inhibition of cluster formation and/or anchorage-independent growth of Nectin-4 expressing tumor cells, resulting in improved tumor penetration of the cytotoxic agent and/or an ADC comprising such agent. Treatment with an ADC of the disclosure is particularly advantageous for the treatment of disease with lower or heterogeneous Nectin-4 expression, for patients having particularly resistant disease or for whom other ADCs are not suitable, and/or for use in combination with additional therapeutic agents that mediate toxicity as single agents (e.g., chemotherapeutic agents).

In one embodiment, an anti-Nectin-4 antibody or antibody fragment may be useful to sensitize a tumor to a cytotoxic or chemotherapeutic agent. The anti-Nectin-4 antibody may be useful in the reinforcement or enhancement of toxicity of an anti-cancer agent against cancer, as compared to the anti-cancer agent itself, by reducing the chemoresistance of cancerous cells. A cancer-sensitizing composition comprising anti-Nectin-4 antibody or antibody fragment may be useful to sensitize a tumor (e.g. a Pgp-expressing tumor) to an anti-cancer agent, to reduce the chemoresistance and to improve the therapeutic effect of the anti-cancer agent.

In one embodiment, an anti-Nectin-4 antibody or antibody fragment of the disclosure can be specified as being for use in treating a tumor in a human individual in combination with a chemotherapeutic agent, wherein the anti-Nectin-4 antibody, antibody fragment and the chemotherapeutic agent are formulated for separate administration and are administered concurrently or sequentially. For example, the treatment can comprising administering to the individual an effective amount of each of: (a) an anti-Nectin-4 antibody or antibody fragment of the disclosure, and (b) an anti-cancer agent, optionally a chemotherapeutic agent, optionally a chemotherapeutic agent known to be capable of being transported by P-glycoprotein (Pgp), optionally an anthracycline, a vinca alkaloid, an etoposide, a taxane, a platinum compound or optionally a camptothecin.

In one embodiment, the antibody or antibody fragment is conjugated to a cytotoxic agent, e.g., a camptothecin, an exatecan or SN-38 molecule. The antibody or antibody fragment will typically be conjugated to a plurality of molecules of a cytotoxic agent, e.g. a camptothecin analogue, an exatecan. A cytotoxic agent, for example a camptothecin or exatecan, can be conjugated to the antibody via a linker comprising a protease-cleavable oligopeptide linker, e.g., a linker-toxin of any of Formulae VII-XIV. An exemplary pharmaceutical composition can comprise on average from 1 to 8 cytotoxic agent molecules (e.g. camptothecin derivative, exatecan, linker-toxin of any of Formulae VII-XIV) per antibody molecule, optionally from 2-8, from 4-8, from 6-8 cytotoxic agent molecules per antibody molecule, or for example about 2, 4, 5, 6, 7 or 8 cytotoxic agent molecules per antibody molecule. When the antibody or antibody fragment is conjugated to exatecan (i.e. via a linker, e.g. a linker-toxin of any of Formulae VII-XIV), such immunoconjugate is particularly advantageous to treat a Pgp-expressing tumor (e.g. a tumor characterized by expression of Pgp (MDR1). Tumor characterized by expression of Pgp include tumors following treatment with chemotherapeutic agents and ADCs, for example it has been shown that treatment with ADCs comprising auristatin (MMAE) payloads induce Pgp expression on tumors. Additionally, a range of tumors characterized by natural Pgp/MDR1 expression are known, for example significant levels of MDR1 are expressed by kidney cancer, adrenocortical carcinoma, cholangiocarcinoma, liver cancer, rectal cancer, colon cancer, brain cancer, pancreatic cancer, prostate cancer, mesothelioma, stomach cancer, AML, thyroid cancer, DLBC, esophageal cancer, breast cancer, sarcoma, testical cancer, uterine cancer, thymoma, cervical cancer, lung cancer, bladder cancer (e.g., urothelial carcinoma), and head and neck squamous cell carcinoma.

The Nectin-4 binding agent (e.g. anti-Nectin-4 antibody or antibody fragment) conjugated to a cytotoxic agent can be used advantageously to treat an individual having a Nectin-4-expressing cancer characterized by tumor cells that express Nectin-4 (e.g. at the tumor cell membrane or cell surface). Example of such cancers are urothelial cancer, breast cancer (e.g. triple-negative breast cancer; HER2-positive breast cancer), non-small cell lung cancer, pancreatic cancer, ovarian cancer, gastric cancer, colorectal cancer (e.g. colon cancer), head and neck squamous cell carcinoma and esophageal cancer.

The Nectin-4 binding agent conjugated to a cytotoxic agent can be used in Nectin-4 high-expressing tumors.

The Nectin-4 binding agent conjugated to a cytotoxic agent can also be used in heterogeneous and/or low Nectin-4-expressing tumors. In such tumors, the immunoconjugates of the disclosure can provide advantageous efficacy, optionally via avoidance of MDR1-mediated resistance and/or bystander anti-tumor effects.

The Nectin-4 binding agent conjugated to a cytotoxic agent can be used advantageously to treat an individual regardless of Nectin-4 expression levels, regardless of heterogeneity of Nectin-4 expression levels on tumor cells within an individual, and/or regardless of whether or not the individual has been previously treated with enfortumab vedotin. Where the cytotoxic agent is an exatecan, the Nectin-4 binding agent conjugated to a cytotoxic agent can also be used advantageously to treat an individual regardless of tumor Pgp expression and/or regardless of whether or not the individual has been previously treated with an ADC comprising a cytotoxic agent that is capable of being transported by Pgp (for example an ADC comprising an anti-Nectin-4 antibody conjugated to a camptothecin analogue other than exatecan, an ADC comprising an anti-Nectin-4 antibody conjugated to Dxd (e.g. via a GGFG-containing linker), or an anti-Nectin-4 antibody conjugated to an auristatin, an anthracycline, a vinca alkaloid, an etoposide, a taxane or a platinum compound).

In one embodiment, the Nectin-4 binding agent conjugated to a camptothecin derivative molecule can be used advantageously to treat an individual who has been previously treated with enfortumab vedotin. Such an individual may optionally have a cancer characterized by heterogeneous and/or low Nectin4-expressing tumors following enfortumab vedotin treatment. Such an individual may optionally have a cancer characterized by Pgp-expressing tumors following enfortumab vedotin treatment. An individual may have a cancer that is resistant, has not responded, has relapsed and/or progressed despite (e.g. during or following) treatment with an antibody conjugated to an auristatin or MMAE molecule (e.g., enfortumab vedotin). For example, the individual may have a locally advanced or metastatic urothelial cancer and has previously received treatment with an antibody conjugated to an auristatin or MMAE molecule (e.g., enfortumab vedotin).

In one embodiment, an immunoconjugate comprising a Nectin-4 binding agent conjugated via a linker to an exatecan is used advantageously to treat an individual who has been previously treated with an immunoconjugate comprising a Nectin-4 binding agent conjugated via a linker to a cytotoxic agent that is capable of being transported by Pgp. In one embodiment, an immunoconjugate comprising an Nectin-4 binding agent conjugated via a linker to an exatecan is used advantageously to treat an individual having a cancer characterized by heterogeneous and/or low Nectin4-expressing tumors following treatment with an immunoconjugate comprising a Nectin-4 binding agent conjugated via a linker to a cytotoxic agent that is capable of being transported by Pgp. In one embodiment, an immunoconjugate comprising a Nectin-4 binding agent conjugated via a linker to an exatecan is used advantageously to treat an individual having a cancer that is resistant, has not responded, has relapsed and/or progressed despite (e.g. during or following) treatment with an Nectin-4 binding agent conjugated via a linker to an cytotoxic agent that is capable of being transported by Pgp. Optionally, the Nectin-4 binding agent conjugated via a linker to a cytotoxic agent that is capable of being transported by Pgp comprises an anti-Nectin-4 antibody conjugated via a linker to a camptothecin analogue so as to release, upon cleavage of the linker, a camptothecin analogue other than exatecan (e.g. a Dxd or deruxtecan). Optionally, the Nectin-4 binding agent conjugated via a linker to a cytotoxic agent that is capable of being transported by Pgp comprises an anti-Nectin-4 antibody conjugated to Dxd (e.g. via a GGFG-containing linker). Optionally, the Nectin-4 binding agent conjugated via a linker to a cytotoxic agent that is capable of being transported by Pgp comprises an anti-Nectin-4 antibody conjugated to an auristatin, an anthracycline, a vinca alkaloid, an etoposide, a taxane or a platinum compound). Optionally, the Nectin-4 binding agent conjugated via a linker to a cytotoxic agent that is capable of being transported by Pgp comprises an anti-Nectin-4 antibody that binds to the IgV domain of Nectin-4. Optionally, the Nectin-4 binding agent conjugated via a linker to a cytotoxic agent that is capable of being transported by Pgp comprises an anti-Nectin-4 antibody that binds to the VC1 bridging domain of Nectin-4. In one embodiment, the immunoconjugate comprising an Nectin-4 binding agent conjugated via a linker to an exatecan comprises a linker having an enzymatically cleavable moiety (and optionally a self-immolating spacer) that results in the release of exatecan upon cleavage.

In advanced recurrent or metastatic urothelial cancer, a significant proportion of individuals will express high levels of Nectin-4 on tumor cells, e.g. H-score of at least 290 (See EV-201 clinical trial Cohort 1 Nectin-4 expression). However, a subset of patients have an H-score of less than 250, and some less than 200. A minority of patient had an H-score of less than 150, with some having an H-score of less than 100. In triple negative breast cancer (TNBC), it has been reported that 62% of patients have high Nectin-4 expression on tumor cells and 38% have low Nectin-4 expression on tumor cells (Rabat et al., 2017 Annals Onc. 28:769-776). In other cancer types, median H-score values for Nectin-4 expression have typically been lower than that observed in UC, notably in non-small cell lung cancer, pancreatic cancer, ovarian cancer, head and neck squamous cell carcinoma and esophageal cancer.

In one embodiment, an individual treated according to the disclosure has an advanced recurrent or metastatic cancer, optionally an advanced recurrent or metastatic urothelial cancer.

In one embodiment, an individual treated according to the disclosure has a cancer (e.g., a breast cancer) that tests positive for estrogen receptors and/or progesterone receptors, and tests negative for epidermal growth factor receptor 2 (HER2) or excess HER2 protein, optionally the cancer test positive for HER2 but HER2 is expressed at low levels.

In one embodiment, an individual treated according to the disclosure has a triple-negative breast cancer (TNBC), e.g., a breast cancer that tests negative for estrogen receptors, progesterone receptors, and excess HER2 protein.

In one embodiment, an individual treated according to the disclosure has a cancer (e.g. a breast cancer) that tests positive for HER2 protein, optionally wherein the cancer is expresses excess HER2 protein (HER2 over-expression), optionally the cancer is expresses low levels of HER2 protein (lower than excess HER2 expression). In one embodiment, the individual is treated with an anti-Nectin-4 ADC, in combination with an agent (e.g. antibody) that binds a HER2 polypeptide (e.g. trastuzumab, pertuzumab); optionally wherein the agent that binds HER2 is an ADC; optionally wherein the antibody that binds HER2 is conjugated to a cytotoxic agent, optionally an auristatin, a maytansinoid (e.g. DM1) or a camptothecin analogue (e.g. Compound 1, 2 or 13); optionally wherein the antibody that binds HER2 is trastuzumab emtansine or trastuzumab deruxtecan (DS-8201a; Enhertu™).

In one embodiment, an individual treated according to the disclosure has a non-small cell lung cancer, optionally a lung adenocarcinoma.

In one embodiment, an individual treated according to the disclosure has a pancreatic cancer.

In one embodiment, an individual treated according to the disclosure has an ovarian cancer.

In one embodiment, an individual treated according to the disclosure has a head and neck squamous cell carcinoma.

In one embodiment, an individual treated according to the disclosure has an oesophageal cancer.

In one embodiment, an individual treated according to the disclosure has a colorectal cancer. Colorectal cancer (CRC) as used herein refers to colon cancer, rectal cancer, and colorectal cancer (cancer of both the colon and rectal areas).

In one embodiment, an individual treated according to the disclosure has a NSCLC or lung adenocarcinoma, a gastric cancer, a colorectal carcinoma, a pancreatic cancer, a urothelial carcinoma or bladder cancer that tests positive for HER2 protein, optionally wherein the cancer expresses excess HER2 protein (HER2 over-expression), optionally the cancer is expresses low levels of HER2 protein (lower than excess HER2 expression). In one embodiment, the individual is treated with an anti-Nectin-4 ADC according to disclosure, in combination with an agent (e.g. antibody) that binds HER2 polypeptides (e.g. an antibody comprising the heavy and light chains CDRs or variable regions of trastuzumab or pertuzumab); optionally wherein the agent that binds HER2 is an ADC; optionally wherein the antibody that binds HER2 is conjugated to a cytotoxic agent, optionally an auristatin, a maytansinoid (e.g. DM1) or a camptothecin analogue (e.g. Compound 1, 2 or 13); optionally wherein the antibody that binds Her2 is trastuzumab emtansine or trastuzumab deruxtecan (DS-8201a).

As shown herein, an ADC that releases exatecan upon cleavage (e.g. upon cleavage of the intracellularly cleavable linker that comprises the cytotoxic agent Z) is particularly advantageous in treatment of tumors that are resistant to ADCs with linkers that upon cleavage release cytotoxic agents (other than exatecan) that are transported by Pgp, for example auristatins or Dxd. Accordingly, in one embodiment, where the HER2 positive cancer is treated with a combination of anti-Nectin-4 binding agent (e.g. anti-Nectin-4 ADC) and anti-HER2 ADC, the anti-HER2 ADC can be designed to release exatecan upon cleavage (e.g. upon cleavage of the intracellularly cleavable linker that comprises the cytotoxic agent Z). The anti-Nectin-4 binding agent (e.g. anti-Nectin-4 ADC) can be designed to release exatecan upon cleavage (e.g. upon cleavage of the intracellularly cleavable linker that comprises the cytotoxic agent Z, or can release any other cytotoxic agent (Z), for example where Z is a taxane, anthracycline, camptothecin, epothilone, mytomycin, combretastatin, vinca alkaloid, nitrogen mustard, maytansinoid, duocarmycin, tubulysin, dolastatin, auristatin, enediyne, pyrrolobenzodiazepine, amatoxin or ethylenimine.

Accordingly, in one aspect, the present disclosure provides an immunoconjugate that binds a human Nectin-4 polypeptide, for use in the treatment of cancer (e.g., a HER2 positive Nectin-4-positive cancer), wherein the immunoconjugate that binds a human Nectin-4 polypeptide is represented by the formula:

Ab_{N4}-(X_{N4}-(Z_{N4}))
wherein,
Ab_{N4} is a polypeptide, peptide or antibody that specifically binds to a human Nectin-4 polypeptide;
X_{N4} is a molecule which connects Ab_{N4} and Z_{N4}, wherein X_{N4} comprises a cleavable moiety, e.g., under physiological conditions, optionally under intracellular conditions, optionally a protease-cleavable di-, tri-, tetra- or penta-peptide; and
Z_{N4} comprises a cytotoxic agent;
wherein the immunoconjugate that binds a human Nectin-4 polypeptide is for use in combination with an immunoconjugate that binds a human HER2 polypeptide represented by the formula:

Ab_{HER2}-(X_{HER2}-(Z_{HER2}))
wherein,
Ab_{HER2} is a polypeptide, peptide or antibody that specifically binds to a human HER2 polypeptide, optionally wherein the Ab_{HER2} comprises the heavy and light chain CDRs or variable regions of trastuzumab or pertuzumab;
X_{HER2} is a molecule which connects Ab_{HER2} and Z_{HER2}, wherein X_{HER2} comprises a cleavable moiety, e.g., under physiological conditions, optionally under intracellular conditions, optionally a protease-cleavable di-, tri-, tetra- or penta-peptide; and
Z_{HER2} is an exatecan. The immunoconjugate that binds a human HER2 polypeptide releases exatecan upon cleavage of the cleavable moiety. Optionally, Z_{N4} is an exatecan, optionally the immunoconjugate that binds a human Nectin-4 polypeptide releases exatecan upon cleavage of the cleavable moiety.

Also, in one aspect, the present disclosure provides an immunoconjugate that binds a human HER2 polypeptide, for use in the treatment of cancer e.g., a HER2 positive Nectin-4-positive cancer), wherein the immunoconjugate that binds a human HER2 polypeptide is represented by the formula:

Ab_{HER2}-(X_{HER2}-(Z_{HER2}))
wherein,
Ab_{HER2} is a polypeptide, peptide or antibody that specifically binds to a human HER2 polypeptide, optionally wherein the Ab_{HER2} comprises the heavy and light chain CDRs or variable regions of trastuzumab or pertuzumab;
X_{HER2} is a molecule which connects Ab_{HER2} and Z_{HER2}, wherein X_{HER2} comprises a cleavable moiety, e.g., under physiological conditions, optionally under intracellular conditions, optionally a protease-cleavable di-, tri-, tetra- or penta-peptide; and Z_{HER2} is an exatecan;
wherein the immunoconjugate that binds a human HER2 polypeptide is for use in combination with an immunoconjugate that binds a human Nectin-4 polypeptide represented by the formula:

Ab_{N4}-(X_{N4}-(Z_{N4}))
wherein,
Ab_{N4} is a polypeptide, peptide or antibody that specifically binds to a human Nectin-4 polypeptide;
X_{N4} is a molecule which connects Ab_{N4} and Z_{N4}, wherein X_{N4} comprises a cleavable moiety, e.g., under physiological conditions, optionally under intracellular conditions, optionally a protease-cleavable di-, tri-, tetra- or penta-peptide; and
Z_{N4} comprises a cytotoxic agent. The immunoconjugate that binds a human HER2 polypeptide releases exatecan upon cleavage of the cleavable moiety. Optionally, Z_{N4} is an exatecan, optionally the immunoconjugate that binds a human Nectin-4 polypeptide releases exatecan upon cleavage of the cleavable moiety.

Accordingly, in one aspect, the present disclosure provides an immunoconjugate that binds a human Nectin-4 polypeptide, for use in the treatment of cancer (e.g., a TROP-2 positive Nectin-4-positive cancer), wherein the immunoconjugate that binds a human Nectin-4 polypeptide is represented by the formula:

Ab_{N4}-(X_{N4}-(Z_{N4})
wherein,
Ab_{N4} is a polypeptide, peptide or antibody that specifically binds to a human Nectin-4 polypeptide;
X_{N4} is a molecule which connects Ab_{N4} and Z_{N4}, wherein X_{N4} comprises a cleavable moiety, e.g., under physiological conditions, optionally under intracellular conditions, optionally a protease-cleavable di-, tri-, tetra- or penta-peptide; and
Z_{N4} comprises a cytotoxic agent;
wherein the immunoconjugate that binds a human Nectin-4 polypeptide is for use in combination with an immunoconjugate that binds a human TROP-2 polypeptide represented by the formula:

Ab_{TROP-2}-(X_{TROP-2}-(Z_{TROP-2}))
wherein,
Ab_{TROP-2} is a polypeptide, peptide or antibody that specifically binds to a human TROP-2 polypeptide, optionally wherein the Ab_{TROP-2} comprises the heavy and light chain CDRs or variable regions of datopotamab or sacituzumab;
X_{TROP-2} is a molecule which connects Ab_{TROP-2} and Z_{TROP-2}, wherein X_{TROP-2} comprises a cleavable moiety, e.g., under physiological conditions, optionally under intracellular conditions, optionally a protease-cleavable di-, tri-, tetra- or penta-peptide; and
Z_{TROP-2} is an exatecan. The immunoconjugate that binds a human TROP-2 polypeptide releases exatecan upon cleavage of the cleavable moiety. Optionally, Z_{N4} is an exatecan, optionally the immunoconjugate that binds a human Nectin-4 polypeptide releases exatecan upon cleavage of the cleavable moiety.

Also, in one aspect, the present disclosure provides an immunoconjugate that binds a human TROP-2 polypeptide, for use in the treatment of cancer e.g., a TROP-2 positive Nectin-4-positive cancer), wherein the immunoconjugate that binds a human TROP-2 polypeptide is represented by the formula:

Ab_{TROP-2}-(X_{TROP-2}-(Z_{TROP-2}))
wherein,
Ab_{TROP-2} is a polypeptide, peptide or antibody that specifically binds to a human TROP-2 polypeptide, optionally wherein the Ab TROP-2 comprises the heavy and light chain CDRs or variable regions of datopotamab or sacituzumab;
X_{TROP-2} is a molecule which connects Ab_{TROP-2} and Z_{TROP-2}, wherein X_{TROP-2} comprises a cleavable moiety, e.g., under physiological conditions, optionally under intracellular conditions, optionally a protease-cleavable di-, tri-, tetra- or penta-peptide; and
Z_{TROP-2} is an exatecan;
wherein the immunoconjugate that binds a human TROP-2 polypeptide is for use in combination with an immunoconjugate that binds a human Nectin-4 polypeptide represented by the formula:

Ab_{N4}-(X_{N4}-(Z_{N4}))
wherein,
Ab_{N4} is a polypeptide, peptide or antibody that specifically binds to a human Nectin-4 polypeptide;
X_{N4} is a molecule which connects Ab_{N4} and Z_{N4}, wherein X_{N4} comprises a cleavable moiety, e.g., under physiological conditions, optionally under intracellular conditions, optionally a protease-cleavable di-, tri-, tetra- or penta-peptide; and
Z_{N4} comprises a cytotoxic agent. The immunoconjugate that binds a human TROP-2 polypeptide releases exatecan upon cleavage of the cleavable moiety. Optionally, Z_{N4} is an exatecan, optionally the immunoconjugate that binds a human Nectin-4 polypeptide releases exatecan upon cleavage of the cleavable moiety.

Accordingly, in one aspect, the present disclosure provides an immunoconjugate that binds a human Nectin-4 polypeptide, for use in the treatment of cancer (e.g., a B7H3 positive Nectin-4-positive cancer), wherein the immunoconjugate that binds a human Nectin-4 polypeptide is represented by the formula:

AD_{N4}-(X_{N4}-(Z_{N4})
wherein,
Ab_{N4} is a polypeptide, peptide or antibody that specifically binds to a human Nectin-4 polypeptide;
X_{N4} is a molecule which connects Ab_{N4} and Z_{N4}, wherein X_{N4} comprises a cleavable moiety, e.g., under physiological conditions, optionally under intracellular conditions, optionally a protease-cleavable di-, tri-, tetra- or penta-peptide; and
Z_{N4} comprises a cytotoxic agent;
wherein the immunoconjugate that binds a human Nectin-4 polypeptide is for use in combination with an immunoconjugate that binds a human B7H3 polypeptide represented by the formula:

Ab_{B7H3}-(X_{B7H3}-(Z_{B7H3}))
wherein,
Ab_{B7H3} is a polypeptide, peptide or antibody that specifically binds to a human B7H3 polypeptide, optionally wherein the Ab_{B7H3} comprises the heavy and light chain CDRs or variable regions of enoblituzumab, ifinatamab, mirzotamab, obrindatamab, omburtamab or vobramitamab;
X_{B7H3} is a molecule which connects Ab_{B7H3} and Z_{B7H3}, wherein X_{B7H3} comprises a cleavable moiety, e.g., under physiological conditions, optionally under intracellular conditions, optionally a protease-cleavable di-, tri-, tetra- or penta-peptide; and
Z_{B7H3} is an exatecan. The immunoconjugate that binds a human B7H3 polypeptide releases exatecan upon cleavage of the cleavable moiety. Optionally, Z_{N4} is an exatecan, optionally the immunoconjugate that binds a human Nectin-4 polypeptide releases exatecan upon cleavage of the cleavable moiety.

Also, in one aspect, the present disclosure provides an immunoconjugate that binds a human B7H3 polypeptide, for use in the treatment of cancer e.g., a B7H3 positive Nectin-4-positive cancer), wherein the immunoconjugate that binds a human B7H3 polypeptide is represented by the formula:

Ab_{B7H3}-(X_{B7H3}-(Z_{B7H3}))
wherein,
Ab_{B7H3} is a polypeptide, peptide or antibody that specifically binds to a human B7H3 polypeptide, optionally wherein the Ab_{B7H3} comprises the heavy and light chain CDRs or variable regions of enoblituzumab, ifinatamab, mirzotamab, obrindatamab, omburtamab or vobramitamab;
X_{B7H3} is a molecule which connects Ab_{B7H3} and Z_{B7H3}, wherein X_{B7H3} comprises a cleavable moiety, e.g., under physiological conditions, optionally under intracellular conditions, optionally a protease-cleavable di-, tri-, tetra- or penta-peptide; and
Z_{B7H3} is an exatecan;
wherein the immunoconjugate that binds a human B7H3 polypeptide is for use in combination with an immunoconjugate that binds a human Nectin-4 polypeptide represented by the formula:

Ab_{N4}-(X_{N4}-(Z_{N4}))
wherein,
Ab_{N4} is a polypeptide, peptide or antibody that specifically binds to a human Nectin-4 polypeptide;
X_{N4} is a molecule which connects Ab_{N4} and Z_{N4}, wherein X_{N4} comprises a cleavable moiety, e.g., under physiological conditions, optionally under intracellular conditions, optionally a protease-cleavable di-, tri-, tetra- or penta-peptide; and
Z_{N4} comprises a cytotoxic agent. The immunoconjugate that binds a human B7H3 polypeptide releases exatecan upon cleavage of the cleavable moiety. Optionally, Z_{N4} is an exatecan, optionally the immunoconjugate that binds a human Nectin-4 polypeptide releases exatecan upon cleavage of the cleavable moiety.

In one aspect, the treatment methods of the disclosure are independent of the assessment or detection of Nectin-4 expression in tumor tissue and/or independent of the expression level of Nectin-4 on tumor cells and/or the frequency or number of Nectin-4-expressing tumor cells in a tissue sample from said individual. In one aspect, the treatment methods of the disclosure are independent of the assessment or detection of tumor Pgp (MDR1) expression.

In one aspect, the present invention provides methods of treating a cancer and/or eliciting an anti-tumor immune response in an individual in need thereof, wherein said individual has advanced recurrent or metastatic urothelial cancer or breast cancer (e.g. TNBC), wherein said methods do not necessitate the pre-determination of whether the individual has tumor tissue comprising cells (e.g. tumor cells) that express Nectin-4 or not.

In one aspect, the present invention provides methods of treating a cancer and/or killing tumor cells in an individual in need thereof, wherein said individual has advanced recurrent or metastatic urothelial cancer or breast cancer (e.g. TNBC), wherein said methods do not necessitate the pre-determination of whether or not the individual has tumor tissue comprising cells (e.g. tumor cells) that express high-levels of Nectin-4, e.g. as defined by an immunohistochemistry assessment (e.g. an H-score or other appropriate IHC scoring method).

In one aspect, the methods of treating a cancer and/or killing tumor cells in an individual do not necessitate the pre-determination of the level of Nectin-4 expression of tumor cells.

In one aspect, the methods of treating a cancer in an individual, optionally an advanced recurrent or metastatic urothelial cancer or a breast cancer (e.g. TNBC, HER2 positive cancer), comprise treating an individual having a cancer characterized by an H-score for Nectin-4 expression of no more than, or less than, 290, 250, 200, 150 or 100. In any embodiment for treating or preventing a cancer in an individual, the method can be specified as comprising the steps of: (i) identifying an individual whose tumor cells express Nectin-4 (e.g. as determined by immunohistochemistry), and (ii) administering to the individual an effective amount of an anti-Nectin-4 antibody drug conjugate of the disclosure.

In any embodiment for treating or preventing a cancer in an individual, the method can be specified as comprising the steps of (i) identifying an individual whose tumor cells express (a) Nectin-4 (e.g. as determined by immunohistochemistry), and (b) HER2, optionally wherein the tumor cells express low levels of HER2 (e.g. as determined by immunohistochemistry; as determined by Herceptest™) and (ii) administering to the individual an effective amount of an anti-Nectin-4 antibody drug conjugate of the disclosure, optionally in combination with an agent (e.g. antibody) that binds Her2 polypeptides (e.g. trastuzumab, pertuzumab); optionally wherein the antibody that binds Her2 is an ADC; optionally wherein the antibody that binds Her2 is conjugated to a cytotoxic agent, optionally an auristatin, a maytansinoid (e.g. DM1) or a camptothecin derivative (e.g. Compound 1, 2 or 13); optionally wherein the antibody that binds Her2 is trastuzumab emtansine or trastuzumab deruxtecan (DS-8201a).

In any embodiment for treating or preventing a cancer in an individual, the method can be specified as comprising the steps of: (i) identifying an individual whose tumor cells have a low or moderate level of Nectin-4 expression (e.g. as determined by immunohistochemistry), and (ii) administering, to the individual identified in step (i), an effective amount of an anti-Nectin-4 antibody drug conjugate of the disclosure.

In any embodiment for treating or preventing a cancer (e.g. a Nectin-4 positive cancer) in an individual, the method can be specified as comprising the steps of: (i) identifying an individual whose cancer is characterized by a low level of Nectin-4 expression (e.g. as determined by immunohistochemistry), and (ii) administering, to the individual identified in step (i), an effective amount of an anti-Nectin-4 antibody drug conjugate of the disclosure. In one embodiment, the individual has a cancer characterized by an H-score for Nectin-4 expression of no more than, or less than, 150 or 100.

In any embodiment for treating or preventing a cancer (e.g. a Nectin-4 positive cancer) in an individual, the method can be specified as comprising the steps of: (i) identifying an individual whose cancer is characterized by a moderate level of tumor Nectin-4 expression (e.g. as determined by immunohistochemistry), and (ii) administering to the individual an effective amount of an anti-Nectin-4 antibody drug conjugate of the disclosure. In one embodiment, the individual has a cancer characterized by an H-score by an H-score for Nectin-4 expression of no more than, or less than, 290, 250, 200, 150, optionally further wherein the cancer is characterized by an H-score for Nectin-4 expression of at least 100.

In a still further embodiment, provided is a method for treating or preventing a cancer (e.g. a Nectin-4 positive cancer) in an individual comprising: (i) identifying an individual whose cancer is characterized by an H-score for tumor Nectin-4 expression of no more than, or less than, 290, 250, 200, 150, 120 or 100, and (ii) administering to the individual an effective amount of an anti-Nectin-4 antibody drug conjugate of the disclosure. Optionally, step (i) may be specified as comprising a step of assessing Nectin-4 expression on tumor cells by histochemistry (e.g. IHC).

In a still further embodiment, provided is a method for treating or preventing a cancer (e.g. a Nectin-4 positive cancer; a breast cancer) in an individual comprising: (i) identifying an individual whose cancer is characterized by a QS-score for tumor Nectin-4 expression of no more than, or less than, 200, 150, 120 or 100, and (ii) administering to the individual an effective amount of an anti-Nectin-4 antibody drug conjugate of the disclosure. Optionally, step (i) may be specified as comprising a step of assessing Nectin-4 expression on tumor cells by histochemistry (e.g. IHC).

A biological sample from an individual, for example from a biopsy, can be obtained and assessed. Optionally, the sample is preserved as formaldehyde (e.g. formalin)-fixed paraffin embedded (FFPE) samples. Following deparaffination, the slides are amenable to methods to detect the expression of Nectin-4 (and/or HER2, TROP-2, B7H3).

Expression of Nectin-4, TROP-2, B7H3 and/or HER2 in tumor cells can be determined by any methods known in the art. In certain embodiments, assays include immunohistochemistry (IHC) assays, fluorescence activated cell sorting (FACS) assays, for example quantitative FACS, ELISA, immunoblotting (e.g. western blotting, dot blotting, or in-cell western blotting), and other immunoassays.

IHC staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Thus, antibodies or antisera, in some embodiments, polyclonal antisera, and in some embodiments, monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

In some embodiments, the IHC assay is a direct assay, wherein binding of antibody to the target antigen is determined directly. This direct assay uses a labeled reagent, such as a fluorescent tag or an enzyme-labeled primary antibody, which can be visualized without further antibody interaction. In some embodiments, the IHC assay is an indirect assay. In a typical indirect assay, unconjugated primary antibody binds to the antigen and then a labeled secondary antibody binds to the primary antibody. Where the secondary antibody is conjugated to an enzymatic label, a chromagenic or fluorogenic substrate is added to provide visualization of the antigen. Signal amplification occurs because several secondary antibodies may react with different epitopes on the primary antibody. The primary and/or secondary antibody used for immunohistochemistry typically will be labeled with a detectable molecule. Numerous labels are available, including radioisotopes, colloidal gold particles, fluorescent labels, and enzyme-substrate labels.

Strongly staining, moderately staining, and weakly staining are descriptions well known to those in the art. In some aspects strongly staining, moderately staining, and weakly staining are calibrated levels of staining, wherein a range is established and the intensity of staining is binned within the range. In some embodiments, strong staining is staining above the 75th percentile of the intensity range, moderate staining is staining from the 25th to the 75th percentile of the intensity range, and low staining is staining is staining below the 25th percentile of the intensity range. In some aspects one skilled in the art, and familiar with a particular staining technique, adjusts the bin size and defines the staining categories.

Control cell lines (e.g., centrifuged into a pellet and formalin fixed and paraffin embedded, e.g., and prepared as a tissue microarray, and e.g., stained with anti-Nectin-4 antibodies) with various staining intensities (e.g., when stained with anti-Nectin-4 antibodies) may be utilized as controls for IHC analysis. One of ordinary skill understands that other control cell pellets with negative, weak, moderate and high c-met staining intensity may readily be identified using the teachings of the present application and methods well known in the art and disclosed herein.

In some embodiments, a cancer or tumor is considered to be a Nectin-4-expressing cancer tumor when it is (e.g. is determined to be using an IHC assay) Nectin-4 positive.

In some embodiments, an individual's cancer or tumor is Nectin-4 positive when 5% or more of the tumor cells in the sample express Nectin-4 protein (e.g., express Nectin-4 protein at any intensity). In some embodiments, an individual's cancer or tumor is Nectin-4 positive when 10% or more of the tumor cells in the sample express Nectin-4 protein (e.g., express Nectin-4 protein at any intensity). In some embodiments, an individual's cancer or tumor is Nectin-4 positive when 20% or more of the tumor cells in the sample express Nectin-4 protein (e.g., express Nectin-4 protein at any intensity). In some embodiments, an individual's cancer or tumor is Nectin-4 positive when 30% or more of the tumor cells in the sample express Nectin-4 protein (e.g., express Nectin-4 protein at any intensity). In some embodiments, an individual's cancer or tumor is Nectin-4 positive when 40% or more of the tumor cells in the sample express Nectin-4 protein (e.g., express Nectin-4 protein at any intensity). In some embodiments, an individual's cancer or tumor is Nectin-4 positive when 50% or more of the tumor cells in the sample express Nectin-4 protein (e.g., express Nectin-4 protein at any intensity). In some embodiments, an individual's cancer or tumor is Nectin-4 positive when 60% or more of the tumor cells in the sample express Nectin-4 protein (e.g., express Nectin-4 protein at any intensity). In some embodiments, an individual's cancer or tumor is Nectin-4 positive when 70% or more of the tumor cells in the sample express Nectin-4 protein (e.g., express Nectin-4 protein at any intensity). In some embodiments, an individual's cancer or tumor is Nectin-4 positive when 80% or more of the tumor cells in the sample express Nectin-4 protein (e.g., express Nectin-4 protein at any intensity). In some embodiments, an individual's cancer or tumor is Nectin-4 positive when 90% or more of the tumor cells in the sample express Nectin-4 protein (e.g., express Nectin-4 protein at any intensity).

In some embodiments, an individual's cancer or tumor is Nectin-4 positive when 5% or more of the tumor cells in the sample express Nectin-4 protein with a moderate and/or strong staining intensity. In some embodiments, an individual's cancer or tumor is Nectin-4 positive when 10% or more of the tumor cells in the sample express Nectin-4 protein with a moderate and/or strong staining intensity. In some embodiments, an individual's cancer or tumor is Nectin-4 positive when 20% or more of the tumor cells in the sample express Nectin-4 protein with a moderate and/or strong staining intensity. In some embodiments, an individual's cancer or tumor is Nectin-4 positive when 30% or more of the tumor cells in the sample express Nectin-4 protein with a moderate and/or strong staining intensity. In some embodiments, an individual's cancer or tumor is Nectin-4 positive when 40% or more of the tumor cells in the sample express Nectin-4 protein with a moderate and/or strong staining intensity. In some embodiments, an individual's cancer or tumor is Nectin-4 positive when 50% or more of the tumor cells in the sample express Nectin-4 protein with a moderate and/or strong staining intensity. In some embodiments, an individual's cancer or tumor is Nectin-4 positive when 60% or more of the tumor cells in the sample express Nectin-4 protein with a moderate and/or strong staining intensity. In some embodiments, an individual's cancer or tumor is Nectin-4 positive when 70% or more of the tumor cells in the sample express Nectin-4 protein with a moderate and/or strong staining intensity. In some embodiments, an individual's cancer or tumor is Nectin-4 positive when 80% or more of the tumor cells in the sample express Nectin-4 protein with a moderate and/or strong staining intensity. In some embodiments, an individual's cancer or tumor is Nectin-4 positive when 90% or more of the tumor cells in the sample express Nectin-4 protein with a moderate and/or strong staining intensity.

Assessing immunohistochemistry assays in order to determine whether an individual's cancer or tumor is characterized by high Nectin-4 expression or not (e.g. low or moderate Nectin-4 expression) will typically involve application of a known scoring method. Low, moderate and high tumor Nectin-4 expression can be determined based on an “H-score” as described in US Pat. Pub. No. 2013/0005678. An H-score is obtained by the formula: (3×percentage of strongly staining cells)+ (2×percentage of moderately staining cells)+ (percentage of weakly staining cells), giving a range of 0 to 300. H-score has been used in particular in UC.

In some embodiments of any of the methods herein, low or moderate Nectin-4 expression (e.g., tumors or tumor cells having a low or moderate level of Nectin-4 expression) corresponds to an H-score of about 250 or lower, about 220 or lower, about 200 or lower, about 180 or lower, about 160 or lower, about 150 or lower, about 140 or lower, about 130 or lower, about 120 or lower, about 110 or lower or about 100 or lower.

In some embodiments of any of the methods herein, low Nectin-4 expression (e.g., tumors or tumor cells having a low level of Nectin-4 expression) corresponds to an H-score of 200 or lower, about 180 or lower, about 160 or lower, about 150 or lower, about 140 or lower, about 130 or lower, about 120 or lower, about 110 or lower or about 100 or lower.

In some embodiments of any of the methods herein, high Nectin-4 expression (e.g. tumors or tumor cells having a high level of Nectin-4 expression) corresponds to an H-score of about 290 or higher.

In another example, Nectin-4 staining can be scored according to the Quick score (QS) by using the following formula: QS=P (percentage of positive cells)×I (intensity), the maximum score being 300. QS has been used for example in breast cancer. For example, in TNBC some research groups have defined low Nectin-4 expression group as a QS= or <100. In some embodiments of any of the methods herein, low or moderate Nectin-4 expression (e.g., tumors or tumor cells having a low or moderate level of Nectin-4 expression) corresponds to QS-score of about 200 or lower, about 180 or lower, about 160 or lower, about 150 or lower, about 140 or lower, about 130 or lower, about 120 or lower, about 110 or lower or about 100 or lower.

Assays for assessing tumor cell expression of HER2 are well-known in the art. For example, assays such as the FDA-approved SPOT-Light HER2 CISH can be used to detect HER2 over-expression. Chromogenic in situ hybridization (CISH) detects HER2 gene amplification. This technique, also referred to as Subtraction Probe Technology Chromogenic In Situ Hybridization, is a test used see if breast cancer cells overexpress HER2 receptor proteins at the cell surface.

Another widely used assay for HER2 is the HercepTest™ (Dako North America, Inc.), a semiquantitative immunohistochemical assay used to determine HER2 protein overexpression in in formalin-fixed, paraffin-embedded cancer tissue. For example, tumors expressing low levels of HER2 can be identified by a score of +1 to +2 via HercepTest™. In one aspect, the treatment is used in an individual who has existing neuropathy, diabetes or hyperglycemia, cardiac insufficiency, an ocular pathology. Such conditions can render the individual unsuitable for treatment with anti-Nectin-4 ADCs such as enfortumab vedotin having higher toxicity or a more narrow therapeutic window than the anti-Nectin-4 antibody drug conjugates of the disclosure.

In any embodiment, the method of treatment may optionally comprise the steps of (a) assessing the cancer stage and/or disease progression in the individual; and (b) if the individual has recurrent, metastatic and/or progressing cancer, administering to the individual an effective amount of an anti-Nectin-4 antibody drug conjugate of the disclosure.

In some embodiments, the invention includes a method of treating a tumor in an individual having a urothelial cancer, comprising: (a) assessing the cancer stage and/or disease progression in the individual; and (b) if the individual has recurrent, metastatic and/or progressing cancer, administering to the individual an effective amount of an anti-Nectin-4 antibody drug conjugate of the disclosure.

Optionally, an individual treated with an anti-Nectin-4 antibody drug conjugate of the disclosure may have a cancer (e.g., a urothelial cancer, breast cancer (e.g. triple-negative breast cancer; HER2-positive cancer), non-small cell lung cancer, pancreatic cancer, ovarian cancer, gastric cancer, colorectal cancer, head and neck squamous cell carcinoma or esophageal cancer) that is resistant, has not responded, or has relapsed and/or progressed despite (e.g. during or following) surgery and/or treatment with a therapeutic agent, e.g. a chemotherapeutic agent, an antibody, an ADC or radiotherapy.

In any embodiment herein, treatment response can be defined and/or assessed according to well-known criteria, e.g. Response Evaluation Criteria In Solid Tumors (RECIST), such as version 1.1, see Eisenhauer et al. (2009) Eur. J. Cancer 45:228-247, or Immune-Related Response Criteria (irRC), see Wolchock et al. (2009) Clinical Cancer Research 15:7412-7420.

Optionally, an individual treated with an anti-Nectin-4 antibody drug conjugate of the disclosure has a tumor or cancer that displays resistance, that is not responsive to or that has progressed following treatment with a chemotherapeutic agent (e.g. a chemotherapeutic agent known to be capable of being transported by P-glycoprotein (Pgp), for example anthracyclines (doxorubicin, daunorubicin, taxanes (paclitaxel, docetaxel), Vinca alkaloids (vincristine, vinblastine, vindesine), and etoposides. Compounds recognized by Pgp are typically characterized as modestly hydrophobic (octanol-to-water partitioning coefficient, log P>1), often contain titratable protons with a net cationic charge under physiological conditions, and are predominately “natural products” with an aromatic moiety.

In some embodiments, an ADC comprising an anti-Nectin-4 antibody, antibody fragment is used or administered in the absence of combined administration of a chemotherapeutic agent.

In other embodiments, the anti-Nectin-4 antibody, antibody fragment or ADC comprising such is optionally used or administered in combination with a chemotherapeutic agent. Exemplary chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxy carbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. In one embodiment, the anti-Nectin-4 antibody, antibody fragment (or ADC comprising such) and the chemotherapeutic agent are formulated for separate administration and are administered concurrently or sequentially.

Optionally, the individual can be characterized as having cancer which has progressed, relapsed or not responded to prior treatment with a prior therapy, optionally further wherein the prior therapy comprises administration of enfortumab vedotin and/or administration of a PD-1 neutralizing agent (e.g., pembrolizumab, atezolizumab, nivolumab), optionally wherein the prior therapy is a chemotherapeutic agent.

Optionally, in any embodiment, the individual can be characterized being ineligible for treatment with enfortumab vedotin, and/or as having cancer which is not suitable or indicated for treatment with enfortumab vedotin.

Exemplary treatment protocols for treating a human with an anti-Nectin-4 antibody conjugated to an camptothecin derivative molecule include, for example, administering to the patient an effective amount of an anti-Nectin-4 antibody drug conjugate of the disclosure, wherein the method comprises at least one administration cycle in which at least one dose of the anti-Nectin-4 antibody conjugated to a camptothecin derivative molecule is administered at a dose of 0.1-10 mg/kg body weight, 0.1-5 mg/kg body weight, 0.1-1 mg/kg body weight, 1-10 mg/kg body weight or 1-5 mg/kg body weight. In one embodiment, a plurality of doses are administered, e.g. at least 2, 3, 4, 5, 6, 8, 10 doses. In one embodiment, administration of doses are separated by at least 2, 3 or 4 weeks. In one embodiment, the administration is every week, every two weeks, every three weeks or every four weeks.

In one embodiment the anti-Nectin-4 antibody drug conjugate of the disclosure is administered by i.v.

### EXAMPLES

### Example 1: Human Tumor Cells Co-Expressing Her2, TROP-2, B7H3 and Nectin-4

A study of HER2 and Nectin-4 gene expression was carried out using The Cancer Genome Atlas (a collaboration between the National Cancer Institute and National Human Genome Research Institute) based on multi-dimensional maps of the key genomic changes in different types of cancer. Significant correlations of HER2 and Nectin-4 expression was observed in particular in samples from pancreatic cancer, lung adenocarcinoma patients, breast cancer and bladder cancer patients. The highest correlation observed was pancreatic cancer, with correlation values: Spearman 0.71 and Pearson 0.78.

HER2 and Nectin-4 expression on SUM185 and SUM190 human breast cancer tumor cell lines (Biovit Inc.) was determined by flow cytometry. SUM185 originates from a pleural effusion of a patient with ER negative, PR negative and HER2 positive anaplastic carcinoma of the breast. The cell line over expresses Her2. SUM190 originates from a primary tumor from a patient with ER negative, PR negative and HER2 positive (amplified) breast cancer. Tumor cells were stained with anti-Nectin-4 antibody (ASG-22ME modified as a human IgG1 isotype containing a N297Q mutation having reduced Fc gamma receptor binding), anti-TROP-2 antibody, anti-B7H3 antibody or Anti-Her2 antibody (trastuzumab modified as human IgG1 isotype containing a N297Q mutation reduced Fc gamma receptor binding), as well as isotype control, at 10 μg/ml (at 4° C.), followed by PE conjugated polyclonal goat anti human antibodies at a dilution of 1:200. Samples were analyzed by cytofluorometric analysis with Canto II (HTS).

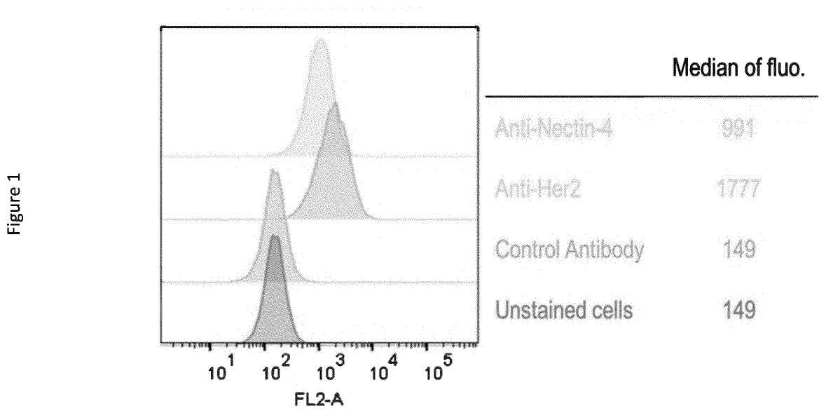

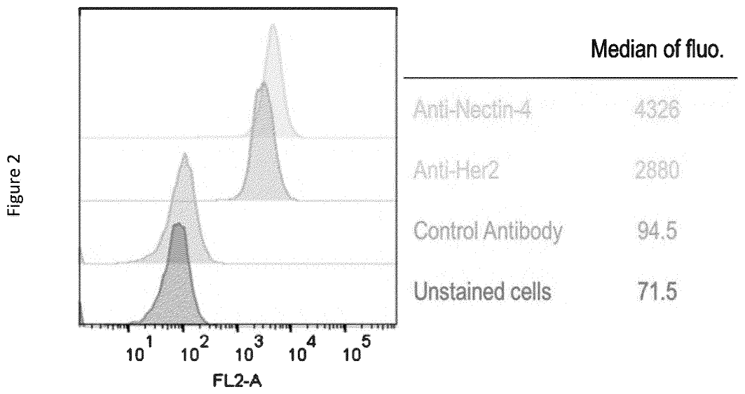

Representative results for HER2 and Nectin-4 are shown in FIG. 1 for SUM190 human breast cancer tumor cells and in FIG. 2 for SUM185 human breast cancer tumor cells. MFI: Median of fluorescence intensity. The SUM190 tumor cells expressed HER2 at low to moderate levels (median fluorescence units 1777) as well as Nectin-4 at lower levels (median 991 fluorescence units). The SUM185 cells expressed HER2 at moderate to high levels (median fluorescence units 2880) as well as Nectin-4 at higher levels (median 4326 fluorescence units). Additionally, the SUM185 cells expressed TROP-2 and B7H3 at high levels (median fluorescence units 17327 and 11481 respectively). Expression data are presented in Table 1 below.

TABLE 1

| | HER2 (MFI) | Nectin-4 (MFI) | TROP-2 (MFI) | B7H3 (MFI) |
|---|---|---|---|---|
| SUM185 | 2880 | 4326 | 17327 | 11481 |
| SUM190 | 1777 | 991 | | |

### Example 2: Efficacy of Anti-Nectin-4 Camptothecin-Derivative ADCs in Combination with Anti-HER2 ADCs

Anti-Nectin-4 antibody-drug conjugates were prepared and compared to trastuzumab antibody-drug conjugates for efficacy on HER2+ Nectin4+ human tumor cells. The anti-Nectin-4 antibody-drug conjugates were prepared having the VH and VL of SEQ ID NOS: 6 and 7 as human IgG1 isotype.

N41 VH:
(SEQ ID NO: 6)
QVQLKQSGPGLVQPSQSLSITCTVSGFSLTNYGVHWVRQSPGKGLEWL
GVIWSGGSTDYNAAFISRLSISKDTSKSQVFFKMNSLQADDTAIYYCA
RELIHAMDNWGQGTSVTVSS
N41 VL:
(SEQ ID NO: 7)
DIQMTQSPASLSVSVGETVTITCRASENIYSNLAWYQQKQGNSPQLLV
FAATNLADGVPSRFSGSGSGTQYSLKINSLQSEDEGTYYCQHFWGTPT
FGGGTKLEIK.

Anti-HER2 antibody-drug conjugates were prepared having the heavy and light chains of trastuzumab (human IgG1 isotype). Both the anti-Nectin-4 and the anti-HER2 antibodies were each conjugated stochastically to a linker-camptothecin derivative via cysteine residues in the antibody after partial reduction of interchain disulfide. A range of 2-10 molar equivalents of reducing agent tris(2-carboxyethyl) phosphine hydrochloride was incubated with antibody (3 mg/mL) for 2 h under agitation (350-400 rpm, +37° C.) to reduce disulfides. Conjugation of the linker-toxin was carried out by addition of a molar excess of linker-toxin at 9.2 or 12 molar equivalents incubated overnight on a stirring wheel at +37° C. The resulting ADCs had an average drug loading (drug: antibody ratio) of about 8. In a further example, anti-Nectin-4 antibodies were also conjugated to a second camptothecin (SN-38) containing linker using the same methods. The ADCs used in this Example are as follows.

N4 ADC1: Anti-Nectin-4 conjugated to a linker having the structure:

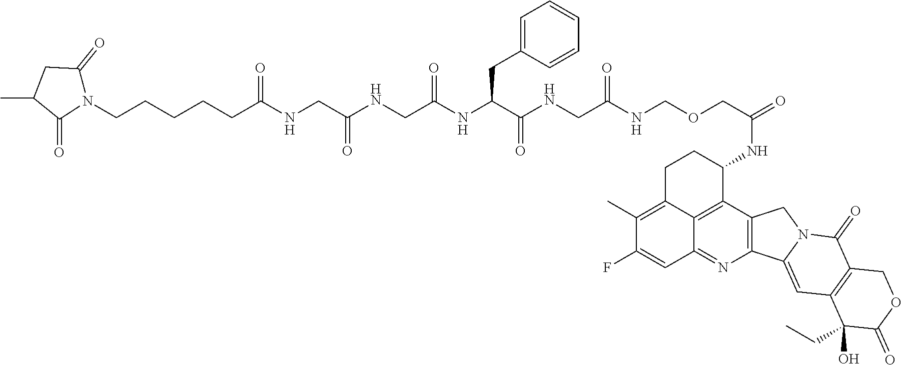 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

Her2 ADC1: Anti-HER2 conjugated to a linker having the structure:

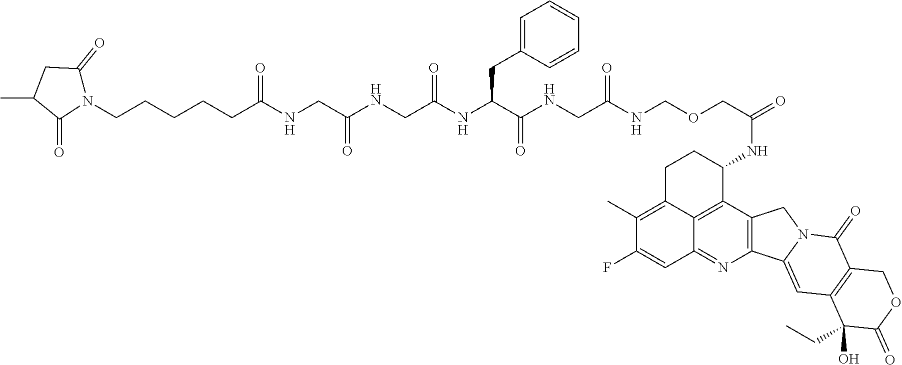 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

N4 ADC2: Anti-Nectin-4 conjugated to a linker having the structure:

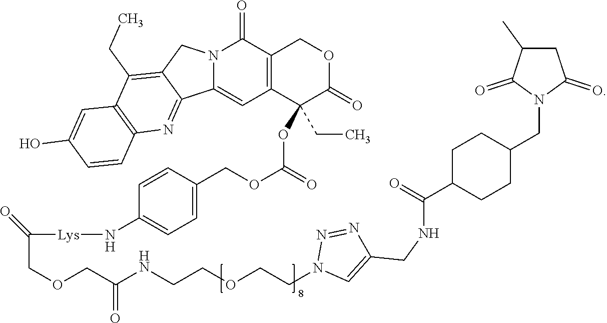

The resulting ADCs were tested for their ability to induce death of Nectin-4/HER2 expressing SUM190 when used in combination, MCF-7 tumor cells of Example 1. Briefly, cells were plated in 96 well plates (V=80 μl). N4 ADC1 and HER2 ADC1 or human IgG1-isotype control (IC)-linker-toxin or medium (conc. 5×) were tested in 1:2 serial dilution starting from (530 nM to 30 nM) and in a 1:5 serial dilution (7 nM to 7×10−2 nM) for the N4 ADC1 and isotype control. N4 ADC2 and isotype control were tested in 1:10 serial dilution starting from (530 nM to 5.3 10-2). The ADCs' ability to cause cell death was determined by assessing confluence using Incucyte S3-2 apparatus; viability at day 6 after treatment was determined using the Cell Titer Glo™ (CTG) assay with an Enspire2 apparatus. IC50 values for each ADC was determined using Luminescent Cell Viability at day 6 data with GraphPad Prism8. Experiments were repeated twice.

Results showed that the combination of anti-Nectin-4 ADCs and anti-Her2 ADCs had an improved potency (lower IC50) in causing the death of the Nectin-4+Her2+ tumor cells compared to either ADC used alone.

### Example 3: Modelling and Generation of a First Set of Anti-huNectin-4 Antibodies with Human Framework Sequences

Human VH and VK templates were identified for the introduction of CDRs of antibody 5E7. Each VH, VJ, VK and JK framework was analysed individually. Parental antibody having VH and VL amino acid sequences of SEQ ID NOS: 19 and 20, respectively, was then modified by the introduction into the VH of heavy chain frameworks (FR1, FR2, FR3) from the human subgroup IGHV1-46*01 together with IGHJ4*01 (FR4), and the introduction into the VL of light chain frameworks (FR1, FR2, FR3) from the human subgroup IGKV2-28*01, together with IGKJ4*01 (FR4).

A parental chimeric antibody Fab (HPLP) was modelled using the following heavy and light chain sequences:

HP heavy chain (variable domain underlined):
(SEQ ID NO: 67)
QVQLQQPGAELVKPGASVKLSCKASGYIFTSYWMHWVKQRPGQGLEWI
GEIDPSDSYTNYNQKFKGKATLTLDKSSSTTYMQLSSLTSEDSAVYYC
VRGYGNYGDYWGQGTTLTVSSASTKGPSVFPLAPSSKSTSGGTAALGC
LVKDYFPEPVTVSWNSGALTSGVHTFPAVLQSSGLYSLSSVVTVPSSS
LGTQTYICNVNHKPSNTKVDKRVEPKSCDK
LP heavy chain (variable domain underlined):
(SEQ ID NO: 68)
DVVMTQAAFSNPVTLGTSASISCRSSKSLLHSNGITYLYWYLQKPGQS
PQLLIYQMSNLASGVPDRFSSSGSGTDETLRISRVEAEDVGVYYCAQN
LELPWTFGGGTKLEIKRTVAAPSVFIFPPSDEQLKSGTASVVCLLNNF
YPREAKVQWKVDNALQSGNSQESVTEQDSKDSTYSLSSTLTLSKADYE
KHKVYACEVTHQGLSSPVTKSFNRGEC
A humanized antibody Fab HOLO was modelled using
the following heavy and light chain sequences.
H0 heavy chain:
(SEQ ID NO: 69)
QVQLVQSGAEVKKPGASVKVSCKASGYTFTSYWMHWVRQAPGQGLEWM
GEIDPSDSYTNYNQKFKGRVTMTRDTSTSTVYMELSSLRSEDTAVYYC
ARGYGNYGDYWGQGTLVTVSSASTKGPSVFPLAPSSKSTSGGTAALGC
LVKDYFPEPVTVSWNSGALTSGVHTFPAVLQSSGLYSLSSVVTVPSSS
LGTQTYICNVNHKPSNTKVDKRVEPKSCDK
L0 heavy chain:
(SEQ ID NO: 70)
DIVMTQSPLSLPVTPGEPASISCRSSKSLLHSNGITYLYWYLQKPGQS
PQLLIYQMSNLASGVPDRFSGSGSGTDFTLKISRVEAEDVGVYYCAQN
LELPWTFGGGTKVEIKRTVAAPSVFIFPPSDEQLKSGTASVVCLLNNF
YPREAKVQWKVDNALQSGNSQESVTEQDSKDSTYSLSSTLTLSKADYE
KHKVYACEVTHQGLSSPVTKSFNRGEC

For the design of light and heavy chain humanized variants, 5E7 HPLP and HOLO three-dimensional models were superimposed, and all amino acid differences were scrutinized one by one. Intrachain and extrachain connections between residues were also assessed in order to identify and to avoid disruption of any important low energy bond by introducing back-mutations in a given chain.

Starting from raw CDR-grafting, the potential back mutations were identified as followed by looking at framework differential residues between human and mouse.

### Heavy Chain Design:

In order to investigate the role of residue at position 72 (residue 71 according to Kabat numbering), this residue was back-mutated thereby conserving the leucine present in the parental antibody. The parental antibody has the sequence TLD compared to TRD in the H0L0 antibody. This back mutation should be coupled with the one on residue 79 in order to avoid a steric clash.

In order to investigate the role of residue at position 79, this residue was back-mutated thereby conserving the threonine present in the parental antibody. The parental antibody has the sequence TTY compared to TVY in the H0L0 antibody. Threonine in position 79 is oriented inside the VH domain and does not contact any other residue. Although valine in position 79 is well superimposed to threonine, this residue forms many bonds with R72, M34, C22 and I51. The rotameric position of I51 is explained by the contact with V79 (V78 according to Kabat numbering). The introduction of back mutation on residue 72 (R72L; residue 71 according to kabat numbering) would introduce a steric clash between I51 and L72 if V79 would be present.

In order to investigate the role of residue at position 74, this residue was back-mutated thereby conserving the lysine present in the parental antibody. The parental antibody has the sequence DKS compared to DTS in the H0L0 antibody. Lysine residue in position 74 is exposed at the molecular surface and occupy a critical position, although it is not that close to the potential binding site.

In order to investigate the role of residue at position 28, this residue was back-mutated thereby conserving the isoleucine present in the parental antibody. The parental antibody has the sequence YIF compared to YTF in the H0L0 antibody. Isoleucine and threonine at position 28 are well superimposed. They are exposed at the molecular surface and located at the top of the antibody, close to the paratope. It cannot be excluded that this residue may participate to the binding. Moreover, this residue is included in the CDR-H1 according to IMGT definition.

A first heavy chain variant (H1) having the amino acid sequence shown in SEQ ID NO: 39 had a R71L and V78T substitution. A second heavy chain variant (H2) having the amino acid sequence shown in SEQ ID NO: 41 had a R71L, T73K and V78T substitution. A third heavy chain variant (H3) having the amino acid sequence shown in SEQ ID NO: 43 had a T281, R71L, T73K and V78T substitution. Numbering of substitution is according to Kabat.

### Light Chain Design:

In order to investigate the role of the residue at position 2, this residue was back-mutated thereby conserving the valine residue of the parental antibody. The parental antibody has the sequence DVV compared to DIV in the H0L0 antibody. Residue V2 interacts with residue K27, located in the CDR-L1, which further interacts with residue E98 located in the CDR-L3.

In order to investigate the role of the residue at position 69, this residue was back-mutated thereby conserving the serine residue of the parental antibody. The parental antibody has the sequence SSS compared to SGS in the H0L0 antibody. Residue S69 is oriented inside the V_{L} domain and forms many bonds with adjacent residues W40 and M56 (located in CDR-L2), whereas G69 (G64 according to Kabat numbering) forms a h-bound with M56. All these residues are well superimposed. Residue W40 is the center of the entire internal network.

In order to investigate the role of the residue at position 11, this residue was back-mutated thereby conserving the asparagine residue of the parental antibody. The parental antibody has the sequence SNP compared to SLP in the H0L0 antibody. Residue L11 interacts with residue P8 and is likely to rigidify the P8 beta strand.

In order to investigate the role of the residue at position 8, this residue was back-mutated thereby conserving the alanine residue of the parental antibody. The parental antibody has the sequence SAL compared to SPL in the H0L0 antibody. As stated above, residue P8 interacts with residue L11 and such interaction is likely to rigidify the P8 beta strand.

A first light chain variant (L1) having the amino acid sequence of SEQ ID NO: 61 had a 12V and G64S substitution. A second light chain variant (L2) having the amino acid sequence of SEQ ID NO: 63 had a 12V, L11N and G64S substitution. A third light chain variant (L3) having the amino acid sequence of SEQ ID NO: 65 had a 12V, P8A, L11N and G64S substitution. Numbering of substitution is according to Kabat.

The amino acid sequences of respective heavy (“H” chains in Table 2) and light (“L” chains in Table 2) chain variable regions are shown in the Table 2 below.

TABLE 2

| | SEQ | |
|---|---|---|
| | ID | |
| Chain | NO | Sequence (amino acid substitutions in bold, Kabat CDRs underlined) |
| H1 | 39 | QVQLVQSGAEVKKPGASVKVSCKASGYTFTSYWMHWVRQAPGQGLEWMGEIDPSDSYTNY |
| | | NQKFKGRVTMTLDTSTSTTYMELSSLRSEDTAVYYCARGYGNYGDYWGQGTLVTVSS |
| H2 | 41 | QVQLVQSGAEVKKPGASVKVSCKASGYTFTSYWMHWVRQAPGQGLEWMGEIDPSDSYTNY |
| | | NQKFKGRVTMTLDKSTSTTYMELSSLRSEDTAVYYCARGYGNYGDYWGQGTLVTVSS |
| H3 | 43 | QVQLVQSGAEVKKPGASVKVSCKASGYIFTSYWMHWVRQAPGQGLEWMGEIDPSDSYTNY |
| | | NQKFKGRVTMTLDKSTSTTYMELSSLRSEDTAVYYCARGYGNYGDYWGQGTLVTVSS |
| L1 | 61 | DVVMTQSPLSLPVTPGEPASISCRSSKSLLHSNGITYLYWYLQKPGQSPQLLIYQMSNLAS |
| | | GVPDRFSSSGSGTDFTLKISRVEAEDVGVYYCAQNLELPWTFGGGTKVEIK |
| L2 | 63 | DVVMTQSPLSNPVTPGEPASISCRSSKSLLHSNGITYLYWYLQKPGQSPQQLIYQMSNLAS |
| | | GVPDRFSSSGSGTDFTLKISRVEAEDVGVYYCAQNLELPWTFGGGTKVEIK |
| L3 | 65 | DVVMTQSALSNPVTPGEPASISCRSSKSLLHSNGITYLYWYLQKPGQSPQLLIYQMSNLAS |
| | | GVPDRFSSSGSGTDFTLKISRVEAEDVGVYYCAQNLELPWTFGGGTKVEIK |

The antibodies having the heavy and light chain combinations shown in Table 3 below were produced.

TABLE 3

Light chains

| | | L0 | L1 | L2 | L3 |
|---|---|---|---|---|---|
| Heavy | H0 | H0L0 | H0L1 | H0L2 | H0L3 |
| chains | H1 | H1L0 | H1L1 | H1L2 | H1L3 |
| | H2 | H2L0 | H2L1 | H2L2 | H2L3 |
| | H3 | H3L0 | H3L1 | H3L2 | H3L3 |

### Example 4: Characterization of Binding to Nectin-4 by SPR

The antibodies in Table 3 of Example 3 were cloned as human IgG1 isotype antibodies, produced and purified, then tested for binding to human Nectin-4. Affinity of the 16 humanized variants, as well as their association and dissociation constants were assessed by SPR analysis. Table 4 summarizes all the calculated constants (Affinity constant KD (nM), association constant ka (1/Ms) and dissociation constant kd (1/s)).

The H0L0 antibody with an entirely human IGKHV-46*01 and IGHJ4*01 heavy chain framework and entirely human IGKV-28*01 and IGKJ4*01 light chain framework resulted in a kD of 80.2 nM. As shown in Table 4, other variants exhibited a lower KD than HOLO.

Heavy chain H3 recovers KD values close to the parental antibody. Back mutations introduced in H3 are therefore important for the stabilization of the humanized antibodies. A graduation of the dissociation constant values is noticed from H0 to H3.

TABLE 4

| Antibody | KD (nM) | ka (1/Ms) | kd (1/s) |
|---|---|---|---|
| 5E7 | 5.9 ± 1.2 | 4.8E+5 ± 1.0E−5 | 2.7E−3 ± 0.3E−3 |
| H0L0 | 80.2 | 1.7E+5 | 1.4E−2 |
| H1L0 | 67.3 | 1.1E+5 | 7.2E−3 |
| H2L0 | 46.4 | 1.3E+5 | 6.2E−3 |
| H3L0 | 35.8 | 1.1E+5 | 4.1E−3 |
| H3L1 | 22.0 ± 0.4 | 4.0E+5 ± 0.3E+5 | 8.7E−3 ± 0.4E−3 |
| H3L2 | 25.9 | 3.1E+5 | 8.1E−3 |
| H3L3 | 27.0 | 2.6E+5 | 7.0E−3 |

### Example 5: Modelling and Generation of a Second Set of Anti-huNectin-4 Antibodies with Human Framework Sequences

On the basis of the SPR data of the humanized antibodies generated in Example 3, new antibodies were designed.

### Heavy Chain Design

In order to investigate the role of the residue in position 38, this residue was back-mutated thereby conserving the lysine residue of the parental antibody. The parental antibody has the sequence VKQ compared to VRQ in the H0L0 antibody. K38 would be part of a salt bridge with residue E46.

In order to investigate the role of the residue in position 40, this residue was back-mutated thereby conserving the arginine residue of the parental antibody. The parental antibody has the sequence QRP compared to QAP in the H0L0 antibody. Residue R⁴⁰ contacts Q43, which residue contacts Q39 that form two hydrogen bonds with residue Q43 of the light chain. With such a back mutation, residue Q43 of the heavy chain occupies a divergent position but contact with Q39 is maintained and the two hydrogen bonds with L0-Q43 as well.

In order to investigate the role of the residue in position 48, this residue was back-mutated thereby conserving the isoleucine residue of the parental antibody. The parental antibody has the sequence WIG compared to WMG in the H0L0 antibody. Residue 148 interacts with A68, M81 and F64 (located in CDR-H2).

In order to investigate the role of the residue in position 70, this residue was back-mutated thereby conserving the leucine residue of the parental antibody. The parental antibody has the sequence TLT compared to TMT in the H0L0 antibody. Residue L70 interacts with residues M81, 151 (which is located in CDR-H2), Y60 (located in CDR-H2) and W36. Residue M70 (M69 according to Kabat numbering) interacts with residues 151 (located in CDR-H2), Y60 (located in CDR-H2) and W36. Except that residue 151 occupies different rotameric positions, all other residues are perfectly well superimposed, and the two networks are almost equivalent.

A fourth heavy chain variant (H4) having the amino acid sequence shown in SEQ ID NO: 45 had a R38K substitution. A fifth heavy chain variant (H5) having the amino acid sequence of SEQ ID NO: 47 had a R38K and A40R substitution. A sixth heavy chain variant (H6) having the amino acid sequence of SEQ ID NO: 49 had a R38K, A40R and M481 substitution. A seventh heavy chain variant (H7) having the amino acid sequence of SEQ ID NO: 51 had a R38K, A40R, M481 and M69L substitution. Further eight (H8; SEQ ID NO: 53), ninth (H9; SEQ ID NO: 55) and tenth (H10; SEQ ID NO: 57) chains were designed with different combinations of the substitutions. Numbering of amino acid residues is according to Kabat.

The amino acid sequences of respective heavy (“H” chains in Table 5) and light (“L” chains in Table 5) chain variable regions are shown in the Table 5 below.

TABLE 5

| | SEQ | |
|---|---|---|
| | ID | |
| Chain | NO | Sequence (amino acid substitutions in bold, Kabat CDRs underlined) |
| H4 | 45 | QVQLVQSGAEVKKPGASVKVSCKASGYIFTSYWMHWVKQAPGQGLEWMGEIDPSDSYTNY |
| | | NQKFKGRVTMTLDKSTSTTYMELSSLRSEDTAVYYCARGYGNYGDYWGQGTLVTVSS |
| H5 | 47 | QVQLVQSGAEVKKPGASVKVSCKASGYIFTSYWMHWVKQRPGQGLEWMGEIDPSDSYTNY |
| | | NQKFKGRVTMTLDKSTSTTYMELSSLRSEDTAVYYCARGYGNYGDYWGQGTLVTVSS |
| H6 | 49 | QVQLVQSGAEVKKPGASVKVSCKASGYIFTSYWMHWVKQRPGQGLEWIGEIDPSDSYTNY |
| | | NQKFKGRVTMTLDKSTSTTYMELSSLRSEDTAVYYCARGYGNYGDYWGQGTLVTVSS |
| H7 | 51 | QVQLVQSGAEVKKPGASVKVSCKASGYIFTSYWMHWVKQRPGQGLEWIGEIDPSDSYTNY |
| | | NQKFKGRVTLTLDKSTSTTYMELSSLRSEDTAVYYCARGYGNYGDYWGQGTLVTVSS |
| H8 | 53 | QVQLVQSGAEVKKPGASVKVSCKASGYIFTSYWMHWVKQAPGQGLEWIGEIDPSDSYTNY |
| | | NQKFKGRVTMTLDKSTSTTYMELSSLRSEDTAVYYCARGYGNYGDYWGQGTLVTVSS |

The antibodies having the heavy and light chain combinations shown in Table 6 below were produced.

TABLE 6

Light chains

| | | L0 | L1 | L2 | L3 |
|---|---|---|---|---|---|
| Heavy | H4 | H4L0 | H4L1 | H4L2 | H4L3 |
| chains | H5 | H5L0 | H5L1 | H5L2 | H5L3 |
| | H6 | H6L0 | H6L1 | H6L2 | H6L3 |
| | H7 | H7L0 | H7L1 | H7L2 | H7L3 |
| | H8 | | H8L1 | | |

### Example 6: Characterization of Binding to Nectin-4 by SPR (Second Set)

The antibodies in Table 6 of Example 5 were cloned as human IgG1 isotype antibodies, produced and purified, then tested for binding to human Nectin-4. Affinity of the 16 humanized variants, as well as their association and dissociation constants were assessed by SPR analysis. Table 7 summarizes all the calculated constants (Affinity constant KD (nM), association constant ka (1/Ms) and dissociation constant kd (1/s)).

The H0L0 antibody with an entirely human IGKHV-46*01 and IGHJ4*01 heavy chain framework and entirely human IGKV-28*01 and IGKJ4*01 light chain framework resulted in a KD of 80.2 nM. As shown in Table 7, other variants exhibited a lower KD than HOLO. Particularly variants H4L1, H5L3 and H8L1 had a KD closer to the chimeric parental antibody (which had a KD of 5.9 nM±1.2).

TABLE 7

| Humanized | | | |
|---|---|---|---|
| variants | KD (nM) | ka (1/Ms) | kd (1/s) |
| ch5E7 | 5.9 ± 1.2 | 4.8E+5 ± 1.0E−5 | 2.7E−3 ± 0.3E−3 |
| H0L0 | 80.2 | 1.7E+5 | 1.4E−2 |
| H4L1 | 19.5 ± 0.8 | 3.1E+5 ± 0.3E+5 | 5.9E−3 ± 0.3E−3 |
| H4L2 | 26.8 | 2.0E+5 | 5.4E−3 |
| H4L3 | 21.6 ± 1.1 | 2.5E+5 ± 0.3E+5 | 5.2E−3 ± 0.2E−3 |
| H5L1 | 24.5 | 2.3E+5 | 5.6E−3 |
| H5L2 | 23.8 | 2.2E+5 | 5.3E−3 |
| H5L3 | 21.5 ± 0.4 | 2.5E+5 ± 0.1E+5 | 5.3E−3 ± 0.0E−3 |
| H6L1 | 32.1 | 1.9E+5 | 6.2E−3 |
| H6L2 | 41.3 | 1.4E+5 | 5.8E−3 |
| H6L3 | 38.6 | 1.3E+5 | 5.1E−3 |
| H7L1 | 29.7 | 2.0E+5 | 6.0E−3 |
| H7L2 | 39.5 | 1.6E+5 | 6.3E−3 |
| H7L3 | 49.0 | 1.1E+5 | 5.5E−3 |
| H8L1 | 19.2 | 2.5E+5 | 4.8E−3 |

### Example 7: Characterization of Binding to Nectin-4 by Flow Cytometry Assay

The humanized antibodies in Table 3 of Example 3 and in Table 6 of Example 5 were cloned, produced and purified, then tested for binding to Nectin-4 expressing cells by flow cytometry. Binding of humanized variants in human IgG1 format was determined on the SUM190 cell line expressing high levels of Nectin-4. EC50 and Maximum MedFI representing MedFi at saturation phase are show in Table 8 below.

All the humanized variants possessing the L0 or L1 chains have a lower binding capacity than the others (lower plateau phase). By contrast, all humanized variants bearing the L2 or L3 chains have similar binding capacity compared to the parental chimeric 5E7 antibody (ch5E7). Variant H5L3 exhibits the highest MedFi value.

TABLE 8

| | | Max |
|---|---|---|
| Antibodies | EC₅₀ | MedFi |
| ch5E7 | 1.10 | 25213 |
| H0L0 | 0.542 | 15507 |
| H1L0 | 0.64 | 17251 |
| H2L0 | 0.45 | 16704 |
| H3L0 | 0.88 | 17596 |
| H3L1 | 1.79 | 17208 |
| H3L2 | 1.19 | 22887 |
| H3L3 | 1.45 | 23814 |
| H4L1 | 1.49 | 18399 |
| H4L2 | 1.44 | 24352 |
| H4L3 | 1.60 | 24840 |
| H5L1 | 1.43 | 18263 |
| H5L2 | 1.14 | 25276 |
| H5L3 | 1.29 | 27095 |
| H6L1 | 1.15 | 18263 |
| H6L2 | 1.30 | 24779 |
| H6L3 | 1.01 | 24840 |
| H7L1 | 1.27 | 17816 |
| H7L2 | 1.30 | 23404 |
| H7L3 | 1.23 | 25026 |
| H8L1 | 1.63 | 17251 |

### Example 8: Intracellular Internalization Assay

The humanized variants in Table 3 of Example 3 and in Table 6 of Example 5 were cloned, produced and purified, then tested for their ability to induce Nectin-4 internalization. Chimeric parental 5E7 antibody and isotype controls were used as negative control. This analysis was performed with the Fab-ZAP human Internalization kit assay with the Cell Titer Glo™ (CTG) assay used as readout. Experiments were performed on two cell lines expressing different levels of Nectin-4. MDA-MB-468 have a lower expression of Nectin-4 than SUM190.

### Internalization Assay on SUM190 Cell Line

Results of the internalization assays on SUM190 cell line are shown in Table 9 below, that presents the internalization efficiency (normalized to the chimeric parental antibody internalization efficiency on SUM190 cell line). The experiment was conducted in duplicate (two independent experiments).

TABLE 9

| | |
|---|---|
| | Internalization |
| | efficiency |
| | (normalized to the |
| | chimeric parental |
| | antibody |
| | internalization |
| | potential) on |
| Antibodies | SUM190 |

| | | |
|---|---|---|
| Chimeric | 100.0 | 100.0 |
| parental 5E7 | | |
| H0L0 | 65.9 | 35.4 |
| H1L0 | 83.7 | 58.6 |
| H2L0 | 74.2 | 61.8 |
| H3L0 | 81.4 | 69.7 |
| H3L1 | 82.1 | 41.0 |
| H3L2 | 69.8 | 45.5 |
| H3L3 | 65.3 | 59.6 |
| H4L1 | 73.5 | 72.6 |
| H4L2 | 83.6 | 67.6 |
| H4L3 | 81.9 | 61.0 |
| H5L1 | 71.5 | 52.4 |
| H5L2 | 81.3 | 86.0 |
| H5L3 | 82.8 | 74.9 |
| H6L1 | 71.3 | 54.5 |
| H6L2 | 69.7 | 63.5 |
| H6L3 | 78.7 | 92.8 |
| H7L1 | 128.7 | 73.1 |
| H7L2 | 89.6 | 74.8 |
| H7L3 | 88.5 | 61.3 |
| H8L1 | 131.9 | 65.2 |

Internalization efficiencies determined in the experiments performed on SUM190 cell line show that the humanized variants H3L0, H4L2, H5L2, H5L3, H6L3 and H7L2 induce 75% or more internalization compared to the parental antibody (5E7), exhibiting therefore an interesting internalization potential. It should be noted that H8L1 and H7L1 variants were more potent than the chimeric 5E7 in the 1^{st} experiment but this result was not confirmed in the 2^{nd} experiment.

### Internalization Assay on MDA-M-468 Cell Line

Results of the internalization assays on SUM190 cell line are shown in Table 10 below that presents the internalization efficiency (normalized to the chimeric parental antibody internalization efficiency on MDA-M-468 cell line). The experiment was conducted in duplicate (two independent experiments).

TABLE 10

| | |
|---|---|
| | Internalization |
| | efficiency |
| | (normalized to the |
| | chimeric parental |
| | antibody |
| | internalization |
| | efficiency) on MDA- |
| Antibodies | MB-468 |

| | | |
|---|---|---|
| Chimeric | 100.0 | 100.0 |
| parental 5E7 | | |
| H0L0 | −36.2 | −36.2 |
| H1L0 | −7.4 | −7.4 |
| H2L0 | −35.5 | −35.5 |
| H3L0 | 1.2 | 1.2 |
| H3L1 | 48.2 | 48.2 |
| H3L2 | 13.7 | 13.7 |
| H3L3 | 43.8 | 43.8 |
| H4L1 | 31.0 | 31.0 |
| H4L2 | 64.2 | 64.2 |
| H4L3 | 43.8 | 43.8 |
| H5L1 | 33.4 | 33.4 |
| H5L2 | 39.4 | 39.4 |
| H5L3 | 87.8 | 87.8 |
| H6L1 | 61.6 | 61.6 |
| H6L2 | 58.5 | 58.5 |
| H6L3 | 66.3 | 66.3 |
| H7L1 | 79.0 | 79.0 |
| H7L2 | 58.7 | 58.7 |
| H7L3 | 51.8 | 51.8 |
| H8L1 | 71.2 | 71.2 |

The results obtained show that only H5L3 humanized variant consistently reaches well over 75% of efficacy of the parental chimeric 5E7 antibody, whereas all other variants were less efficient and many of them had heterogeneous results between experiments.

### Example 9: In Vitro Cytotoxicity of Antibodies as ADCs on Tumor Cell Lines Nectin-4 Low/SUM190 Breast Cancer Model

We assessed the ability of the 5E7 antibody conjugated with camptothecin analogues (Dxd or exatecan) to kill SUM190 cells. In this experiment, the 5E7 antibody was tested along with anti-Ig-like V domain antibodies enfortumab and N41 and a control antibody conjugated with the same toxin at equivalent drug to antibody ratios. A first ADC was prepared in which antibodies were conjugated (to cysteine residues) at 8 toxins per antibody (DAR=8) to a camptothecin analogue (Dxd) via a linker comprising an intracellularly cleavable tetrapeptide linker (GGFG) having the structure shown below, referred to as ggfg-Dxd:

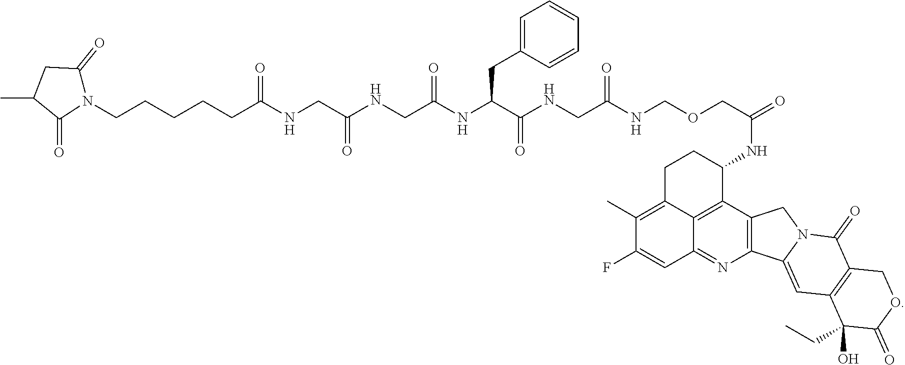 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)CC(C)C1=O)CC3]

A second ADC was prepared in which antibodies were conjugated at 8 toxins per antibody (DAR=8) to another camptothecin analogue (exatecan) via the cleavable linker having the structure below, referred to as PEG (8U)-Val-Ala-PAB-Exatecan:

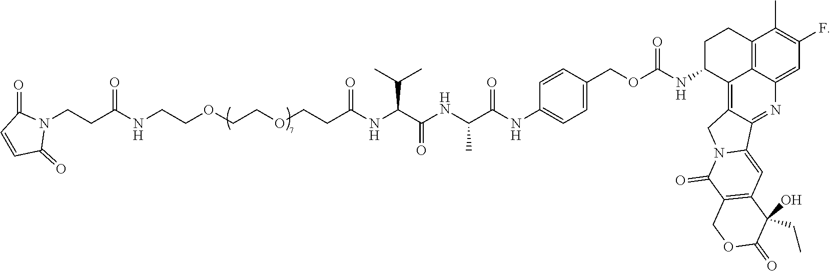

Briefly, dose-ranges of each tested Ab (starting point 150 nM, dilution factor 2 for 3 points, then 5 for 5 points) were incubated with cells for 5 days before cell viability measurement by addition of CTG substrate. Luminescence vs. Ab concentration was plotted on graph.

For each ADC, a concentration range of ADC was incubated with nectin-4-expressing cells. After incubation, CTG substrate was added at 1/1 ratio and the luminescent signal was read with a plate reader (Enspire). It allowed quantifying the ATP present (indicator of metabolically active cells) which was proportional to cell viability.

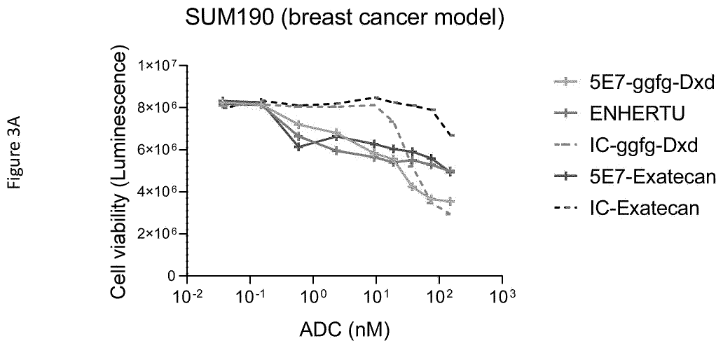

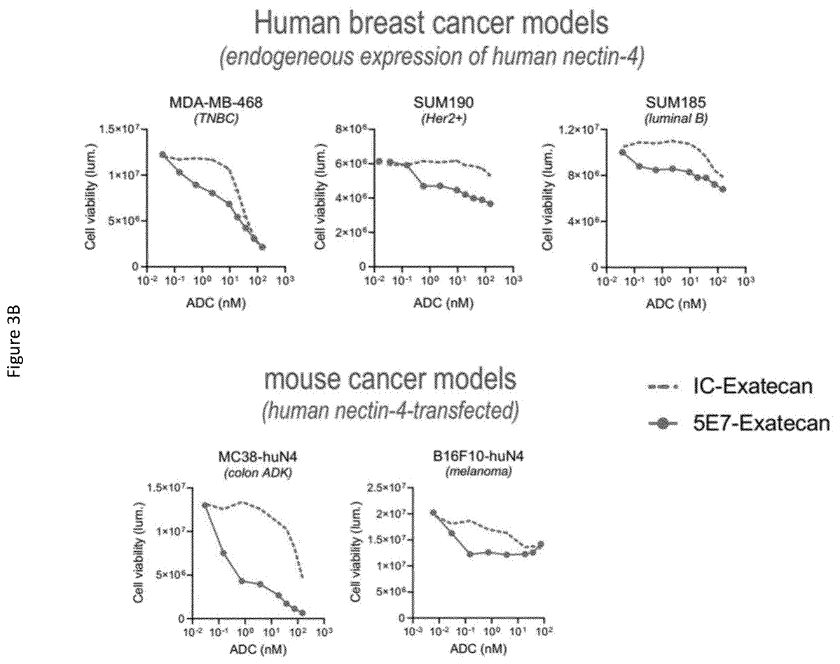

Results are shown in FIG. 3A. FIG. 3A shows killing of human breast cancer cells by the 5E7 antibody conjugated to the camptothecin analogues Dxd (via a GGFG-Dxd linker) or exatecan (via the (PEG (8U)-Val-Ala-PAB-Exatecan linker), along with isotype control antibodies (IC), all at equivalent drug to antibody ratios (DAR=8), and compared to compared to V-domain binding Enhertu™ (trastuzumab deruxtecan (anti-HER2)).

All Nectin-4-targeted ADCs reduced the cell viability more efficiently than the non-targeted ADC (IC-GGFG-camptothecin).

### SUM185, MDA-MB-468, MC38 and B16F10 Cell Lines

Antibody 5E7 conjugated to exatecan (via the (PEG (8U)-Val-Ala-PAB-Exatecan linker) was further tested for cell killing on other cancer cells lines. FIG. 3B shows efficacy of “5E7-exatecan” (5E7 conjugated to the exatecan linker (PEG (8U)-Val-Ala-PAB-Exatecan) is capable of causing the death of HER-2 and Nectin-4 expressing SUM185 and SUM190, as well as MDA-MB-468 (TNBC) human tumor cells and human Nectin-4-expressing MC38 (colon cancer), and B16F10 (melanoma) murine tumor cells. EC50 values for cell viability are shown in Table 11 below.

TABLE 11

| Cell | MDA-MB- | | | MC38- | B16F10- |
|---|---|---|---|---|---|
| line | 468 | SUM185 | SUM190 | huN4 | huN4 |
| EC₅₀ (nM) | 1.5E+05 | 3E+05 | 7E+05 | 2.3E+5 | 9.8E+5 |

### Example 10: In Vivo Efficacy of ADCs in a Mouse Model of Human Breast Cancer (Nectin-4 Low/SUM190 Model)

We compared the efficacy of the 5E7 antibody as a camptothecin ADC, in comparison to enfortumab and N41, in a mouse model of human breast cancer. In this experiment, the 5E7 antibody was tested together with enfortumab and N41 and a control Ab conjugated to the ggfg-Dxd linker at equivalent drug to antibody ratios of 8 toxins per antibody (DAR=8).

SUM190 cells were subcutaneously engrafted in CB17-SCID immunodeficient mice at a dose of 0.5 million cells in 100 μl of Matrigel with growth factor diluted at ½ in PBS. When tumors reached a volume between 195 and 250 mm3, mice were randomized into groups of 9 mice for intravenous treatment with a single injection of 3 mg/kg of camptothecin ADCs. Tumor growths were followed twice a week. Kaplan Meier survival curves were established by using GraphPad Prism V7 software according to the following criteria: When the tumor volume reached 1500 mm3, mice were euthanized and considered dead on the day of sacrifice (D). When tumors showed signs of necrosis, mice were euthanized and considered dead on the same day (D) (indicated with red star on the graphs of individual tumor growth).

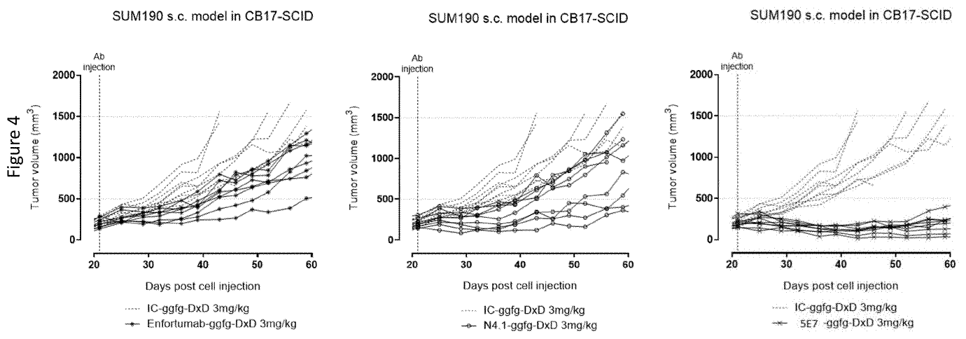

Results showed that at the 10 mg/kg dose all ADC were similarly efficient in preventing increase in tumor volume. However, at the lower dose of ADC (3 mg/kg), antibody 5E7 showed a strong ability to prevent tumor growth while both N41 and enfortumab no longer showed the ability to control tumor growth. Results for the 3 mg/kg dose are shown in FIG. 4.

### Example 11: Comparative In Vitro Efficacy of ADCs in a Breast Cancer Model of Drug Resistance (Human Breast Cancer, HER2/Nectin-4 High/SUM185 Model)

We next assessed the ability of the 5E7 Ab conjugated with a camptothecin analogue to kill SUM185 cells, compared to PADCEV™ (enfortumab vedotin) and ENHERTU™. This setting was used as a model of anti-HER2 resistance. SUM185 cells express Nectin-4 at relatively high levels, with Nectin-4 expression levels about twice that of HER2 in these cells (see Example 1). In this experiment, the 5E7 antibody was tested as an ADC having DAR=8 along with PADCEV™ (DAR=4, specification for FDA-approved PADCEV™) and control antibodies all conjugated with the same toxin at equivalent drug to antibody ratios. 5E7 was conjugated with a camptothecin analogue Dxd via the ggfg-Dxd linker.

Briefly, dose-ranges of each tested Ab (starting point 150 nM, dilution factor 2 for 3 points, then 5 for 5 points) were incubated on cells for 5 days before cell viability measurement by addition of CTG substrate. Luminescence vs. Ab concentration was plotted on graph.

For each ADC, a concentration range of ADC was incubated with nectin-4-expressing cells. After incubation, CTG substrate was added at 1:1 ratio and the luminescent signal was read with a plate reader (Enspire), allowing quantification of the ATP present (indicator of metabolically active cells) which was proportional to cell viability.

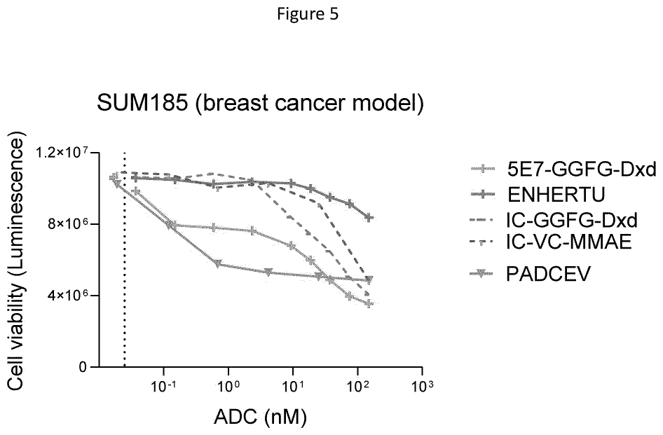

Results are shown in FIG. 5. 5E7-ggfg-Dxd and PADCEV™ were able to kill the SUM185 more efficiently than ENHERTU™. In each case, the Nectin 4-targeted ADCs reduced the cell viability more efficiently than their non-targeted ADC (IC) counterpart.

### Example 12: Characterization of Species Cross-Reactivity of Anti-Nectin-4 Antibodies

Anti-Nectin-4 antibodies were tested by flow cytometry for binding to different CHO cell lines made to express respectively the mouse, cynomolgus and rat Nectin-4 protein (including an N-terminal V5 tag not shown in the sequences below).

The mature amino acid sequence of the proteins expressed by the cells were as follows:

Mouse Nectin-4:
(SEQ ID NO: 12)
ELETSDVVTVVLGQDAKLPCFYRGDPDEQVGQVAWARVDPNEGIRELALLHSKYGLHVNPAYEDRVEQ
PPPPRDPLDGSVLLRNAVQADEGEYECRVSTFPAGSFQARMRLRVLVPPLPSLNPGPPLEEGQGLTLA
ASCTAEGSPAPSVTWDTEVKGTQSSRSFTHPRSAAVTSEFHLVPSRSMNGQPLTCVVSHPGLLQDRRI
THTLQVAFLAEASVRGLEDQNLWQVGREGATLKCLSEGQPPPKYNWTRLDGPLPSGVRVKGDTLGFPP
LTTEHSGVYVCHVSNELSSRDSQVTVEVLDPEDPGKQVDLVSASVIIVGVIAALLFCLLVVVVVLMSR
YHRRKAQQMTQKYEEELTLTRENSIRRLHSHHSDPRSQPEESVGLRAEGHPDSLKDNSSCSVMSEEPE
GRSYSTLTTVREIETQTELLSPGSGRTEEDDDQDEGIKQAMNHFVQENGTLRAKPTGNGIYINGRGHL
V
Rat Nectin-4:
(SEQ ID NO: 13)
MPLSLGAEMWGPEAWLLLLFLASFTGRYSAGELETSDLVTVVLGQDAKLPCFYRGDPDEQVGQVAWAR
VDPNEGTRELALLHSKYGLHVSPAYEDRVEQPPPPRDPLDGSILLRNAVQADEGEYECRVSTFPAGSF
QARMRLRVLVPPLPSLNPGPPLEEGQGLTLAASCTAEGSPAPSVTWDTEVKGTQSSRSFKHSRSAAVT
SEFHLVPSRSMNGQPLTCVVSHPGLLQDQRITHTLQVAFLAEASVRGLEDQNLWHVGREGATLKCLSE
GQPPPKYNWTRLDGPLPSGVRVKGDTLGFPPLTTEHSGVYVCHVSNELSSRASQVTVEVLDPEDPGKQ
VDLVSASVVVVGVIAALLFCLLVVVVVLMSRYHRRKAQQMTQKYEEELTLTRENSIRRLHSHHTDPRS
QPEESVGLRAEGHPDSLKDNSSCSVMSEEPEGRSYSTLTTVREIETQTELLSPGSGRTEEEDDQDEGI
KQAMNHFVQENGTLRAKPTGNGIYINGRGHLV
Cynomolgus Nectin-4:
(SEQ ID NO: 14)
GELETSDVVTVVLGQDAKLPCFYRGDSGEQVGQVAWARADAGEGAQELALLHSKYGLHVSPAYEGRVE
QPPPPRNPLDGSVLLRNAVQADEGEYECRVSTFPAGSFQARLRLRVLVPPLPSLNPGPALEEGQGLTL
AASCTAEGSPAPSVTWDTEVKGTTSSRSFKHSRSAAVTSEFHLVPSRSMNGQPLTCVVSHPGLLQDQR
ITHILHVSFLAEASVRGLEDQNLWHVGREGAMLKCLSEGQPPPSYNWTRLDGPLPSGVRVDGDTLGFP
PLTTEHSGIYVCHVSNEFSSRDSQVTVDVLDPQEDSGKQVDLVSASVVVVGVIAALLFCLLVVVVVLM
SRYHRRKAQQMTQKYEEELTLTRENSIRRLHSHHTDPRSQPEESVGLRAEGHPDSLKDNSSCSVMSEE
PEGRSYSTLTTVREIETQTELLSPGSGRTEEEEDQDEGIKQAMNHFVQENGTLRAKPTGNGIYINGRG
HLV

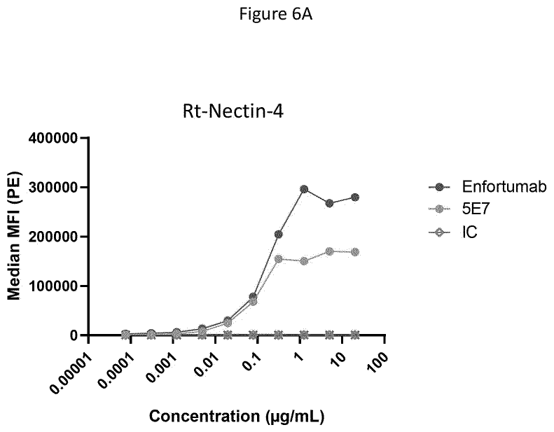

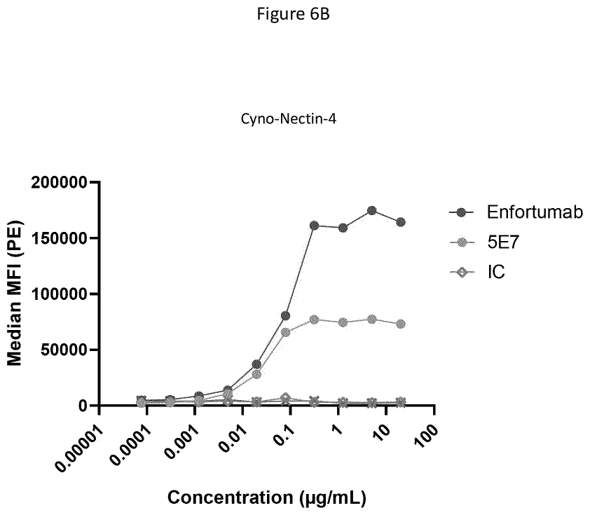

Antibody 5E7 bound human, cynomolgus, as well as rat Nectin 4 proteins but lack binding to the mouse Nectin 4 protein. FIGS. 6A and 6B show binding of anti-Nectin-4 antibodies on rat and cynomolgus Nectin-4-expressing CHO cell lines.

### Example 13: Characterization of Nectin Family Cross Reactivity

Anti-Nectin-4 antibodies were tested by flow cytometry for binding to different CHO cell lines made to express respectively the human Nectin 1, Nectin 2, Nectin 3 and PVR proteins. The expression of each cell lines was controlled and validated with known respectively the anti-human Nectin1, anti-human Nectin2, anti-human Nectin3 and anti-human PVR antibodies. The mature amino acid sequence of the proteins expressed by the cells were as follows:

Nectin-1:
(SEQ ID NO: 15)
MGLAGAAGRWWGLALGLTAFFLPGVHSQVVQVNDSMYGFIGTDVVLHCSFANPLPSVKITQVTWQKST
NGSKQNVAIYNPSMGVSVLAPYRERVEFLRPSFTDGTIRLSRLELEDEGVYICEFATFPTGNRESQLN
LTVMAKPTNWIEGTQAVLRAKKGQDDKVLVATCTSANGKPPSVVSWETRLKGEAEYQEIRNPNGTVTV
ISRYRLVPSREAHQQSLACIVNYHMDRFKESLTLNVQYEPEVTIEGFDGNWYLQRMDVKLTCKADANP
PATEYHWTTLNGSLPKGVEAQNRTLFFKGPINYSLAGTYICEATNPIGTRSGQVEVNITEFPYTPSPP
EHGRRAGPVPTAIIGGVAGSILLVLIVVGGIVVALRRRRHTFKGDYSTKKHVYGNGYSKAGIPQHHPP
MAQNLQYPDDSDDEKKAGPLGGSSYEEEEEEEEGGGGGERKVGGPHPKYDEDAKRPYFTVDEAEARQD
GYGDRTLGYQYDPEQLDLAENMVSQNDGSFISKKEWYV
Nectin-2:
(SEQ ID NO: 16)
MARAAALLPSRSPPTPLLWPLLLLLLLETGAQDVRVQVLPEVRGQLGGTVELPCHLLPPVPGLYISLV
TWQRPDAPANHQNVAAFHPKMGPSFPSPKPGSERLSFVSAKQSTGQDTEAELQDATLALHGLTVEDEG
NYTCEFATFPKGSVRGMTWLRVIAKPKNQAEAQKVTFSQDPTTVALCISKEGRPPARISWLSSLDWEA
KETQVSGTLAGTVTVTSRFTLVPSGRADGVTVTCKVEHESFEEPALIPVTLSVRYPPEVSISGYDDNW
YLGRTDATLSCDVRSNPEPTGYDWSTTSGTFPTSAVAQGSQLVIHAVDSLFNTTFVCTVTNAVGMGRA
EQVIFVRETPNTAGAGATGGIIGGIIAAIIATAVAATGILICRQQRKEQTLQGAEEDEDLEGPPSYKP
PTPKAKLEAQEMPSQLFTLGASEHSPLKTPYFDAGASCTEQEMPRYHELPTLEERSGPLHPGATSLGS
PIPVPPGPPAVEDVSLDLEDEEGEEEEEYLDKINPIYDALSYSSPSDSYQGKGFVMSRAMYV
Nectin-3:
(SEQ ID NO: 17)
MARTLRPSPLCPGGGKAQLSSASLLGAGLLLQPPTPPPLLLLLFPLLLFSRLCGALAGPIIVEPHVTA
VWGKNVSLKCLIEVNETITQISWEKIHGKSSQTVAVHHPQYGFSVQGEYQGRVLFKNYSLNDATITLH
NIGFSDSGKYICKAVTFPLGNAQSSTTVTVLVEPTVSLIKGPDSLIDGGNETVAAICIAATGKPVAHI
DWEGDLGEMESTTTSFPNETATIISQYKLEPTRFARGRRITCVVKHPALEKDIRYSFILDIQYAPEVS
VTGYDGNWFVGRKGVNLKCNADANPPPFKSVWSRLDGQWPDGLLASDNTLHFVHPLTFNYSGVYICKV
TNSLGQRSDQKVIYISDPPTTTTLQPTIQWHPSTADIEDLATEPKKLPFPLSTLATIKDDTIATIIAS
VVGGALFIVLVSVLAGIFCYRRRRTFRGDYFAKNYIPPSDMQKESQIDVLQQDELDSYPDSVKKENKN
PVNNLIRKDYLEEPEKTQWNNVENLNRFERPMDYYEDLKMGMKFVSDEHYDENEDDLVSHVDGSVISR
REWYV
PVR (Nectin-5):
(SEQ ID NO: 18)
DVVVQAPTQVPGFLGDSVTLPCYLQVPNMEVTHVSQLTWARHGESGSMAVFHQTQGPSYSESKRLEFV
AARLGAELRNASLRMFGLRVEDEGNYTCLFVTFPQGSRSVDIWLRVLAKPQNTAEVQKVQLTGEPVPM
ARCVSTGGRPPAQITWHSDLGGMPNTSQVPGFLSGTVTVTSLWILVPSSQVDGKNVTCKVEHESFEKP
QLLTVNLTVYYPPEVSISGYDNNWYLGQNEATLTCDARSNPEPTGYNWSTTMGPLPPFAVAQGAQLLI
RPVDKPINTTLICNVTNALGARQAELTVQVKEGPPSEHSGISRNAIIFLVLGILVFLILLGIGIYFYW
SKCSREVLWHCHLCPSSTEHASASANGHVSYSAVSRENSSSQDPQTEGTR

Results showed that there were no cross reactivity of the anti-Nectin-4 antibodies on members of the human Nectin family. The figure shows flow cytometry results of anti-Nectin-4 antibodies on human Nectin 1, human Nectin 2, human Nectin 3 and human PVR expressing CHO cells. No antibodies presented bind to each cell lines.

### Example 14: Epitope Mapping by Competition for Binding to Nectin-4 by SPR

Chimeric antibody 5E7, together with previously reported antibodies N4.1 (N41), antibody 14A5 and enfortumab, were tested for their ability to compete with one another for binding to the wild-type human Nectin-4 protein by SPR (Surface Plasmon Resonance) methods, with OCTET analysis using Ni-NTA (NTA) Biosensors (Fortebio). Briefly, human Nectin4-His-BirA protein diluted to 5 μg/mL in Kinetic buffer 10× were captured onto the biosensors. The first antibody was injected diluted to 10 μg/mL in Kinetic buffer 10×, followed by a second injection of the first antibody diluted to 10 μg/mL in Kinetic buffer 10× in order to saturate the signal. The second antibody was injected diluted to 10 μg/mL in Kinetic buffer 10×.

Results are shown in Table 12 below. Squares with “O” indicate substantial or direct competition between the 1^{st} and 2^{nd} antibody (1^{st} antibody prevents/causes loss of binding of 2^{nd} antibody), squares with “X” indicate potential partial competition (1^{st} antibody causes a potential reduction but not loss of binding of 2^{nd} antibody), squares with “N/A” indicate no competition. Antibody 5E7 competed with one another for binding to Nectin-4.

TABLE 12

| | | 2^{nd} mab injected | | | | |
|---|---|---|---|---|---|---|
| | | 5E7 | enfortumab | N41 | 14A5 | MABT64 |
| 1st mAb | 5E7 | ◯ | N/A | X | X | X |
| injected | Enfortumab | X | ◯ | N/A | N/A | N/A |
| | N41 | N/A | N/A | ◯ | ◯ | ◯ |
| | 14A5 | N/A | N/A | ◯ | ◯ | ◯ |
| | MABT64 | N/A | N/A | ◯ | ◯ | ◯ |

### Example 15: Epitope Mapping of Antibodies Using Nectin-4 Point Mutants Cell Surface Expressed Human Nectin-4 Point Mutants

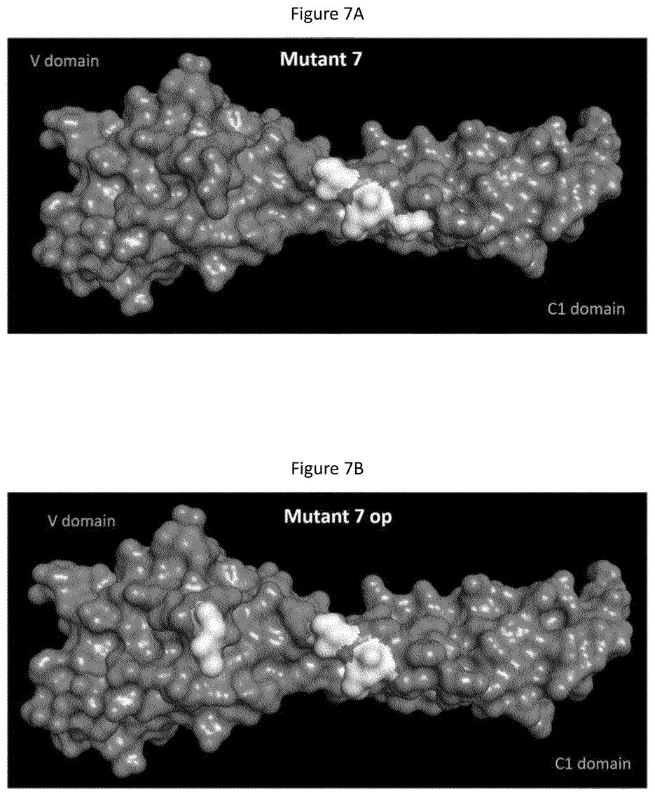

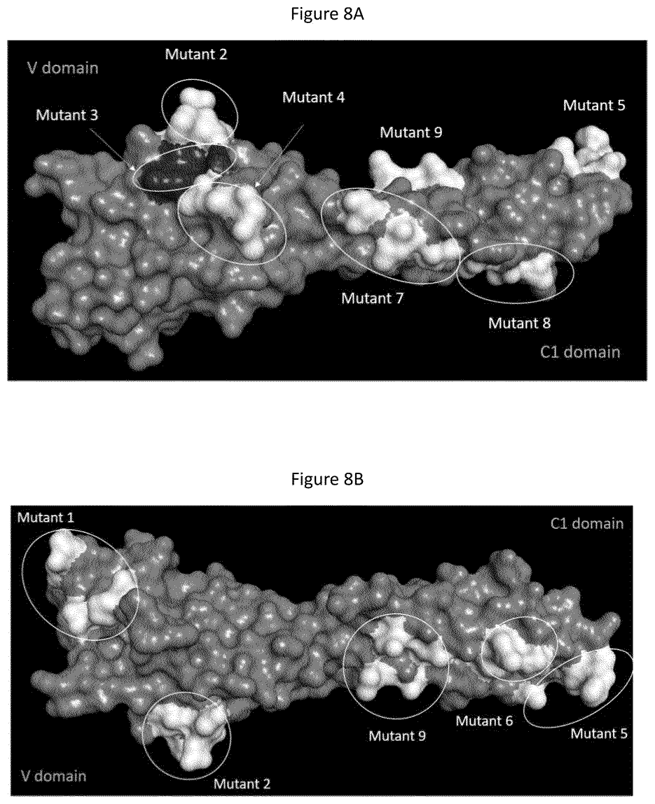

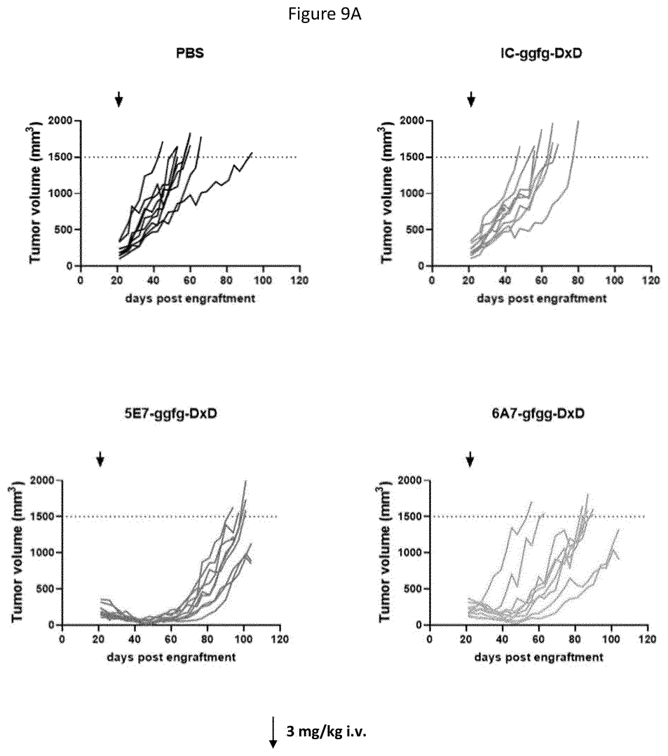

The binding profile of the anti-Nectin-4 antibodies on full-length and Ig-like V domain-deleted proteins, together with species binding profile of the antibodies (binding to human, cynomolgus and rat Nectin-4 but not to mouse Nectin-4), together with inter-species differences among the non-human Nectin-4 proteins permitted the identification of residues the junction of the Ig-like V domain and the Ig-like C2 type 1 domain (also referred to as “C1”). Combined with the published structures of the Nectin-4 domains, Nectin-4 mutations at surface exposed amino acids residues were designed. Nectin-4 domain structure was modelled based on Protein Data Bank reference: 4FRW (domains V et C1). Human Nectin-4 used was NCBI Reference Sequence: NP_112178.2, mouse Nectin-4 used was NCBI Reference Sequence: AAL79833.1, cynomolgus Nectin-4 used was NCBI Reference Sequence: XP_005541277.1, rat Nectin-4 used was NCBI Reference Sequence: NP_001102546.1. The cells expressing the Nectin-4 mutants are then be used for testing anti-Nectin-4 antibodies for loss of binding to different Nectin-4 mutants to identify antibodies that bind to the same site on Nectin-4. In particular, mutants having C1-V junction substitutions at residues K197T and/or S199A, or mutants 7, 7bis and 9 having additional and/or adjacent substitutions at the junction of domain C1 and V, can identify antibodies having advantageous application as immunoconjugates. Mutant 7 had substitutions S195A/K197T/S199A. Mutant 7bis had substitutions A72P/G73N/K197T/S199A. Mutant 9 included the key residue Q234 substitution, and had substitutions L150S/S152A/Q234R/1236S. FIGS. 7A and 7B show a molecular model of the human Nectin-4 protein, indicating the position of substituted residues in mutants 7 (7A) and in mutants 7bis (7B); these mutants are in the C1 domain identify two sites at the junction of domain C₁ and V domain that are on opposing faces of the Nectin-4 protein. FIGS. 8A and 8B show different view of a molecular model of the human Nectin-4 protein, indicating the position of substituted residues in mutants 1, 2, 3, 4, 5, 6, 7, 8 and 9.

Nectin-4 mutants were generated by PCR. The sequences amplified were run on agarose gel and purified using the Macherey Nagel PCR Clean-Up Gel Extraction kit. The purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO₂ incubator for 48 hours prior to testing for transgene expression. Mutants were transfected in Hek-293T cells, as shown in the table below. The targeted amino acid mutations are shown in Table 13 below, listing the residue present in wild-type Nectin-4/position of residue/residue present in mutant Nectin-4, with position reference being to the Nectin-4 protein with leader peptide shown in SEQ ID NO: 1.

TABLE 13

| | Amino acid substitutions with reference |
|---|---|
| Mutant | to Nectin-4 of SEQ ID NO: 1 |
| 1 | S58P/G59D/Q61A/N106D/P107A |
| 2 | V90S/P92A/A93S/E95A/G96D |
| 3 | A76S/Q77R/E78A/S91N |
| 4 | A72P/G73N/G75S |
| 5 | A158P/E160G/S243A/F244S |
| 6 | H238A/I239T/H241Q |
| 7 | S195A/K197T/S199A |
| 7bis | A72P/G73N/K197T/S199A |
| 8 | T190A/T191Q/S193A |
| 9 | L150S/S152A/Q234R/I236S |

Results of the association between antibodies and Nectin-4 mutant are shown in Table 14 below. (+) means that antibodies-Nectin-4 association occurs. (−) means antibodies-Nectin-4 association does not occur. Results related with mutant 5 are irrelevant, 10 due to an absence of expression of said protein.

TABLE 14

| | EXPI | | | | | | | | | | |
|---|---|---|---|---|---|---|---|---|---|---|---|
| | medium | | | | | | | | | | |
| | (negative | Mutant | Mutant | Mutant | Mutant | Mutant | Mutant | Mutant | Mutant | Mutant | Mutant |
| | control) | 1 | 2 | 3 | 4 | 5 | 6 | 7 | 7bis | 8 | 9 |
| CHG1-M- | − | + | + | + | + | − | + | − | − | + | + |
| 1N4-5E7 | | | | | | | | | | | |
| Enfortumab | − | + | − | − | + | − | + | + | + | + | + |
| N41 | − | − | + | + | + | − | + | + | + | + | + |
| 14A5.2 | − | − | + | + | + | − | + | + | + | + | + |

Antibody 5E7 therefore has a binding site on Nectin-4 encompassing C1 domain residues that are mutated in Mutant 7 and 7bis (S195A/K197T/S199A and A72P/G73N/K197T/S199A). Thus, antibody 5E7 binds epitopes on Nectin-4 that are different than Enfortumab, N41 and 14A5 (that bind epitopes on the V domain of Nectin-4).

### Example 16: In Vivo Efficacy of Exatecan ADC

Human breast cancer cell line SUM190PT was cultured in the following cell culture medium containing Ham's F12, FBS 1 g/L, HEPES 10 mM, Ethanolamine 5 mM, Insulin 5 μg/mL, Hydrocortisone 1 μg/mL, Apo-Transferrin 5 μg/mL, Triiodo Thyronine (T3) 6.7 ng/ml, Sodium selenite 8.7 ng/ml.

Immunodeficient CB17-SCID mice were used at 7 to 8 weeks of age. Anti-Nectin-4 antibodies enfortumab, 5E7 and 6A7 each having a human Fc region were produced and conjugated to payloads Dxd (deruxtecan) or Exatecan via intracellularly cleavable linkers ggfg-Dxd or PEG (8U)-Val-Ala-PAB-Exatecan. Antibodies 6A7 are 5E7 share most CDRs, have comparable Nectin-4 binding affinity and bind the same site on Nectin-4 (for 6A7 amino acid sequences see PCT/EP2021/082872 filed 24 Nov. 2021). The ggfg-Dxd linker will release Dxd (deruxtecan) upon cleavage and the PEG (8U)-Val-Ala-PAB-Exatecan linker will release exatecan upon cleavage.

SUM190 cells were subcutaneously engrafted in CB17-SCID immunodeficient mice at the dose of 0.5 million in 100 μl of Matrigel containing growth factor diluted at ½ in PBS. At day 21, when tumors reached a mean volume at 146.9±63.2 mm³ or 213.2±77.5 mm³ depending on the experiment, mice were randomized into groups of 8 or 9 mice depending on the experiment and treated intravenously with a single injection of 3 or 10 mg/kg body weight ADC or PBS as control. Tumor growths were followed twice a week. Kaplan Meier survival curves were established by using GraphPad Prism V7 software according to the following criteria: when the tumor volume reached 1500 mm³, mice were euthanized and considered dead on the day of sacrifice (D). When tumors were highly necrotic, mice were euthanized and considered dead on the same day (D).

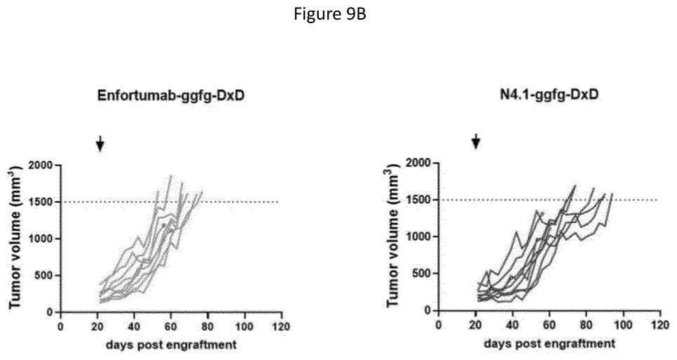

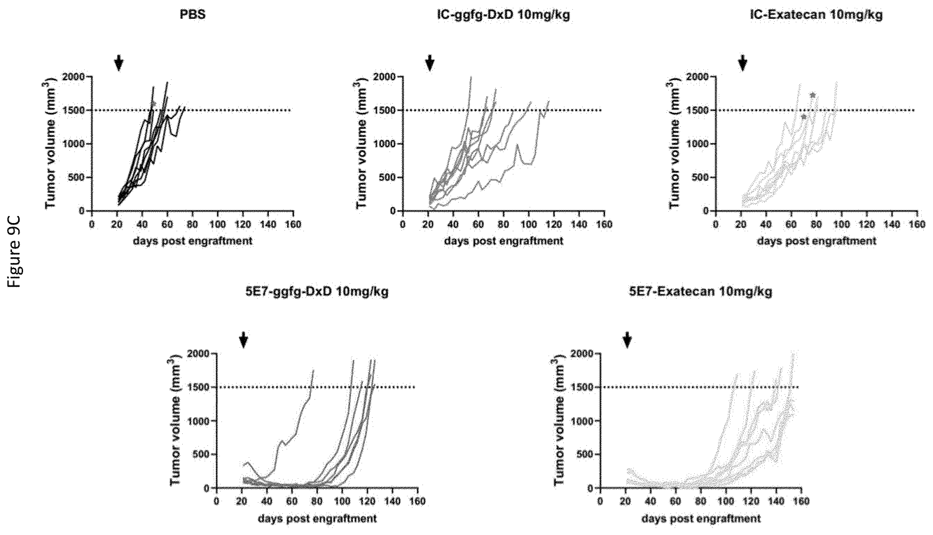

Results are presented in FIGS. 9A and 9B for the 3 mg/kg dose, where it can be seen that the anti-IgVC1 ADCs 5E7-ggfg-Dxd that was found to lose binding to Nectin-4 having K197T/S199A mutations exhibit the highest efficiency in limiting the tumor growth in mice. Furthermore, 5E7 conjugated with the PEG (8U)-Val-Ala-PAB-Exatecan designed to release exatecan upon linker cleavage shows a higher efficiency in controlling tumor growth in mice compared 5E7 conjugated with ggfg-Dxd designed to release Dxd upon linker cleavage, as shown in FIG. 9C for the 10 mg/kg dose.

### Example 17: In Vitro Efficacy of ADCs in Pg-p-Expressing Cancer Model

MC-38 cells that endogenously express MDR1 P-glycoprotein (Pgp) were engineered to express Nectin-4 and cultured in DMEM+10% FBS. Cells were either treated with vehicle (DSMO) or with cyclosporin A (5 μM, stock solution in DMSO) known to act as an inhibitor of Pgp, in presence of ADCs. The ADCs tested were as follows:
(a) PADCEV™,
(b) Antibody 5E7 conjugated to deruxtecan (Dxd) via the ggfg-Dxd linker which releases Dxd upon cleavage (5E7-GGFG-DxD), and
(c) Antibody 5E7 conjugated to exatecan via the PEG (8U)-Val-Ala-PAB-Exatecan linker which releases exatecan upon cleavage (5E7-exatecan).

The ADCs and the equivalent isotype control ADCs were used at dose range from 150 to 2.3 10⁻³ nM. After five days of co-incubation with cells, cell viability was measured by addition of Cell Titer Glo™ (CTG) substrate. Luminescence vs. Ab concentration was plotted.

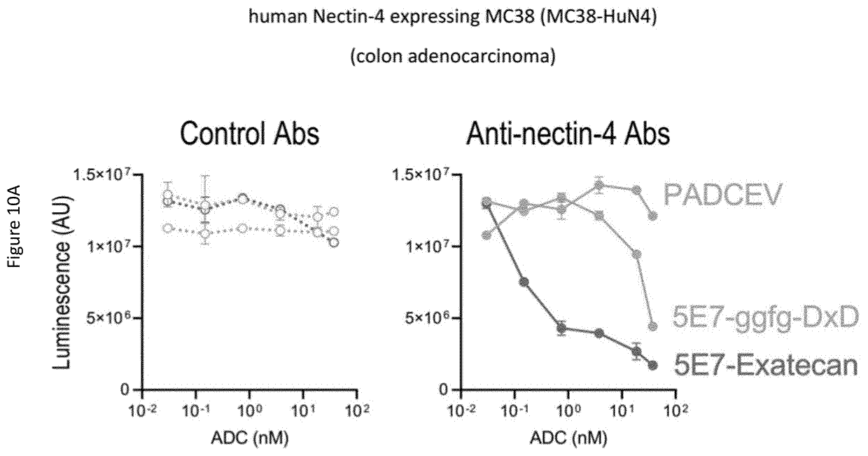

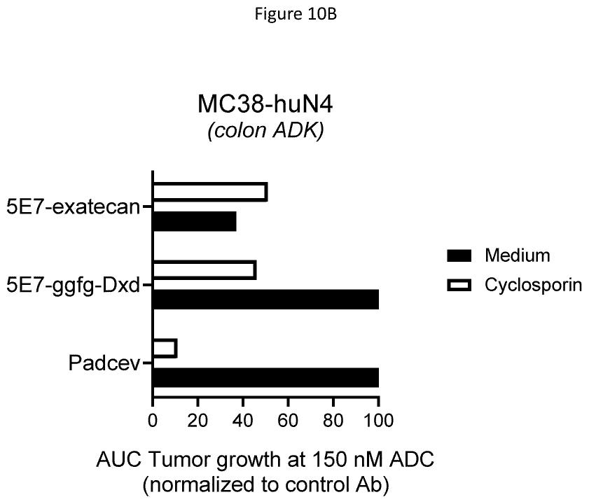

FIG. 10A shows luminescence (indicating cell viability) of cells treated with Padcev™ (enfortumab vedotin), antibody 5E7 conjugated to Dxd or 5E7 conjugated to exatecan. The ADC with exatecan (5E7-exatecan) as payload was highly potent to decrease cell viability in this setting of drug resistance. FIG. 10B shows tumor growth (area under the curve) of the MC38 cells treated, in the presence or absence of the Pgp inhibitor cyclosporine, with Padcev™ (enfortumab vedotin), antibody 5E7 conjugated to Dxd or 5E7 conjugated to exatecan, at 150 nM ADC and normalized to control antibody. The results suggest that the anti-tumor activity of Padcev™ and antibody 5E7 conjugated to Dxd are negatively affected by Pgp at concentrations where 5E7 conjugated to exatecan is highly effective.

### Example 18: In Vivo Efficacy of Branched PEG-Dipeptide-Exatecan ADCs

Anti-VC domain anti-Nectin-4 antibody was conjugated to exatecan via different branched PEG linkers and the resulting ADCs were assessed in an in vivo SUM190 tumor model (Nectin-4 low/SUM190 breast cancer model).

ADCs were prepared in which anti-VC domain anti-Nectin-4 (N4) human IgG1 isotype antibody 6A7 was conjugated (at cysteine residues) at 8 toxins per antibody (DAR=8) to exatecan via different linkers. The linker-toxins tested as follows:

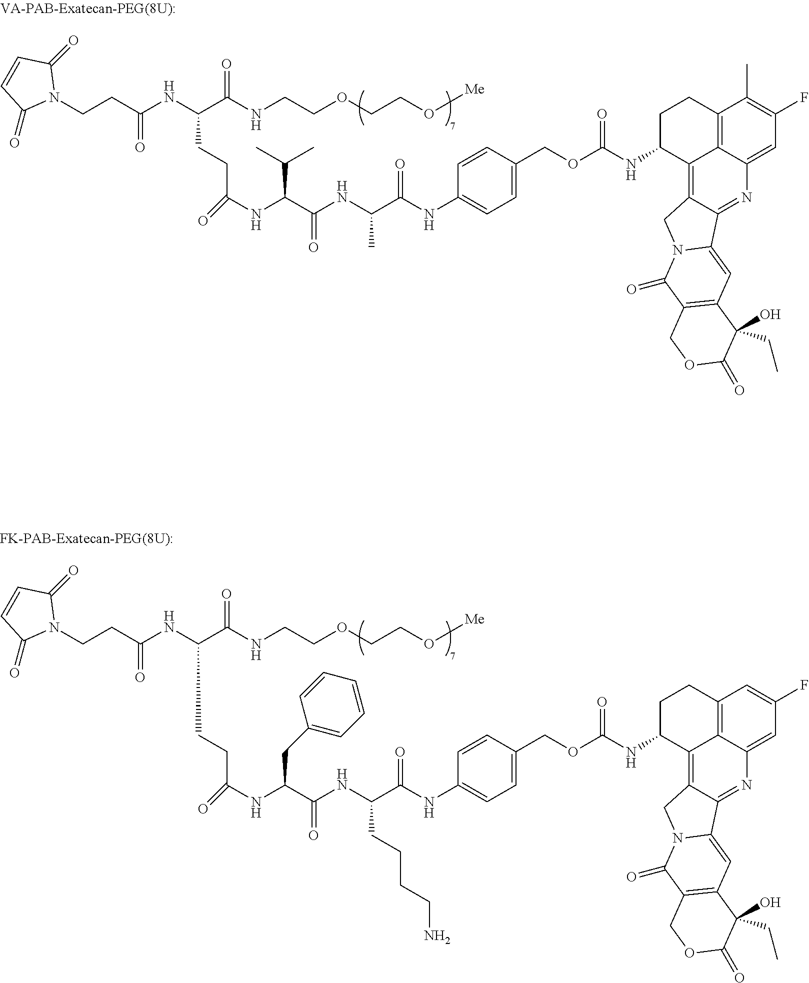

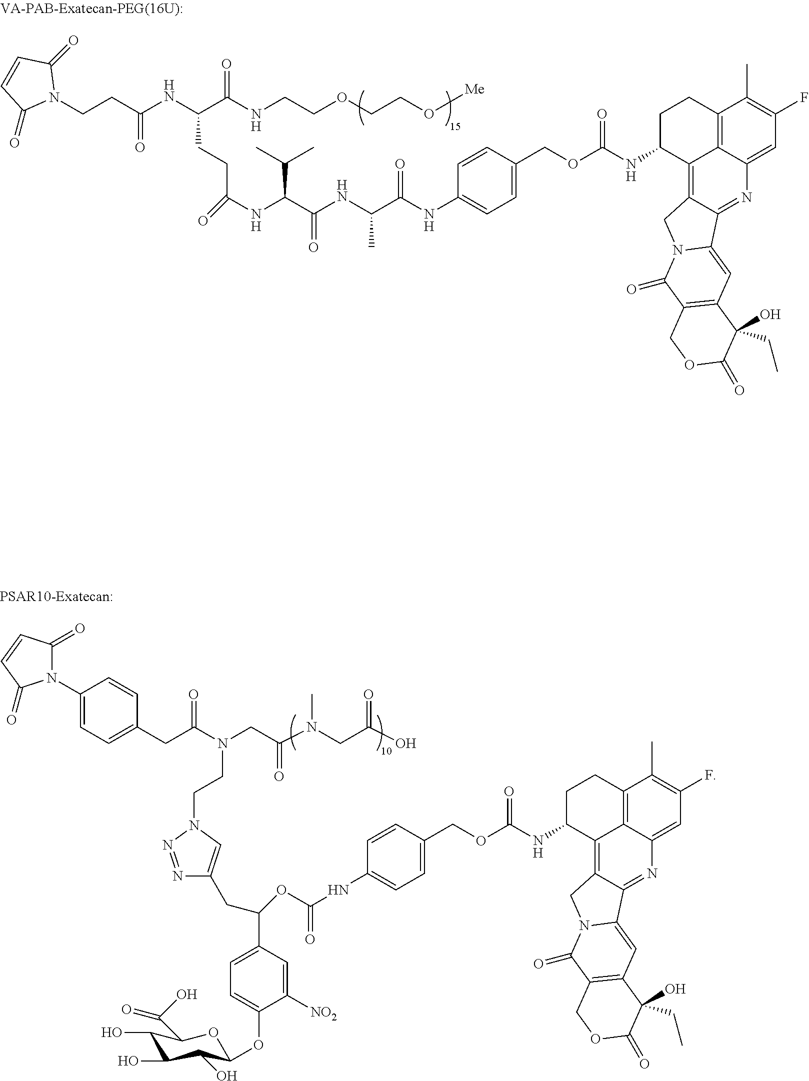

SUM190 cells were subcutaneously engrafted in CB17-SCID immunodeficient mice at a dose of 0.5 million cells in 100 μl of Matrigel with growth factor diluted at ½ in PBS. When tumors reached a volume between 200 and 250 mm³, mice were randomized into groups of 10 mice for intravenous treatment with a single injection of 3 mg/kg of ADCs. Tumor growths were followed twice a week. Kaplan Meier survival curves were established by using GraphPad Prism V7 software according to the following criteria: When the tumor volume reached 1500 mm³, mice were euthanized and considered dead on the day of sacrifice (D). When tumors showed signs of necrosis, mice were euthanized and considered dead on the same day (D) (indicated with red star on the graphs of individual tumor growth).

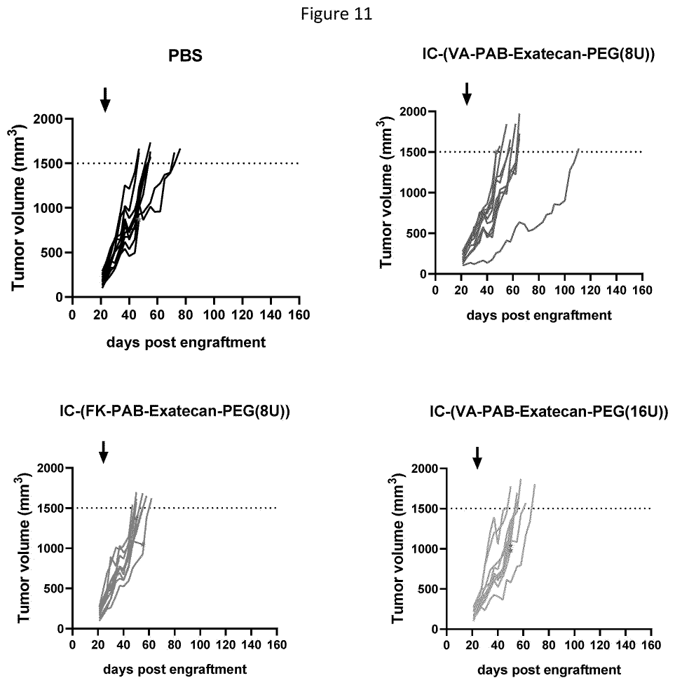

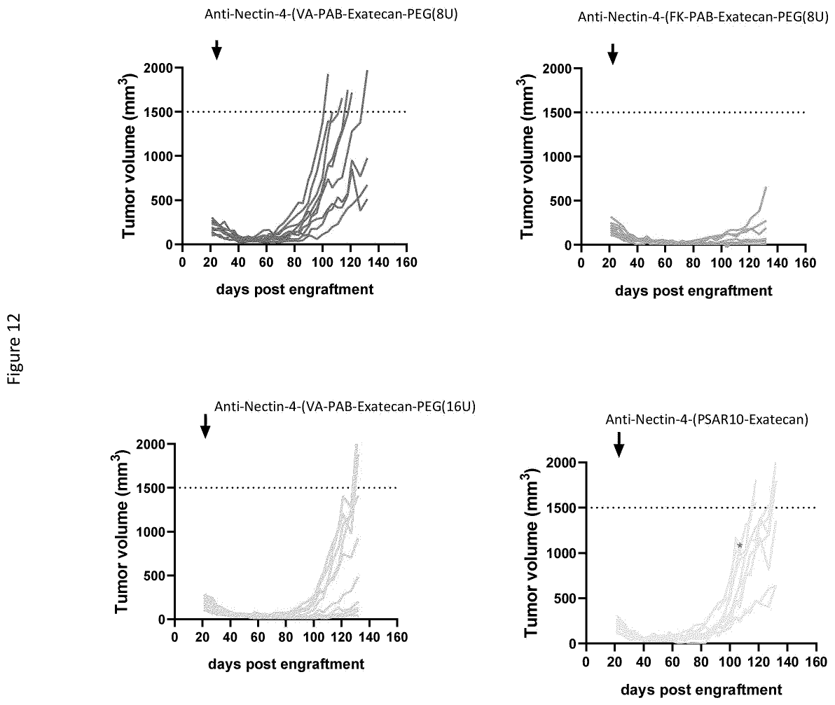

Results showed that at the 3 mg/kg dose all ADC were efficient in preventing increase in tumor volume. The different linker-toxins administered as free toxin (not conjugated to anti-Nectin-4 antibody, indicated as “IC”, were less efficient in preventing tumor growth. Results for the IC (free toxins) are shown in FIG. 11. Results for the ADCs are shown in FIG. 12.

### Example 19: In Vivo Pharmacokinetics and Efficacy of Branched PEG-Dipeptide-Exatecan ADCs

Anti-VC domain anti-Nectin-4 antibody 6A7 was conjugated at (DAR=8) to exatecan via the VA-PAB-Exatecan-PEG (16U) linker (structure shown below) and assessed in an in vivo SUM190 tumor model (Nectin-4 low/SUM190 breast cancer model) at different lower doses. This experiment tested different dosing regimens of up to 1 mg/kg body weight and evaluated the correlation of anti-tumor efficacy with ADC concentration in circulation.

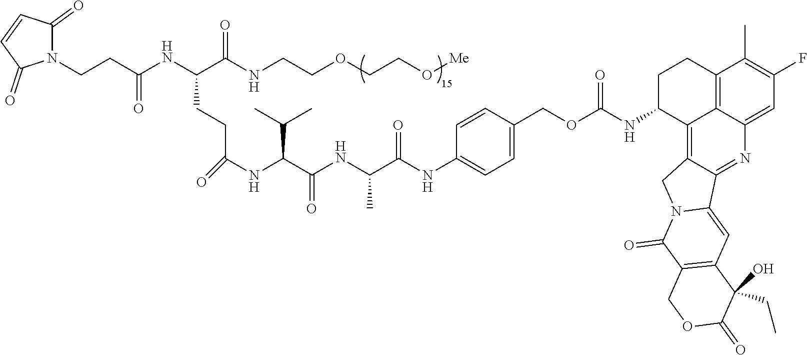

SUM190 cells were subcutaneously engrafted in CB17-SCID immunodeficient mice at a dose of 0.5 million cells in 100 μl of Matrigel with growth factor diluted at ½ in PBS. When tumors reached a volume between 150 and 250 mm³, mice were randomized into groups of 20 mice for intravenous treatment with either PBS as control, or with a single injection of ADCs at either 0.11 mg/kg, 0.33 mg/kg, 0.66 mg/kg or 1 mg/kg body weight (2.2 μg, 6.6 μg, 13.2 μg, and 20 μg doses, respectively). Tumor growth was measured and plasma samples obtained on the same day, with plasma samples post treatment at 5 minutes, 5 h (hours), 24 h and then both plasma samples and tumor growth measurement at 72 h, day 7 and then a weekly basis thereafter.

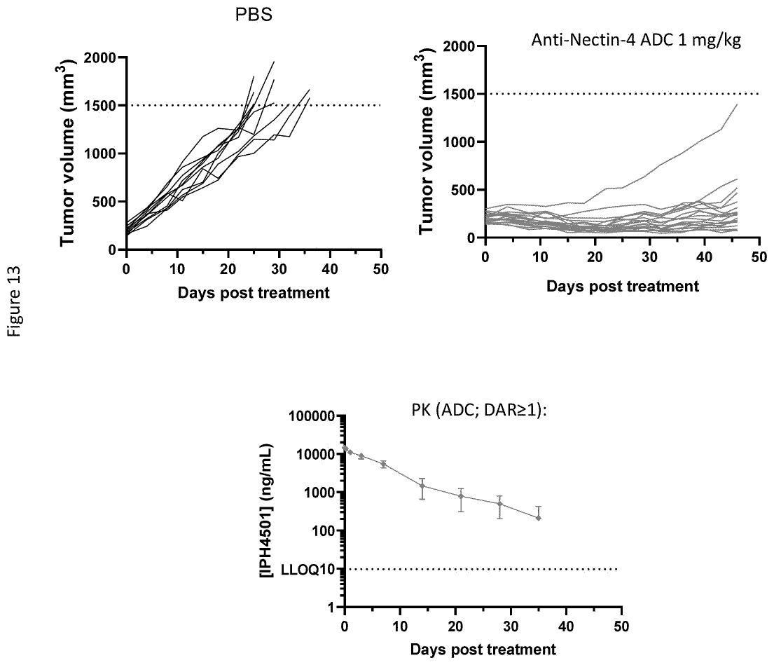

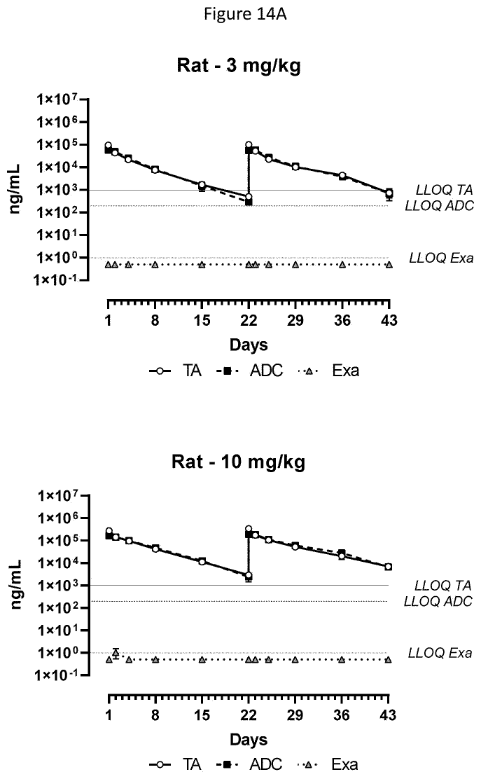

Results are shown in FIG. 13. FIG. 13, top left panel, shows that PBS did not prevent an increase in tumor volume. FIG. 13, top right panel, shows that result for the 1 mg/kg dose of ADC, which showed strong anti-tumor efficacy. FIG. 13, bottom panel, shows concentration of ADC in plasma over time, showing that ADC remained detectable for the period over which the ADC showed anti-tumor activity.

### Example 20: In Vivo Safety of Branched PEG-Dipeptide-Exatecan ADCs

A dose range study of anti-VC domain anti-Nectin-4 antibody conjugated to the VA-PAB-Exatecan-PEG (16U) linker at DAR-8 was carried out at doses ranging from 3 mg/kg body weight to 30 mg/kg body weight.

In a first experiment, the ADC was injected by intravenous bolus in Sprague Dawley rats at 3, 10, or 30 mg/kg body weight on days 1 and 22. Plasma samples were collected along the course of the study (n=3 males per timepoint per dose-level), and the concentration of total anti-Nectin-4 antibody (Total Antibody; TA) (DAR≥0) and ADC (DAR≥1) were measured by ELISA, and the free exatecan (Exa) was measured by LC-MS.

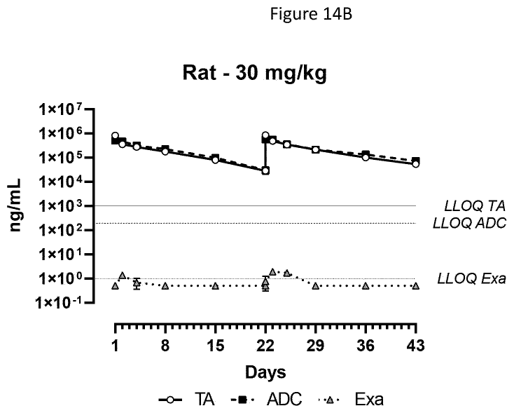

FIG. 14A shows the results for 3 mg/kg dose (top panel) and 10 mg/kg dose (bottom panel) and FIG. 14B shows the results for the 30 mg/kg dose. For each analyte, concentration values below the lower limit of quantification (LLOQ) were plotted at LLOQ/2. The Y axis represents the analyte plasma concentrations in ng/ml, and the X axis represents the time in days. Symbols and bars show the mean and standard deviation of each group.

In a second experiment, the ADC was injected by intravenous bolus in Mauritian cynomolgus monkeys at 3, 10, or 30 mg/kg body weight on days 1 and 22. Plasma samples were collected along the course of the study (n=1 male and 1 female per dose-level), and the concentration of total anti-Nectin-4 antibody (Total Antibody; TA) (DAR≥0) and ADC (DAR≥1) were measured by ELISA, and the free exatecan (Exa) was measured by LC-MS.

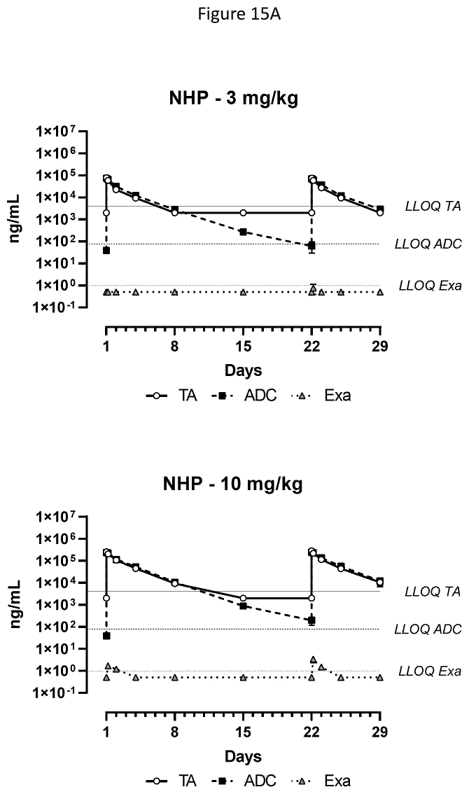

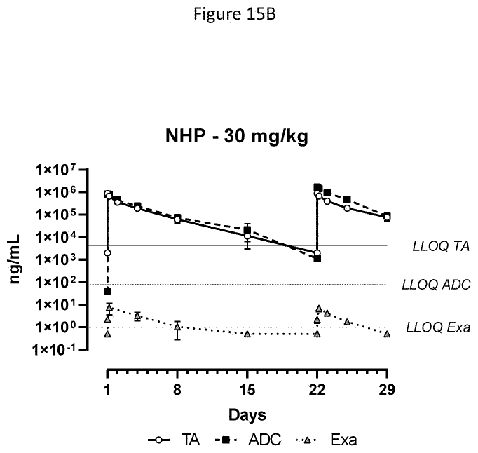

FIG. 15A shows the results for 3 mg/kg dose (top panel) and 10 mg/kg dose (bottom panel) and FIG. 15B shows the results for the 30 mg/kg dose. For each analyte, concentration values below the lower limit of quantification (LLOQ) were plotted at LLOQ/2. The Y axis represents the analyte plasma concentrations in ng/ml, and the X axis represents the time in days. Symbols and bars show the mean and standard deviation of each group.

The results in both rats and non-human primates demonstrated that the ADCs were safe and well-tolerated at the highest dose tested (30 mg/kg body weight).

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). Where “about” is used in connection with a number, this can be specified as including values corresponding to +/−10% of the specified number.

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

TABLE 15

| SEQ | | |
|---|---|---|
| ID | | |
| NO: | Description | Sequence |
| 1 | hNectin-4 with | MPLSLGAEMW GPEAWLLLLL LLASFTGRCP AGELETSDVV TVVLGQDAKL |
| | signal peptide | PCFYRGDSGE QVGQVAWARV DAGEGAQELA LLHSKYGLHV SPAYEGRVEQ |
| | | PPPPRNPLDG SVLLRNAVQA DEGEYECRVS TFPAGSFQAR LRLRVLVPPL |
| | | PSLNPGPALE EGQGLTLAAS CTAEGSPAPS VTWDTEVKGT TSSRSFKHSR |
| | | SAAVTSEFHL VPSRSMNGQP LTCVVSHPGL LQDQRITHIL HVSFLAEASV |
| | | RGLEDQNLWH IGREGAMLKC LSEGQPPPSY NWTRLDGPLP SGVRVDGDTL |
| | | GFPPLTTEHS GIYVCHVSNE FSSRDSQVTV DVLDPQEDSG KQVDLVSASV |
| | | VVVGVIAALL FCLLVVVVVL MSRYHRRKAQ QMTQKYEEEL TLTRENSIRR |
| | | LHSHHTDPRS QPEESVGLRA EGHPDSLKDN SSCSVMSEEP EGRSYSTLTT |
| | | VREIETQTEL LSPGSGRAEE EEDQDEGIKQ AMNHFVQENG TLRAKPTGNG |
| | | IYINGRGHLV |
| 2 | hNectin-4 used | GRCPAGELETSDVVTVVLGQDAKLPCFYRGDSGEQVGQVAWARVDAGEGAQELAL |
| | in immunization | LHSKYGLHVSPAYEGRVEQPPPPRNPLDGSVLLRNAVQADEGEYECRVSTFPAGS |
| | | FQARLRLRVLVPPLPSLNPGPALEEGQGLTLAASCTAEGSPAPSVTWDTEVKGTT |
| | | SSRSFKHSRSAAVTSEFHLVPSRSMNGQPLTCVVSHPGLLQDQRITHILHVSFLA |
| | | EASVRGLEDQNLWHIGREGAMLKCLSEGQPPPSYNWTRLDGPLPSGVRVDGDTLG |
| | | FPPLTTEHSGIYVCHVSNEFSSRDSQVTVDVLDPQEDSGKQVDLVSASVV |
| 3 | V Domain | GELETSDVVTVVLGQDAKLPCFYRGDSGEQVGQVAWARVDAGEGAQELALLHSKY |
| | | GLHVSPAYEGRVEQPPPPRNPLDGSVLLRNAVQADEGEYECRVSTFPAGSFQARL |
| | | RLR |
| 4 | Ig-like C2 type | PPLPSLNPGPALEEGQGLTLAASCTAEGSPAPSVTWDTEVKGTTSSRSFKHSRSA |
| | 1 | AVTSEFHLVPSRSMNGQPLTCVVSHPGLLQDQRIT |
| 5 | Ig-like C2 type | ASVRGLEDQNLWHIGREGAMLKCLSEGQPPPSYNWTRLDGPLPSGVRVDGDTLGF |
| | 2 | PPLTTEHSGIYVCHVSNEFSSRDSQVTVDVLDPQEDSGKQVDLVSASV |
| 6 | N41 VH | QVQLKQSGPGLVQPSQSLSITCTVSGFSLTNYGVHWVRQSPGKGLEWLGVIWSGG |
| | | STDYNAAFISRLSISKDTSKSQVFFKMNSLQADDTAIYYCARELIHAMDNWGQGT |
| | | SVTVSS |
| 7 | N41 VL | DIQMTQSPASLSVSVGETVTITCRASENIYSNLAWYQQKQGNSPQLLVFAATNLA |
| | | DGVPSRFSGSGSGTQYSLKINSLQSEDFGTYYCQHFWGTPTFGGGTKLEIK |
| 8 | Human Nectin4- | GELETSDVVTVVLGQDAKLPCFYRGDSGEQVGQVAWARVDAGEGAQELALLHSKY |
| | His-BirA | GLHVSPAYEGRVEQPPPPRNPLDGSVLLRNAVQADEGEYECRVSTFPAGSFQARL |
| | | RLRVLVPPLPSLNPGPALEEGQGLTLAASCTAEGSPAPSVTWDTEVKGTTSSRSF |
| | | KHSRSAAVTSEFHLVPSRSMNGQPLTCVVSHPGLLQDQRITHILHVSFLAEASVR |
| | | GLEDQNLWHIGREGAMLKCLSEGQPPPSYNWTRLDGPLPSGVRVDGDTLGFPPLT |
| | | TEHSGIYVCHVSNEFSSRDSQVTVDVLDPQEDSGKQVDLVSASHHHHHHLHHILD |
| | | AQKMVWNHR |
| 9 | Wildtype N4 | GELETSDVVTVVLGQDAKLPCFYRGDSGEQVGQVAWARVDAGEGAQELALLHSKY |
| | construct | GLHVSPAYEGRVEQPPPPRNPLDGSVLLRNAVQADEGEYECRVSTFPAGSFQARL |
| | | RLRVLVPPLPSLNPGPALEEGQGLTLAASCTAEGSPAPSVTWDTEVKGTTSSRSF |
| | | KHSRSAAVTSEFHLVPSRSMNGQPLTCVVSHPGLLQDQRITHILHVSFLAEASVR |
| | | GLEDQNLWHIGREGAMLKCLSEGQPPPSYNWTRLDGPLPSGVRVDGDTLGFPPLT |
| | | TEHSGIYVCHVSNEFSSRDSQVTVDVLDPQEDSGKQVDLVSASVVVVGVIAALLF |
| | | CLLVVVVVLMSRYHRRKAQQMTQKYEEELTLTRENSIRRLHSHHTDPRSQPEESV |
| | | GLRAEGHPDSLKDNSSCSVMSEEPEGRSYSTLTTVREIETQTELLSPGSGRAEEE |
| | | EDQDEGIKQAMNHFVQENGTLRAKPTGNGIYINGRGHLV |
| 10 | C1C2 construct | PPLPSLNPGPALEEGQGLTLAASCTAEGSPAPSVTWDTEVKGTTSSRSFKHSRSA |
| | | AVTSEFHLVPSRSMNGQPLTCVVSHPGLLQDQRITHILHVSFLAEASVRGLEDQN |
| | | LWHIGREGAMLKCLSEGQPPPSYNWTRLDGPLPSGVRVDGDTLGFPPLTTEHSGI |
| | | YVCHVSNEFSSRDSQVTVDVLDPQEDSGKQVDLVSASVV |
| | | VVGVIAALLFCLLVVVVVLMSRYHRRKAQQMTQKYEEELTLTRENSIRRLHSHHT |
| | | DPRSQPEESVGLRAEGHPDSLKDNSSCSVMSEEPEGRSYSTLTTVREIETQTELL |
| | | SPGSGRAEEEEDQDEGIKQAMNHFVQENGTLRAKPT |
| | | GNGIYINGRGHLV |
| 11 | C2 construct | ASVRGLEDQNLWHIGREGAMLKCLSEGQPPPSYNWTRLDGPLPSGVRVDGDTLGF |
| | | PPLTTEHSGIYVCHVSNEFSSRDSQVTVDVLDPQEDSGKQVDLVSASVVVVGVIA |
| | | ALLFCLLVVVVVLMSRYHRRKAQQMTQKYEEELTLTRENSIRRLHSHHTDPRSQP |
| | | EESVGLRAEGHPDSLKDNSSCSVMSEEPEGRSYSTLTTVREIETQTELLSPGSGR |
| | | AEEEEDQDEGIKQAMNHFVQENGTLRAKPTGNGIYINGRGHLV |
| 12 | Mouse Nectin-4- | ELETSDVVTVVLGQDAKLPCFYRGDPDEQVGQVAWARVDPNEGIRELALLHSKYG |
| | V5 | LHVNPAYEDRVEQPPPPRDPLDGSVLLRNAVQADEGEYECRVSTFPAGSFQARMR |
| | | LRVLVPPLPSLNPGPPLEEGQGLTLAASCTAEGSPAPSVTWDTEVKGTQSSRSFT |
| | | HPRSAAVTSEFHLVPSRSMNGQPLTCVVSHPGLLQDRRITHTLQVAFLAEASVRG |
| | | LEDQNLWQVGREGATLKCLSEGQPPPKYNWTRLDGPLPSGVRVKGDTLGFPPLTT |
| | | EHSGVYVCHVSNELSSRDSQVTVEVLDPEDPGKQVDLVSASVIIVGVIAALLFCL |
| | | LVVVVVLMSRYHRRKAQQMTQKYEEELTLTRENSIRRLHSHHSDPRSQPEESVGL |
| | | RAEGHPDSLKDNSSCSVMSEEPEGRSYSTLTTVREIETQTELLSPGSGRTEEDDD |
| | | QDEGIKQAMNHFVQENGTLRAKPTGNGIYINGRGHLV |
| 13 | Rat Nectin-4 V5 | MPLSLGAEMWGPEAWLLLLFLASFTGRYSAGELETSDLVTVVLGQDAKLPCFYRG |
| | | DPDEQVGQVAWARVDPNEGTRELALLHSKYGLHVSPAYEDRVEQPPPPRDPLDGS |
| | | ILLRNAVQADEGEYECRVSTFPAGSFQARMRLRVLVPPLPSLNPGPPLEEGQGLT |
| | | LAASCTAEGSPAPSVTWDTEVKGTQSSRSFKHSRSAAVTSEFHLVPSRSMNGQPL |
| | | TCVVSHPGLLQDQRITHTLQVAFLAEASVRGLEDQNLWHVGREGATLKCLSEGQP |
| | | PPKYNWTRLDGPLPSGVRVKGDTLGFPPLTTEHSGVYVCHVSNELSSRASQVTVE |
| | | VLDPEDPGKQVDLVSASVVVVGVIAALLFCLLVVVVVLMSRYHRRKAQQMTQKYE |
| | | EELTLTRENSIRRLHSHHTDPRSQPEESVGLRAEGHPDSLKDNSSCSVMSEEPEG |
| | | RSYSTLTTVREIETQTELLSPGSGRTEEEDDQDEGIKQAMNHFVQENGTLRAKPT |
| | | GNGIYINGRGHLV |
| 14 | Cyno Nectin-4 | GELETSDVVTVVLGQDAKLPCFYRGDSGEQVGQVAWARADAGEGAQELALLHSKY |
| | V5 | GLHVSPAYEGRVEQPPPPRNPLDGSVLLRNAVQADEGEYECRVSTFPAGSFQARL |
| | | RLRVLVPPLPSLNPGPALEEGQGLTLAASCTAEGSPAPSVTWDTEVKGTTSSRSF |
| | | KHSRSAAVTSEFHLVPSRSMNGQPLTCVVSHPGLLQDQRITHILHVSFLAEASVR |
| | | GLEDQNLWHVGREGAMLKCLSEGQPPPSYNWTRLDGPLPSGVRVDGDTLGFPPLT |
| | | TEHSGIYVCHVSNEFSSRDSQVTVDVLDPQEDSGKQVDLVSASVVVVGVIAALLF |
| | | CLLVVVVVLMSRYHRRKAQQMTQKYEEELTLTRENSIRRLHSHHTDPRSQPEESV |
| | | GLRAEGHPDSLKDNSSCSVMSEEPEGRSYSTLTTVREIETQTELLSPGSGRTEEE |
| | | EDQDEGIKQAMNHFVQENGTLRAKPTGNGIYINGRGHLV |
| 15 | Nectin-1 | MGLAGAAGRWWGLALGLTAFFLPGVHSQVVQVNDSMYGFIGTDVVLHCSFANPLP |
| | | SVKITQVTWQKSTNGSKQNVAIYNPSMGVSVLAPYRERVEFLRPSFTDGTIRLSR |
| | | LELEDEGVYICEFATFPTGNRESQLNLTVMAKPTNWIEGTQAVLRAKKGQDDKVL |
| | | VATCTSANGKPPSVVSWETRLKGEAEYQEIRNPNGTVTVISRYRLVPSREAHQQS |
| | | LACIVNYHMDRFKESLTLNVQYEPEVTIEGFDGNWYLQRMDVKLTCKADANPPAT |
| | | EYHWTTLNGSLPKGVEAQNRTLFFKGPINYSLAGTYICEATNPIGTRSGQVEVNI |
| | | TEFPYTPSPPEHGRRAGPVPTAIIGGVAGSILLVLIVVGGIVVALRRRRHTFKGD |
| | | YSTKKHVYGNGYSKAGIPQHHPPMAQNLQYPDDSDDEKKAGPLGGSSYEEEEEEE |
| | | EGGGGGERKVGGPHPKYDEDAKRPYFTVDEAEARQDGYGDRTLGYQYDPEQLDLA |
| | | ENMVSQNDGSFISKKEWYV |
| 16 | Nectin-2 | MARAAALLPSRSPPTPLLWPLLLLLLLETGAQDVRVQVLPEVRGQLGGTVELPCH |
| | | LLPPVPGLYISLVTWQRPDAPANHQNVAAFHPKMGPSFPSPKPGSERLSFVSAKQ |
| | | STGQDTEAELQDATLALHGLTVEDEGNYTCEFATFPKGSVRGMTWLRVIAKPKNQ |
| | | AEAQKVTFSQDPTTVALCISKEGRPPARISWLSSLDWEAKETQVSGTLAGTVTVT |
| | | SRFTLVPSGRADGVTVTCKVEHESFEEPALIPVTLSVRYPPEVSISGYDDNWYLG |
| | | RTDATLSCDVRSNPEPTGYDWSTTSGTFPTSAVAQGSQLVIHAVDSLENTTFVCT |
| | | VTNAVGMGRAEQVIFVRETPNTAGAGATGGIIGGIIAAIIATAVAATGILICRQQ |
| | | RKEQTLQGAEEDEDLEGPPSYKPPTPKAKLEAQEMPSQLFTLGASEHSPLKTPYF |
| | | DAGASCTEQEMPRYHELPTLEERSGPLHPGATSLGSPIPVPPGPPAVEDVSLDLE |
| | | DEEGEEEEEYLDKINPIYDALSYSSPSDSYQGKGFVMSRAMYV |
| 17 | Nectin-3 | MARTLRPSPLCPGGGKAQLSSASLLGAGLLLQPPTPPPLLLLLFPLLLFSRLCGA |
| | | LAGPIIVEPHVTAVWGKNVSLKCLIEVNETITQISWEKIHGKSSQTVAVHHPQYG |
| | | FSVQGEYQGRVLFKNYSLNDATITLHNIGFSDSGKYICKAVTFPLGNAQSSTTVT |
| | | VLVEPTVSLIKGPDSLIDGGNETVAAICIAATGKPVAHIDWEGDLGEMESTTTSF |
| | | PNETATIISQYKLFPTRFARGRRITCVVKHPALEKDIRYSFILDIQYAPEVSVTG |
| | | YDGNWFVGRKGVNLKCNADANPPPFKSVWSRLDGQWPDGLLASDNTLHFVHPLTF |
| | | NYSGVYICKVTNSLGQRSDQKVIYISDPPTTTTLQPTIQWHPSTADIEDLATEPK |
| | | KLPFPLSTLATIKDDTIATIIASVVGGALFIVLVSVLAGIFCYRRRRTFRGDYFA |
| | | KNYIPPSDMQKESQIDVLQQDELDSYPDSVKKENKNPVNNLIRKDYLEEPEKTQW |
| | | NNVENLNRFERPMDYYEDLKMGMKFVSDEHYDENEDDLVSHVDGSVISRREWYV |
| 18 | PVR Nectin-5 | DVVVQAPTQVPGFLGDSVTLPCYLQVPNMEVTHVSQLTWARHGESGSMAVFHQTQ |
| | | GPSYSESKRLEFVAARLGAELRNASLRMFGLRVEDEGNYTCLFVTFPQGSRSVDI |
| | | WLRVLAKPQNTAEVQKVQLTGEPVPMARCVSTGGRPPAQITWHSDLGGMPNTSQV |
| | | PGFLSGTVTVTSLWILVPSSQVDGKNVTCKVEHESFEKPQLLTVNLTVYYPPEVS |
| | | ISGYDNNWYLGQNEATLTCDARSNPEPTGYNWSTTMGPLPPFAVAQGAQLLIRPV |
| | | DKPINTTLICNVTNALGARQAELTVQVKEGPPSEHSGISRNAIIFLVLGILVELI |
| | | LLGIGIYFYWSKCSREVLWHCHLCPSSTEHASASANGHVSYSAVSRENSSSQDPQ |
| | | TEGTR |
| 19 | 5E7 VH | QVQLQQPGAELVKPGASVKLSCKASGYIFTSYWMHWVKQRPGQGLEWIGEIDPSD |
| | | SYTNYNQKFKGKATLTLDKSSSTTYMQLSSLTSEDSAVYYCVRGYGNYGDYWGQG |
| | | TTLTVSSASTKGP |
| 20 | 5E7 VL | DVVMTQAAFSNPVTLGTSASISCRSSKSLLHSNGITYLYWYLQKPGQSPQLLIYQ |
| | | MSNLASGVPDRESSSGSGTDFTLRISRVEAEDVGVYYCAQNLELPWTFGGGTKLE |
| | | IKRTVAAP |
| 21 | 5E7 CDR-H1 | SYWMH |
| | Kabat | |
| 22 | 5E7 CDR-H2 | EIDPSDSYTNYNQKFKG |
| | Kabat | |
| 23 | 5E7 CDR-H3 | GYGNYGDY |
| | Kabat | |
| 24 | 5E7 CDR-L1 | RSSKSLLHSNGITYLY |
| | Kabat | |
| 25 | 5E7 CDR-L2 | QMSNLAS |
| | Kabat | |
| 26 | 5E7 CDR-L3 | AQNLELPWT |
| | Kabat | |
| 27 | 5E7 CDR-H1 IMGT | GYIFTSYW |
| 28 | 5E7 CDR-H2 IMGT | IDPSDSYT |
| 29 | 5E7 CDR-H3 IMGT | VRGYGNYGDY |
| 30 | 5E7 CDR-L1 IMGT | KSLLHSNGITY |
| 31 | 5E7 CDR-L2 IMGT | QMS |
| 32 | 5E7 CDR-H1 | GYIFTSY |
| | CHotia | |
| 33 | 5E7 CDR-H2 | PSDS |
| | Chotia | |
| 34 | 5E7 CDR-H3 | YGNYGD |
| | Chotia | |
| 35 | 5E7 CDR-L1 | SKSLLHSNGITY |
| | Chotia | |
| 36 | 5E7 CDR-L3 | NLELPW |
| | Chotia | |
| 37 | 5E7 H0 | QVQLVQSGAEVKKPGASVKVSCKASGYTFTSYWMHWVRQAPGQGLEWMGEIDPSD |
| | | SYTNYNQKFKGRVTMTRDTSTSTVYMELSSLRSEDTAVYYCARGYGNYGDYWGQG |
| | | TLVTVSS |
| 38 | 5E7 H0 DNA | CAG GTT CAG CTG GTT CAG TCA GGT GCT GAG GTG AAG AAG CCT |
| | | GGA GCT AGC GTG AAA GTG TCC TGC AAA GCC TCT GGC TAC ACC |
| | | TTT ACC TCC TAT TGG ATG CAC TGG GTA CGA CAG GCA CCA GGA |
| | | CAA GGG CTG GAA TGG ATG GGC GAA ATC GAT CCC TCT GAC AGC |
| | | TAC ACG AAC TAC AAC CAG AAG TTC AAG GGT AGG GTC ACT ATG |
| | | ACT CGC GAC ACA TCC ACC AGT ACC GTC TAC ATG GAG CTC TCC |
| | | AGT TTG CGG TCT GAG GAT ACA GCC GTG TAC TAC TGT GCC AGA |
| | | GGC TAT GGC AAT TAT GGG GAC TAT TGG GGA CAA GGG ACA CTG |
| | | GTC ACT GTG AGC TCA |
| 39 | 5E7 H1 | QVQLVQSGAEVKKPGASVKVSCKASGYTFTSYWMHWVRQAPGQGLEWMGEIDPSD |
| | | SYTNYNQKFKGRVTMTLDTSTSTTYMELSSLRSEDTAVYYCARGYGNYGDYWGQG |
| | | TLVTVSS |
| 40 | 5E7 H1 DNA | CAG GTT CAG CTG GTC CAG AGT GGT GCA GAA GTG AAG AAA CCT |
| | | GGC GCT TCA GTG AAG GTA TCC TGC AAA GCC TCT GGG TAC ACC |
| | | TTT ACC TCC TAT TGG ATG CAC TGG GTT CGC CAA GCT CCA GGA |
| | | CAG GGC TTG GAA TGG ATG GGA GAG ATC GAT CCC TCT GAC TCC |
| | | TAC ACC AAC TAC AAC CAG AAG TTC AAG GGA AGG GTG ACG ATG |
| | | ACT CTC GAC ACT AGC ACC AGC ACA ACC TAC ATG GAG CTG AGT |
| | | TCA CTT CGG TCT GAG GAT ACT GCC GTC TAC TAC TGT GCC AGA |
| | | GGC TAT GGG AAT TAT GGG GAC TAT TGG GGT CAA GGC ACA CTG |
| | | GTG ACA GTG TCC AGC |
| 41 | 5E7 H2 | QVQLVQSGAEVKKPGASVKVSCKASGYTFTSYWMHWVRQAPGQGLEWMGEIDPSD |
| | | SYTNYNQKFKGRVTMTLDKSTSTTYMELSSLRSEDTAVYYCARGYGNYGDYWGQG |
| | | TLVTVSS |
| 42 | 5E7 H2 DNA | CAG GTG CAG CTG GTT CAG TCA GGA GCA GAG GTC AAG AAA CCT |
| | | GGT GCT TCT GTG AAG GTG TCC TGC AAA GCC AGT GGC TAT ACC |
| | | TTT ACC AGC TAC TGG ATG CAC TGG GTT CGG CAA GCT CCA GGT |
| | | CAG GGC TTG GAA TGG ATG GGA GAG ATC GAT CCC TCA GAC TCC |
| | | TAC ACG AAC TAC AAC CAG AAG TTC AAA GGG AGG GTA ACC ATG |
| | | ACA CTG GAC AAG TCT ACA TCC ACC ACT TAC ATG GAG CTG AGT |
| | | TCT CTT CGC TCC GAA GAT ACT GCC GTG TAC TAC TGT GCC AGA |
| | | GGC TAT GGG AAT TAT GGC GAC TAT TGG GGA CAA GGG ACT CTC |
| | | GTG ACA GTC AGC AGC |
| 43 | 5E7 H3 | QVQLVQSGAEVKKPGASVKVSCKASGYIFTSYWMHWVRQAPGQGLEWMGEIDPSD |
| | | SYTNYNQKFKGRVTMTLDKSTSTTYMELSSLRSEDTAVYYCARGYGNYGDYWGQG |
| | | TLVTVSS |
| 44 | 5E7 H3 DNA | CAG GTT CAG CTG GTC CAG TCA GGT GCA GAA GTG AAG AAA CCT |
| | | GGA GCT AGC GTG AAG GTG TCC TGC AAA GCC TCT GGC TAC ATC |
| | | TTT ACC TCC TAT TGG ATG CAC TGG GTA CGC CAA GCT CCA GGT |
| | | CAG GGG TTG GAA TGG ATG GGA GAG ATT GAT CCC TCT GAC AGC |
| | | TAC ACC AAC TAC AAC CAG AAG TTC AAA GGA CGG GTT ACC ATG |
| | | ACA CTG GAC AAG TCC ACT TCA ACG ACC TAC ATG GAG CTT TCC |
| | | TCT CTG AGG AGT GAG GAT ACT GCC GTG TAC TAC TGT GCC AGA |
| | | GGG TAT GGC AAT TAT GGG GAC TAT TGG GGC CAA GGC ACA CTC |
| | | GTC ACA GTG AGC AGT |
| 45 | 5E7 H4 | QVQLVQSGAEVKKPGASVKVSCKASGYIFTSYWMHWVKQAPGQGLEWMGEIDPSD |
| | | SYTNYNQKFKGRVTMTLDKSTSTTYMELSSLRSEDTAVYYCARGYGNYGDYWGQG |
| | | TLVTVSS |
| 46 | 5E7 H4 DNA | CAG GTT CAG CTC GTT CAG TCT GGA GCC GAA GTG AAG AAA CCA |
| | | GGT GCT AGC GTG AAA GTG AGT TGC AAG GCT TCC GGC TAC ATC |
| | | TTT ACC AGC TAT TGG ATG CAC TGG GTC AAA CAG GCA CCT GGA |
| | | CAA GGG TTG GAG TGG ATG GGA GAG ATT GAT CCC TCA GAC TCC |
| | | TAC ACC AAC TAC AAC CAG AAG TTC AAG GGC AGG GTA ACC ATG |
| | | ACA CTG GAC AAG TCC ACG AGT ACT ACC TAC ATG GAA CTG TCC |
| | | TCT CTT CGG TCT GAG GAT ACA GCC GTG TAC TAC TGT GCC AGA |
| | | GGG TAT GGC AAT TAT GGG GAC TAT TGG GGT CAA GGC ACT CTG |
| | | GTC ACA GTG AGC TCA |
| 47 | 5E7 H5 | QVQLVQSGAEVKKPGASVKVSCKASGYIFTSYWMHWVKQRPGQGLEWMGEIDPSD |
| | | SYTNYNQKFKGRVTMTLDKSTSTTYMELSSLRSEDTAVYYCARGYGNYGDYWGQG |
| | | TLVTVSS |
| 48 | 5E7 H5 DNA | CAG GTT CAG CTC GTT CAG TCA GGT GCA GAA GTC AAG AAA CCA |
| | | GGA GCT AGC GTG AAG GTT TCC TGC AAG GCT TCT GGG TAC ATC |
| | | TTT ACC AGC TAC TGG ATG CAC TGG GTG AAA CAG CGC CCT GGT |
| | | CAA GGA CTG GAA TGG ATG GGC GAG ATT GAT CCC AGT GAC AGC |
| | | TAC ACC AAC TAC AAC CAG AAG TTC AAA GGC AGG GTG ACC ATG |
| | | ACA CTG GAC AAG TCC ACT TCA ACC ACG TAC ATG GAG CTC AGT |
| | | TCC CTT CGG TCT GAG GAT ACA GCC GTC TAC TAT TGT GCC AGA |
| | | GGG TAT GGC AAT TAT GGG GAC TAT TGG GGA CAA GGC ACA CTG |
| | | GTC ACA GTG AGC TCA |
| 49 | 5E7 H6 | QVQLVQSGAEVKKPGASVKVSCKASGYIFTSYWMHWVKQRPGQGLEWIGEIDPSD |
| | | SYTNYNQKFKGRVTMTLDKSTSTTYMELSSLRSEDTAVYYCARGYGNYGDYWGQG |
| | | TLVTVSS |
| 50 | 5E7 H6 DNA | CAG GTT CAG CTC GTT CAG AGT GGA GCA GAG GTG AAG AAA CCA |
| | | GGA GCT TCA GTC AAG GTA TCC TGC AAA GCC TCT GGC TAC ATC |
| | | TTT ACC AGC TAC TGG ATG CAC TGG GTG AAA CAA CGC CCT GGA |
| | | CAA GGG TTG GAG TGG ATT GGG GAA ATC GAT CCC TCC GAT TCC |
| | | TAC ACC AAC TAC AAC CAG AAG TTC AAG GGC AGA GTG ACG ATG |
| | | ACA CTG GAC AAG TCT ACA TCC ACT ACC TAC ATG GAA CTC AGC |
| | | TCA CTT CGG AGT GAG GAC ACA GCC GTC TAT TAC TGT GCT AGG |
| | | GGC TAT GGG AAT TAT GGC GAC TAT TGG GGT CAG GGT ACT CTG |
| | | GTC ACA GTG AGC TCA |
| 51 | 5E7 H7 | QVQLVQSGAEVKKPGASVKVSCKASGYIFTSYWMHWVKQRPGQGLEWIGEIDPSD |
| | | SYTNYNQKFKGRVTLTLDKSTSTTYMELSSLRSEDTAVYYCARGYGNYGDYWGQG |
| | | TLVTVSS |
| 52 | 5E7 H7 DNA | CAG GTT CAG CTC GTT CAG TCA GGC GCT GAA GTC AAG AAA CCC |
| | | GGA GCA TCC GTG AAG GTG TCC TGT AAG GCC TCT GGG TAC ATC |
| | | TTT ACG AGC TAC TGG ATG CAC TGG GTG AAA CAG AGG CCT GGT |
| | | CAA GGG CTT GAG TGG ATT GGA GAG ATC GAT CCA AGC GAC TCA |
| | | TAC ACC AAC TAC AAC CAG AAG TTC AAA GGT CGC GTT ACA CTG |
| | | ACT CTG GAC AAG AGT ACC TCC ACC ACC TAC ATG GAA CTG AGT |
| | | TCT TTG CGG TCT GAG GAT ACA GCC GTG TAT TAC TGC GCT AGA |
| | | GGG TAT GGC AAT TAT GGC GAC TAT TGG GGA CAA GGC ACA CTG |
| | | GTC ACA GTG AGC TCA |
| 53 | 5E7 H8 | QVQLVQSGAEVKKPGASVKVSCKASGYIFTSYWMHWVKQAPGQGLEWIGEIDPSD |
| | | SYTNYNQKFKGRVTMTLDKSTSTTYMELSSLRSEDTAVYYCARGYGNYGDYWGQG |
| | | TLVTVSS |
| 54 | 5E7 H8 DNA | ACA GGC GTG CAT TCG CAG GTT CAG CTG GTA CAG TCA GGT GCC |
| | | GAA GTG AAG AAA CCT GGT GCA TCC GTC AAA GTG AGC TGC AAA |
| | | GCC AGT GGG TAC ATC TTT ACC TCC TAT TGG ATG CAC TGG GTT |
| | | AAG CAG GCT CCA GGA CAA GGG TTG GAG TGG ATT GGC GAA ATC |
| | | GAT CCC TCA GAC TCC TAC ACG AAC TAC AAC CAG AAG TTC AAG |
| | | GGC AGA GTG ACA ATG ACT CTG GAC AAG TCT ACC TCT ACC ACC |
| | | TAT ATG GAG CTG TCT AGC CTT CGG AGT GAG GAT ACA GCC GTG |
| | | TAC TAC TGT GCT AGG GGA TAC GGG AAT TAT GGC GAC TAT TGG |
| | | GGA CAA GGC ACA CTC GTC ACT GTG TCC AGC GCG AGC ACC AAG |
| | | GGC |
| 55 | 5E7 H9 | QVQLVQSGAEVKKPGASVKVSCKASGYIFTSYWMHWVKQAPGQGLEWIGEIDPSD |
| | | SYTNYNQKFKGRVTMTLDKSTSTVYMELSSLRSEDTAVYYCARGYGNYGDYWGQG |
| | | TLVTVSS |
| 56 | 5E7 H9 DNA | ACA GGC GTG CAT TCG CAG GTT CAG CTG GTT CAG TCT GGC GCT |
| | | GAA GTC AAG AAA CCT GGA GCC AGT GTC AAG GTG TCA TGC AAA |
| | | GCT TCC GGG TAC ATC TTT ACG AGC TAT TGG ATG CAC TGG GTG |
| | | AAA CAG GCA CCA GGA CAA GGG TTG GAG TGG ATT GGT GAG ATC |
| | | GAT CCC TCA GAC AGC TAC ACC AAC TAC AAC CAG AAG TTC AAG |
| | | GGT AGG GTA ACC ATG ACA CTG GAC AAG TCC ACA AGC ACC GTG |
| | | TAC ATG GAA CTC TCC TCT CTT CGG AGT GAG GAT ACT GCC GTG |
| | | TAC TAC TGT GCC AGA GGC TAT GGC AAT TAT GGG GAC TAT TGG |
| | | GGA CAA GGC ACT CTG GTC ACA GTG TCC TCT GCG AGC ACC AAG |
| | | GGC |
| 57 | 5E7 H10 | QVQLVQSGAEVKKPGASVKVSCKASGYIFTSYWMHWVKQAPGQGLEWVGEIDPSD |
| | | SYTNYNQKFKGRVTMTLDKSTSTTYMELSSLRSEDTAVYYCARGYGNYGDYWGQG |
| | | TLVTVSS |
| 58 | 5E7 H10 DNA | ACA GGC GTG CAT TCG CAG GTA CAG CTG GTT CAG TCA GGT GCT |
| | | GAG GTG AAG AAG CCA GGT GCA TCC GTG AAG GTC AGC TGC AAA |
| | | GCC TCT GGG TAC ATC TTT ACC AGC TAT TGG ATG CAC TGG GTG |
| | | AAA CAG GCT CCT GGA CAA GGA CTG GAA TGG GTT GGG GAG ATT |
| | | GAC CCC AGT GAC TCC TAT ACC AAC TAC AAC CAG AAG TTC AAA |
| | | GGG AGG GTC ACG ATG ACT CTC GAC AAG TCA ACC TCC ACA ACC |
| | | TAC ATG GAA CTG TCC TCT TTG CGG AGT GAG GAT ACA GCC GTG |
| | | TAC TAC TGT GCC AGA GGC TAT GGC AAT TAT GGC GAT TAC TGG |
| | | GGA CAA GGC ACT CTT GTC ACA GTG AGC TCT GCG AGC ACC AAG |
| | | GGC |
| 59 | 5E7 L0 | DIVMTQSPLSLPVTPGEPASISCRSSKSLLHSNGITYLYWYLQKPGQSPQLLIYQ |
| | | MSNLASGVPDRFSGSGSGTDFTLKISRVEAEDVGVYYCAQNLELPWTFGGGTKVE |
| | | IK |
| 60 | 5E7 L0 DNA | GAC ATC GTG ATG ACA CAG AGT CCC TTG TCC TTG CCT GTG ACT |
| | | CCT GGA GAA CCC GCT AGC ATT AGC TGC AGA AGC AGC AAG TCC |
| | | CTT CTC CAC TCT AAC GGC ATA ACC TAT CTG TAC TGG TAT CTG |
| | | CAG AAA CCA GGG CAG AGT CCC CAA CTC CTG ATC TAC CAG ATG |
| | | TCC AAC CTG GCA TCT GGC GTT CCT GAT CGG TTC TCA GGG TCA |
| | | GGT TCT GGC ACT GAC TTC ACA CTG AAG ATC TCC AGG GTA GAG |
| | | GCC GAA GAT GTC GGA GTG TAC TAT TGT GCC CAG AAT CTG GAG |
| | | CTT CCA TGG ACC TTT GGA GGT GGC ACC AAA GTC GAG ATT AAG |
| 61 | 5E7 L1 | DVVMTQSPLSLPVTPGEPASISCRSSKSLLHSNGITYLYWYLQKPGQSPQLLIYQ |
| | | MSNLASGVPDRFSSSGSGTDFTLKISRVEAEDVGVYYCAQNLELPWTFGGGTKVE |
| | | IK |
| 62 | 5E7 L1 DNA | GAT GTC GTG ATG ACC CAG AGT CCA CTG TCC CTT CCT GTC ACT |
| | | CCT GGT GAA CCA GCC AGT ATT TCC TGT CGG TCA AGC AAA TCC |
| | | CTC CTT CAC TCC AAC GGG ATT ACC TAT CTG TAC TGG TAT CTG |
| | | CAG AAA CCC GGC CAA TCT CCC CAG TTG CTC ATC TAC CAG ATG |
| | | AGC AAT CTG GCC TCT GGA GTA CCC GAC AGG TTC AGC TCT AGT |
| | | GGA TCA GGG ACA GAC TTC ACA CTG AAG ATC AGC AGA GTG GAA |
| | | GCT GAG GAT GTT GGC GTG TAC TAT TGC GCA CAG AAC CTG GAG |
| | | TTG CCT TGG ACC TTT GGA GGT GGC ACT AAG GTG GAG ATA AAG |
| 63 | 5E7 L2 | DVVMTQSPLSNPVTPGEPASISCRSSKSLLHSNGITYLYWYLQKPGQSPQLLIYQ |
| | | MSNLASGVPDRFSSSGSGTDFTLKISRVEAEDVGVYYCAQNLELPWTFGGGTKVE |
| | | IK |
| 64 | 5E7 L2 DNA | GAC GTC GTG ATG ACT CAG TCC CCT CTC TCT AAT CCC GTT ACT |
| | | CCT GGA GAA CCA GCC AGC ATT TCC TGC AGA AGC AGC AAA TCC |
| | | CTG CTG CAC AGT AAC GGC ATA ACC TAT CTG TAC TGG TAT CTG |
| | | CAG AAA CCA GGG CAA AGT CCC CAG TTG CTG ATC TAC CAG ATG |
| | | TCC AAC TTG GCT TCA GGT GTG CCT GAT CGG TTC AGT AGC TCT |
| | | GGG TCT GGC ACA GAC TTC ACC CTG AAG ATC TCA AGG GTC GAA |
| | | GCA GAG GAT GTA GGC GTG TAT TAC TGT GCC CAG AAT CTC GAG |
| | | CTT CCC TGG ACC TTT GGA GGT GGA ACT AAG GTG GAG ATA AAG |
| 65 | 5E7 L3 | DVVMTQSALSNPVTPGEPASISCRSSKSLLHSNGITYLYWYLQKPGQSPQLLIYQ |
| | | MSNLASGVPDRFSSSGSGTDFTLKISRVEAEDVGVYYCAQNLELPWTFGGGTKVE |
| | | IK |
| 66 | 5E7 L3 DNA | GAT GTG GTC ATG ACT CAG TCT GCC TTG AGC AAT CCC GTA ACT |
| | | CCA GGT GAA CCA GCC AGC ATA AGT TGT CGG TCA AGC AAG AGT |
| | | CTC CTT CAC TCC AAT GGC ATT ACC TAT CTC TAC TGG TAT CTG |
| | | CAG AAA CCT GGT CAG TCT CCC CAA CTG CTG ATC TAC CAG ATG |
| | | AGC AAC CTG GCA AGT GGA GTC CCT GAC AGG TTC TCC TCC TCA |
| | | GGA TCT GGG ACA GAC TTC ACC CTG AAG ATC TCC AGA GTG GAA |
| | | GCT GAG GAT GTT GGC GTG TAC TAT TGC GCT CAG AAC CTG GAG |
| | | TTG CCC TGG ACC TTT GGC GGA GGG ACT AAG GTG GAG ATA AAG |
| 67 | 5E7 HP FAB | QVQLQQPGAELVKPGASVKLSCKASGYIFTSYWMHWVKQRPGQGLEWIGEIDPSD |
| | | SYTNYNQKFKGKATLTLDKSSSTTYMQLSSLTSEDSAVYYCVRGYGNYGDYWGQG |
| | | TTLTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DK |
| 68 | 5E7 LP FAB | DVVMTQAAFSNPVTLGTSASISCRSSKSLLHSNGITYLYWYLQKPGQSPQLLIYQ |
| | | MSNLASGVPDRFSSSGSGTDFTLRISRVEAEDVGVYYCAQNLELPWTFGGGTKLE |
| | | IKRTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| 69 | 5E7 H0 FAB | QVQLVQSGAEVKKPGASVKVSCKASGYTFTSYWMHWVRQAPGQGLEWMGEIDPSD |
| | | SYTNYNQKFKGRVTMTRDTSTSTVYMELSSLRSEDTAVYYCARGYGNYGDYWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DK |
| 70 | 5E7 L0 FAB | DIVMTQSPLSLPVTPGEPASISCRSSKSLLHSNGITYLYWYLQKPGQSPQLLIYQ |
| | | MSNLASGVPDRFSGSGSGTDFTLKISRVEAEDVGVYYCAQNLELPWTFGGGTKVE |
| | | IKRTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |
| 71 | Linker DGGFG | DGGFG |
| 72 | Linker DDGGFG | DDGGFG |
| 73 | Linker KDGGFG | KDGGFG |
| 74 | Linker GGFGGGF | GGFGGGF |
| 75 | Linker GGFGG | GGFGG |
| 76 | Linker GGFGGG | GGFGGG |
| 77 | Full Chain 5E7 | QVQLVQSGAEVKKPGASVKVSCKASGYIFTSYWMHWVKQRPGQGLEWMGEIDPSD |
| | H5 | SYTNYNQKFKGRVTMTLDKSTSTTYMELSSLRSEDTAVYYCARGYGNYGDYWGQG |
| | | TLVTVSSASTKGPSVFPLAPSSKSTSGGTAALGCLVKDYFPEPVTVSWNSGALTS |
| | | GVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKF |
| | | NWYVDGVEVHNAKTKPREEQYNSTYRVVSVLTVLHQDWLNGKEYKCKVSNKALPA |
| | | PIEKTISKAKGQPREPQVYTLPPSREEMTKNQVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |
| 78 | Full chain 5E7 | DVVMTQSALSNPVTPGEPASISCRSSKSLLHSNGITYLYWYLQKPGQSPQLLIYQ |
| | L3 | MSNLASGVPDRFSSSGSGTDFTLKISRVEAEDVGVYYCAQNLELPWTFGGGTKVE |
| | | IKRTVAAPSVFIFPPSDEQLKSGTASVVCLLNNFYPREAKVQWKVDNALQSGNSQ |
| | | ESVTEQDSKDSTYSLSSTLTLSKADYEKHKVYACEVTHQGLSSPVTKSFNRGEC |

1-37. (canceled)

38. An antibody or antibody fragment thereof that specifically binds a human Nectin-4 polypeptide, wherein the antibody or antibody fragment comprises a heavy chain variable region (VH) comprising an amino acid sequence at least 95% identical to an amino acid sequence selected from the group consisting of SEQ ID NOS: 47, 49, 51, 37, 39, 41, 43, and 45; and a light chain variable region (VL) comprising an amino acid sequence at least 95% identical to an amino acid sequence selected from the group consisting of SEQ ID NOS: 65, 63, 61, and 59, wherein the antibody or antibody fragment comprises a heavy chain variable region (VH) comprising a CDR1, CDR2, CDR3 having the respective amino acid sequences shown in SEQ ID NOS: 21, 22 and 23; and a light chain variable region CDR1, CDR2, CDR3 having the respective amino acid sequences shown in SEQ ID NOS: 24, 25, and 26.

39. The antibody or antibody fragment of claim 38, wherein:
(a) the amino acid at Kabat heavy chain position 71 is a leucine and the amino acid at Kabat heavy chain position 78 is a threonine;
(b) the amino acid at Kabat heavy chain position 73 is a lysine;
(c) the amino acid at Kabat heavy chain position 28 is an isoleucine;
(d) the amino acid at Kabat heavy chain position 38 is a lysine; and/or
(e) the amino acid at Kabat heavy chain position 40 is an arginine.

40. The antibody or antibody fragment of claim 38, wherein:
(a) the amino acid at Kabat light chain position 2 is a valine and the amino acid at Kabat light chain position 64 is a serine;
(b) the amino acid at Kabat light chain position 11 is an asparagine; and/or
(c) the amino acid at Kabat light chain position 8 is an alanine.

41. The antibody or antibody fragment of claim 38, wherein said antibody or antibody fragment comprises a heavy chain variable region (VH) comprising the amino acid sequence of SEQ ID NO: 47; and a light chain variable region (VL) comprising the amino acid sequence of SEQ ID NO: 65.

42. The antibody or antibody fragment of claim 38, wherein said antibody or antibody fragment is conjugated to a linker-payload moiety (X-Z), wherein the antibody-linker-payload has the structure: 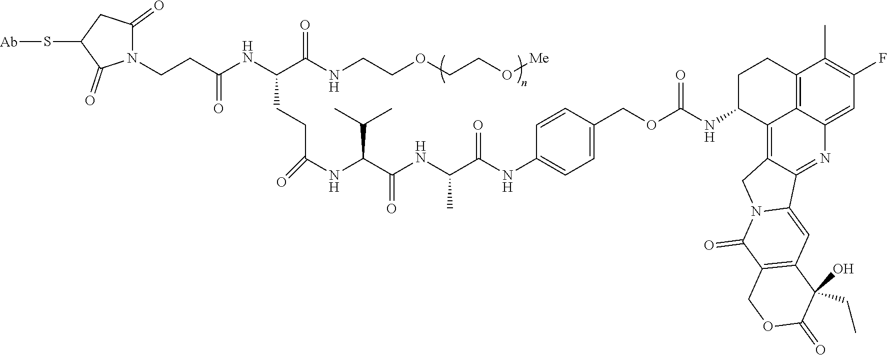
wherein n is 15.

43. An immunoconjugate that binds a human Nectin-4 polypeptide, represented by formula (I):
Ab-(X-(Z)) Formula (I)
wherein,
Ab is an antibody or antibody fragment of claim 38;
X is a molecule which connects Ab and Z, wherein X comprises a cleavable moiety; and
Z comprises a cytotoxic agent, optionally a camptothecin analogue, optionally a five-ring camptothecin analogue, optionally a six-ring camptothecin analogue, optionally exatecan, SN-38 or Dxd.

44. The immunoconjugate of claim 43, wherein the immunoconjugate is represented by any one of the formulae: 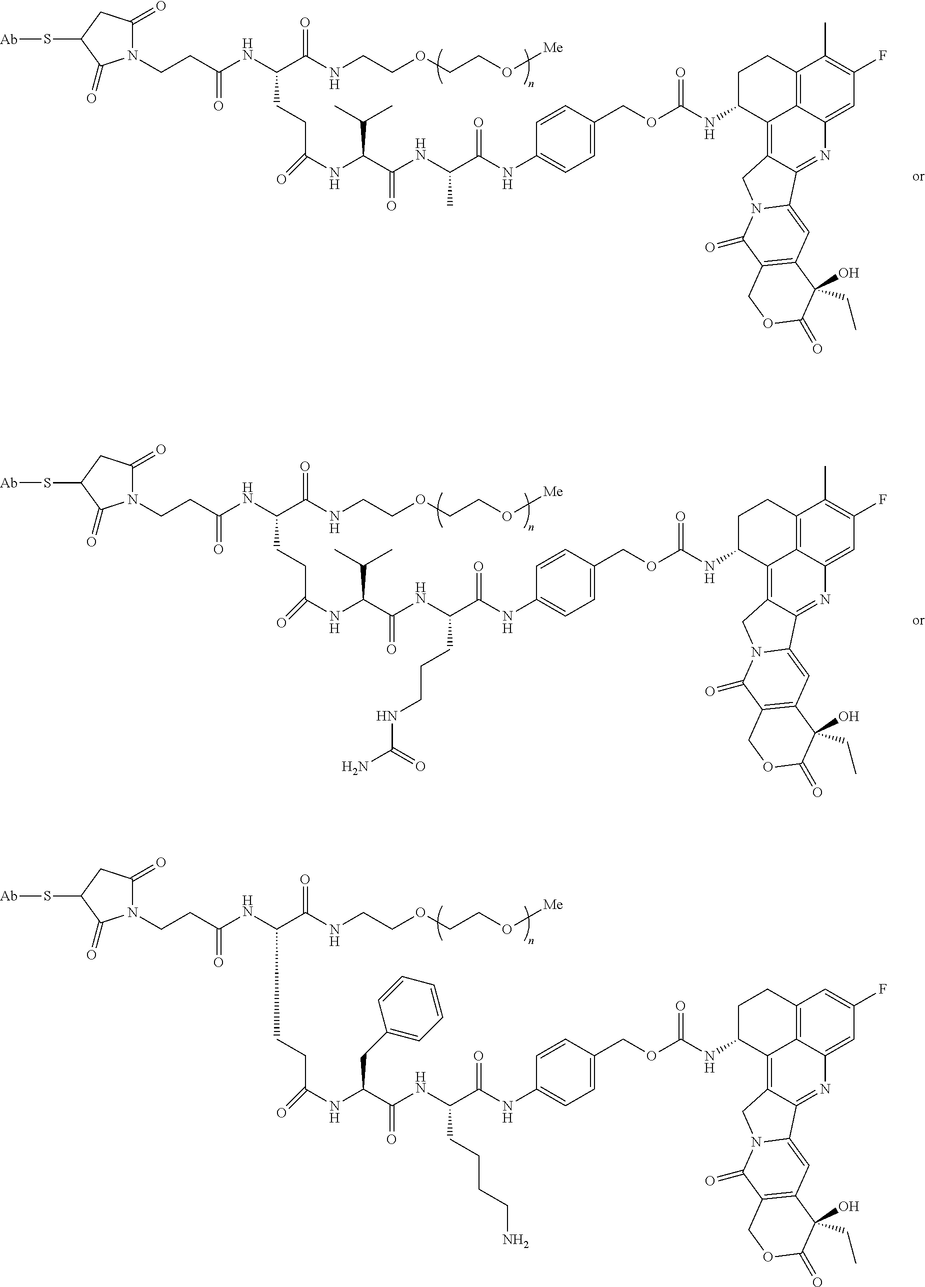
wherein n is 5-23; and
wherein Ab is an antibody or antibody fragment thereof that specifically binds a human Nectin-4 polypeptide.

45. The immunoconjugate of claim 43, wherein the antibody or antibody fragment comprises a heavy chain variable region (VH) comprising the amino acid sequence of SEQ ID NO: 47; and a light chain variable region (VL) comprising the amino acid sequence of SEQ ID NO: 65.

46. The immunoconjugate of claim 43, wherein the antibody or antibody fragment comprises a heavy chain comprising the amino acid sequence of SEQ ID NO: 77; and a light chain comprising the amino acid sequence of SEQ ID NO: 78.

47. The immunoconjugate of claim 43, wherein n is 15 and the immunoconjugate is characterized by a DAR of between 6 and 8, optionally wherein the DAR is 6, optionally wherein the DAR is 8.

48. A method of making an antibody-drug-conjugate (ADC), said method comprising conjugating a cytotoxic agent (Z) to an anti-Nectin-4 antibody of claim 38, wherein a linker (X) connects the anti-Nectin-4 antibody (Ab) and the cytotoxic agent (Z).

49. The method of claim 48, wherein said method comprises providing said anti-Nectin-4 antibody, contacting and/or reacting said anti-Nectin-4 antibody with a cytotoxic agent (Z) under conditions suitable such that antibody drug conjugate is formed, and isolated the antibody drug conjugate.

50. The method of claim 48, wherein said method comprises providing an anti-Nectin-4 antibody, contacting and/or reacting said anti-Nectin-4 antibody with a linker payload moiety (X-Z) having the formula:

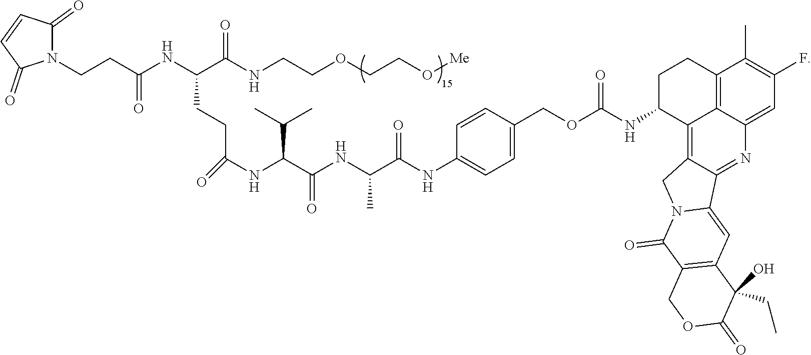

51. A method of treating a cancer, killing tumor cells and/or delivering a cytotoxic agent to a tumor, in an individual in need thereof, comprising administering to said individual a therapeutically effective amount of an immunoconjugate according to claim 43.

52. The method of claim 51, wherein said individual has a Nectin-4 expressing cancer characterized by low or moderate Nectin-4 expression on tumor cells, optionally as determined by immunochemistry.

53. The method of claim 51, wherein said individual has an urothelial cancer, a TNBC, a non-small cell lung cancer, a pancreatic cancer, an ovarian cancer, a head and neck squamous cell carcinoma or an esophageal cancer.

54. The method of claim 51, wherein said individual has received prior treatment with a chemotherapeutic agent, optionally a chemotherapeutic agent transported by P-glycoprotein (Pgp), a platinum agent or a taxane.

55. The method of claim 51, wherein said individual has received prior treatment with a Nectin-4 binding agent conjugated to an auristatin, optionally enfortumab vedotin.

56. A pharmaceutical composition comprising an antibody according to claim 38 or an immunoconjugate comprising said antibody (Ab) and represented by formula (I): Ab-(X-(Z)) Formula (I) and a pharmaceutically acceptable carrier, wherein X is a molecule which connects Ab and Z, wherein X comprises a cleavable moiety; and Z comprises a cytotoxic agent, optionally a camptothecin analogue, optionally a five-ring camptothecin analogue, optionally a six-ring camptothecin analogue, optionally exatecan, SN-38 or Dxd.

57. A nucleic acid or set of nucleic acids encoding a heavy and/or light chain of an antibody or antibody fragment of claim 38.

58. A hybridoma or recombinant host cell producing the antibody or antibody fragment of claim 38.

59. A method of making an antibody or antibody fragment comprising culturing a cell of claim 58, and recovering the antibody or antibody fragment.
